(19) United States
(12) Patent Application Publication
(10) Pub. No.: US 2025/0144109 A1
(43) Pub. Date: May 8, 2025

(54) Compositions for preventing or treating pulmonary arterial hypertension

(71) Applicant: CHONG KUN DANG PHARMACEUTICAL CORP., Seoul (KR)

(72) Inventors: Ju Hee LEE, Yongin-si, Gyeonggi-do (KR); Beom Seob LEE, Yongin-si, Gyeonggi-do (KR); Jiyeon KANG, Yongin-si, Gyeonggi-do (KR)

(21) Appl. No.: 18/833,742

(22) PCT Filed: Jan. 26, 2023
(86) PCT No.: PCT/IB2023/050658

§ 371 Date: Jul. 26, 2024

(30) Foreign Application Priority Data
Jan. 28, 2022 (KR) 10-2022-0013635

(51) Int. Cl. A61K 31/541; A61K 9/00; A61P 9/12; A61P 11/00

(52) U.S. Cl. CPC A61K 31/541; A61K 9/0053; A61P 9/12; A61P 11/00

(57) ABSTRACT

The present invention relates to a pharmaceutical composition for preventing or treating pulmonary arterial hypertension, comprising a compound represented by formula I, optical isomers thereof or pharmaceutically acceptable salts thereof as an active ingredient, a method for preventing or treating pulmonary arterial hypertension using the compound, a use of the compound for preventing or treating pulmonary arterial hypertension, and a use of the compound in preparing a medicament for preventing or treating pulmonary arterial hypertension.

### TECHNICAL FIELD

The present disclosure relates to a pharmaceutical composition for preventing or treating pulmonary arterial hypertension, comprising a compound represented by formula I, optical isomers thereof or pharmaceutically acceptable salts thereof as an active ingredient, a method for preventing or treating pulmonary arterial hypertension using the compound, optical isomers thereof or pharmaceutically acceptable salts thereof, a use of the compound, optical isomers thereof or pharmaceutically acceptable salts thereof for preventing or treating pulmonary arterial hypertension, and a use of the compound, optical isomers thereof or pharmaceutically acceptable salts thereof in preparing a medicament for preventing or treating pulmonary arterial hypertension.

### BACKGROUND

Pulmonary arterial hypertension (PAH) is a disease in which a blood pressure in the pulmonary artery for supplying blood from the heart to the lungs increases, and is a fatal disease which may lead to premature death. The main symptoms are dyspnea, general weakness, dizziness, chest pain, etc., which may frequently appear in everyday life, thus making early diagnosis difficult. The PAH is a rare disease which affects mainly women in their 30s to 50s and occurs in 15 to 50 per million adults (Levine D J et al., Am J Manag Care. 2021; 27(3):35-41).

It is estimated that there are about 100,000 patients worldwide and about 1,500 patients in Korea. Currently, the treatment of pulmonary arterial hypertension is performed with adjuvant therapy such as diuretics, anticoagulation therapy, etc., drug therapy with vasodilator function, and surgical treatment (lung transplantation) (Rebecca L et al., US Cardiology Review. 2016; 10(2):78-84). Although there are studies showing that the early initiation of combination therapy ameliorates a patient's symptoms and improves survival rate, the conditions for combination therapy are strict, and there is currently no drug which is targeted to treat patients while targeting an underlying disease mechanism. To date, the therapeutic agents for pulmonary arterial hypertension ameliorate pulmonary arterial hypertension symptoms by using drugs which prevent an inflammatory mechanism and fibrosis and improve vascular structure remodeling along with drugs such as Bosentan, Macitentan, and Sildenafil, which are focused on preventing vascular obstruction caused by pulmonary vasoconstriction and thrombosis.

However, the above therapeutic agents still do not have a sufficient effect on treating pulmonary arterial hypertension, and often fail to show a sufficient treatment effect for patients with hereditary pulmonary arterial hypertension caused by genetic factors such as BMPR2 gene mutation, etc. (Atkinson C et al., Circulation. 2002; 105:1672-1678).

Thus, there is a great need to develop a therapeutic agent capable of more fundamentally treating various pulmonary arterial hypertensions, including hereditary pulmonary arterial hypertension.

### RELATED ART REFERENCE

### Patent Documents

(Patent Document 1) Korean Unexamined Patent Application Publication No. 2017-0017792

### Non-Patent Documents

(non-Patent Document 1) Levine D J et al., Am J Manag Care. 2021; 27(3):35-41
(non-Patent Document 2) Rebecca L et al., US Cardiol Review. 2016; 10(2):78-84
(non-Patent Document 3) Boucherat O et al., Sci Rep. 2017 Jul. 3; 7(1):4546
(non-Patent Document 4) Atkinson C et al., Circulation. 2002; 105:1672-1678
(non-Patent Document 5) Rabionovitch M., J Clin Invest. 2008; 118:2372-2379
(non-Patent Document 6) Morrell N W., Proc Am Thorac Soc. 2006 November; 3(8):680-6

### DETAILED DESCRIPTION OF THE INVENTION

### Technical Problem

The present disclosure may provide a pharmaceutical composition for preventing or treating pulmonary arterial hypertension, containing a compound represented by formula I, optical isomers thereof or pharmaceutically acceptable salts thereof as an active ingredient.

The present disclosure may provide a method for preventing or treating pulmonary arterial hypertension, including administering the compound represented by above formula I, optical isomers thereof or pharmaceutically acceptable salts thereof into an individual.

The present disclosure may provide a use of the compound represented by above formula I, optical isomers thereof or pharmaceutically acceptable salts thereof for preventing or treating pulmonary arterial hypertension.

The present disclosure may provide a use of the compound represented by above formula I, optical isomers thereof or pharmaceutically acceptable salts thereof in preparing a medicament for preventing or treating pulmonary arterial hypertension.

### Technical Solution

This is described in detail as follows. Meanwhile, each description and embodiment disclosed in the present invention may be also applied to other descriptions and embodiments thereof, respectively. In other words, all the combinations of various elements disclosed in the present invention fall within the scope of the present invention. Also, it cannot be seen that the scope of the present invention is limited to the specific description described below.

The present disclosure provides a pharmaceutical composition for preventing or treating pulmonary arterial hypertension, including a compound represented by formula I below, optical isomers thereof or pharmaceutically acceptable salts thereof as an active ingredient.

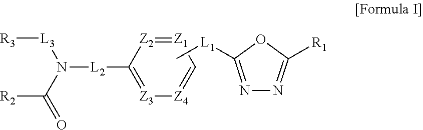

In Formula I,
wherein L₁, L₂ or L₃ are each independently a bond or —(C₁-C₂ alkylene)-;
R₁ is —CX₂H or —CX₃;
R₂ is —NR^{A}R^{B}, —OR^{C},

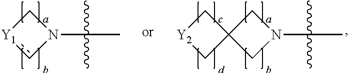

{wherein at least one H of

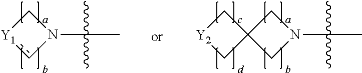

may be substituted with —X, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be substituted with —X, —OH, —CF₃ or —CF₂H]};
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl,

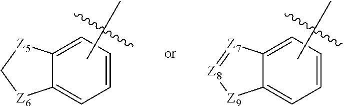

{wherein, at least one H of —(C₁-C₄ alkyl) may be substituted with —X or —OH,
at least one H of -aryl or -heteroaryl each independently may be substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), aryl, heteroaryl,

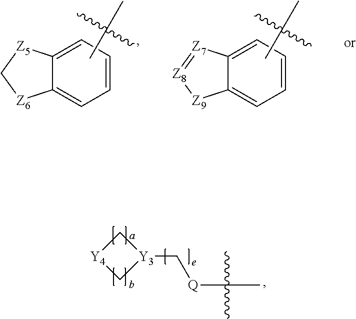

[wherein, at least one H of

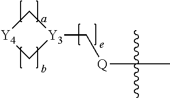

may be substituted with —X, —(C₁-C₄ alkyl), —NR^{D}R^{E}, —CF₃ or —CF₂H], at least one H of —(C₃-C₇ cycloalkyl), —(C₂-C₆ cycloheteroalkyl), adamantyl,

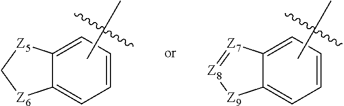

may be each independently substituted with —X, —OH or (C₁-C₄ alkyl)};
Y₁, Y₂ and Y₄ are each independently —CH₂—, —NR^{F}—, —O—, —C(═O)— or —S(═O)₂—;
Y₃ is —CH— or —N—;
Z₁ to Z₄ are each independently N or CR^{Z}, {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ aryl)-heteroaryl, —(C₃-C₇ cycloalkyl), —(C₂-C₆ heterocycloalkyl) or

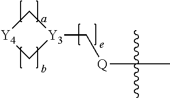

{wherein, at least one H of the —(C₁-C₄ alkyl), —(C₁-C₄ alkyl)-OH or —(C₁-C₄ alkyl)-NR^{D}R^{E} may be substituted with —X,
at least one H of the -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with —X, —OH, —O(C₁-C₄ alkyl), —(C₁-C₄ alkyl), —CF₃, —CF₂H or —CN,
at least one H of

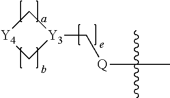

may be substituted with —X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl)};
R^{C} is —(C₁-C₄ alkyl), -aryl, —(C₁-C₄ alkyl)-aryl, -heteroaryl or —(C₁-C₄ alkyl)-heteroaryl {wherein, at least one H of —(C₁-C₄ alkyl) may be substituted with —X or —OH, at least one H of -aryl, —(C₁-C₄ alkyl)-aryl, -heteroaryl or —(C₁-C₄ alkyl)-heteroaryl may be substituted with —X, —OH, —CF₃ or —CF₂H};
R^{D} and R^{E} are each independently —H, —(C₁-C₄ alkyl), -aryl or —(C₁-C₄ alkyl)-aryl {wherein, at least one H of —(C₁-C₄ alkyl) may be substituted with —X or —OH, at least one H of -aryl or —(C₁-C₄ alkyl)-aryl may be substituted with —X, —OH, —CF₃ or —CF₂H};
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(—O)—(C₃-C₇ cycloalkyl), —(C₂-C₆ heterocycloalkyl) or —(C₁-C₄ alkyl)-C(═O)—(C₂-C₆ heterocycloalkyl)
{wherein, at least one H of —(C₁-C₄ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), (C₁-C₄ alkyl)-NR^{D}R^{E} or —S(═O)₂—(C₁-C₄ alkyl) may be substituted with —X,
at least one H of -aryl, —(C₁-C₄alkyl)-aryl, —(C₂-C₄ alkenyl)-aryl, -heteroaryl, —(C₁-C₄ alkyl)-heteroaryl, —C(—O)—(C₃-C₇ cycloalkyl), —C₂-C₆ heterocycloalkyl or —(C₁-C₄alkyl)-C(═O)—(C₂-C₆heterocycloalkyl) may be substituted with —X, —OH, —CF₃ or —CF₂H};
R^{G} is —H or —(C₁-C₄ alkyl);
Q is —O— or a bond;
[symbol] is a single bond or double bond {provided that, [symbol] is a double bond, Y₁ is ═CH—};
a to e are each independently an integer of 0, 1, 2, 3 or 4 {provided that, a and b may not be simultaneously 0, and c and d may not be simultaneously 0};
X is each independently F, Cl, Br or I.

In the pharmaceutical composition according to the present disclosure, the compound represented by formula I may be below:
L₁, L₂ or L₃ are each independently a bond or —(C₁-C₂alkylene)-;
R₁ is —CX₂H or —CX₃;
R₂ is —NR^{A}R^{B}, —OR^{C},

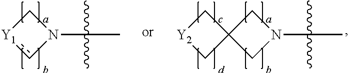

{wherein at least one of H of

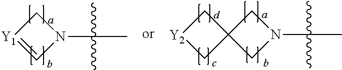

may be substituted with —X, —OH, —NR^{D}R^{E}, —(C₁-C₄ alkyl)};
R₃ is —(C₁-C₄ alkyl), —(C₃-C₇ cycloalkyl), -aryl, -heteroaryl, -adamantyl,

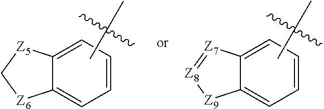

{wherein at least one H of -aryl or -heteroaryl may be each independently substituted with —X, —O(C₁-C₄alkyl), —OCF₃, —O-aryl, —NR^{D}R^{E}, —(C₁-C₄ alkyl), —CF₃, —S(═O)₂—(C₁-C₄alkyl), -aryl, -heteroaryl,

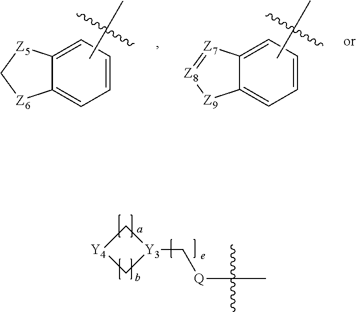

[wherein at least one H of

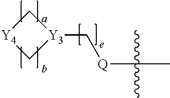

may be substituted with —NR^{D}R^{E} or —(C₁-C₄ alkyl)],
at least one H of

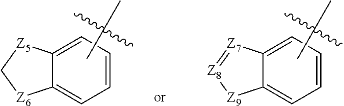

maybe each independently substituted with —(C₁-C₄ alkyl)};
Y₁, Y₂ and Y₄ are each independently —CH₂—, —NR^{F}—, —O—, —C(═O)— or —S(═O)₂—; Y₃ is —CH— or —N—;
Z₁ to Z₄ is each independently N or CR^{Z} {wherein at least three of Z₁ to Z₄ may not be simultaneously N, and R^{Z} is —H, —X or —O(C₁-C₄ alkyl)};
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, —(C₃-C₇ cycloalkyl) or

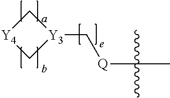

{wherein at least one H of

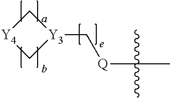

may be substituted with —X, —(C₁-C₄alkyl), —CF₃, —(C₂-C₆ heterocycloalkyl), —(C₁-C₄ alkyl)-aryl, -heteroaryl or heteroaryl-(C₁-C₄ alkyl)};
R^{C} is —(C₁-C₄ alkyl) or -aryl;
R^{D} and R^{E} are each independently —H, —(C₁-C₄alkyl) or —(C₁-C₄ alkyl)-aryl;
R^{F} is —H, —(C₁-C₆alkyl), —(C₁-C₄alkyl)-OH, —(C₁-C₄ alkyl)-O—(C₁-C₄ alkyl), —C(═O)—(C₁-C₄alkyl), —C(═O)—O(C₁-C₄ alkyl), —(C₁-C₄ alkyl)-C(═O)—O(C₁-C₄alkyl), —(C₁-C₄alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
{wherein at least one H of —(C₁-C₄alkyl) or —C(═O)—O(C₁-C₄alkyl) may be substituted with —X,
at least one H of -aryl may be substituted with —X};
R^{G} is —(C₁-C₄ alkyl);
Q is —O— or a bond;
[symbol] is a single bond or a double bond {provided that [symbol] is a double bond, Y₁ is —CH—};
a to e are each independently an integer of 0, 1, 2, 3 or 4 {provided that a and b may not be simultaneously 0, and c and d may not be simultaneously 0};
X is each independently F, Cl, Br or I.

In the pharmaceutical composition according to the present disclosure, the compound represented by the formula I may be the compound represented by formula Ia:

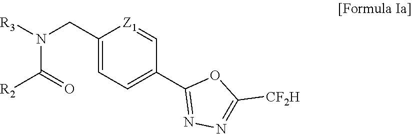

in Formula Ia,
R₂ is

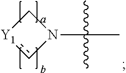

R₃ is -aryl {wherein at least one H of -aryl may be each independently substituted with —X};
Y₁ is —O— or —S(═O)₂—;
Z₁ is N or CR^{Z} {wherein R^{Z} is —X};
a and b are each independently an integer of 0, 1, 2, 3 or 4 {wherein a and b may not be simultaneously 0};
X is each independently F, Cl, Br or I.

In the pharmaceutical composition according to the present disclosure, the compound represented by formula Ia may be below:
R₂ is

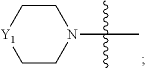

R₃ is -phenyl {wherein at least one H of -phenyl each independently may be substituted with —F or —Cl};
Y₁ is —O— or —S(═O)₂—;
Z₁ is N or CF.

In the pharmaceutical composition according to the present disclosure, the compounds represented by formula I may be shown in Table A below:

TABLE A

| Compound | Structure |
|---|---|
| 1 | 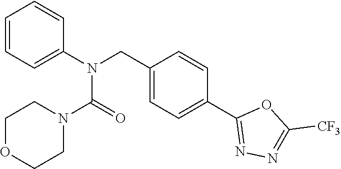 [SMILES: O=C(N1CCOCC1)N(Cc1ccc(-c2nnc(C(F)(F)F)o2)cc1)c1ccccc1] |
| 2 | 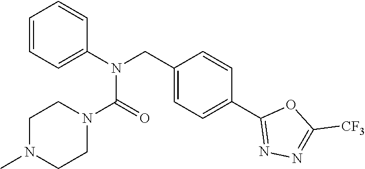 [SMILES: CN1CCN(C(=O)N(Cc2ccc(-c3nnc(C(F)(F)F)o3)cc2)c2ccccc2)CC1] |
| 3 | 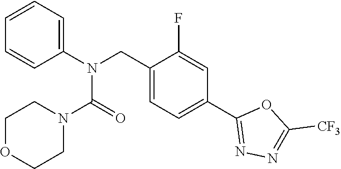 [SMILES: O=C(N1CCOCC1)N(Cc1ccc(-c2nnc(C(F)(F)F)o2)cc1F)c1ccccc1] |
| 4 | 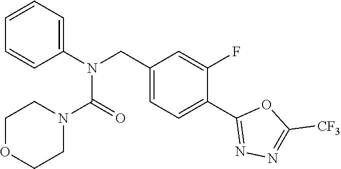 [SMILES: O=C(N1CCOCC1)N(Cc1ccc(-c2nnc(C(F)(F)F)o2)c(F)c1)c1ccccc1] |
| 5 | 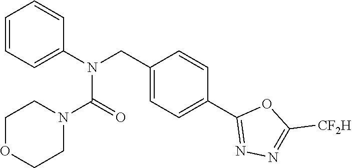 [SMILES: O=C(N1CCOCC1)N(Cc1ccc(-c2nnc(C(F)F)o2)cc1)c1ccccc1] |
| 6 | 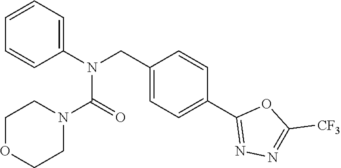 [SMILES: O=C(N1CCOCC1)N(Cc1ccc(-c2nnc(C(F)(F)F)o2)cc1)c1ccccc1] |
| 7 | 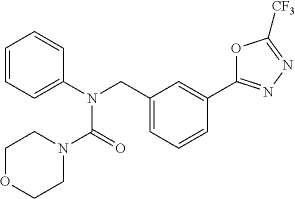 [SMILES: O=C(N1CCOCC1)N(Cc1cccc(-c2nnc(C(F)(F)F)o2)c1)c1ccccc1] |
| 8 | 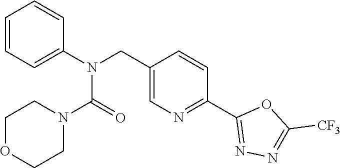 [SMILES: O=C(N1CCOCC1)N(Cc1ccc(-c2nnc(C(F)(F)F)o2)nc1)c1ccccc1] |
| 9 | 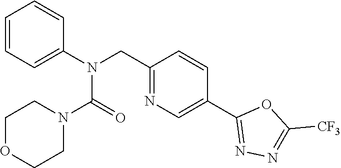 [SMILES: O=C(N1CCOCC1)N(Cc1ccc(-c2nnc(C(F)(F)F)o2)cn1)c1ccccc1] |
| 10 | 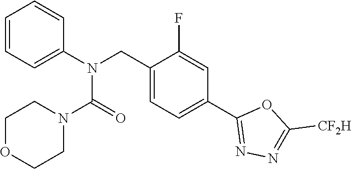 [SMILES: O=C(N1CCOCC1)N(Cc1ccc(-c2nnc(C(F)F)o2)cc1F)c1ccccc1] |
| 11 | 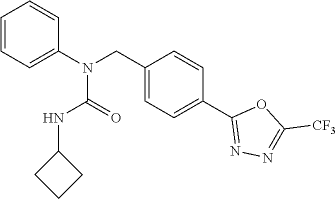 [SMILES: O=C(NC1CCC1)N(Cc1ccc(-c2nnc(C(F)(F)F)o2)cc1)c1ccccc1] |
| 12 | 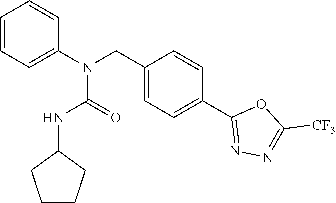 [SMILES: O=C(NC1CCCC1)N(Cc1ccc(-c2nnc(C(F)(F)F)o2)cc1)c1ccccc1] |
| 13 | 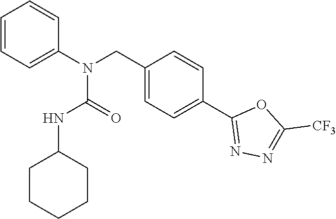 [SMILES: O=C(NC1CCCCC1)N(Cc1ccc(-c2nnc(C(F)(F)F)o2)cc1)c1ccccc1] |
| 14 | 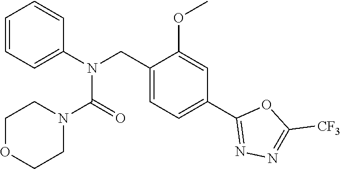 [SMILES: COc1cc(-c2nnc(C(F)(F)F)o2)ccc1CN(C(=O)N1CCOCC1)c1ccccc1] |
| 15 | 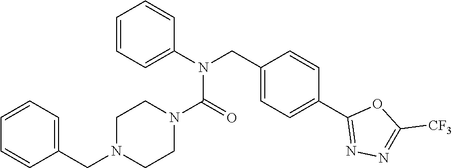 [SMILES: O=C(N1CCN(Cc2ccccc2)CC1)N(Cc1ccc(-c2nnc(C(F)(F)F)o2)cc1)c1ccccc1] |
| 16 | 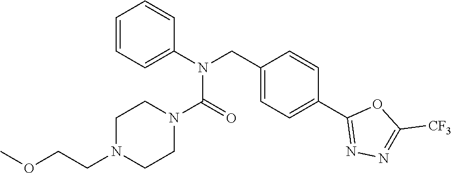 [SMILES: COCCN1CCN(C(=O)N(Cc2ccc(-c3nnc(C(F)(F)F)o3)cc2)c2ccccc2)CC1] |
| 17 | 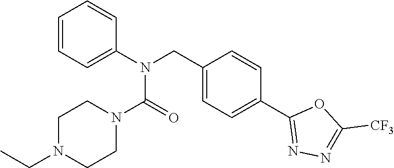 [SMILES: CCN1CCN(C(=O)N(Cc2ccc(-c3nnc(C(F)(F)F)o3)cc2)c2ccccc2)CC1] |
| 18 | 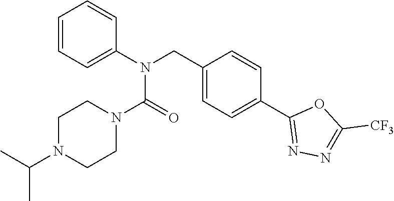 [SMILES: CC(C)N1CCN(C(=O)N(Cc2ccc(-c3nnc(C(F)(F)F)o3)cc2)c2ccccc2)CC1] |
| 19 | 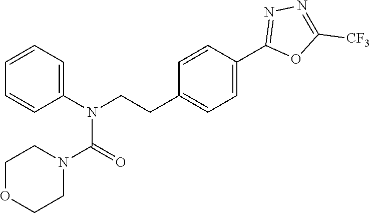 [SMILES: O=C(N1CCOCC1)N(CCc1ccc(-c2nnc(C(F)(F)F)o2)cc1)c1ccccc1] |
| 20 | 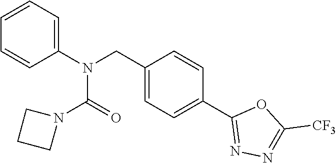 [SMILES: O=C(N1CCC1)N(Cc1ccc(-c2nnc(C(F)(F)F)o2)cc1)c1ccccc1] |
| 21 | 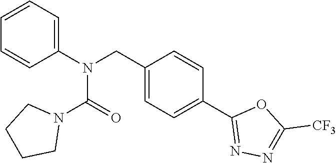 [SMILES: O=C(N1CCCC1)N(Cc1ccc(-c2nnc(C(F)(F)F)o2)cc1)c1ccccc1] |
| 22 | 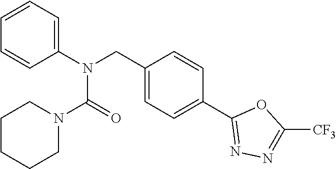 [SMILES: O=C(N1CCCCC1)N(Cc1ccc(-c2nnc(C(F)(F)F)o2)cc1)c1ccccc1] |
| 23 | 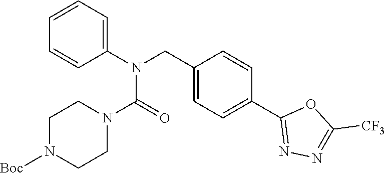 [SMILES: CC(C)(C)OC(=O)N1CCN(C(=O)N(Cc2ccc(-c3nnc(C(F)(F)F)o3)cc2)c2ccccc2)CC1] |
| 24 | 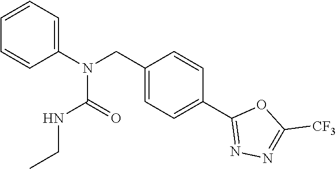 [SMILES: CCNC(=O)N(Cc1ccc(-c2nnc(C(F)(F)F)o2)cc1)c1ccccc1] |
| 25 | 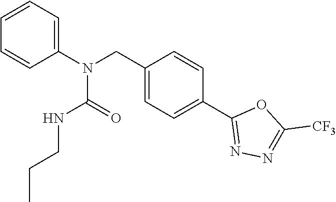 [SMILES: CCCNC(=O)N(Cc1ccc(-c2nnc(C(F)(F)F)o2)cc1)c1ccccc1] |
| 26 | 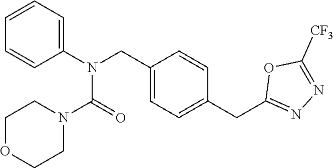 [SMILES: O=C(N1CCOCC1)N(Cc1ccc(Cc2nnc(C(F)(F)F)o2)cc1)c1ccccc1] |
| 27 | 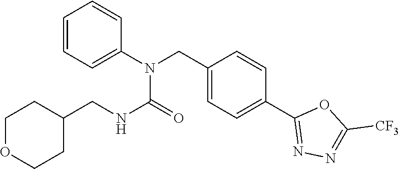 [SMILES: O=C(NCC1CCOCC1)N(Cc1ccc(-c2nnc(C(F)(F)F)o2)cc1)c1ccccc1] |
| 28 | 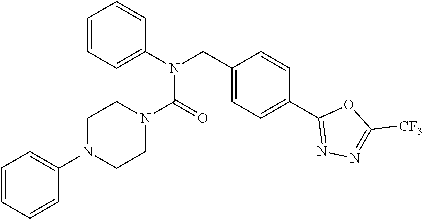 [SMILES: O=C(N1CCN(c2ccccc2)CC1)N(Cc1ccc(-c2nnc(C(F)(F)F)o2)cc1)c1ccccc1] |
| 29 | 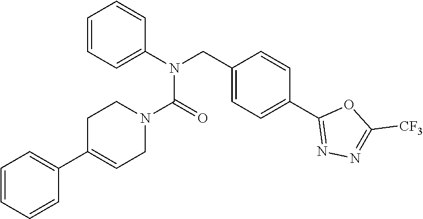 [SMILES: O=C(N1CC=C(c2ccccc2)CC1)N(Cc1ccc(-c2nnc(C(F)(F)F)o2)cc1)c1ccccc1] |
| 30 | 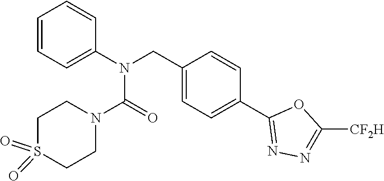 [SMILES: O=C(N1CCS(=O)(=O)CC1)N(Cc1ccc(-c2nnc(C(F)F)o2)cc1)c1ccccc1] |
| 31 | 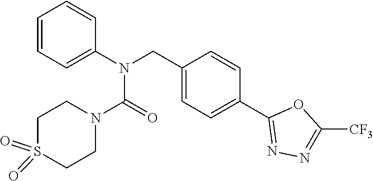 [SMILES: O=C(N1CCS(=O)(=O)CC1)N(Cc1ccc(-c2nnc(C(F)(F)F)o2)cc1)c1ccccc1] |
| 32 | 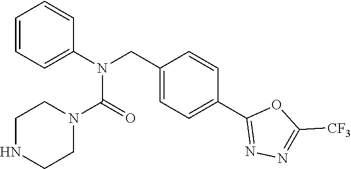 [SMILES: O=C(N1CCNCC1)N(Cc1ccc(-c2nnc(C(F)(F)F)o2)cc1)c1ccccc1] |
| 33 | 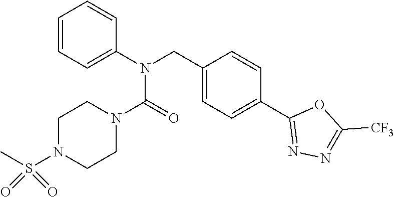 [SMILES: CS(=O)(=O)N1CCN(C(=O)N(Cc2ccc(-c3nnc(C(F)(F)F)o3)cc2)c2ccccc2)CC1] |
| 34 | 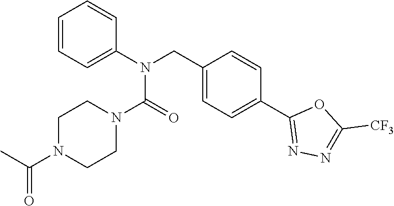 [SMILES: CC(=O)N1CCN(C(=O)N(Cc2ccc(-c3nnc(C(F)(F)F)o3)cc2)c2ccccc2)CC1] |
| 35 | 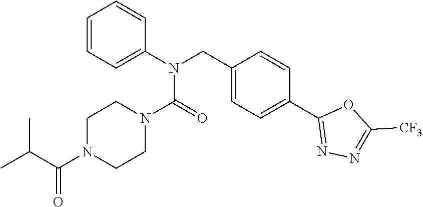 [SMILES: CC(C)C(=O)N1CCN(C(=O)N(Cc2ccc(-c3nnc(C(F)(F)F)o3)cc2)c2ccccc2)CC1] |
| 36 | 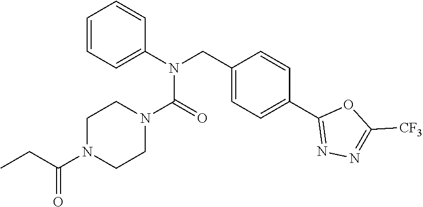 [SMILES: CCC(=O)N1CCN(C(=O)N(Cc2ccc(-c3nnc(C(F)(F)F)o3)cc2)c2ccccc2)CC1] |
| 37 | 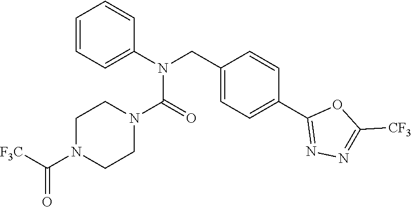 [SMILES: O=C(N1CCN(C(=O)C(F)(F)F)CC1)N(Cc1ccc(-c2nnc(C(F)(F)F)o2)cc1)c1ccccc1] |
| 38 | 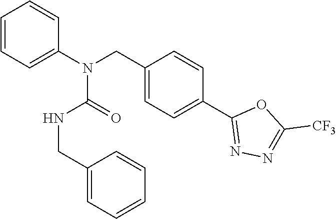 [SMILES: O=C(NCc1ccccc1)N(Cc1ccc(-c2nnc(C(F)(F)F)o2)cc1)c1ccccc1] |
| 39 | 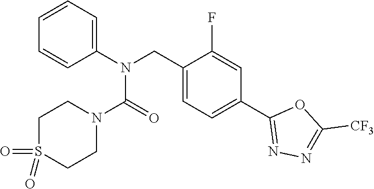 [SMILES: O=C(N1CCS(=O)(=O)CC1)N(Cc1ccc(-c2nnc(C(F)(F)F)o2)cc1F)c1ccccc1] |
| 40 | 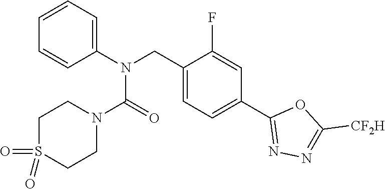 [SMILES: O=C(N1CCS(=O)(=O)CC1)N(Cc1ccc(-c2nnc(C(F)F)o2)cc1F)c1ccccc1] |
| 41 | 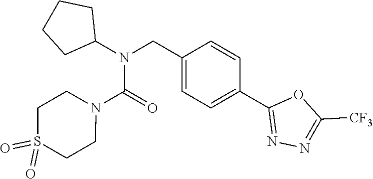 [SMILES: O=C(N1CCS(=O)(=O)CC1)N(Cc1ccc(-c2nnc(C(F)(F)F)o2)cc1)C1CCCC1] |
| 42 | 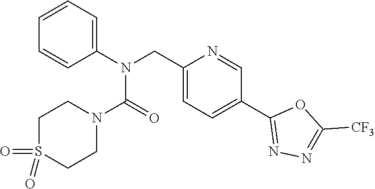 [SMILES: O=C(N1CCS(=O)(=O)CC1)N(Cc1ccc(-c2nnc(C(F)(F)F)o2)cn1)c1ccccc1] |
| 43 | 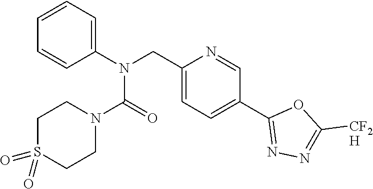 [SMILES: O=C(N1CCS(=O)(=O)CC1)N(Cc1ccc(-c2nnc(C(F)F)o2)cn1)c1ccccc1] |
| 44 | 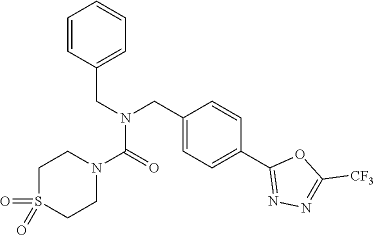 [SMILES: O=C(N1CCS(=O)(=O)CC1)N(Cc1ccccc1)Cc1ccc(-c2nnc(C(F)(F)F)o2)cc1] |
| 45 | 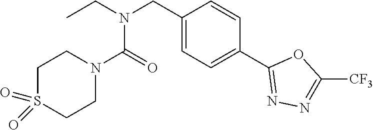 [SMILES: CCN(Cc1ccc(-c2nnc(C(F)(F)F)o2)cc1)C(=O)N1CCS(=O)(=O)CC1] |
| 46 | 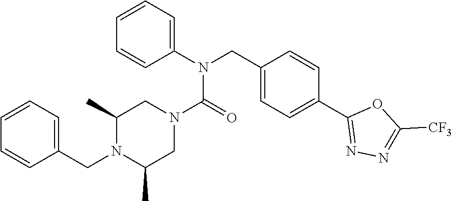 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 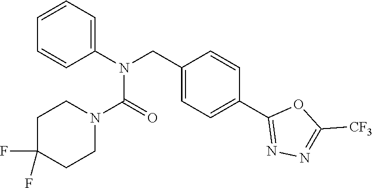 [SMILES: O=C(N1CCC(F)(F)CC1)N(Cc1ccc(-c2nnc(C(F)(F)F)o2)cc1)c1ccccc1] |
| 48 | 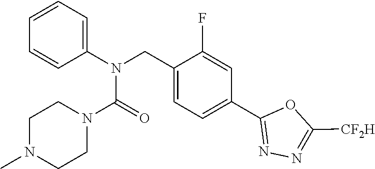 [SMILES: CN1CCN(C(=O)N(Cc2ccc(-c3nnc(C(F)F)o3)cc2F)c2ccccc2)CC1] |
| 49 | 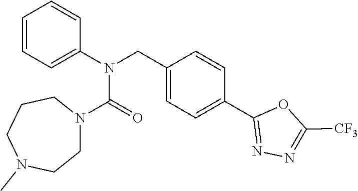 [SMILES: CN1CCCN(C(=O)N(Cc2ccc(-c3nnc(C(F)(F)F)o3)cc2)c2ccccc2)CC1] |
| 50 | 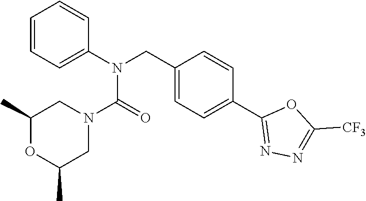 [SMILES: C[C@@H]1CN(C(=O)N(Cc2ccc(-c3nnc(C(F)(F)F)o3)cc2)c2ccccc2)C[C@H](C)O1] |
| 51 | 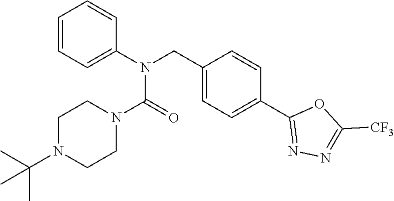 [SMILES: CC(C)(C)N1CCN(C(=O)N(Cc2ccc(-c3nnc(C(F)(F)F)o3)cc2)c2ccccc2)CC1] |
| 52 | 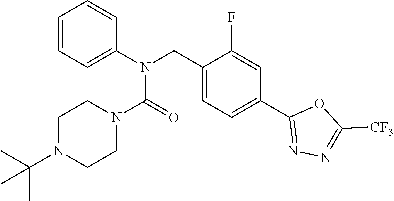 [SMILES: CC(C)(C)N1CCN(C(=O)N(Cc2ccc(-c3nnc(C(F)(F)F)o3)cc2F)c2ccccc2)CC1] |
| 53 | 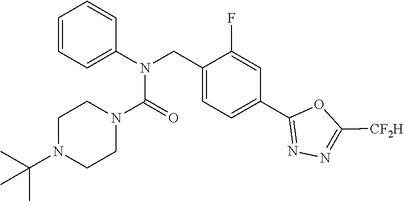 [SMILES: CC(C)(C)N1CCN(C(=O)N(Cc2ccc(-c3nnc(C(F)F)o3)cc2F)c2ccccc2)CC1] |
| 54 | 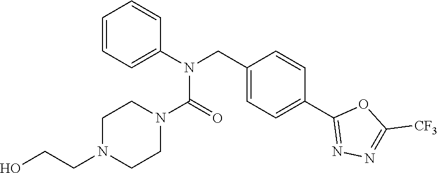 [SMILES: O=C(N1CCN(CCO)CC1)N(Cc1ccc(-c2nnc(C(F)(F)F)o2)cc1)c1ccccc1] |
| 55 | 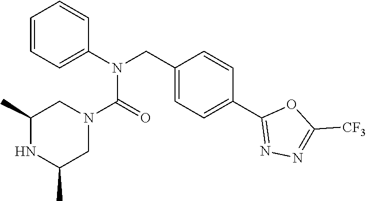 [SMILES: C[C@@H]1CN(C(=O)N(Cc2ccc(-c3nnc(C(F)(F)F)o3)cc2)c2ccccc2)C[C@H](C)N1] |
| 56 | 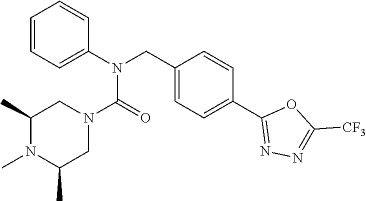 [SMILES: C[C@@H]1CN(C(=O)N(Cc2ccc(-c3nnc(C(F)(F)F)o3)cc2)c2ccccc2)C[C@H](C)N1C] |
| 57 | 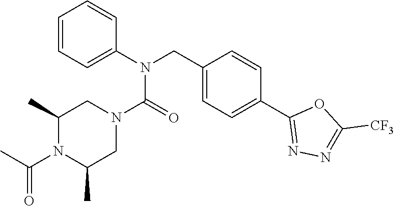 [SMILES: CC(=O)N1[C@H](C)CN(C(=O)N(Cc2ccc(-c3nnc(C(F)(F)F)o3)cc2)c2ccccc2)C[C@@H]1C] |
| 58 | 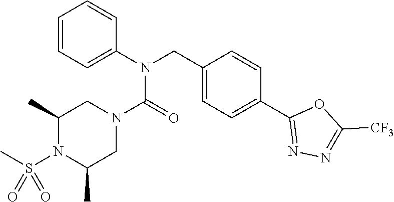 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 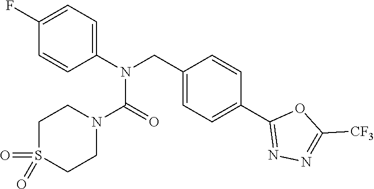 [SMILES: O=C(N1CCS(=O)(=O)CC1)N(Cc1ccc(-c2nnc(C(F)(F)F)o2)cc1)c1ccc(F)cc1] |
| 60 | 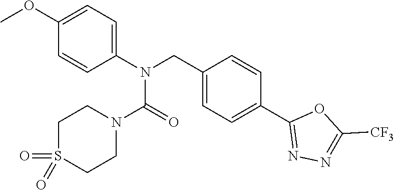 [SMILES: COc1ccc(N(Cc2ccc(-c3nnc(C(F)(F)F)o3)cc2)C(=O)N2CCS(=O)(=O)CC2)cc1] |
| 61 | 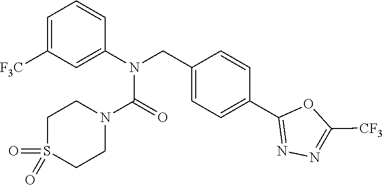 [SMILES: O=C(N1CCS(=O)(=O)CC1)N(Cc1ccc(-c2nnc(C(F)(F)F)o2)cc1)c1cccc(C(F)(F)F)c1] |
| 62 | 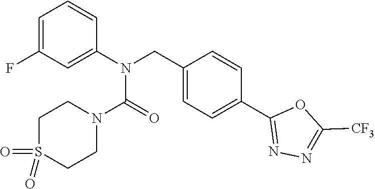 [SMILES: O=C(N1CCS(=O)(=O)CC1)N(Cc1ccc(-c2nnc(C(F)(F)F)o2)cc1)c1cccc(F)c1] |
| 63 | 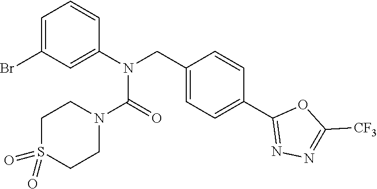 [SMILES: O=C(N1CCS(=O)(=O)CC1)N(Cc1ccc(-c2nnc(C(F)(F)F)o2)cc1)c1cccc(Br)c1] |
| 64 | 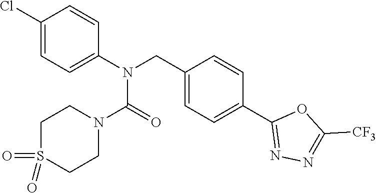 [SMILES: O=C(N1CCS(=O)(=O)CC1)N(Cc1ccc(-c2nnc(C(F)(F)F)o2)cc1)c1ccc(Cl)cc1] |
| 65 | 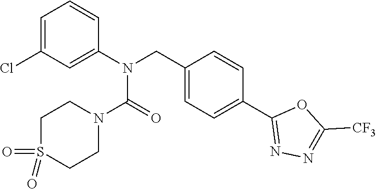 [SMILES: O=C(N1CCS(=O)(=O)CC1)N(Cc1ccc(-c2nnc(C(F)(F)F)o2)cc1)c1cccc(Cl)c1] |
| 66 | 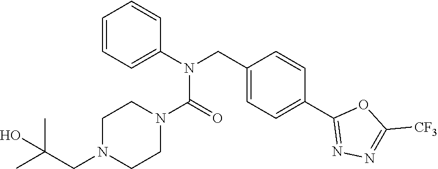 [SMILES: CC(C)(O)CN1CCN(C(=O)N(Cc2ccc(-c3nnc(C(F)(F)F)o3)cc2)c2ccccc2)CC1] |
| 67 | 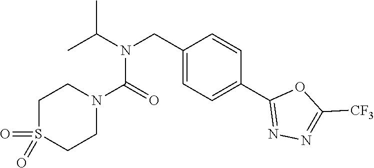 [SMILES: CC(C)N(Cc1ccc(-c2nnc(C(F)(F)F)o2)cc1)C(=O)N1CCS(=O)(=O)CC1] |
| 68 | 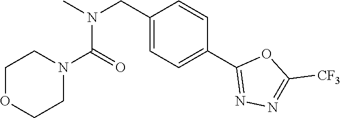 [SMILES: CN(Cc1ccc(-c2nnc(C(F)(F)F)o2)cc1)C(=O)N1CCOCC1] |
| 69 | 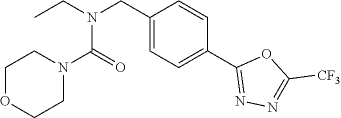 [SMILES: CCN(Cc1ccc(-c2nnc(C(F)(F)F)o2)cc1)C(=O)N1CCOCC1] |
| 70 | 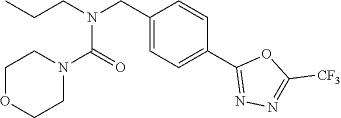 [SMILES: CCCN(Cc1ccc(-c2nnc(C(F)(F)F)o2)cc1)C(=O)N1CCOCC1] |
| 71 | 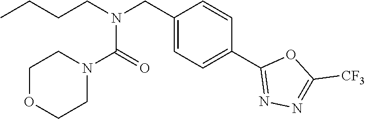 [SMILES: CCCCN(Cc1ccc(-c2nnc(C(F)(F)F)o2)cc1)C(=O)N1CCOCC1] |
| 72 | 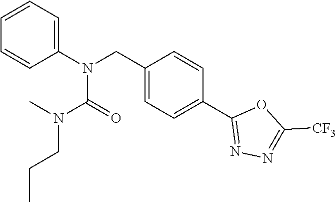 [SMILES: CCCN(C)C(=O)N(Cc1ccc(-c2nnc(C(F)(F)F)o2)cc1)c1ccccc1] |
| 73 | 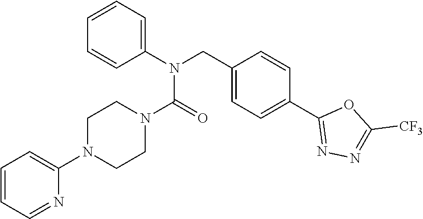 [SMILES: O=C(N1CCN(c2ccccn2)CC1)N(Cc1ccc(-c2nnc(C(F)(F)F)o2)cc1)c1ccccc1] |
| 74 | 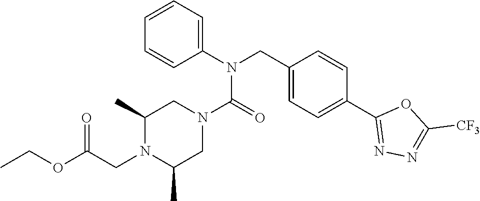 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 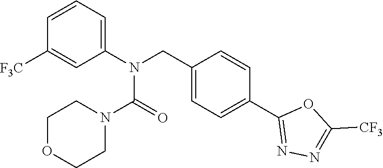 [SMILES: O=C(N1CCOCC1)N(Cc1ccc(-c2nnc(C(F)(F)F)o2)cc1)c1cccc(C(F)(F)F)c1] |
| 76 | 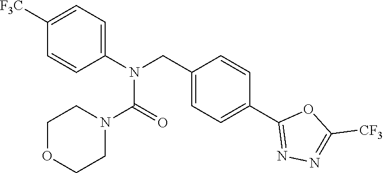 [SMILES: O=C(N1CCOCC1)N(Cc1ccc(-c2nnc(C(F)(F)F)o2)cc1)c1ccc(C(F)(F)F)cc1] |
| 77 | 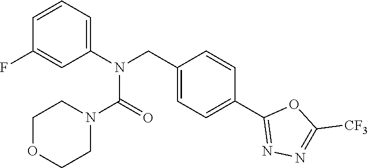 [SMILES: O=C(N1CCOCC1)N(Cc1ccc(-c2nnc(C(F)(F)F)o2)cc1)c1cccc(F)c1] |
| 78 | 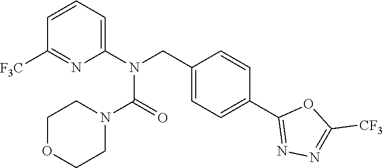 [SMILES: O=C(N1CCOCC1)N(Cc1ccc(-c2nnc(C(F)(F)F)o2)cc1)c1cccc(C(F)(F)F)n1] |
| 79 | 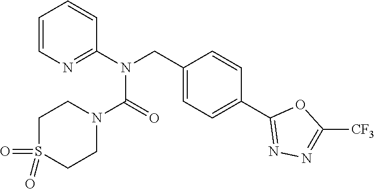 [SMILES: O=C(N1CCS(=O)(=O)CC1)N(Cc1ccc(-c2nnc(C(F)(F)F)o2)cc1)c1ccccn1] |
| 80 | 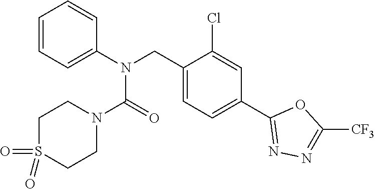 [SMILES: O=C(N1CCS(=O)(=O)CC1)N(Cc1ccc(-c2nnc(C(F)(F)F)o2)cc1Cl)c1ccccc1] |
| 81 | 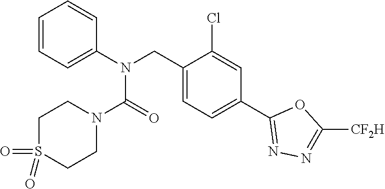 [SMILES: O=C(N1CCS(=O)(=O)CC1)N(Cc1ccc(-c2nnc(C(F)F)o2)cc1Cl)c1ccccc1] |
| 82 | 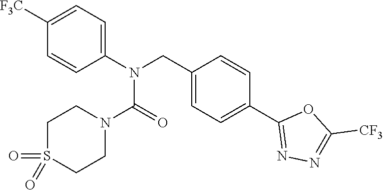 [SMILES: O=C(N1CCS(=O)(=O)CC1)N(Cc1ccc(-c2nnc(C(F)(F)F)o2)cc1)c1ccc(C(F)(F)F)cc1] |
| 83 | 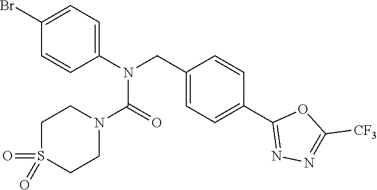 [SMILES: O=C(N1CCS(=O)(=O)CC1)N(Cc1ccc(-c2nnc(C(F)(F)F)o2)cc1)c1ccc(Br)cc1] |
| 84 | 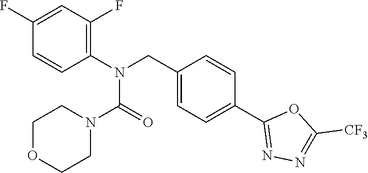 [SMILES: O=C(N1CCOCC1)N(Cc1ccc(-c2nnc(C(F)(F)F)o2)cc1)c1ccc(F)cc1F] |
| 85 | 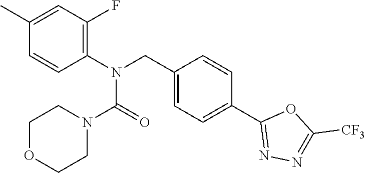 [SMILES: Cc1ccc(N(Cc2ccc(-c3nnc(C(F)(F)F)o3)cc2)C(=O)N2CCOCC2)c(F)c1] |
| 86 | 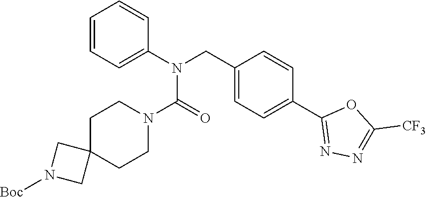 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 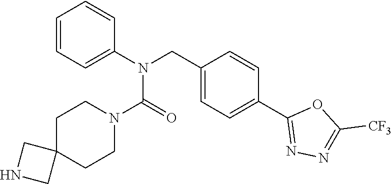 [SMILES: O=C(N1CCC2(CC1)CNC2)N(Cc1ccc(-c2nnc(C(F)(F)F)o2)cc1)c1ccccc1] |
| 88 | 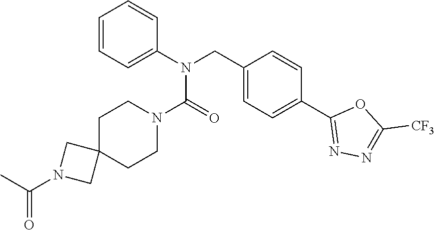 [SMILES: CC(=O)N1CC2(CCN(C(=O)N(Cc3ccc(-c4nnc(C(F)(F)F)o4)cc3)c3ccccc3)CC2)C1] |
| 89 | 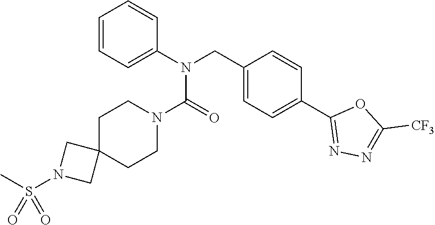 [SMILES: CS(=O)(=O)N1CC2(CCN(C(=O)N(Cc3ccc(-c4nnc(C(F)(F)F)o4)cc3)c3ccccc3)CC2)C1] |
| 90 | 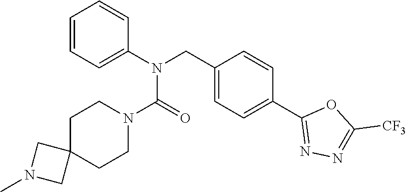 [SMILES: CN1CC2(CCN(C(=O)N(Cc3ccc(-c4nnc(C(F)(F)F)o4)cc3)c3ccccc3)CC2)C1] |
| 91 | 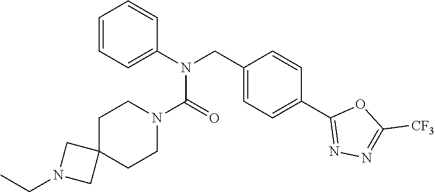 [SMILES: CCN1CC2(CCN(C(=O)N(Cc3ccc(-c4nnc(C(F)(F)F)o4)cc3)c3ccccc3)CC2)C1] |
| 92 | 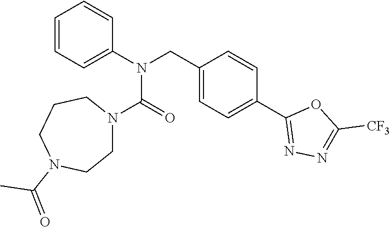 [SMILES: CC(=O)N1CCCN(C(=O)N(Cc2ccc(-c3nnc(C(F)(F)F)o3)cc2)c2ccccc2)CC1] |
| 93 | 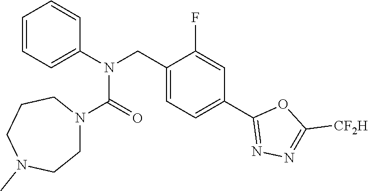 [SMILES: CN1CCCN(C(=O)N(Cc2ccc(-c3nnc(C(F)F)o3)cc2F)c2ccccc2)CC1] |
| 94 | 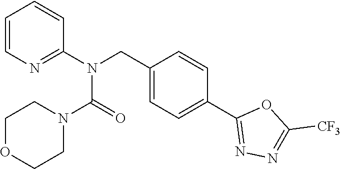 [SMILES: O=C(N1CCOCC1)N(Cc1ccc(-c2nnc(C(F)(F)F)o2)cc1)c1ccccn1] |
| 95 | 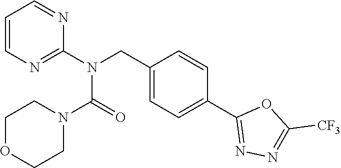 [SMILES: O=C(N1CCOCC1)N(Cc1ccc(-c2nnc(C(F)(F)F)o2)cc1)c1ncccn1] |
| 96 | 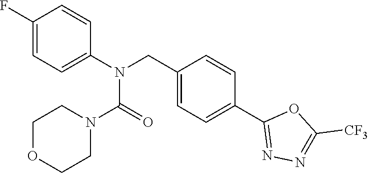 [SMILES: O=C(N1CCOCC1)N(Cc1ccc(-c2nnc(C(F)(F)F)o2)cc1)c1ccc(F)cc1] |
| 97 | 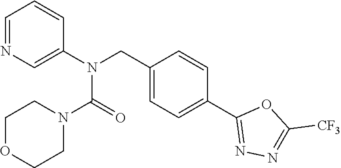 [SMILES: O=C(N1CCOCC1)N(Cc1ccc(-c2nnc(C(F)(F)F)o2)cc1)c1cccnc1] |
| 98 | 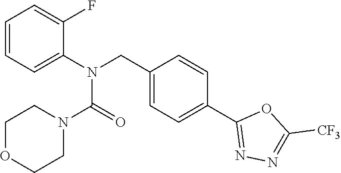 [SMILES: O=C(N1CCOCC1)N(Cc1ccc(-c2nnc(C(F)(F)F)o2)cc1)c1ccccc1F] |
| 99 | 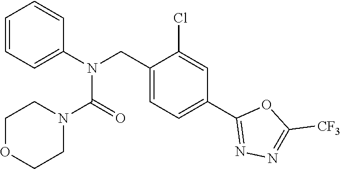 [SMILES: O=C(N1CCOCC1)N(Cc1ccc(-c2nnc(C(F)(F)F)o2)cc1Cl)c1ccccc1] |
| 100 | 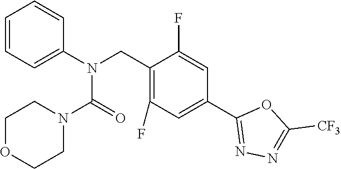 [SMILES: O=C(N1CCOCC1)N(Cc1c(F)cc(-c2nnc(C(F)(F)F)o2)cc1F)c1ccccc1] |
| 101 | 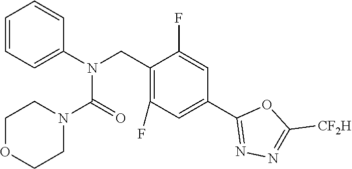 [SMILES: O=C(N1CCOCC1)N(Cc1c(F)cc(-c2nnc(C(F)F)o2)cc1F)c1ccccc1] |
| 102 | 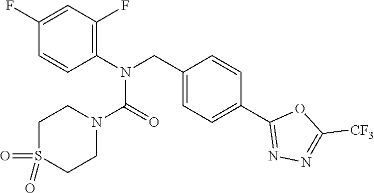 [SMILES: O=C(N1CCS(=O)(=O)CC1)N(Cc1ccc(-c2nnc(C(F)(F)F)o2)cc1)c1ccc(F)cc1F] |
| 103 | 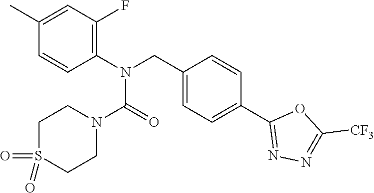 [SMILES: Cc1ccc(N(Cc2ccc(-c3nnc(C(F)(F)F)o3)cc2)C(=O)N2CCS(=O)(=O)CC2)c(F)c1] |
| 104 | 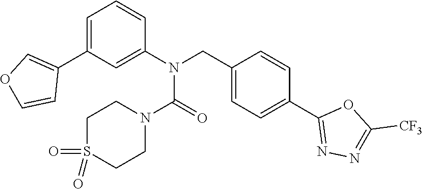 [SMILES: O=C(N1CCS(=O)(=O)CC1)N(Cc1ccc(-c2nnc(C(F)(F)F)o2)cc1)c1cccc(-c2ccoc2)c1] |
| 105 | 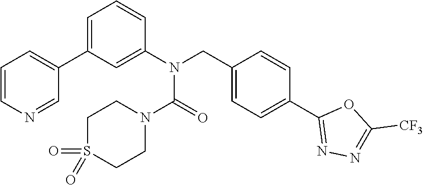 [SMILES: O=C(N1CCS(=O)(=O)CC1)N(Cc1ccc(-c2nnc(C(F)(F)F)o2)cc1)c1cccc(-c2cccnc2)c1] |
| 106 | 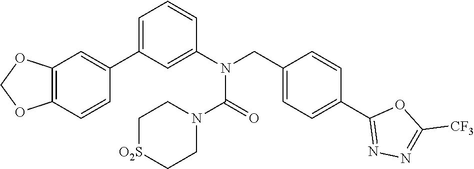 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 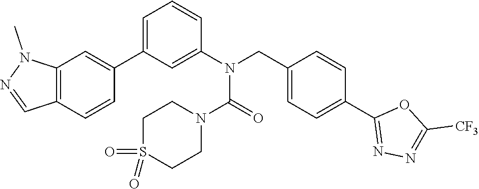 [SMILES: Cn1ncc2ccc(-c3cccc(N(Cc4ccc(-c5nnc(C(F)(F)F)o5)cc4)C(=O)N4CCS(=O)(=O)CC4)c3)cc21] |
| 108 | 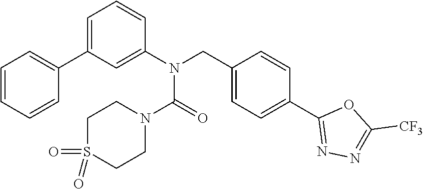 [SMILES: O=C(N1CCS(=O)(=O)CC1)N(Cc1ccc(-c2nnc(C(F)(F)F)o2)cc1)c1cccc(-c2ccccc2)c1] |
| 109 | 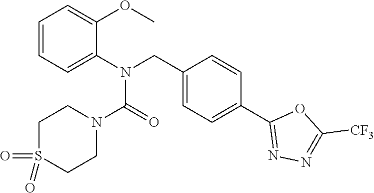 [SMILES: COc1ccccc1N(Cc1ccc(-c2nnc(C(F)(F)F)o2)cc1)C(=O)N1CCS(=O)(=O)CC1] |
| 110 | 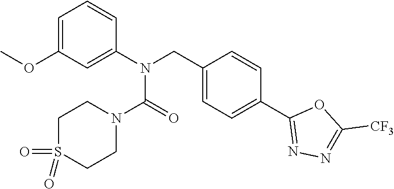 [SMILES: COc1cccc(N(Cc2ccc(-c3nnc(C(F)(F)F)o3)cc2)C(=O)N2CCS(=O)(=O)CC2)c1] |
| 111 | 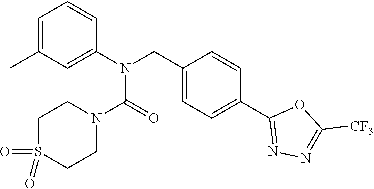 [SMILES: Cc1cccc(N(Cc2ccc(-c3nnc(C(F)(F)F)o3)cc2)C(=O)N2CCS(=O)(=O)CC2)c1] |
| 112 | 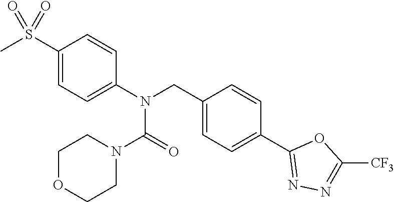 [SMILES: CS(=O)(=O)c1ccc(N(Cc2ccc(-c3nnc(C(F)(F)F)o3)cc2)C(=O)N2CCOCC2)cc1] |
| 113 | 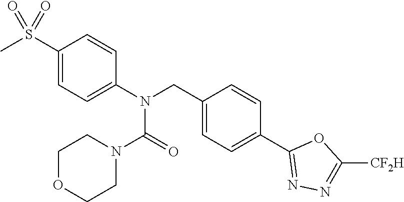 [SMILES: CS(=O)(=O)c1ccc(N(Cc2ccc(-c3nnc(C(F)F)o3)cc2)C(=O)N2CCOCC2)cc1] |
| 114 | 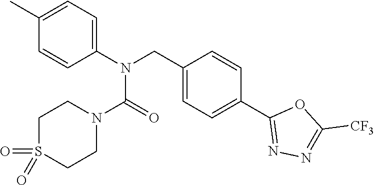 [SMILES: Cc1ccc(N(Cc2ccc(-c3nnc(C(F)(F)F)o3)cc2)C(=O)N2CCS(=O)(=O)CC2)cc1] |
| 115 | 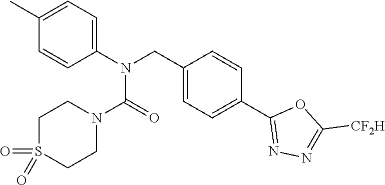 [SMILES: Cc1ccc(N(Cc2ccc(-c3nnc(C(F)F)o3)cc2)C(=O)N2CCS(=O)(=O)CC2)cc1] |
| 116 | 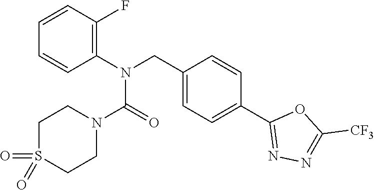 [SMILES: O=C(N1CCS(=O)(=O)CC1)N(Cc1ccc(-c2nnc(C(F)(F)F)o2)cc1)c1ccccc1F] |
| 117 | 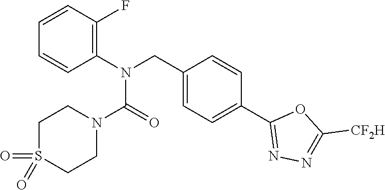 [SMILES: O=C(N1CCS(=O)(=O)CC1)N(Cc1ccc(-c2nnc(C(F)F)o2)cc1)c1ccccc1F] |
| 118 | 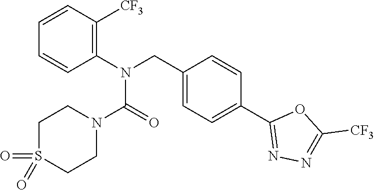 [SMILES: O=C(N1CCS(=O)(=O)CC1)N(Cc1ccc(-c2nnc(C(F)(F)F)o2)cc1)c1ccccc1C(F)(F)F] |
| 119 | 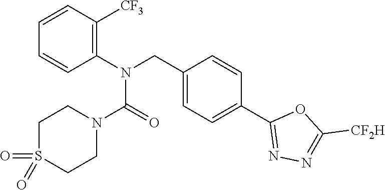 [SMILES: O=C(N1CCS(=O)(=O)CC1)N(Cc1ccc(-c2nnc(C(F)F)o2)cc1)c1ccccc1C(F)(F)F] |
| 120 | 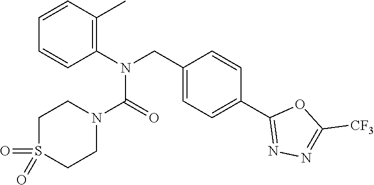 [SMILES: Cc1ccccc1N(Cc1ccc(-c2nnc(C(F)(F)F)o2)cc1)C(=O)N1CCS(=O)(=O)CC1] |
| 121 | 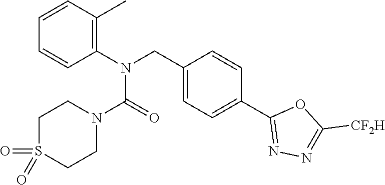 [SMILES: Cc1ccccc1N(Cc1ccc(-c2nnc(C(F)F)o2)cc1)C(=O)N1CCS(=O)(=O)CC1] |
| 122 | 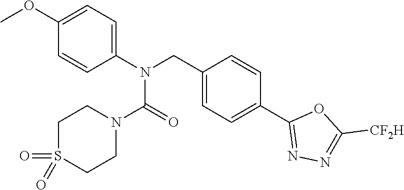 [SMILES: COc1ccc(N(Cc2ccc(-c3nnc(C(F)F)o3)cc2)C(=O)N2CCS(=O)(=O)CC2)cc1] |
| 123 | 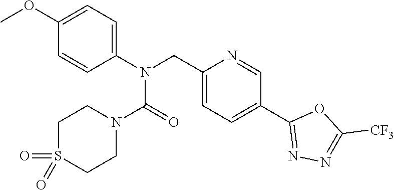 [SMILES: COc1ccc(N(Cc2ccc(-c3nnc(C(F)(F)F)o3)cn2)C(=O)N2CCS(=O)(=O)CC2)cc1] |
| 124 | 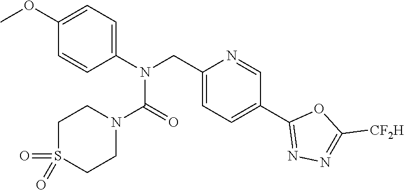 [SMILES: COc1ccc(N(Cc2ccc(-c3nnc(C(F)F)o3)cn2)C(=O)N2CCS(=O)(=O)CC2)cc1] |
| 125 | 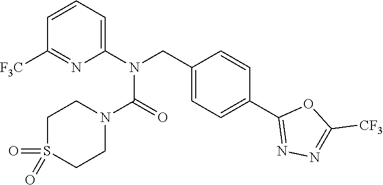 [SMILES: O=C(N1CCS(=O)(=O)CC1)N(Cc1ccc(-c2nnc(C(F)(F)F)o2)cc1)c1cccc(C(F)(F)F)n1] |
| 126 | 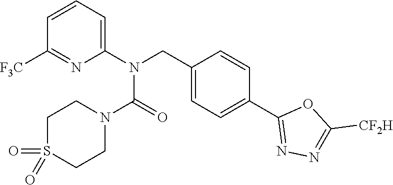 [SMILES: O=C(N1CCS(=O)(=O)CC1)N(Cc1ccc(-c2nnc(C(F)F)o2)cc1)c1cccc(C(F)(F)F)n1] |
| 127 | 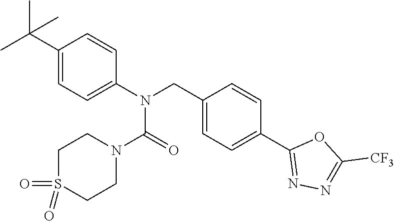 [SMILES: CC(C)(C)c1ccc(N(Cc2ccc(-c3nnc(C(F)(F)F)o3)cc2)C(=O)N2CCS(=O)(=O)CC2)cc1] |
| 128 | 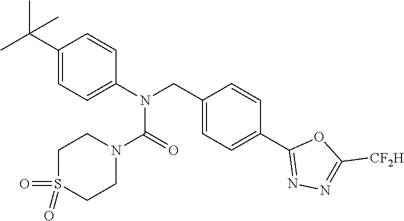 [SMILES: CC(C)(C)c1ccc(N(Cc2ccc(-c3nnc(C(F)F)o3)cc2)C(=O)N2CCS(=O)(=O)CC2)cc1] |
| 129 | 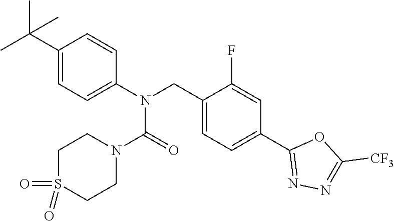 [SMILES: CC(C)(C)c1ccc(N(Cc2ccc(-c3nnc(C(F)(F)F)o3)cc2F)C(=O)N2CCS(=O)(=O)CC2)cc1] |
| 130 | 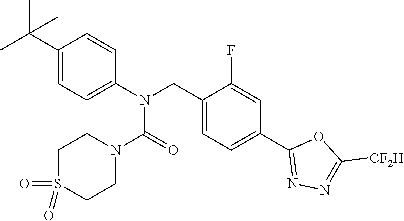 [SMILES: CC(C)(C)c1ccc(N(Cc2ccc(-c3nnc(C(F)F)o3)cc2F)C(=O)N2CCS(=O)(=O)CC2)cc1] |
| 131 | 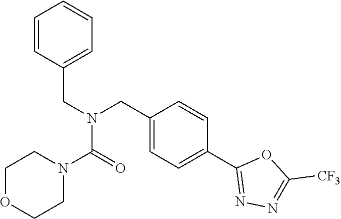 [SMILES: O=C(N1CCOCC1)N(Cc1ccccc1)Cc1ccc(-c2nnc(C(F)(F)F)o2)cc1] |
| 132 | 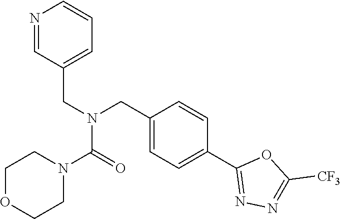 [SMILES: O=C(N1CCOCC1)N(Cc1ccc(-c2nnc(C(F)(F)F)o2)cc1)Cc1cccnc1] |
| 133 | 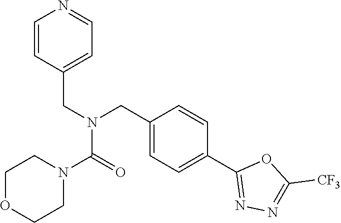 [SMILES: O=C(N1CCOCC1)N(Cc1ccncc1)Cc1ccc(-c2nnc(C(F)(F)F)o2)cc1] |
| 134 | 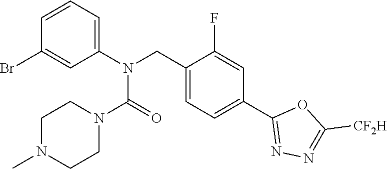 [SMILES: CN1CCN(C(=O)N(Cc2ccc(-c3nnc(C(F)F)o3)cc2F)c2cccc(Br)c2)CC1] |
| 135 | 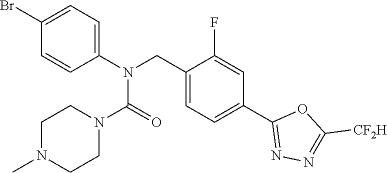 [SMILES: CN1CCN(C(=O)N(Cc2ccc(-c3nnc(C(F)F)o3)cc2F)c2ccc(Br)cc2)CC1] |
| 136 | 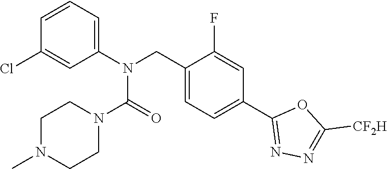 [SMILES: CN1CCN(C(=O)N(Cc2ccc(-c3nnc(C(F)F)o3)cc2F)c2cccc(Cl)c2)CC1] |
| 137 | 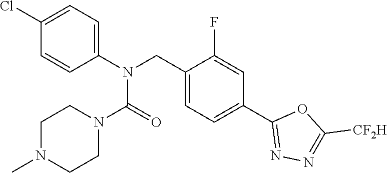 [SMILES: CN1CCN(C(=O)N(Cc2ccc(-c3nnc(C(F)F)o3)cc2F)c2ccc(Cl)cc2)CC1] |
| 138 | 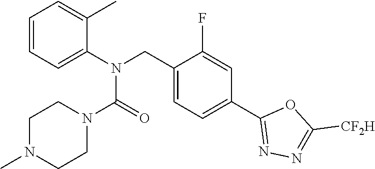 [SMILES: Cc1ccccc1N(Cc1ccc(-c2nnc(C(F)F)o2)cc1F)C(=O)N1CCN(C)CC1] |
| 139 | 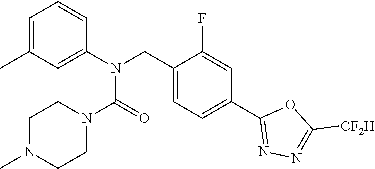 [SMILES: Cc1cccc(N(Cc2ccc(-c3nnc(C(F)F)o3)cc2F)C(=O)N2CCN(C)CC2)c1] |
| 140 | 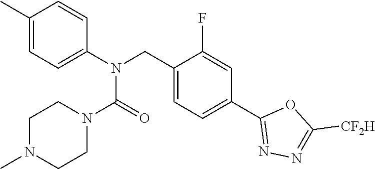 [SMILES: Cc1ccc(N(Cc2ccc(-c3nnc(C(F)F)o3)cc2F)C(=O)N2CCN(C)CC2)cc1] |
| 141 | 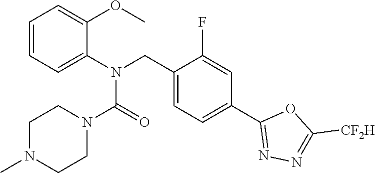 [SMILES: COc1ccccc1N(Cc1ccc(-c2nnc(C(F)F)o2)cc1F)C(=O)N1CCN(C)CC1] |
| 142 | 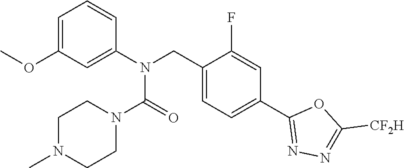 [SMILES: COc1cccc(N(Cc2ccc(-c3nnc(C(F)F)o3)cc2F)C(=O)N2CCN(C)CC2)c1] |
| 143 | 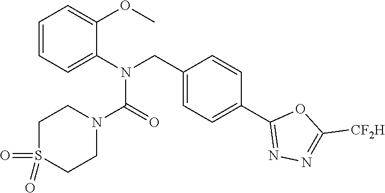 [SMILES: COc1ccccc1N(Cc1ccc(-c2nnc(C(F)F)o2)cc1)C(=O)N1CCS(=O)(=O)CC1] |
| 144 | 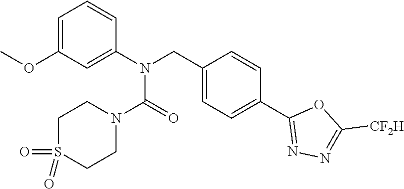 [SMILES: COc1cccc(N(Cc2ccc(-c3nnc(C(F)F)o3)cc2)C(=O)N2CCS(=O)(=O)CC2)c1] |
| 145 | 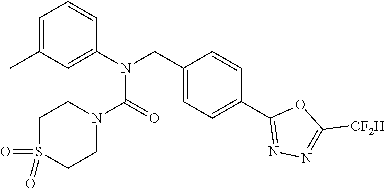 [SMILES: Cc1cccc(N(Cc2ccc(-c3nnc(C(F)F)o3)cc2)C(=O)N2CCS(=O)(=O)CC2)c1] |
| 146 | 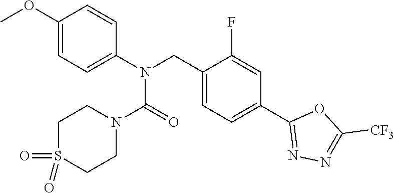 [SMILES: COc1ccc(N(Cc2ccc(-c3nnc(C(F)(F)F)o3)cc2F)C(=O)N2CCS(=O)(=O)CC2)cc1] |
| 147 | 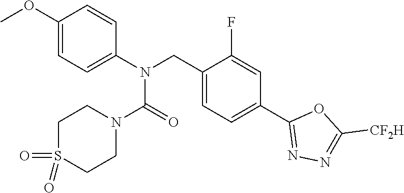 [SMILES: COc1ccc(N(Cc2ccc(-c3nnc(C(F)F)o3)cc2F)C(=O)N2CCS(=O)(=O)CC2)cc1] |
| 148 | 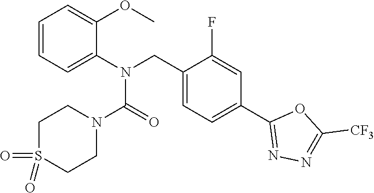 [SMILES: COc1ccccc1N(Cc1ccc(-c2nnc(C(F)(F)F)o2)cc1F)C(=O)N1CCS(=O)(=O)CC1] |
| 149 | 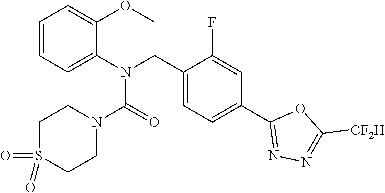 [SMILES: COc1ccccc1N(Cc1ccc(-c2nnc(C(F)F)o2)cc1F)C(=O)N1CCS(=O)(=O)CC1] |
| 150 | 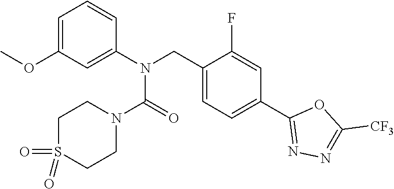 [SMILES: COc1cccc(N(Cc2ccc(-c3nnc(C(F)(F)F)o3)cc2F)C(=O)N2CCS(=O)(=O)CC2)c1] |
| 151 | 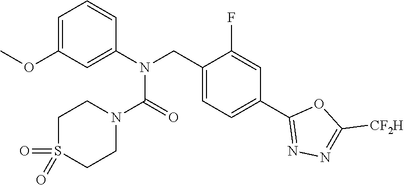 [SMILES: COc1cccc(N(Cc2ccc(-c3nnc(C(F)F)o3)cc2F)C(=O)N2CCS(=O)(=O)CC2)c1] |
| 152 | 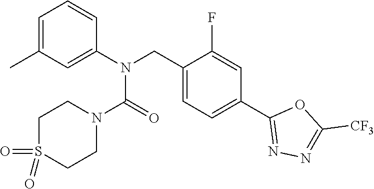 [SMILES: Cc1cccc(N(Cc2ccc(-c3nnc(C(F)(F)F)o3)cc2F)C(=O)N2CCS(=O)(=O)CC2)c1] |
| 153 | 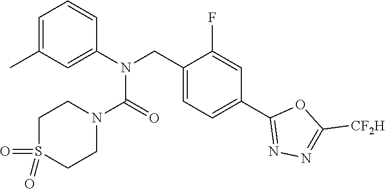 [SMILES: Cc1cccc(N(Cc2ccc(-c3nnc(C(F)F)o3)cc2F)C(=O)N2CCS(=O)(=O)CC2)c1] |
| 154 | 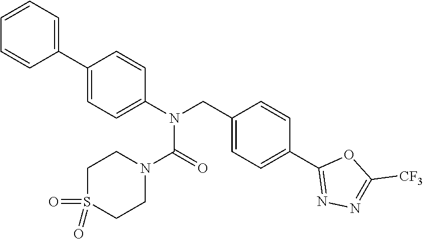 [SMILES: O=C(N1CCS(=O)(=O)CC1)N(Cc1ccc(-c2nnc(C(F)(F)F)o2)cc1)c1ccc(-c2ccccc2)cc1] |
| 155 | 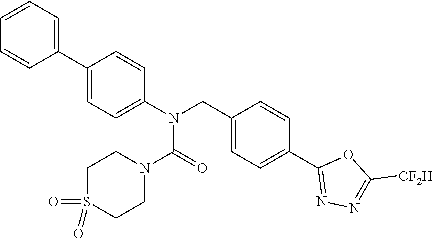 [SMILES: O=C(N1CCS(=O)(=O)CC1)N(Cc1ccc(-c2nnc(C(F)F)o2)cc1)c1ccc(-c2ccccc2)cc1] |
| 156 | 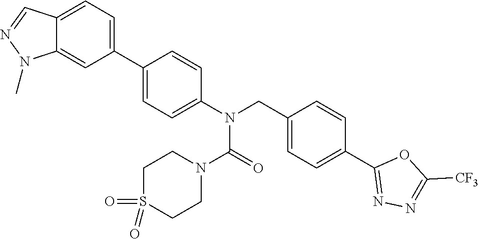 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 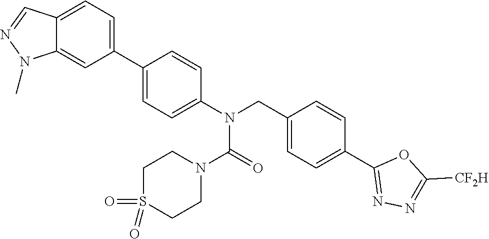 [SMILES: Cn1ncc2ccc(-c3ccc(N(Cc4ccc(-c5nnc(C(F)F)o5)cc4)C(=O)N4CCS(=O)(=O)CC4)cc3)cc21] |
| 158 | 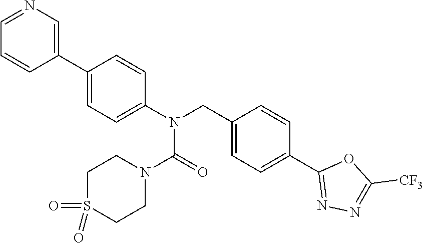 [SMILES: O=C(N1CCS(=O)(=O)CC1)N(Cc1ccc(-c2nnc(C(F)(F)F)o2)cc1)c1ccc(-c2cccnc2)cc1] |
| 159 | 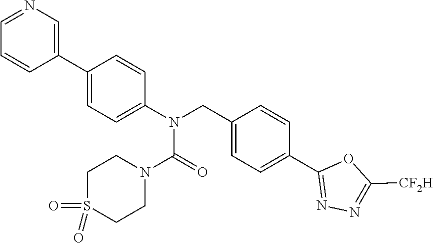 [SMILES: O=C(N1CCS(=O)(=O)CC1)N(Cc1ccc(-c2nnc(C(F)F)o2)cc1)c1ccc(-c2cccnc2)cc1] |
| 160 | 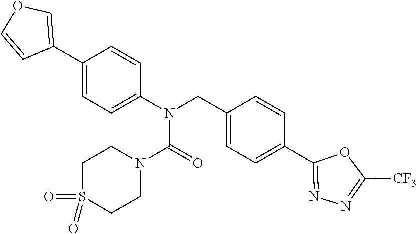 [SMILES: O=C(N1CCS(=O)(=O)CC1)N(Cc1ccc(-c2nnc(C(F)(F)F)o2)cc1)c1ccc(-c2ccoc2)cc1] |
| 161 | 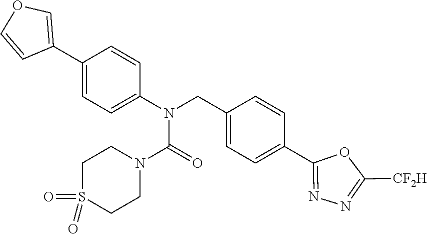 [SMILES: O=C(N1CCS(=O)(=O)CC1)N(Cc1ccc(-c2nnc(C(F)F)o2)cc1)c1ccc(-c2ccoc2)cc1] |
| 162 | 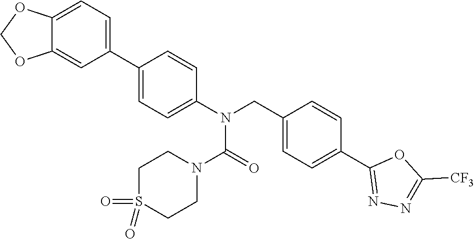 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 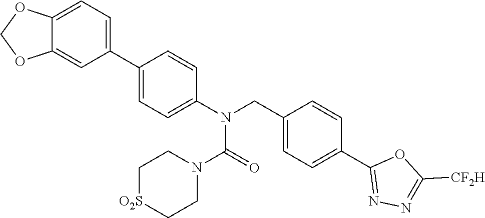 [SMILES: O=C(N1CCS(=O)(=O)CC1)N(Cc1ccc(-c2nnc(C(F)F)o2)cc1)c1ccc(-c2ccc3c(c2)OCO3)cc1] |
| 164 | 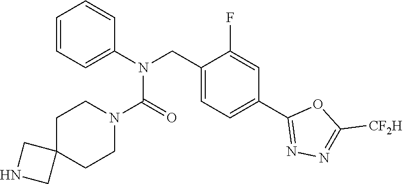 [SMILES: O=C(N1CCC2(CC1)CNC2)N(Cc1ccc(-c2nnc(C(F)F)o2)cc1F)c1ccccc1] |
| 165 | 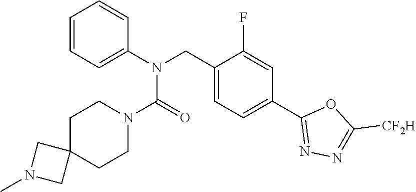 [SMILES: CN1CC2(CCN(C(=O)N(Cc3ccc(-c4nnc(C(F)F)o4)cc3F)c3ccccc3)CC2)C1] |
| 166 | 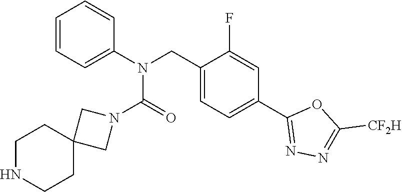 [SMILES: O=C(N1CC2(CCNCC2)C1)N(Cc1ccc(-c2nnc(C(F)F)o2)cc1F)c1ccccc1] |
| 167 | 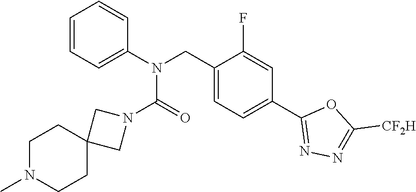 [SMILES: CN1CCC2(CC1)CN(C(=O)N(Cc1ccc(-c3nnc(C(F)F)o3)cc1F)c1ccccc1)C2] |
| 168 | 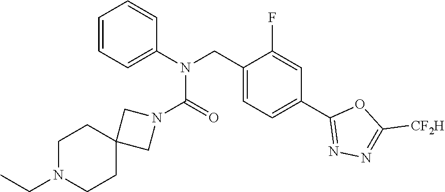 [SMILES: CCN1CCC2(CC1)CN(C(=O)N(Cc1ccc(-c3nnc(C(F)F)o3)cc1F)c1ccccc1)C2] |
| 169 | 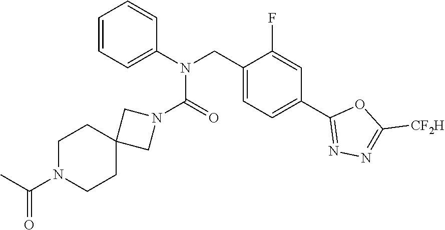 [SMILES: CC(=O)N1CCC2(CC1)CN(C(=O)N(Cc1ccc(-c3nnc(C(F)F)o3)cc1F)c1ccccc1)C2] |
| 170 | 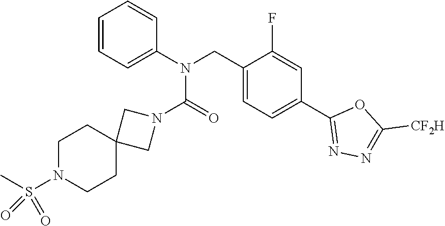 [SMILES: CS(=O)(=O)N1CCC2(CC1)CN(C(=O)N(Cc1ccc(-c3nnc(C(F)F)o3)cc1F)c1ccccc1)C2] |
| 171 | 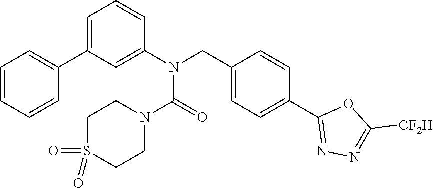 [SMILES: O=C(N1CCS(=O)(=O)CC1)N(Cc1ccc(-c2nnc(C(F)F)o2)cc1)c1cccc(-c2ccccc2)c1] |
| 172 | 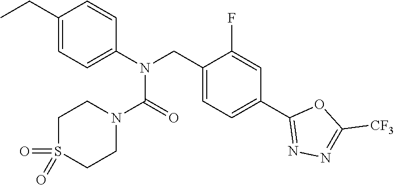 [SMILES: CCc1ccc(N(Cc2ccc(-c3nnc(C(F)(F)F)o3)cc2F)C(=O)N2CCS(=O)(=O)CC2)cc1] |
| 173 | 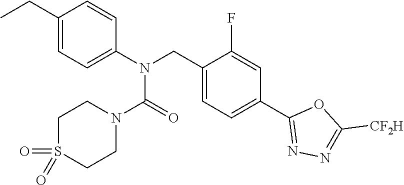 [SMILES: CCc1ccc(N(Cc2ccc(-c3nnc(C(F)F)o3)cc2F)C(=O)N2CCS(=O)(=O)CC2)cc1] |
| 174 | 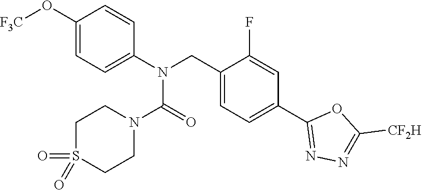 [SMILES: O=C(N1CCS(=O)(=O)CC1)N(Cc1ccc(-c2nnc(C(F)F)o2)cc1F)c1ccc(OC(F)(F)F)cc1] |
| 175 | 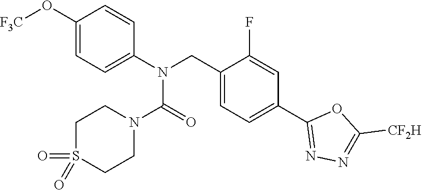 [SMILES: O=C(N1CCS(=O)(=O)CC1)N(Cc1ccc(-c2nnc(C(F)F)o2)cc1F)c1ccc(OC(F)(F)F)cc1] |
| 176 | 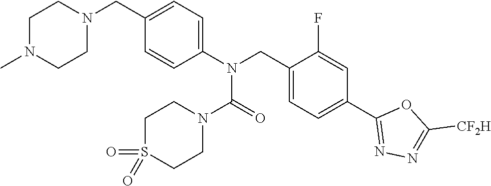 [SMILES: CN1CCN(Cc2ccc(N(Cc3ccc(-c4nnc(C(F)F)o4)cc3F)C(=O)N3CCS(=O)(=O)CC3)cc2)CC1] |
| 177 | 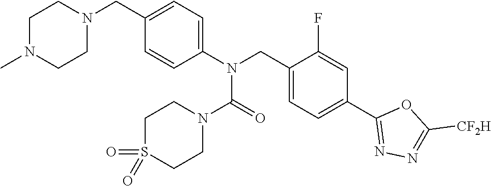 [SMILES: CN1CCN(Cc2ccc(N(Cc3ccc(-c4nnc(C(F)F)o4)cc3F)C(=O)N3CCS(=O)(=O)CC3)cc2)CC1] |
| 178 | 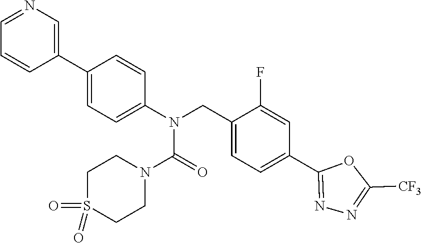 [SMILES: O=C(N1CCS(=O)(=O)CC1)N(Cc1ccc(-c2nnc(C(F)(F)F)o2)cc1F)c1ccc(-c2cccnc2)cc1] |
| 179 | 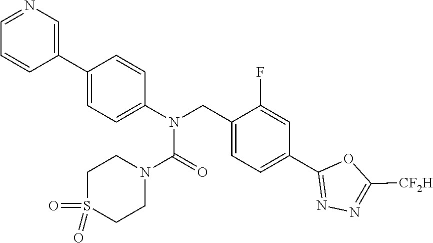 [SMILES: O=C(N1CCS(=O)(=O)CC1)N(Cc1ccc(-c2nnc(C(F)F)o2)cc1F)c1ccc(-c2cccnc2)cc1] |
| 180 | 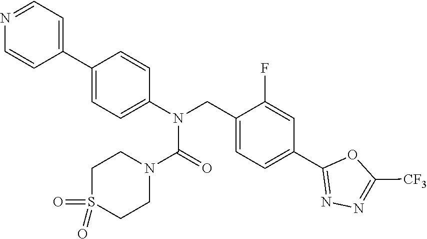 [SMILES: O=C(N1CCS(=O)(=O)CC1)N(Cc1ccc(-c2nnc(C(F)(F)F)o2)cc1F)c1ccc(-c2ccncc2)cc1] |
| 181 | 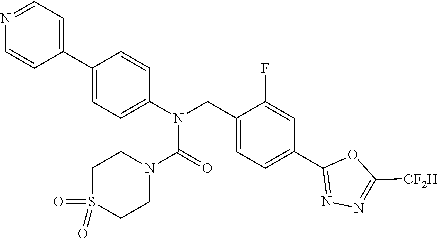 [SMILES: O=C(N1CCS(=O)(=O)CC1)N(Cc1ccc(-c2nnc(C(F)F)o2)cc1F)c1ccc(-c2ccncc2)cc1] |
| 182 | 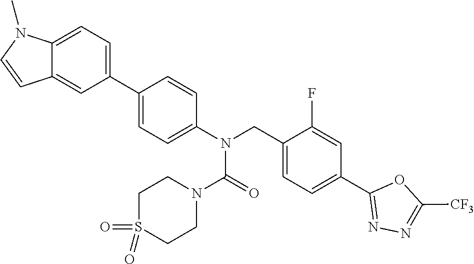 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 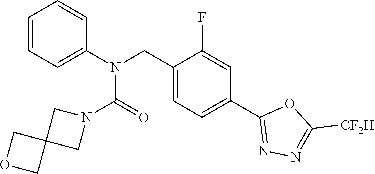 [SMILES: O=C(N1CC2(COC2)C1)N(Cc1ccc(-c2nnc(C(F)F)o2)cc1F)c1ccccc1] |
| 184 | 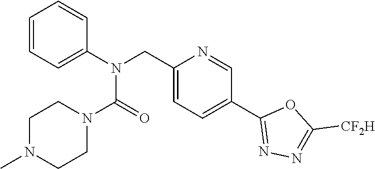 [SMILES: CN1CCN(C(=O)N(Cc2ccc(-c3nnc(C(F)F)o3)cn2)c2ccccc2)CC1] |
| 185 | 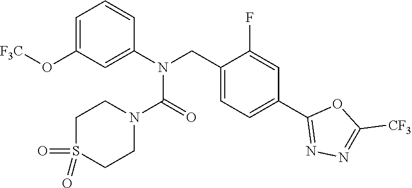 [SMILES: O=C(N1CCS(=O)(=O)CC1)N(Cc1ccc(-c2nnc(C(F)(F)F)o2)cc1F)c1cccc(OC(F)(F)F)c1] |
| 186 | 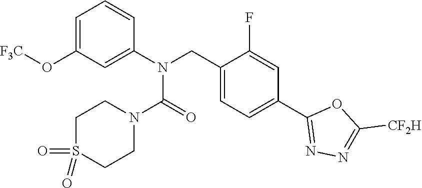 [SMILES: O=C(N1CCS(=O)(=O)CC1)N(Cc1ccc(-c2nnc(C(F)F)o2)cc1F)c1cccc(OC(F)(F)F)c1] |
| 187 | 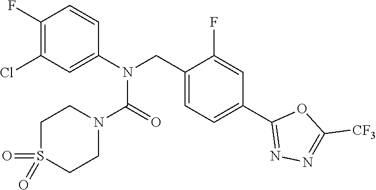 [SMILES: O=C(N1CCS(=O)(=O)CC1)N(Cc1ccc(-c2nnc(C(F)(F)F)o2)cc1F)c1ccc(F)c(Cl)c1] |
| 188 | 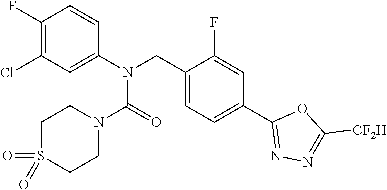 [SMILES: O=C(N1CCS(=O)(=O)CC1)N(Cc1ccc(-c2nnc(C(F)F)o2)cc1F)c1ccc(F)c(Cl)c1] |
| 189 | 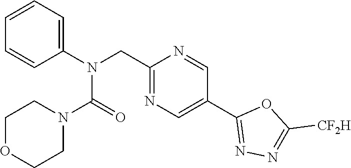 [SMILES: O=C(N1CCOCC1)N(Cc1ncc(-c2nnc(C(F)F)o2)cn1)c1ccccc1] |
| 190 | 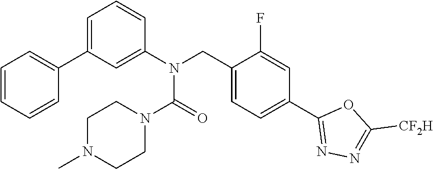 [SMILES: CN1CCN(C(=O)N(Cc2ccc(-c3nnc(C(F)F)o3)cc2F)c2cccc(-c3ccccc3)c2)CC1] |
| 191 | 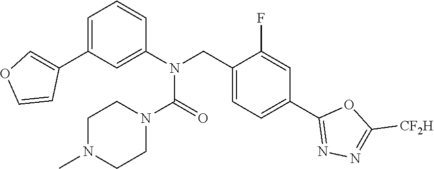 [SMILES: CN1CCN(C(=O)N(Cc2ccc(-c3nnc(C(F)F)o3)cc2F)c2cccc(-c3ccoc3)c2)CC1] |
| 192 | 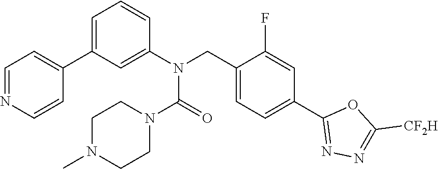 [SMILES: CN1CCN(C(=O)N(Cc2ccc(-c3nnc(C(F)F)o3)cc2F)c2cccc(-c3ccncc3)c2)CC1] |
| 193 | 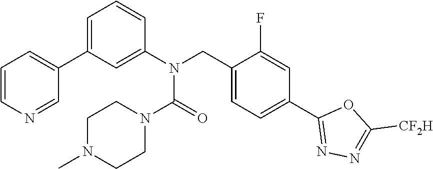 [SMILES: CN1CCN(C(=O)N(Cc2ccc(-c3nnc(C(F)F)o3)cc2F)c2cccc(-c3cccnc3)c2)CC1] |
| 194 | 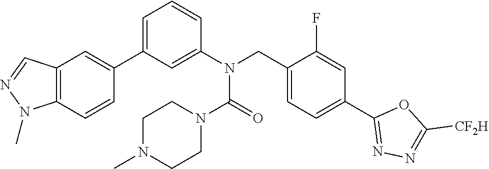 [SMILES: CN1CCN(C(=O)N(Cc2ccc(-c3nnc(C(F)F)o3)cc2F)c2cccc(-c3ccc4c(cnn4C)c3)c2)CC1] |
| 195 | 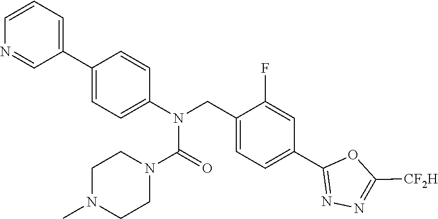 [SMILES: CN1CCN(C(=O)N(Cc2ccc(-c3nnc(C(F)F)o3)cc2F)c2ccc(-c3cccnc3)cc2)CC1] |
| 196 | 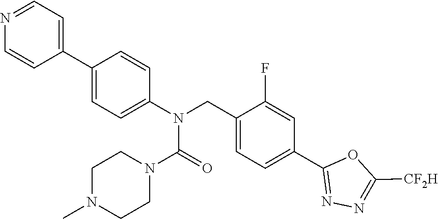 [SMILES: CN1CCN(C(=O)N(Cc2ccc(-c3nnc(C(F)F)o3)cc2F)c2ccc(-c3ccncc3)cc2)CC1] |
| 197 | 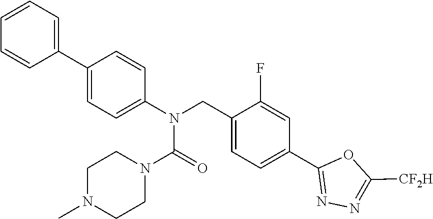 [SMILES: CN1CCN(C(=O)N(Cc2ccc(-c3nnc(C(F)F)o3)cc2F)c2ccc(-c3ccccc3)cc2)CC1] |
| 198 | 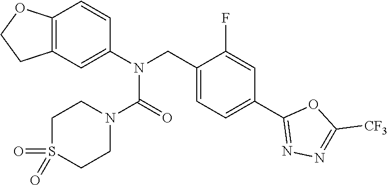 [SMILES: O=C(N1CCS(=O)(=O)CC1)N(Cc1ccc(-c2nnc(C(F)(F)F)o2)cc1F)c1ccc2c(c1)CCO2] |
| 199 | 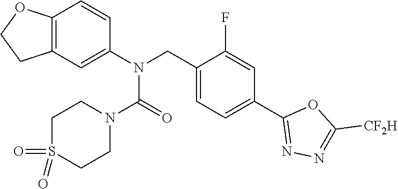 [SMILES: O=C(N1CCS(=O)(=O)CC1)N(Cc1ccc(-c2nnc(C(F)F)o2)cc1F)c1ccc2c(c1)CCO2] |
| 200 | 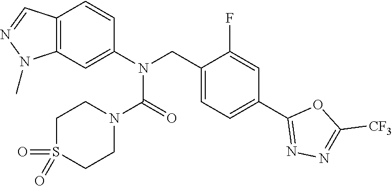 [SMILES: Cn1ncc2ccc(N(Cc3ccc(-c4nnc(C(F)(F)F)o4)cc3F)C(=O)N3CCS(=O)(=O)CC3)cc21] |
| 201 | 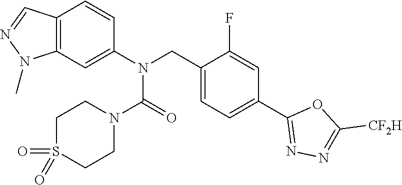 [SMILES: Cn1ncc2ccc(N(Cc3ccc(-c4nnc(C(F)F)o4)cc3F)C(=O)N3CCS(=O)(=O)CC3)cc21] |
| 202 | 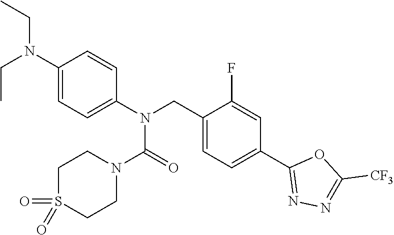 [SMILES: CCN(CC)c1ccc(N(Cc2ccc(-c3nnc(C(F)(F)F)o3)cc2F)C(=O)N2CCS(=O)(=O)CC2)cc1] |
| 203 | 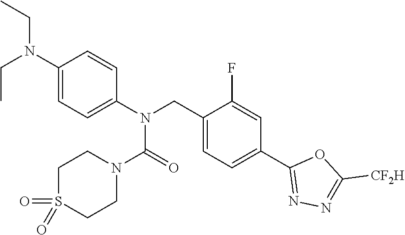 [SMILES: CCN(CC)c1ccc(N(Cc2ccc(-c3nnc(C(F)F)o3)cc2F)C(=O)N2CCS(=O)(=O)CC2)cc1] |
| 204 | 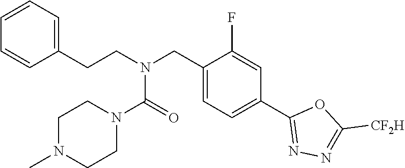 [SMILES: CN1CCN(C(=O)N(CCc2ccccc2)Cc2ccc(-c3nnc(C(F)F)o3)cc2F)CC1] |
| 205 | 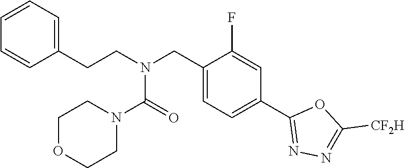 [SMILES: O=C(N1CCOCC1)N(CCc1ccccc1)Cc1ccc(-c2nnc(C(F)F)o2)cc1F] |
| 206 | 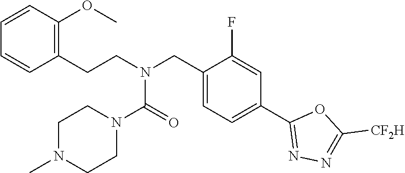 [SMILES: COc1ccccc1CCN(Cc1ccc(-c2nnc(C(F)F)o2)cc1F)C(=O)N1CCN(C)CC1] |
| 207 | 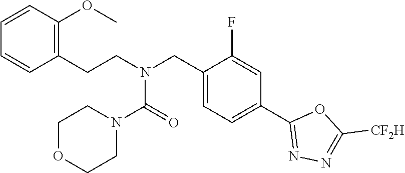 [SMILES: COc1ccccc1CCN(Cc1ccc(-c2nnc(C(F)F)o2)cc1F)C(=O)N1CCOCC1] |
| 208 | 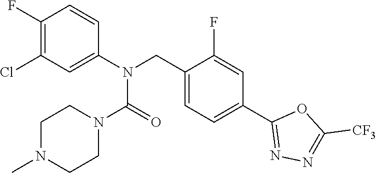 [SMILES: CN1CCN(C(=O)N(Cc2ccc(-c3nnc(C(F)(F)F)o3)cc2F)c2ccc(F)c(Cl)c2)CC1] |
| 209 | 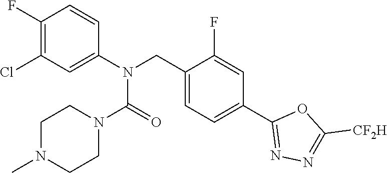 [SMILES: CN1CCN(C(=O)N(Cc2ccc(-c3nnc(C(F)F)o3)cc2F)c2ccc(F)c(Cl)c2)CC1] |
| 210 | 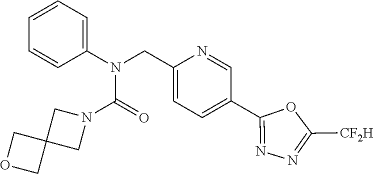 [SMILES: O=C(N1CC2(COC2)C1)N(Cc1ccc(-c2nnc(C(F)F)o2)cn1)c1ccccc1] |
| 211 | 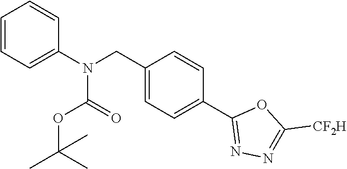 [SMILES: CC(C)(C)OC(=O)N(Cc1ccc(-c2nnc(C(F)F)o2)cc1)c1ccccc1] |
| 212 | 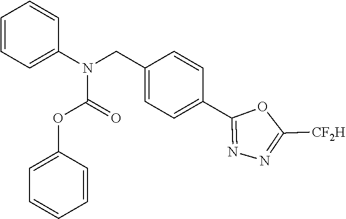 [SMILES: O=C(Oc1ccccc1)N(Cc1ccc(-c2nnc(C(F)F)o2)cc1)c1ccccc1] |
| 213 | 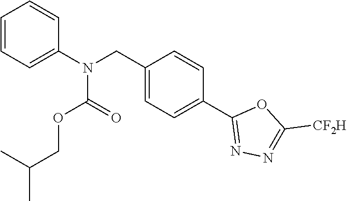 [SMILES: CC(C)COC(=O)N(Cc1ccc(-c2nnc(C(F)F)o2)cc1)c1ccccc1] |
| 214 | 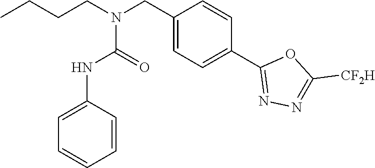 [SMILES: CCCCN(Cc1ccc(-c2nnc(C(F)F)o2)cc1)C(=O)Nc1ccccc1] |
| 215 | 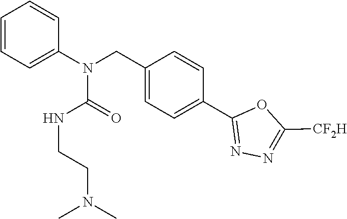 [SMILES: CN(C)CCNC(=O)N(Cc1ccc(-c2nnc(C(F)F)o2)cc1)c1ccccc1] |
| 216 | 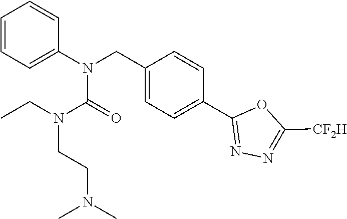 [SMILES: CCN(CCN(C)C)C(=O)N(Cc1ccc(-c2nnc(C(F)F)o2)cc1)c1ccccc1] |
| 217 | 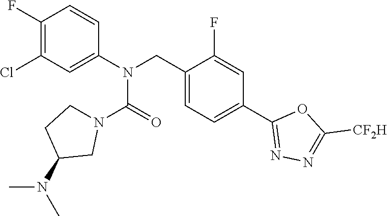 [SMILES: CN(C)[C@H]1CCN(C(=O)N(Cc2ccc(-c3nnc(C(F)F)o3)cc2F)c2ccc(F)c(Cl)c2)C1] |
| 218 | 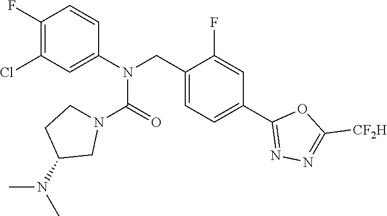 [SMILES: CN(C)[C@@H]1CCN(C(=O)N(Cc2ccc(-c3nnc(C(F)F)o3)cc2F)c2ccc(F)c(Cl)c2)C1] |
| 219 | 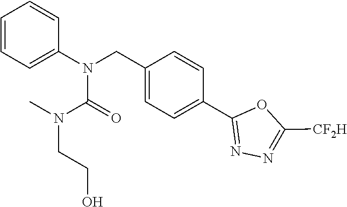 [SMILES: CN(CCO)C(=O)N(Cc1ccc(-c2nnc(C(F)F)o2)cc1)c1ccccc1] |
| 220 | 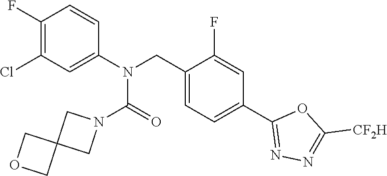 [SMILES: O=C(N1CC2(COC2)C1)N(Cc1ccc(-c2nnc(C(F)F)o2)cc1F)c1ccc(F)c(Cl)c1] |
| 221 | 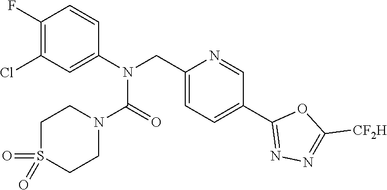 [SMILES: O=C(N1CCS(=O)(=O)CC1)N(Cc1ccc(-c2nnc(C(F)F)o2)cn1)c1ccc(F)c(Cl)c1] |
| 222 | 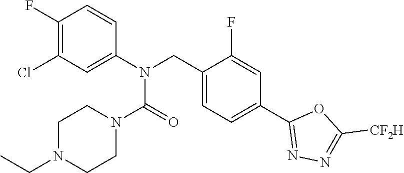 [SMILES: CCN1CCN(C(=O)N(Cc2ccc(-c3nnc(C(F)F)o3)cc2F)c2ccc(F)c(Cl)c2)CC1] |
| 223 | 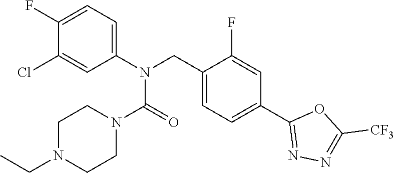 [SMILES: CCN1CCN(C(=O)N(Cc2ccc(-c3nnc(C(F)(F)F)o3)cc2F)c2ccc(F)c(Cl)c2)CC1] |
| 224 | 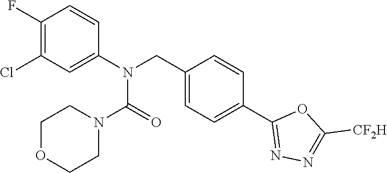 [SMILES: O=C(N1CCOCC1)N(Cc1ccc(-c2nnc(C(F)F)o2)cc1)c1ccc(F)c(Cl)c1] |
| 225 | 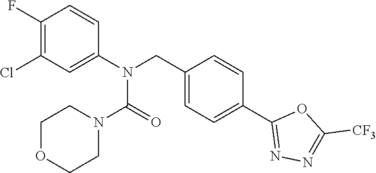 [SMILES: O=C(N1CCOCC1)N(Cc1ccc(-c2nnc(C(F)(F)F)o2)cc1)c1ccc(F)c(Cl)c1] |
| 226 | 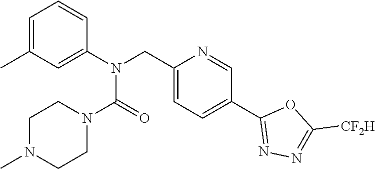 [SMILES: Cc1cccc(N(Cc2ccc(-c3nnc(C(F)F)o3)cn2)C(=O)N2CCN(C)CC2)c1] |
| 227 | 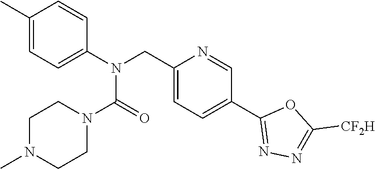 [SMILES: Cc1ccc(N(Cc2ccc(-c3nnc(C(F)F)o3)cn2)C(=O)N2CCN(C)CC2)cc1] |
| 228 | 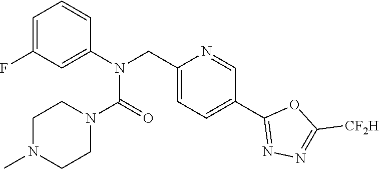 [SMILES: CN1CCN(C(=O)N(Cc2ccc(-c3nnc(C(F)F)o3)cn2)c2cccc(F)c2)CC1] |
| 229 | 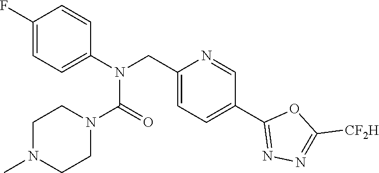 [SMILES: CN1CCN(C(=O)N(Cc2ccc(-c3nnc(C(F)F)o3)cn2)c2ccc(F)cc2)CC1] |
| 230 | 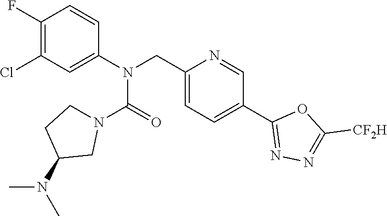 [SMILES: CN(C)[C@H]1CCN(C(=O)N(Cc2ccc(-c3nnc(C(F)F)o3)cn2)c2ccc(F)c(Cl)c2)C1] |
| 231 | 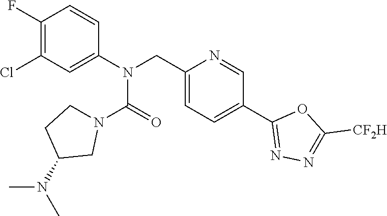 [SMILES: CN(C)[C@@H]1CCN(C(=O)N(Cc2ccc(-c3nnc(C(F)F)o3)cn2)c2ccc(F)c(Cl)c2)C1] |
| 232 | 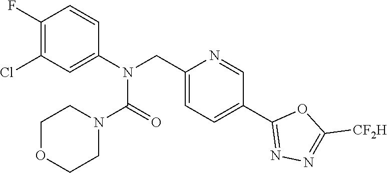 [SMILES: O=C(N1CCOCC1)N(Cc1ccc(-c2nnc(C(F)F)o2)cn1)c1ccc(F)c(Cl)c1] |
| 233 | 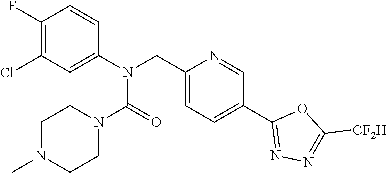 [SMILES: CN1CCN(C(=O)N(Cc2ccc(-c3nnc(C(F)F)o3)cn2)c2ccc(F)c(Cl)c2)CC1] |
| 234 | 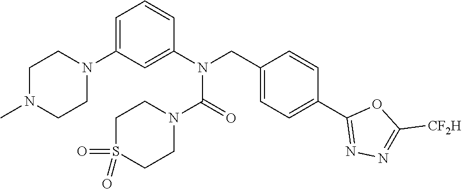 [SMILES: CN1CCN(c2cccc(N(Cc3ccc(-c4nnc(C(F)F)o4)cc3)C(=O)N3CCS(=O)(=O)CC3)c2)CC1] |
| 235 | 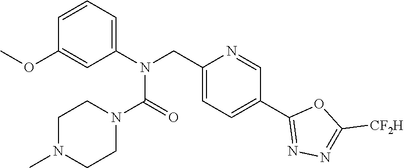 [SMILES: COc1cccc(N(Cc2ccc(-c3nnc(C(F)F)o3)cn2)C(=O)N2CCN(C)CC2)c1] |
| 236 | 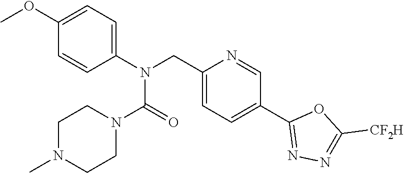 [SMILES: COc1ccc(N(Cc2ccc(-c3nnc(C(F)F)o3)cn2)C(=O)N2CCN(C)CC2)cc1] |
| 237 | 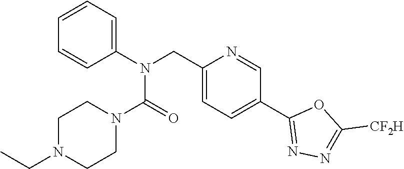 [SMILES: CCN1CCN(C(=O)N(Cc2ccc(-c3nnc(C(F)F)o3)cn2)c2ccccc2)CC1] |
| 238 | 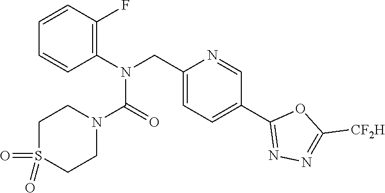 [SMILES: O=C(N1CCS(=O)(=O)CC1)N(Cc1ccc(-c2nnc(C(F)F)o2)cn1)c1ccccc1F] |
| 239 | 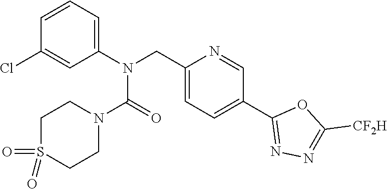 [SMILES: O=C(N1CCS(=O)(=O)CC1)N(Cc1ccc(-c2nnc(C(F)F)o2)cn1)c1cccc(Cl)c1] |
| 240 | 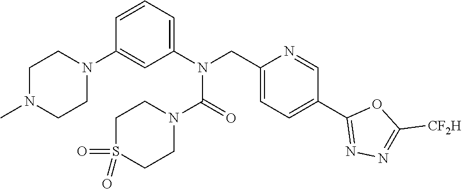 [SMILES: CN1CCN(c2cccc(N(Cc3ccc(-c4nnc(C(F)F)o4)cn3)C(=O)N3CCS(=O)(=O)CC3)c2)CC1] |
| 241 | 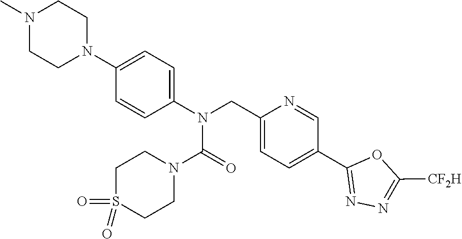 [SMILES: CN1CCN(c2ccc(N(Cc3ccc(-c4nnc(C(F)F)o4)cn3)C(=O)N3CCS(=O)(=O)CC3)cc2)CC1] |
| 242 | 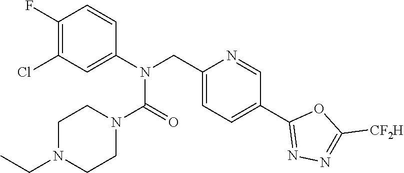 [SMILES: CCN1CCN(C(=O)N(Cc2ccc(-c3nnc(C(F)F)o3)cn2)c2ccc(F)c(Cl)c2)CC1] |
| 243 | 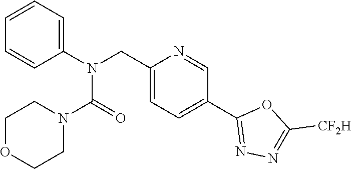 [SMILES: O=C(N1CCOCC1)N(Cc1ccc(-c2nnc(C(F)F)o2)cn1)c1ccccc1] |
| 244 | 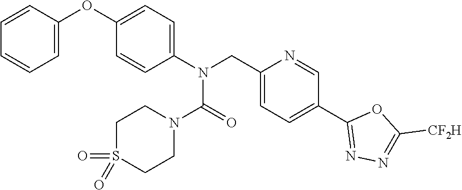 [SMILES: O=C(N1CCS(=O)(=O)CC1)N(Cc1ccc(-c2nnc(C(F)F)o2)cn1)c1ccc(Oc2ccccc2)cc1] |
| 245 | 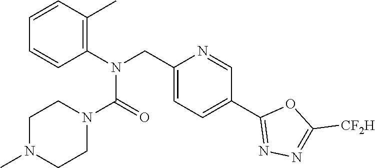 [SMILES: Cc1ccccc1N(Cc1ccc(-c2nnc(C(F)F)o2)cn1)C(=O)N1CCN(C)CC1] |
| 246 | 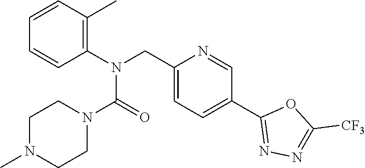 [SMILES: Cc1ccccc1N(Cc1ccc(-c2nnc(C(F)(F)F)o2)cn1)C(=O)N1CCN(C)CC1] |
| 247 | 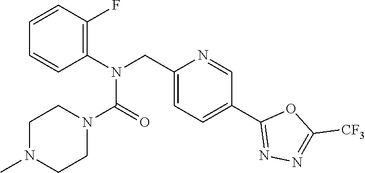 [SMILES: CN1CCN(C(=O)N(Cc2ccc(-c3nnc(C(F)(F)F)o3)cn2)c2ccccc2F)CC1] |
| 248 | 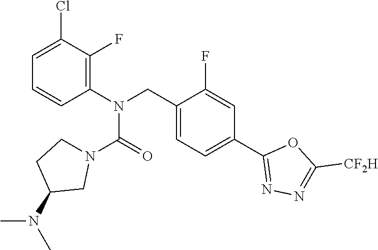 [SMILES: CN(C)[C@H]1CCN(C(=O)N(Cc2ccc(-c3nnc(C(F)F)o3)cc2F)c2cccc(Cl)c2F)C1] |
| 249 | 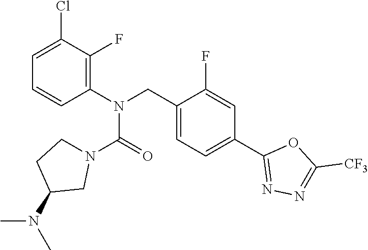 [SMILES: CN(C)[C@H]1CCN(C(=O)N(Cc2ccc(-c3nnc(C(F)(F)F)o3)cc2F)c2cccc(Cl)c2F)C1] |
| 250 | 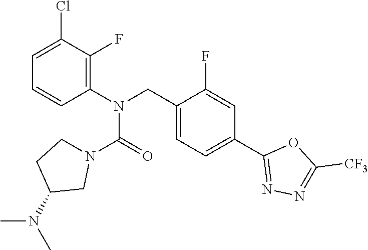 [SMILES: CN(C)[C@@H]1CCN(C(=O)N(Cc2ccc(-c3nnc(C(F)(F)F)o3)cc2F)c2cccc(Cl)c2F)C1] |
| 251 | 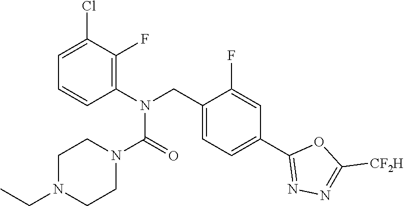 [SMILES: CCN1CCN(C(=O)N(Cc2ccc(-c3nnc(C(F)F)o3)cc2F)c2cccc(Cl)c2F)CC1] |
| 252 | 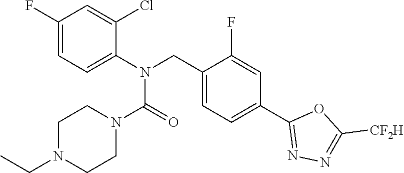 [SMILES: CCN1CCN(C(=O)N(Cc2ccc(-c3nnc(C(F)F)o3)cc2F)c2ccc(F)cc2Cl)CC1] |
| 253 | 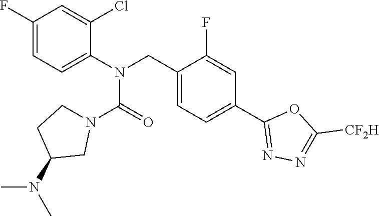 [SMILES: CN(C)[C@H]1CCN(C(=O)N(Cc2ccc(-c3nnc(C(F)F)o3)cc2F)c2ccc(F)cc2Cl)C1] |
| 254 | 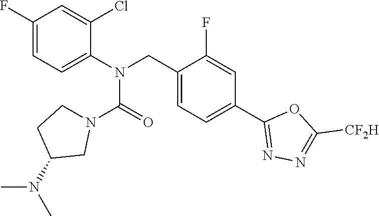 [SMILES: CN(C)[C@@H]1CCN(C(=O)N(Cc2ccc(-c3nnc(C(F)F)o3)cc2F)c2ccc(F)cc2Cl)C1] |
| 255 | 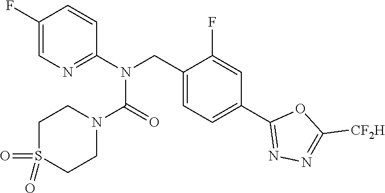 [SMILES: O=C(N1CCS(=O)(=O)CC1)N(Cc1ccc(-c2nnc(C(F)F)o2)cc1F)c1ccc(F)cn1] |
| 256 | 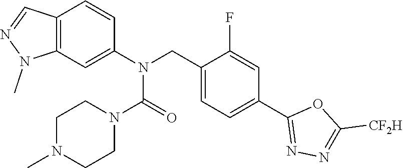 [SMILES: CN1CCN(C(=O)N(Cc2ccc(-c3nnc(C(F)F)o3)cc2F)c2ccc3cnn(C)c3c2)CC1] |
| 257 | 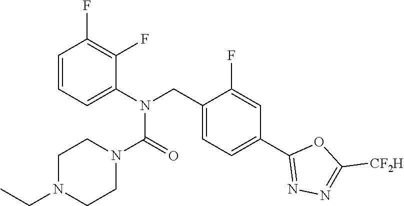 [SMILES: CCN1CCN(C(=O)N(Cc2ccc(-c3nnc(C(F)F)o3)cc2F)c2cccc(F)c2F)CC1] |
| 258 | 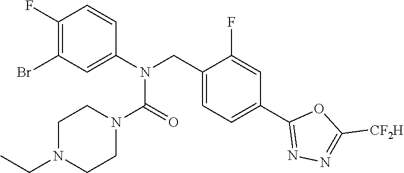 [SMILES: CCN1CCN(C(=O)N(Cc2ccc(-c3nnc(C(F)F)o3)cc2F)c2ccc(F)c(Br)c2)CC1] |
| 259 | 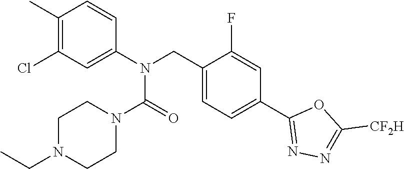 [SMILES: CCN1CCN(C(=O)N(Cc2ccc(-c3nnc(C(F)F)o3)cc2F)c2ccc(C)c(Cl)c2)CC1] |
| 260 | 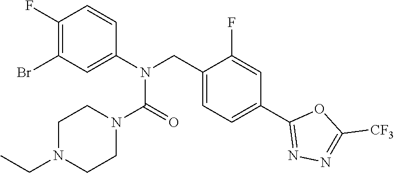 [SMILES: CCN1CCN(C(=O)N(Cc2ccc(-c3nnc(C(F)(F)F)o3)cc2F)c2ccc(F)c(Br)c2)CC1] |
| 261 | 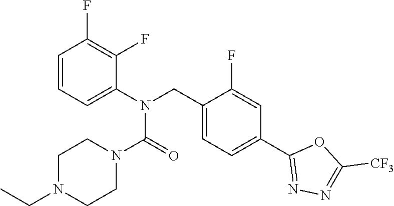 [SMILES: CCN1CCN(C(=O)N(Cc2ccc(-c3nnc(C(F)(F)F)o3)cc2F)c2cccc(F)c2F)CC1] |
| 262 | 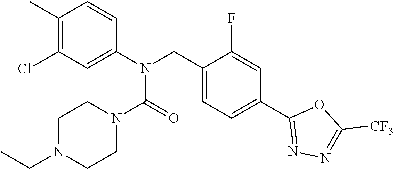 [SMILES: CCN1CCN(C(=O)N(Cc2ccc(-c3nnc(C(F)(F)F)o3)cc2F)c2ccc(C)c(Cl)c2)CC1] |
| 263 | 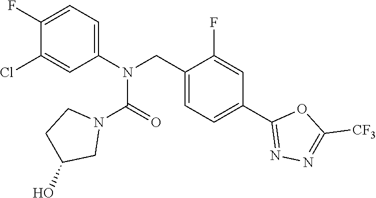 [SMILES: O=C(N1CC[C@@H](O)C1)N(Cc1ccc(-c2nnc(C(F)(F)F)o2)cc1F)c1ccc(F)c(Cl)c1] |
| 264 | 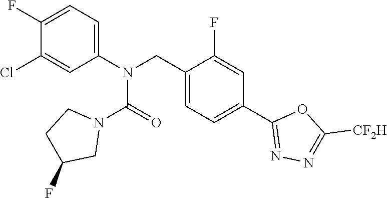 [SMILES: O=C(N1CC[C@H](F)C1)N(Cc1ccc(-c2nnc(C(F)F)o2)cc1F)c1ccc(F)c(Cl)c1] |
| 265 | 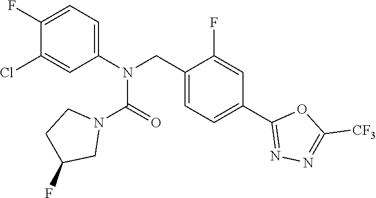 [SMILES: O=C(N1CC[C@H](F)C1)N(Cc1ccc(-c2nnc(C(F)(F)F)o2)cc1F)c1ccc(F)c(Cl)c1] |
| 266 | 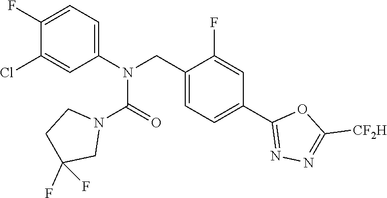 [SMILES: O=C(N1CCC(F)(F)C1)N(Cc1ccc(-c2nnc(C(F)F)o2)cc1F)c1ccc(F)c(Cl)c1] |
| 267 | 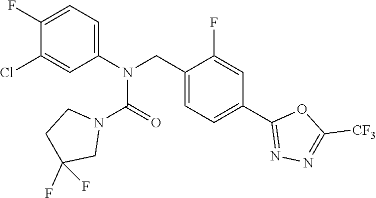 [SMILES: O=C(N1CCC(F)(F)C1)N(Cc1ccc(-c2nnc(C(F)(F)F)o2)cc1F)c1ccc(F)c(Cl)c1] |
| 268 | 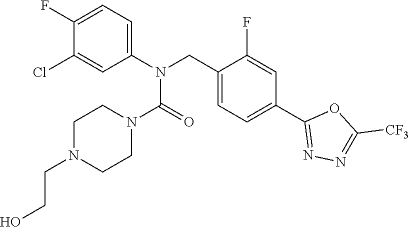 [SMILES: O=C(N1CCN(CCO)CC1)N(Cc1ccc(-c2nnc(C(F)(F)F)o2)cc1F)c1ccc(F)c(Cl)c1] |
| 269 | 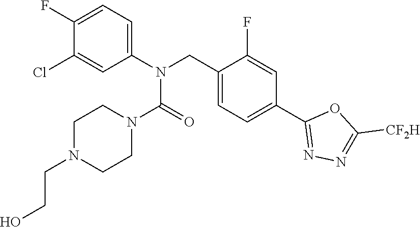 [SMILES: O=C(N1CCN(CCO)CC1)N(Cc1ccc(-c2nnc(C(F)F)o2)cc1F)c1ccc(F)c(Cl)c1] |
| 270 | 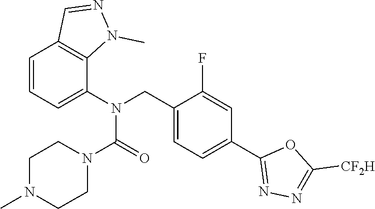 [SMILES: CN1CCN(C(=O)N(Cc2ccc(-c3nnc(C(F)F)o3)cc2F)c2cccc3cnn(C)c23)CC1] |
| 271 | 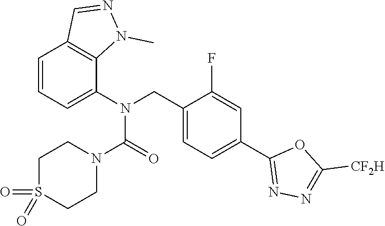 [SMILES: Cn1ncc2cccc(N(Cc3ccc(-c4nnc(C(F)F)o4)cc3F)C(=O)N3CCS(=O)(=O)CC3)c21] |
| 272 | 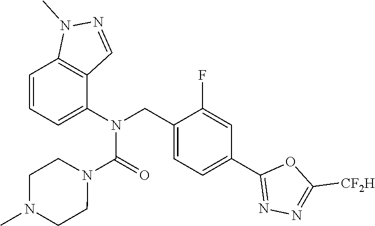 [SMILES: CN1CCN(C(=O)N(Cc2ccc(-c3nnc(C(F)F)o3)cc2F)c2cccc3c2cnn3C)CC1] |
| 273 | 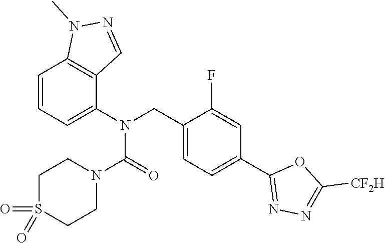 [SMILES: Cn1ncc2c(N(Cc3ccc(-c4nnc(C(F)F)o4)cc3F)C(=O)N3CCS(=O)(=O)CC3)cccc21] |
| 274 | 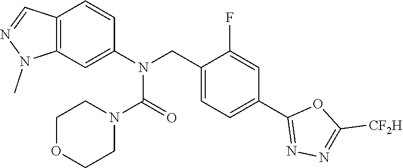 [SMILES: Cn1ncc2ccc(N(Cc3ccc(-c4nnc(C(F)F)o4)cc3F)C(=O)N3CCOCC3)cc21] |
| 275 | 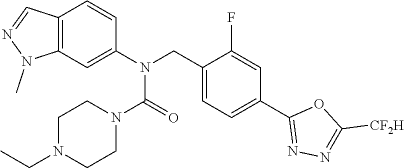 [SMILES: CCN1CCN(C(=O)N(Cc2ccc(-c3nnc(C(F)F)o3)cc2F)c2ccc3cnn(C)c3c2)CC1] |
| 276 | 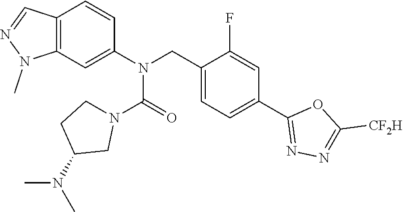 [SMILES: CN(C)[C@@H]1CCN(C(=O)N(Cc2ccc(-c3nnc(C(F)F)o3)cc2F)c2ccc3cnn(C)c3c2)C1] |
| 277 | 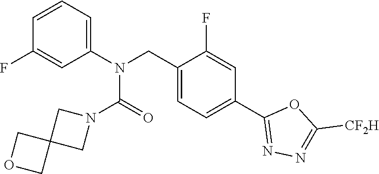 [SMILES: O=C(N1CC2(COC2)C1)N(Cc1ccc(-c2nnc(C(F)F)o2)cc1F)c1cccc(F)c1] |
| 278 | 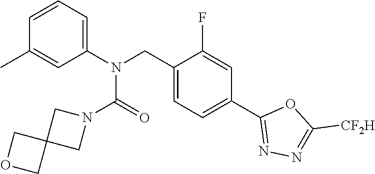 [SMILES: Cc1cccc(N(Cc2ccc(-c3nnc(C(F)F)o3)cc2F)C(=O)N2CC3(COC3)C2)c1] |
| 279 | 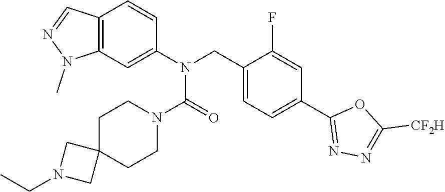 [SMILES: CCN1CC2(CCN(C(=O)N(Cc3ccc(-c4nnc(C(F)F)o4)cc3F)c3ccc4cnn(C)c4c3)CC2)C1] |
| 280 | 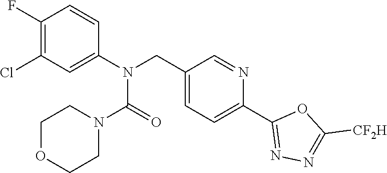 [SMILES: O=C(N1CCOCC1)N(Cc1ccc(-c2nnc(C(F)F)o2)nc1)c1ccc(F)c(Cl)c1] |
| 281 | 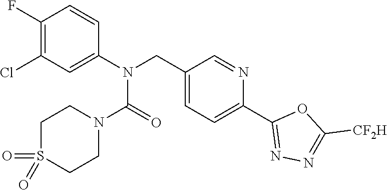 [SMILES: O=C(N1CCS(=O)(=O)CC1)N(Cc1ccc(-c2nnc(C(F)F)o2)nc1)c1ccc(F)c(Cl)c1] |
| 282 | 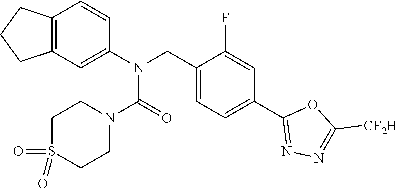 [SMILES: O=C(N1CCS(=O)(=O)CC1)N(Cc1ccc(-c2nnc(C(F)F)o2)cc1F)c1ccc2c(c1)CCC2] |
| 283 | 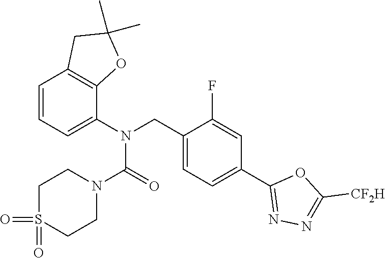 [SMILES: CC1(C)Cc2cccc(N(Cc3ccc(-c4nnc(C(F)F)o4)cc3F)C(=O)N3CCS(=O)(=O)CC3)c2O1] |
| 284 | 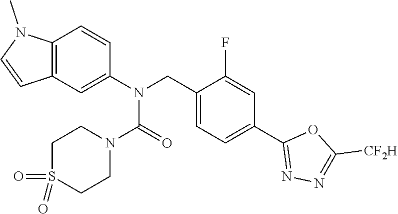 [SMILES: Cn1ccc2cc(N(Cc3ccc(-c4nnc(C(F)F)o4)cc3F)C(=O)N3CCS(=O)(=O)CC3)ccc21] |
| 285 | 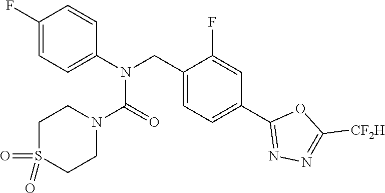 [SMILES: O=C(N1CCS(=O)(=O)CC1)N(Cc1ccc(-c2nnc(C(F)F)o2)cc1F)c1ccc(F)cc1] |
| 286 | 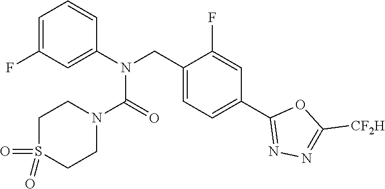 [SMILES: O=C(N1CCS(=O)(=O)CC1)N(Cc1ccc(-c2nnc(C(F)F)o2)cc1F)c1cccc(F)c1] |
| 287 | 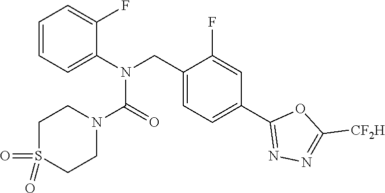 [SMILES: O=C(N1CCS(=O)(=O)CC1)N(Cc1ccc(-c2nnc(C(F)F)o2)cc1F)c1ccccc1F] |
| 288 | 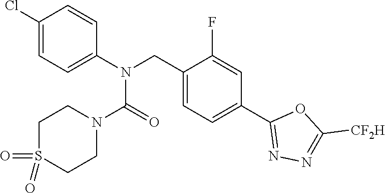 [SMILES: O=C(N1CCS(=O)(=O)CC1)N(Cc1ccc(-c2nnc(C(F)F)o2)cc1F)c1ccc(Cl)cc1] |
| 289 | 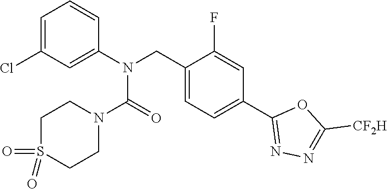 [SMILES: O=C(N1CCS(=O)(=O)CC1)N(Cc1ccc(-c2nnc(C(F)F)o2)cc1F)c1cccc(Cl)c1] |
| 290 | 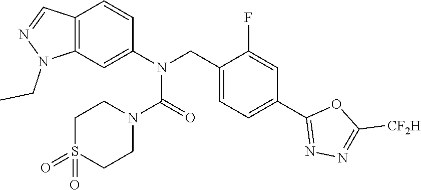 [SMILES: CCn1ncc2ccc(N(Cc3ccc(-c4nnc(C(F)F)o4)cc3F)C(=O)N3CCS(=O)(=O)CC3)cc21] |
| 291 | 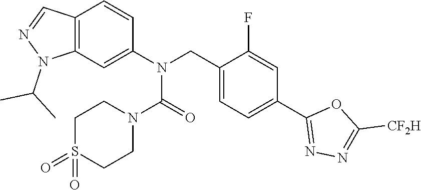 [SMILES: CC(C)n1ncc2ccc(N(Cc3ccc(-c4nnc(C(F)F)o4)cc3F)C(=O)N3CCS(=O)(=O)CC3)cc21] |
| 292 | 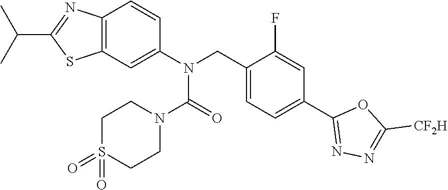 [SMILES: CC(C)c1nc2ccc(N(Cc3ccc(-c4nnc(C(F)F)o4)cc3F)C(=O)N3CCS(=O)(=O)CC3)cc2s1] |
| 293 | 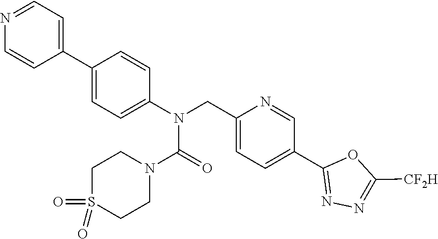 [SMILES: O=C(N1CCS(=O)(=O)CC1)N(Cc1ccc(-c2nnc(C(F)F)o2)cn1)c1ccc(-c2ccncc2)cc1] |
| 294 | 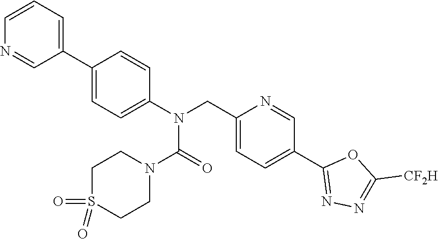 [SMILES: O=C(N1CCS(=O)(=O)CC1)N(Cc1ccc(-c2nnc(C(F)F)o2)cn1)c1ccc(-c2cccnc2)cc1] |
| 295 | 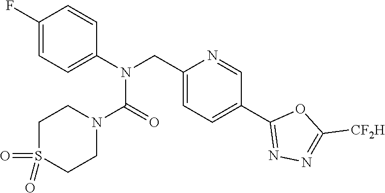 [SMILES: O=C(N1CCS(=O)(=O)CC1)N(Cc1ccc(-c2nnc(C(F)F)o2)cn1)c1ccc(F)cc1] |
| 296 | 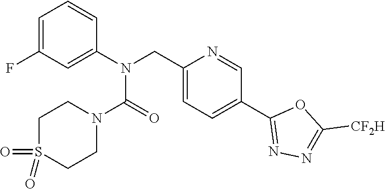 [SMILES: O=C(N1CCS(=O)(=O)CC1)N(Cc1ccc(-c2nnc(C(F)F)o2)cn1)c1cccc(F)c1] |
| 297 | 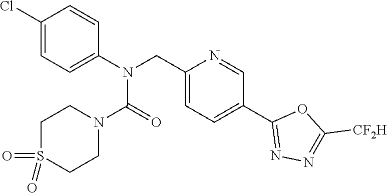 [SMILES: O=C(N1CCS(=O)(=O)CC1)N(Cc1ccc(-c2nnc(C(F)F)o2)cn1)c1ccc(Cl)cc1] |
| 298 | 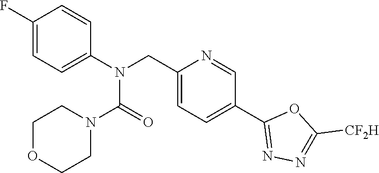 [SMILES: O=C(N1CCOCC1)N(Cc1ccc(-c2nnc(C(F)F)o2)cn1)c1ccc(F)cc1] |
| 299 | 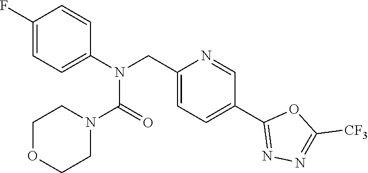 [SMILES: O=C(N1CCOCC1)N(Cc1ccc(-c2nnc(C(F)(F)F)o2)cn1)c1ccc(F)cc1] |
| 300 | 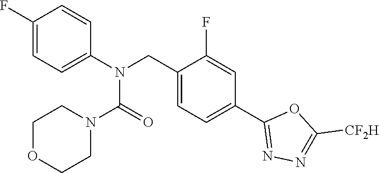 [SMILES: O=C(N1CCOCC1)N(Cc1ccc(-c2nnc(C(F)F)o2)cc1F)c1ccc(F)cc1] |
| 301 | 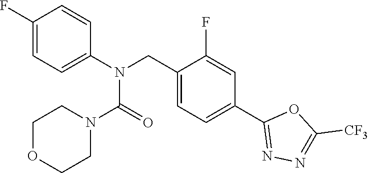 [SMILES: O=C(N1CCOCC1)N(Cc1ccc(-c2nnc(C(F)(F)F)o2)cc1F)c1ccc(F)cc1] |
| 302 | 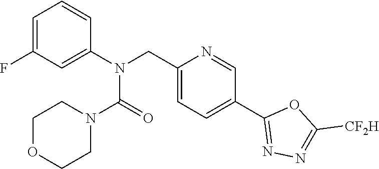 [SMILES: O=C(N1CCOCC1)N(Cc1ccc(-c2nnc(C(F)F)o2)cn1)c1cccc(F)c1] |
| 303 | 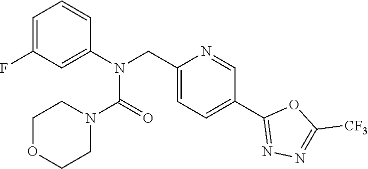 [SMILES: O=C(N1CCOCC1)N(Cc1ccc(-c2nnc(C(F)(F)F)o2)cn1)c1cccc(F)c1] |
| 304 | 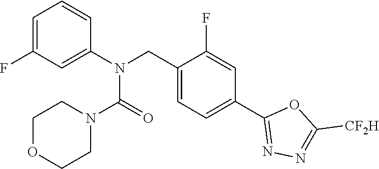 [SMILES: O=C(N1CCOCC1)N(Cc1ccc(-c2nnc(C(F)F)o2)cc1F)c1cccc(F)c1] |
| 305 | 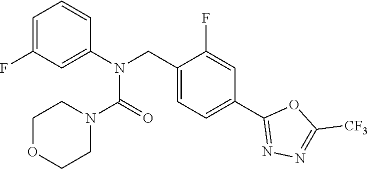 [SMILES: O=C(N1CCOCC1)N(Cc1ccc(-c2nnc(C(F)(F)F)o2)cc1F)c1cccc(F)c1] |
| 306 | 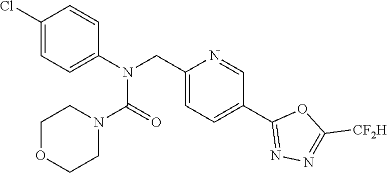 [SMILES: O=C(N1CCOCC1)N(Cc1ccc(-c2nnc(C(F)F)o2)cn1)c1ccc(Cl)cc1] |
| 307 | 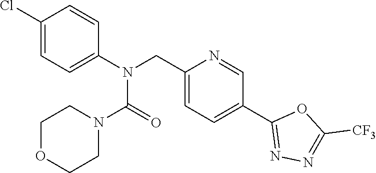 [SMILES: O=C(N1CCOCC1)N(Cc1ccc(-c2nnc(C(F)(F)F)o2)cn1)c1ccc(Cl)cc1] |
| 308 | 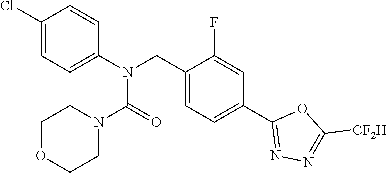 [SMILES: O=C(N1CCOCC1)N(Cc1ccc(-c2nnc(C(F)F)o2)cc1F)c1ccc(Cl)cc1] |
| 309 | 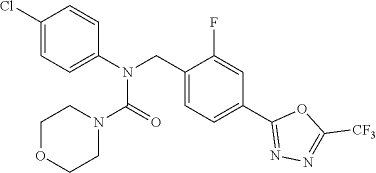 [SMILES: O=C(N1CCOCC1)N(Cc1ccc(-c2nnc(C(F)(F)F)o2)cc1F)c1ccc(Cl)cc1] |
| 310 | 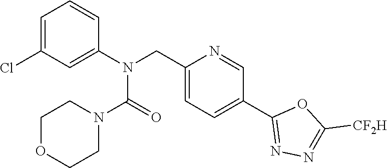 [SMILES: O=C(N1CCOCC1)N(Cc1ccc(-c2nnc(C(F)F)o2)cn1)c1cccc(Cl)c1] |
| 311 | 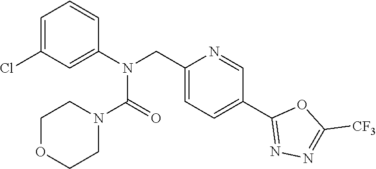 [SMILES: O=C(N1CCOCC1)N(Cc1ccc(-c2nnc(C(F)(F)F)o2)cn1)c1cccc(Cl)c1] |
| 312 | 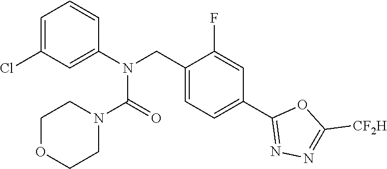 [SMILES: O=C(N1CCOCC1)N(Cc1ccc(-c2nnc(C(F)F)o2)cc1F)c1cccc(Cl)c1] |
| 313 | 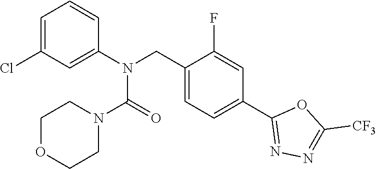 [SMILES: O=C(N1CCOCC1)N(Cc1ccc(-c2nnc(C(F)(F)F)o2)cc1F)c1cccc(Cl)c1] |
| 314 | 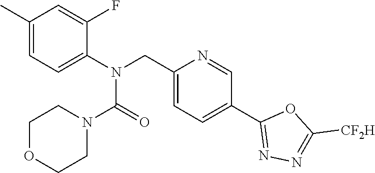 [SMILES: Cc1ccc(N(Cc2ccc(-c3nnc(C(F)F)o3)cn2)C(=O)N2CCOCC2)c(F)c1] |
| 315 | 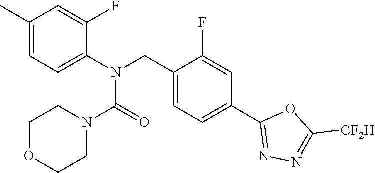 [SMILES: Cc1ccc(N(Cc2ccc(-c3nnc(C(F)F)o3)cc2F)C(=O)N2CCOCC2)c(F)c1] |
| 316 | 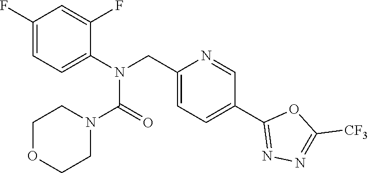 [SMILES: O=C(N1CCOCC1)N(Cc1ccc(-c2nnc(C(F)(F)F)o2)cn1)c1ccc(F)cc1F] |
| 317 | 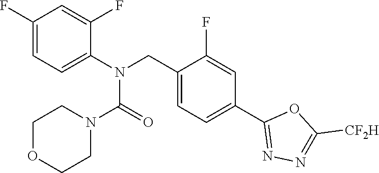 [SMILES: O=C(N1CCOCC1)N(Cc1ccc(-c2nnc(C(F)F)o2)cc1F)c1ccc(F)cc1F] |
| 318 | 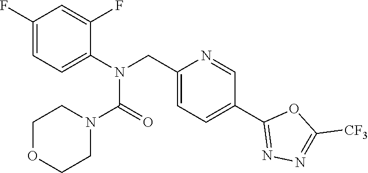 [SMILES: O=C(N1CCOCC1)N(Cc1ccc(-c2nnc(C(F)(F)F)o2)cn1)c1ccc(F)cc1F] |
| 319 | 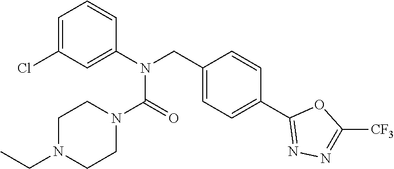 [SMILES: CCN1CCN(C(=O)N(Cc2ccc(-c3nnc(C(F)(F)F)o3)cc2)c2cccc(Cl)c2)CC1] |
| 320 | 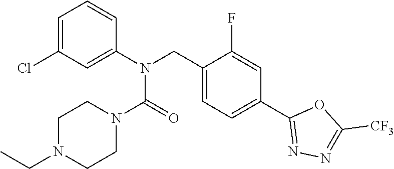 [SMILES: CCN1CCN(C(=O)N(Cc2ccc(-c3nnc(C(F)(F)F)o3)cc2F)c2cccc(Cl)c2)CC1] |
| 321 | 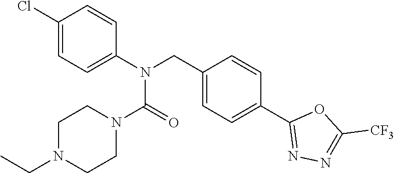 [SMILES: CCN1CCN(C(=O)N(Cc2ccc(-c3nnc(C(F)(F)F)o3)cc2)c2ccc(Cl)cc2)CC1] |
| 322 | 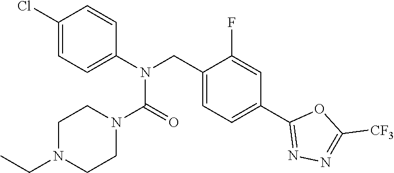 [SMILES: CCN1CCN(C(=O)N(Cc2ccc(-c3nnc(C(F)(F)F)o3)cc2F)c2ccc(Cl)cc2)CC1] |
| 323 | 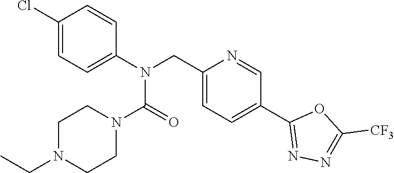 [SMILES: CCN1CCN(C(=O)N(Cc2ccc(-c3nnc(C(F)(F)F)o3)cn2)c2ccc(Cl)cc2)CC1] |
| 324 | 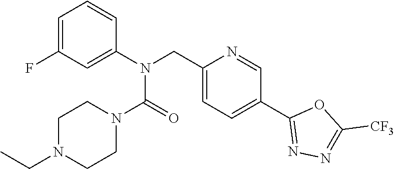 [SMILES: CCN1CCN(C(=O)N(Cc2ccc(-c3nnc(C(F)(F)F)o3)cn2)c2cccc(F)c2)CC1] |
| 325 | 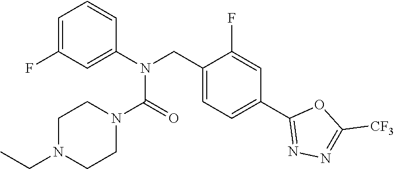 [SMILES: CCN1CCN(C(=O)N(Cc2ccc(-c3nnc(C(F)(F)F)o3)cc2F)c2cccc(F)c2)CC1] |
| 326 | 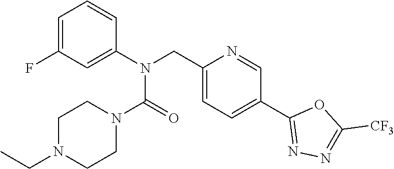 [SMILES: CCN1CCN(C(=O)N(Cc2ccc(-c3nnc(C(F)(F)F)o3)cn2)c2cccc(F)c2)CC1] |
| 327 | 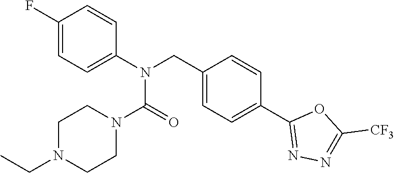 [SMILES: CCN1CCN(C(=O)N(Cc2ccc(-c3nnc(C(F)(F)F)o3)cc2)c2ccc(F)cc2)CC1] |
| 328 | 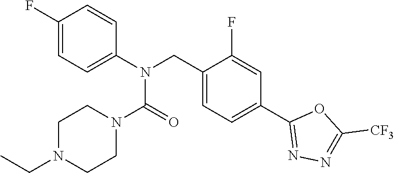 [SMILES: CCN1CCN(C(=O)N(Cc2ccc(-c3nnc(C(F)(F)F)o3)cc2F)c2ccc(F)cc2)CC1] |
| 329 | 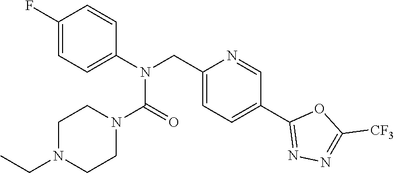 [SMILES: CCN1CCN(C(=O)N(Cc2ccc(-c3nnc(C(F)(F)F)o3)cn2)c2ccc(F)cc2)CC1] |
| 330 | 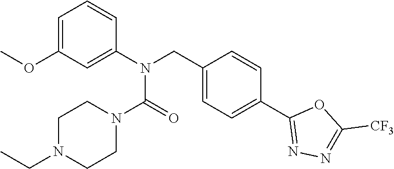 [SMILES: CCN1CCN(C(=O)N(Cc2ccc(-c3nnc(C(F)(F)F)o3)cc2)c2cccc(OC)c2)CC1] |
| 331 | 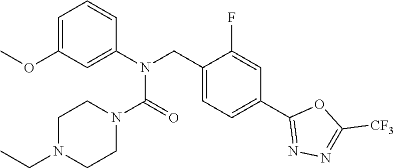 [SMILES: CCN1CCN(C(=O)N(Cc2ccc(-c3nnc(C(F)(F)F)o3)cc2F)c2cccc(OC)c2)CC1] |
| 332 | 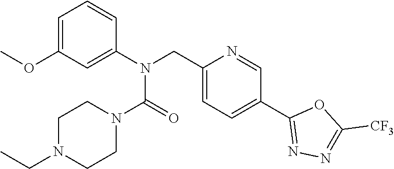 [SMILES: CCN1CCN(C(=O)N(Cc2ccc(-c3nnc(C(F)(F)F)o3)cn2)c2cccc(OC)c2)CC1] |
| 333 | 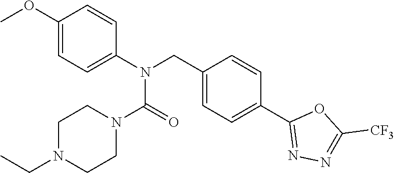 [SMILES: CCN1CCN(C(=O)N(Cc2ccc(-c3nnc(C(F)(F)F)o3)cc2)c2ccc(OC)cc2)CC1] |
| 334 | 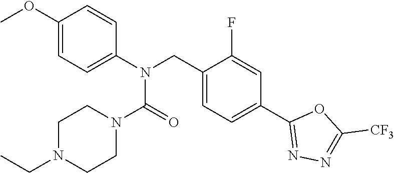 [SMILES: CCN1CCN(C(=O)N(Cc2ccc(-c3nnc(C(F)(F)F)o3)cc2F)c2ccc(OC)cc2)CC1] |
| 335 | 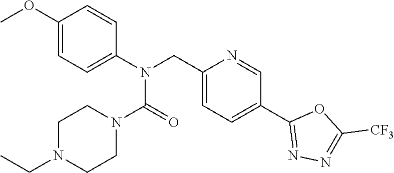 [SMILES: CCN1CCN(C(=O)N(Cc2ccc(-c3nnc(C(F)(F)F)o3)cn2)c2ccc(OC)cc2)CC1] |
| 336 | 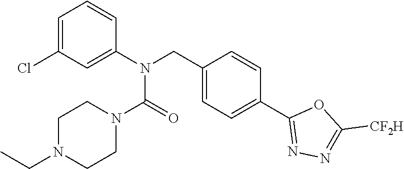 [SMILES: CCN1CCN(C(=O)N(Cc2ccc(-c3nnc(C(F)F)o3)cc2)c2cccc(Cl)c2)CC1] |
| 337 | 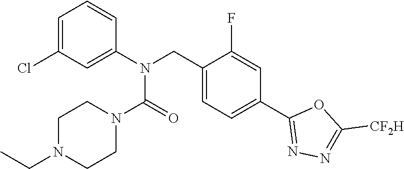 [SMILES: CCN1CCN(C(=O)N(Cc2ccc(-c3nnc(C(F)F)o3)cc2F)c2cccc(Cl)c2)CC1] |
| 338 | 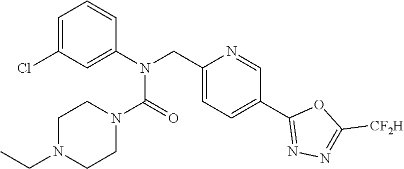 [SMILES: CCN1CCN(C(=O)N(Cc2ccc(-c3nnc(C(F)F)o3)cn2)c2cccc(Cl)c2)CC1] |
| 339 | 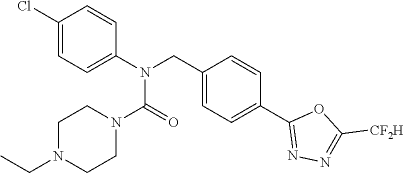 [SMILES: CCN1CCN(C(=O)N(Cc2ccc(-c3nnc(C(F)F)o3)cc2)c2ccc(Cl)cc2)CC1] |
| 340 | 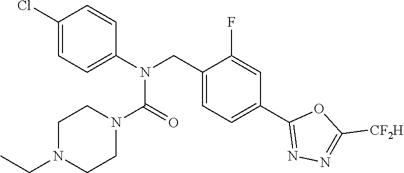 [SMILES: CCN1CCN(C(=O)N(Cc2ccc(-c3nnc(C(F)F)o3)cc2F)c2ccc(Cl)cc2)CC1] |
| 341 | 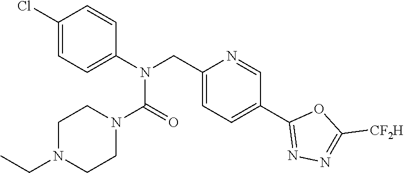 [SMILES: CCN1CCN(C(=O)N(Cc2ccc(-c3nnc(C(F)F)o3)cn2)c2ccc(Cl)cc2)CC1] |
| 342 | 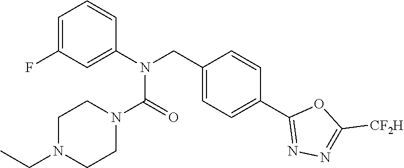 [SMILES: CCN1CCN(C(=O)N(Cc2ccc(-c3nnc(C(F)F)o3)cc2)c2cccc(F)c2)CC1] |
| 343 | 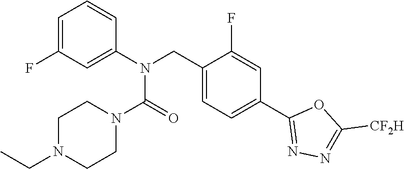 [SMILES: CCN1CCN(C(=O)N(Cc2ccc(-c3nnc(C(F)F)o3)cc2F)c2cccc(F)c2)CC1] |
| 344 | 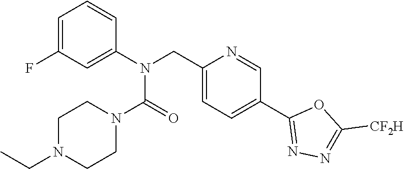 [SMILES: CCN1CCN(C(=O)N(Cc2ccc(-c3nnc(C(F)F)o3)cn2)c2cccc(F)c2)CC1] |
| 345 | 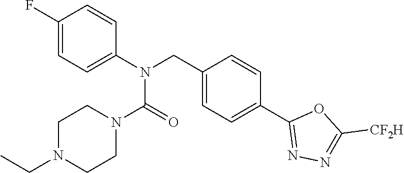 [SMILES: CCN1CCN(C(=O)N(Cc2ccc(-c3nnc(C(F)F)o3)cc2)c2ccc(F)cc2)CC1] |
| 346 | 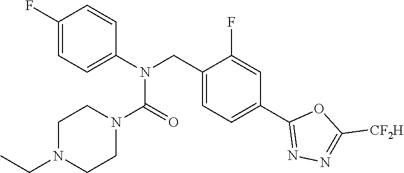 [SMILES: CCN1CCN(C(=O)N(Cc2ccc(-c3nnc(C(F)F)o3)cc2F)c2ccc(F)cc2)CC1] |
| 347 | 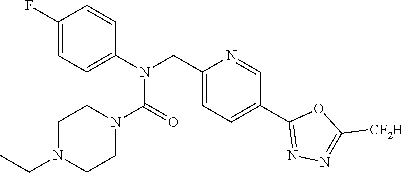 [SMILES: CCN1CCN(C(=O)N(Cc2ccc(-c3nnc(C(F)F)o3)cn2)c2ccc(F)cc2)CC1] |
| 348 | 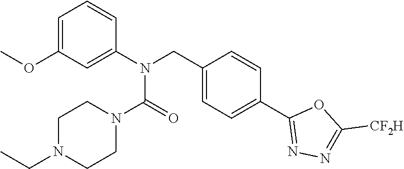 [SMILES: CCN1CCN(C(=O)N(Cc2ccc(-c3nnc(C(F)F)o3)cc2)c2cccc(OC)c2)CC1] |
| 349 | 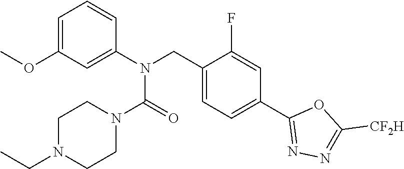 [SMILES: CCN1CCN(C(=O)N(Cc2ccc(-c3nnc(C(F)F)o3)cc2F)c2cccc(OC)c2)CC1] |
| 350 | 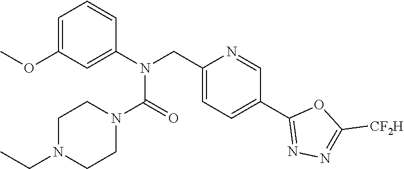 [SMILES: CCN1CCN(C(=O)N(Cc2ccc(-c3nnc(C(F)F)o3)cn2)c2cccc(OC)c2)CC1] |
| 351 | 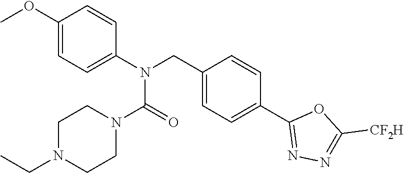 [SMILES: CCN1CCN(C(=O)N(Cc2ccc(-c3nnc(C(F)F)o3)cc2)c2ccc(OC)cc2)CC1] |
| 352 | 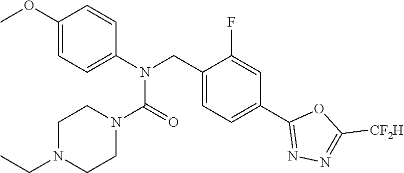 [SMILES: CCN1CCN(C(=O)N(Cc2ccc(-c3nnc(C(F)F)o3)cc2F)c2ccc(OC)cc2)CC1] |
| 353 | 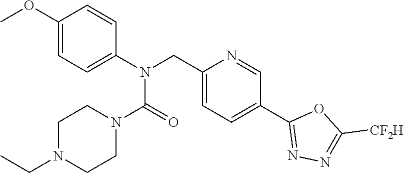 [SMILES: CCN1CCN(C(=O)N(Cc2ccc(-c3nnc(C(F)F)o3)cn2)c2ccc(OC)cc2)CC1] |
| 354 | 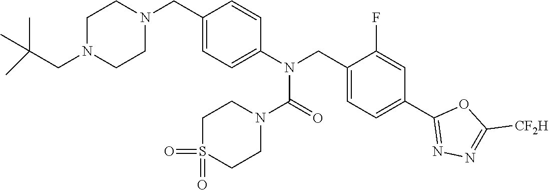 [SMILES: CC(C)(C)CN1CCN(Cc2ccc(N(Cc3ccc(-c4nnc(C(F)F)o4)cc3F)C(=O)N3CCS(=O)(=O)CC3)cc2)CC1] |
| 355 | 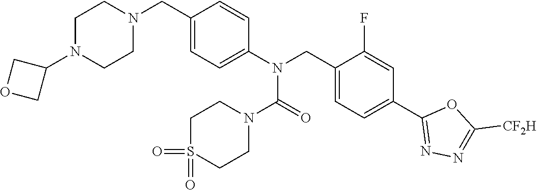 [SMILES: O=C(N1CCS(=O)(=O)CC1)N(Cc1ccc(-c2nnc(C(F)F)o2)cc1F)c1ccc(CN2CCN(C3COC3)CC2)cc1] |
| 356 | 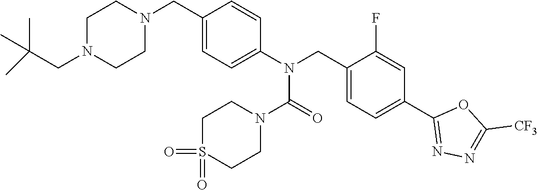 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 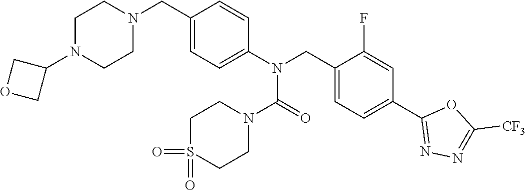 [SMILES: O=C(N1CCS(=O)(=O)CC1)N(Cc1ccc(-c2nnc(C(F)(F)F)o2)cc1F)c1ccc(CN2CCN(C3COC3)CC2)cc1] |
| 358 | 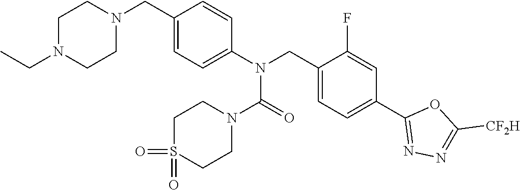 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 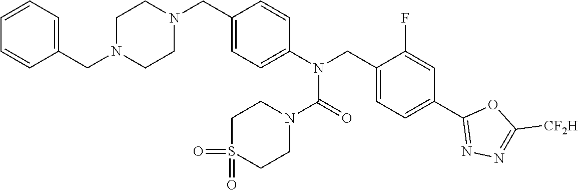 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] |
| 360 | 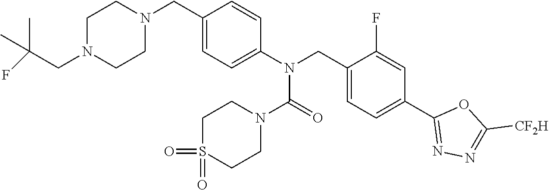 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 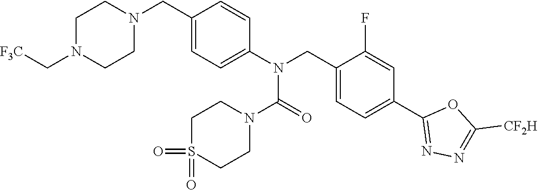 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] |
| 362 | 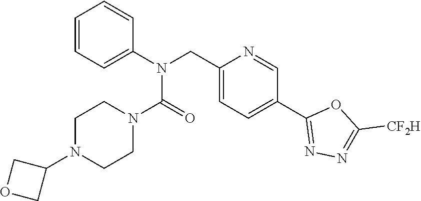 [SMILES: O=C(N1CCN(C2COC2)CC1)N(Cc1ccc(-c2nnc(C(F)F)o2)cn1)c1ccccc1] |
| 363 | 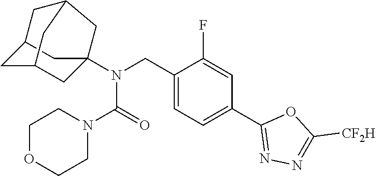 [SMILES: O=C(N1CCOCC1)N(Cc1ccc(-c2nnc(C(F)F)o2)cc1F)C12CC3CC(CC(C3)C1)C2] |
| 364 | 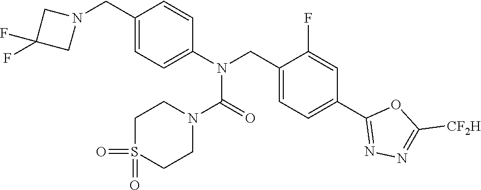 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 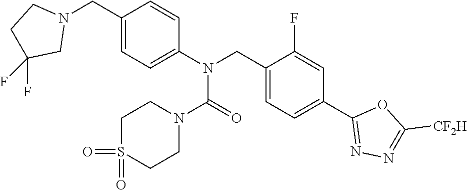 [SMILES: O=C(N1CCS(=O)(=O)CC1)N(Cc1ccc(-c2nnc(C(F)F)o2)cc1F)c1ccc(CN2CCC(F)(F)C2)cc1] |
| 366 | 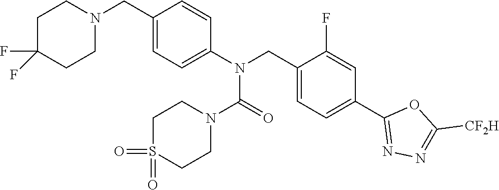 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 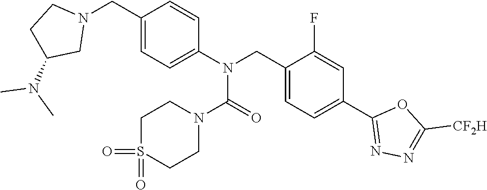 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] |
| 368 | 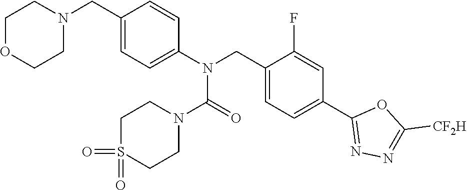 [SMILES: O=C(N1CCS(=O)(=O)CC1)N(Cc1ccc(-c2nnc(C(F)F)o2)cc1F)c1ccc(CN2CCOCC2)cc1] |
| 369 | 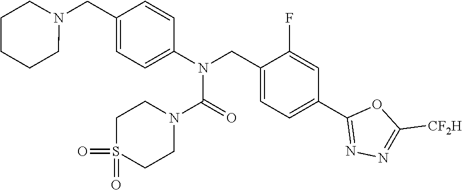 [SMILES: O=C(N1CCS(=O)(=O)CC1)N(Cc1ccc(-c2nnc(C(F)F)o2)cc1F)c1ccc(CN2CCCCC2)cc1] |
| 370 | 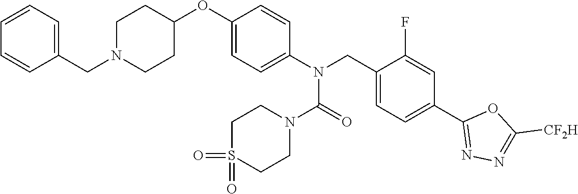 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 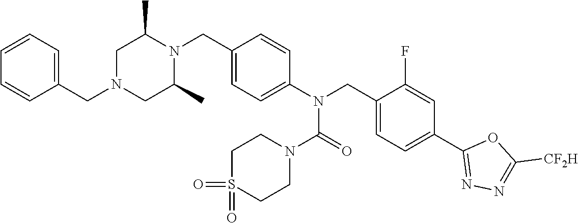 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] |
| 372 | 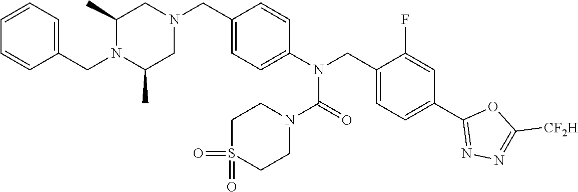 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 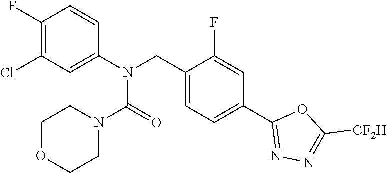 [SMILES: O=C(N1CCOCC1)N(Cc1ccc(-c2nnc(C(F)F)o2)cc1F)c1ccc(F)c(Cl)c1] |
| 374 | 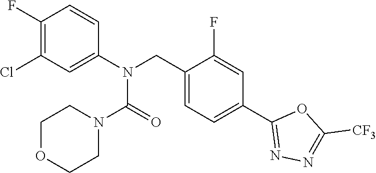 [SMILES: O=C(N1CCOCC1)N(Cc1ccc(-c2nnc(C(F)(F)F)o2)cc1F)c1ccc(F)c(Cl)c1] |
| 375 | 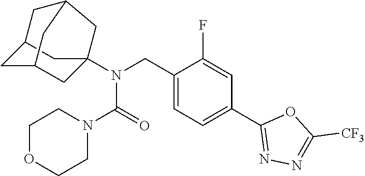 [SMILES: O=C(N1CCOCC1)N(Cc1ccc(-c2nnc(C(F)(F)F)o2)cc1F)C12CC3CC(CC(C3)C1)C2] |
| 376 | 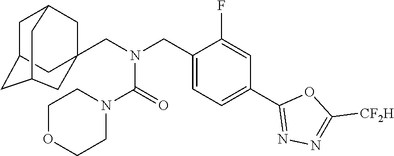 [SMILES: O=C(N1CCOCC1)N(Cc1ccc(-c2nnc(C(F)F)o2)cc1F)CC12CC3CC(CC(C3)C1)C2] |
| 377 | 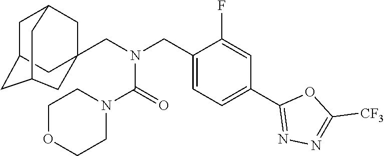 [SMILES: O=C(N1CCOCC1)N(Cc1ccc(-c2nnc(C(F)(F)F)o2)cc1F)CC12CC3CC(CC(C3)C1)C2] |
| 378 | 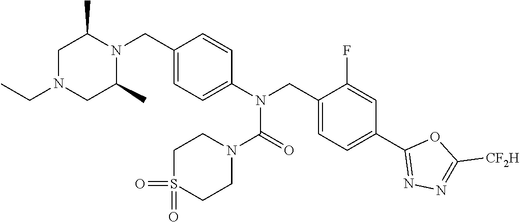 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 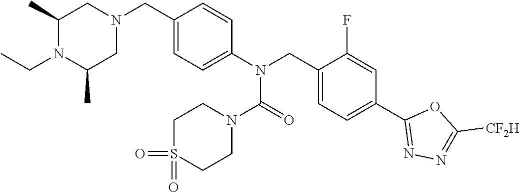 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] |
| 380 | 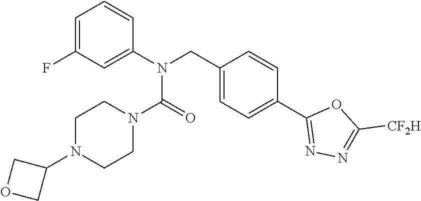 [SMILES: O=C(N1CCN(C2COC2)CC1)N(Cc1ccc(-c2nnc(C(F)F)o2)cc1)c1cccc(F)c1] |
| 381 | 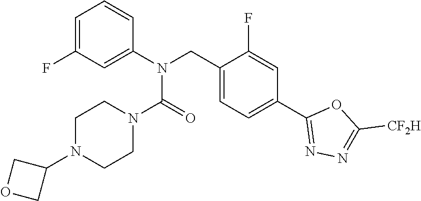 [SMILES: O=C(N1CCN(C2COC2)CC1)N(Cc1ccc(-c2nnc(C(F)F)o2)cc1F)c1cccc(F)c1] |
| 382 | 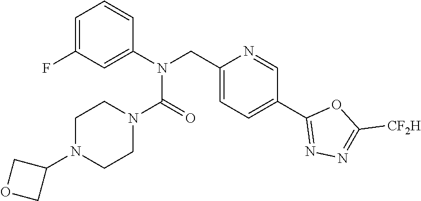 [SMILES: O=C(N1CCN(C2COC2)CC1)N(Cc1ccc(-c2nnc(C(F)F)o2)cn1)c1cccc(F)c1] |
| 383 | 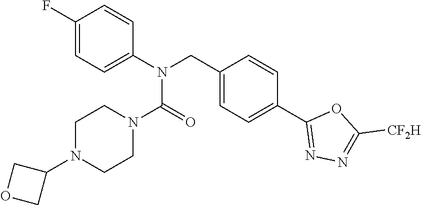 [SMILES: O=C(N1CCN(C2COC2)CC1)N(Cc1ccc(-c2nnc(C(F)F)o2)cc1)c1ccc(F)cc1] |
| 384 | 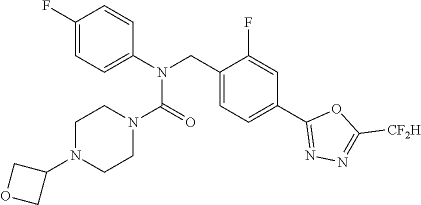 [SMILES: O=C(N1CCN(C2COC2)CC1)N(Cc1ccc(-c2nnc(C(F)F)o2)cc1F)c1ccc(F)cc1] |
| 385 | 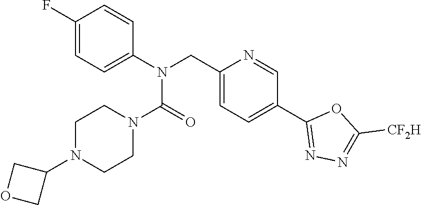 [SMILES: O=C(N1CCN(C2COC2)CC1)N(Cc1ccc(-c2nnc(C(F)F)o2)cn1)c1ccc(F)cc1] |
| 386 | 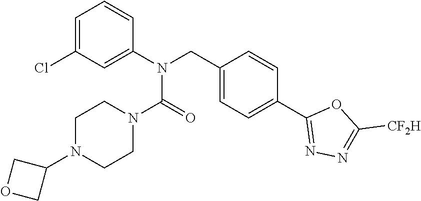 [SMILES: O=C(N1CCN(C2COC2)CC1)N(Cc1ccc(-c2nnc(C(F)F)o2)cc1)c1cccc(Cl)c1] |
| 387 | 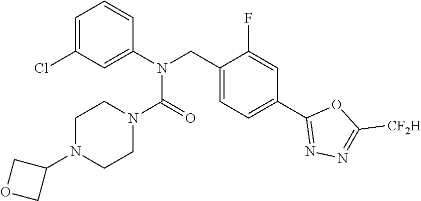 [SMILES: O=C(N1CCN(C2COC2)CC1)N(Cc1ccc(-c2nnc(C(F)F)o2)cc1F)c1cccc(Cl)c1] |
| 388 | 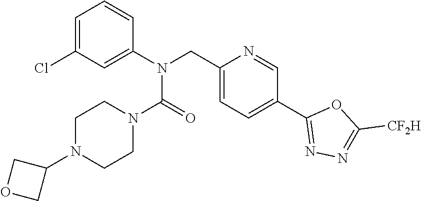 [SMILES: O=C(N1CCN(C2COC2)CC1)N(Cc1ccc(-c2nnc(C(F)F)o2)cn1)c1cccc(Cl)c1] |
| 389 | 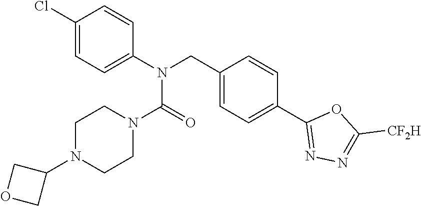 [SMILES: O=C(N1CCN(C2COC2)CC1)N(Cc1ccc(-c2nnc(C(F)F)o2)cc1)c1ccc(Cl)cc1] |
| 390 | 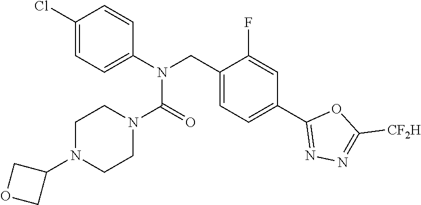 [SMILES: O=C(N1CCN(C2COC2)CC1)N(Cc1ccc(-c2nnc(C(F)F)o2)cc1F)c1ccc(Cl)cc1] |
| 391 | 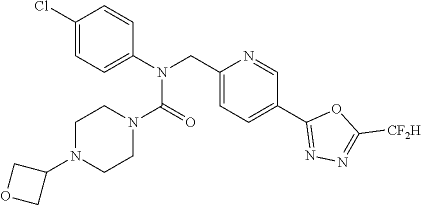 [SMILES: O=C(N1CCN(C2COC2)CC1)N(Cc1ccc(-c2nnc(C(F)F)o2)cn1)c1ccc(Cl)cc1] |
| 392 | 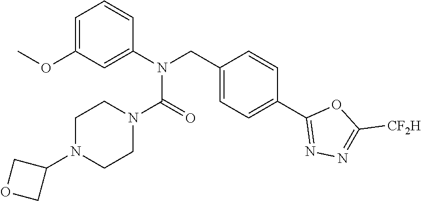 [SMILES: COc1cccc(N(Cc2ccc(-c3nnc(C(F)F)o3)cc2)C(=O)N2CCN(C3COC3)CC2)c1] |
| 393 | 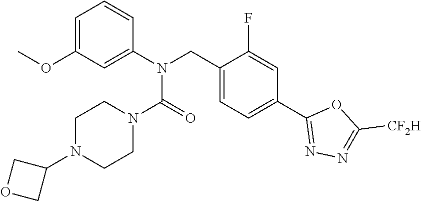 [SMILES: COc1cccc(N(Cc2ccc(-c3nnc(C(F)F)o3)cc2F)C(=O)N2CCN(C3COC3)CC2)c1] |
| 394 | 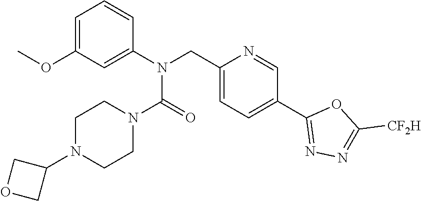 [SMILES: COc1cccc(N(Cc2ccc(-c3nnc(C(F)F)o3)cn2)C(=O)N2CCN(C3COC3)CC2)c1] |
| 395 | 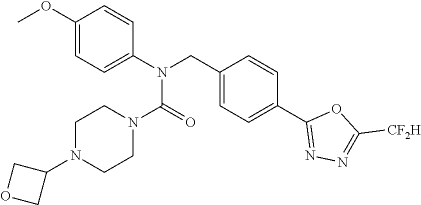 [SMILES: COc1ccc(N(Cc2ccc(-c3nnc(C(F)F)o3)cc2)C(=O)N2CCN(C3COC3)CC2)cc1] |
| 396 | 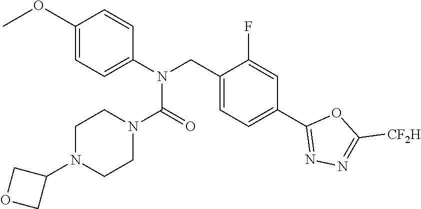 [SMILES: COc1ccc(N(Cc2ccc(-c3nnc(C(F)F)o3)cc2F)C(=O)N2CCN(C3COC3)CC2)cc1] |
| 397 | 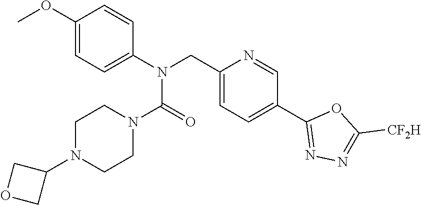 [SMILES: COc1ccc(N(Cc2ccc(-c3nnc(C(F)F)o3)cn2)C(=O)N2CCN(C3COC3)CC2)cc1] |
| 398 | 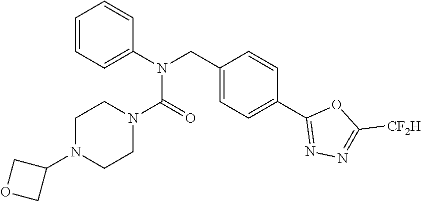 [SMILES: O=C(N1CCN(C2COC2)CC1)N(Cc1ccc(-c2nnc(C(F)F)o2)cc1)c1ccccc1] |
| 399 | 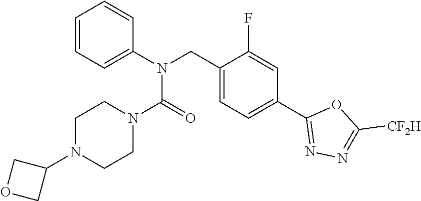 [SMILES: O=C(N1CCN(C2COC2)CC1)N(Cc1ccc(-c2nnc(C(F)F)o2)cc1F)c1ccccc1] |
| 400 | 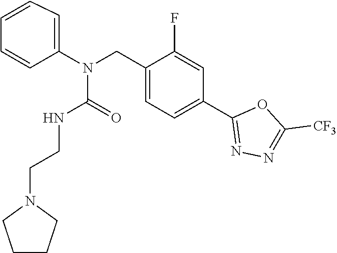 [SMILES: O=C(NCCN1CCCC1)N(Cc1ccc(-c2nnc(C(F)(F)F)o2)cc1F)c1ccccc1] |
| 401 | 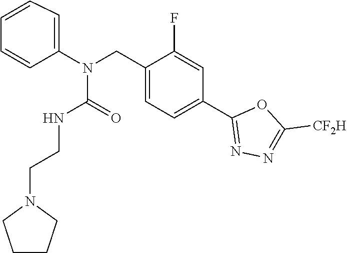 [SMILES: O=C(NCCN1CCCC1)N(Cc1ccc(-c2nnc(C(F)F)o2)cc1F)c1ccccc1] |
| 402 | 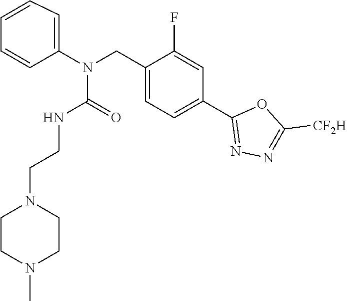 [SMILES: CN1CCN(CCNC(=O)N(Cc2ccc(-c3nnc(C(F)F)o3)cc2F)c2ccccc2)CC1] |
| 403 | 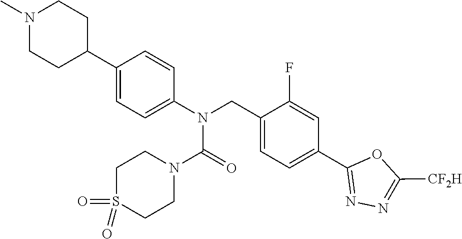 [SMILES: CN1CCC(c2ccc(N(Cc3ccc(-c4nnc(C(F)F)o4)cc3F)C(=O)N3CCS(=O)(=O)CC3)cc2)CC1] |
| 404 | 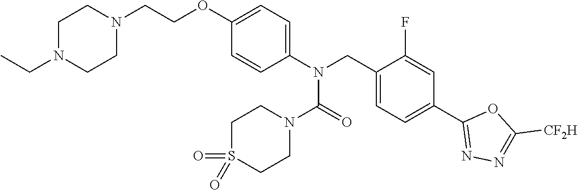 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 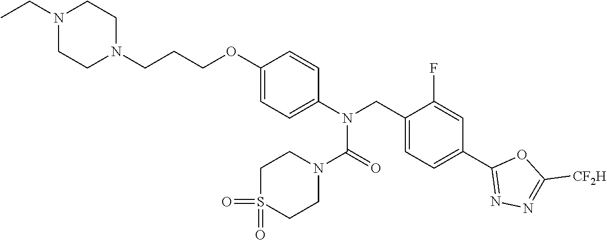 [SMILES: CCN1CCN(CCCOc2ccc(N(Cc3ccc(-c4nnc(C(F)F)o4)cc3F)C(=O)N3CCS(=O)(=O)CC3)cc2)CC1] |
| 406 | 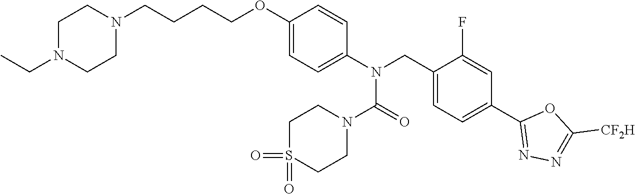 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 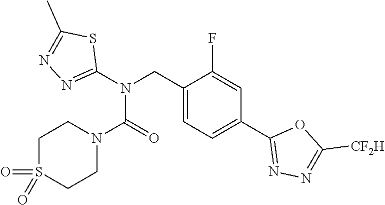 [SMILES: Cc1nnc(N(Cc2ccc(-c3nnc(C(F)F)o3)cc2F)C(=O)N2CCS(=O)(=O)CC2)s1] |
| 408 | 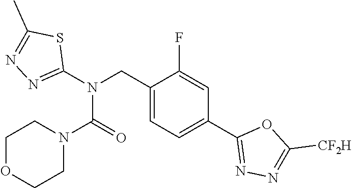 [SMILES: Cc1nnc(N(Cc2ccc(-c3nnc(C(F)F)o3)cc2F)C(=O)N2CCOCC2)s1] |
| 409 | 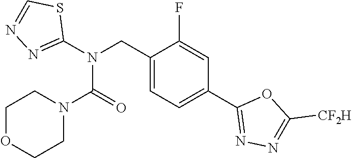 [SMILES: O=C(N1CCOCC1)N(Cc1ccc(-c2nnc(C(F)F)o2)cc1F)c1nncs1] |
| 410 | 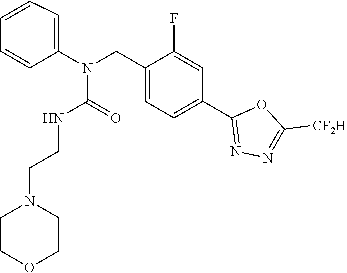 [SMILES: O=C(NCCN1CCOCC1)N(Cc1ccc(-c2nnc(C(F)F)o2)cc1F)c1ccccc1] |
| 411 | 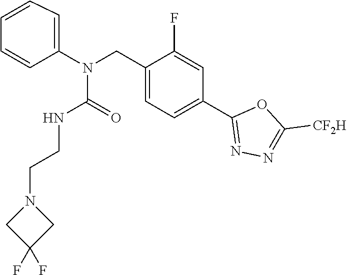 [SMILES: O=C(NCCN1CC(F)(F)C1)N(Cc1ccc(-c2nnc(C(F)F)o2)cc1F)c1ccccc1] |
| 412 | 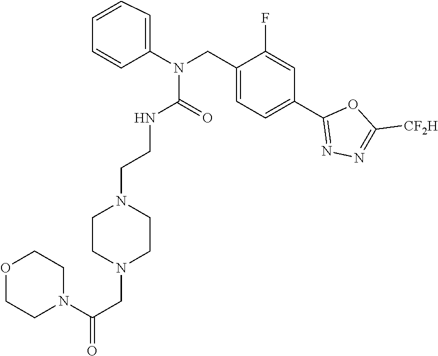 [SMILES: O=C(CN1CCN(CCNC(=O)N(Cc2ccc(-c3nnc(C(F)F)o3)cc2F)c2ccccc2)CC1)N1CCOCC1] |
| 413 | 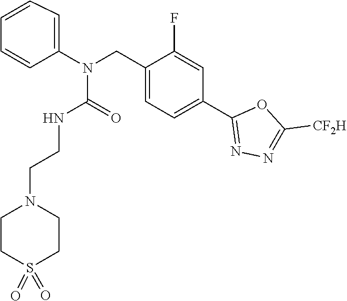 [SMILES: O=C(NCCN1CCS(=O)(=O)CC1)N(Cc1ccc(-c2nnc(C(F)F)o2)cc1F)c1ccccc1] |
| 414 | 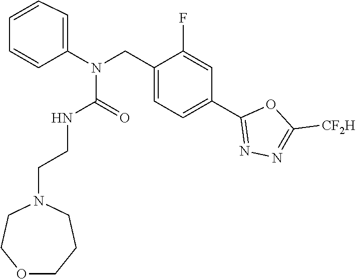 [SMILES: O=C(NCCN1CCCOCC1)N(Cc1ccc(-c2nnc(C(F)F)o2)cc1F)c1ccccc1] |
| 415 | 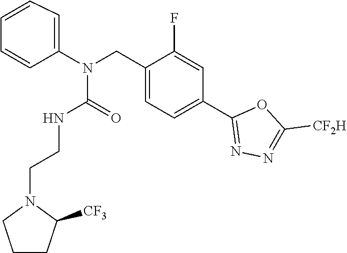 [SMILES: O=C(NCCN1CCC[C@@H]1C(F)(F)F)N(Cc1ccc(-c2nnc(C(F)F)o2)cc1F)c1ccccc1] |
| 416 | 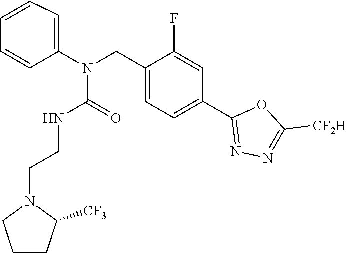 [SMILES: O=C(NCCN1CCC[C@H]1C(F)(F)F)N(Cc1ccc(-c2nnc(C(F)F)o2)cc1F)c1ccccc1] |
| 417 | 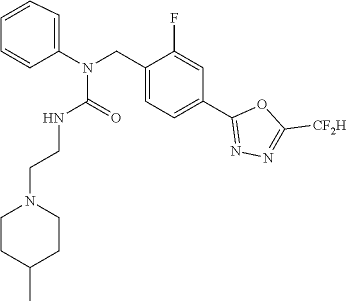 [SMILES: CC1CCN(CCNC(=O)N(Cc2ccc(-c3nnc(C(F)F)o3)cc2F)c2ccccc2)CC1] |
| 418 | 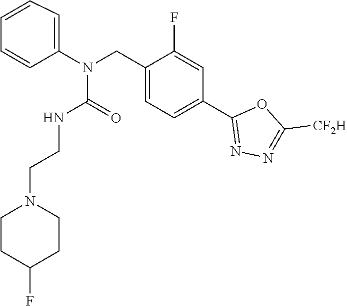 [SMILES: O=C(NCCN1CCC(F)CC1)N(Cc1ccc(-c2nnc(C(F)F)o2)cc1F)c1ccccc1] |
| 419 | 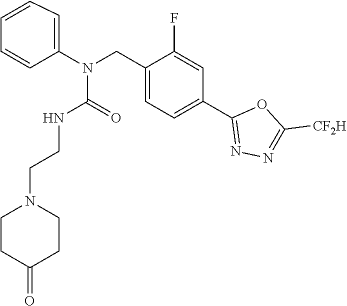 [SMILES: O=C1CCN(CCNC(=O)N(Cc2ccc(-c3nnc(C(F)F)o3)cc2F)c2ccccc2)CC1] |
| 420 | 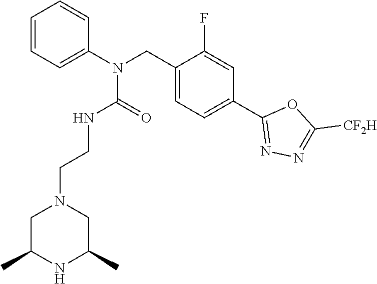 [SMILES: C[C@@H]1CN(CCNC(=O)N(Cc2ccc(-c3nnc(C(F)F)o3)cc2F)c2ccccc2)C[C@H](C)N1] |
| 421 | 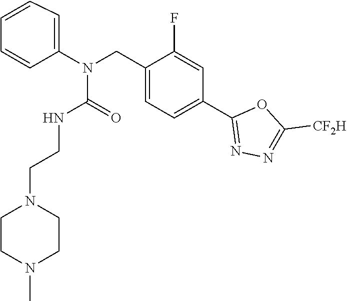 [SMILES: CN1CCN(CCNC(=O)N(Cc2ccc(-c3nnc(C(F)F)o3)cc2F)c2ccccc2)CC1] |
| 422 | 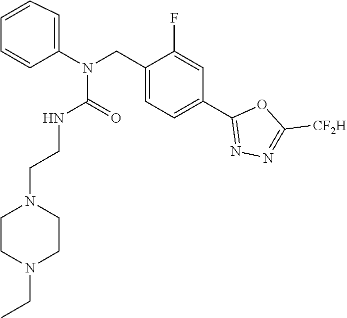 [SMILES: CCN1CCN(CCNC(=O)N(Cc2ccc(-c3nnc(C(F)F)o3)cc2F)c2ccccc2)CC1] |
| 423 | 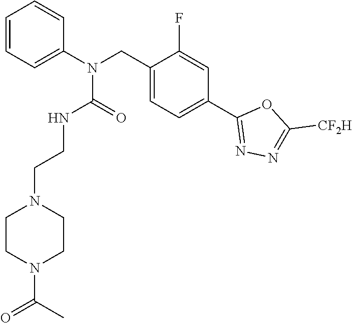 [SMILES: CC(=O)N1CCN(CCNC(=O)N(Cc2ccc(-c3nnc(C(F)F)o3)cc2F)c2ccccc2)CC1] |
| 424 | 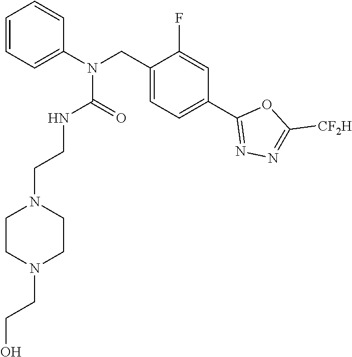 [SMILES: O=C(NCCN1CCN(CCO)CC1)N(Cc1ccc(-c2nnc(C(F)F)o2)cc1F)c1ccccc1] |
| 425 | 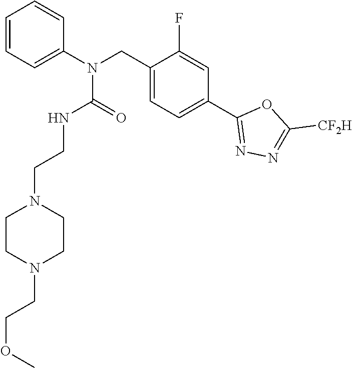 [SMILES: COCCN1CCN(CCNC(=O)N(Cc2ccc(-c3nnc(C(F)F)o3)cc2F)c2ccccc2)CC1] |
| 426 | 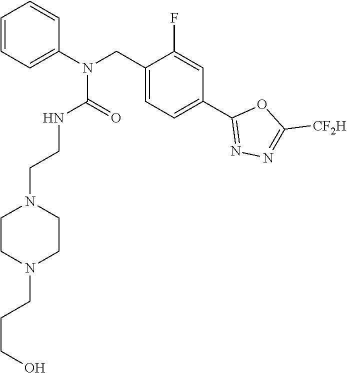 [SMILES: O=C(NCCN1CCN(CCCO)CC1)N(Cc1ccc(-c2nnc(C(F)F)o2)cc1F)c1ccccc1] |
| 427 | 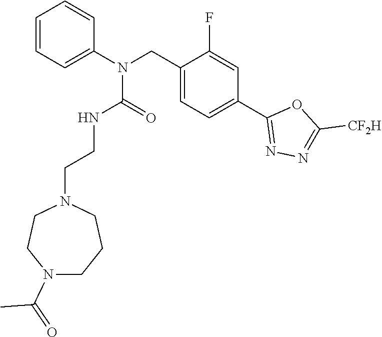 [SMILES: CC(=O)N1CCCN(CCNC(=O)N(Cc2ccc(-c3nnc(C(F)F)o3)cc2F)c2ccccc2)CC1] |
| 428 | 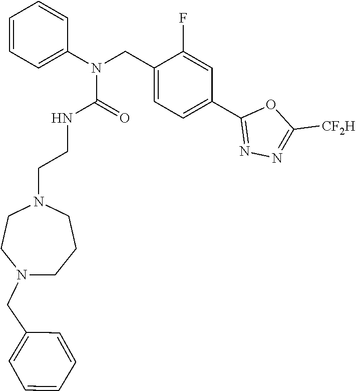 [SMILES: O=C(NCCN1CCCN(Cc2ccccc2)CC1)N(Cc1ccc(-c2nnc(C(F)F)o2)cc1F)c1ccccc1] |
| 429 | 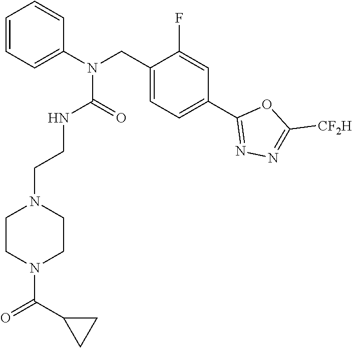 [SMILES: O=C(C1CC1)N1CCN(CCNC(=O)N(Cc2ccc(-c3nnc(C(F)F)o3)cc2F)c2ccccc2)CC1] |
| 430 | 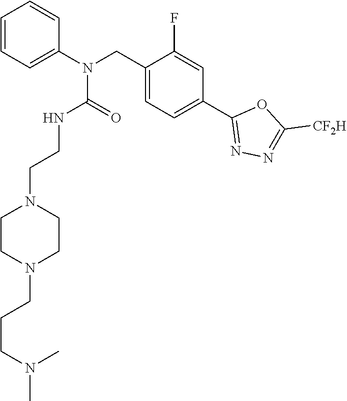 [SMILES: CN(C)CCCN1CCN(CCNC(=O)N(Cc2ccc(-c3nnc(C(F)F)o3)cc2F)c2ccccc2)CC1] |
| 431 | 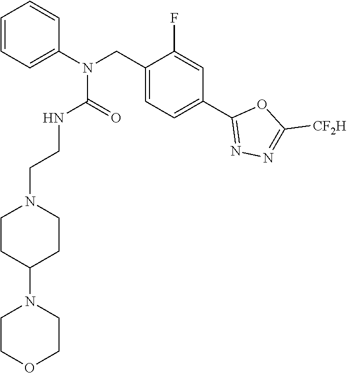 [SMILES: O=C(NCCN1CCC(N2CCOCC2)CC1)N(Cc1ccc(-c2nnc(C(F)F)o2)cc1F)c1ccccc1] |
| 432 | 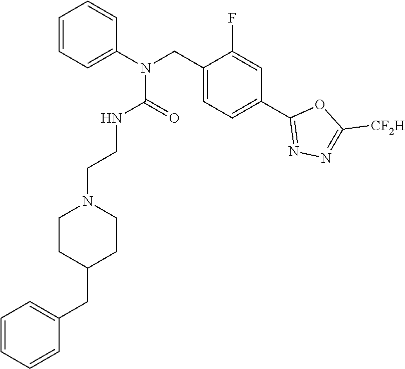 [SMILES: O=C(NCCN1CCC(Cc2ccccc2)CC1)N(Cc1ccc(-c2nnc(C(F)F)o2)cc1F)c1ccccc1] |
| 433 | 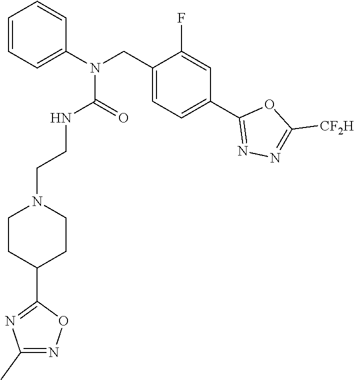 [SMILES: Cc1noc(C2CCN(CCNC(=O)N(Cc3ccc(-c4nnc(C(F)F)o4)cc3F)c3ccccc3)CC2)n1] |
| 434 | 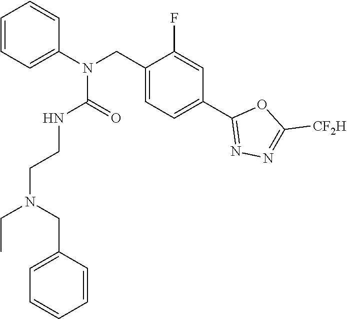 [SMILES: CCN(CCNC(=O)N(Cc1ccc(-c2nnc(C(F)F)o2)cc1F)c1ccccc1)Cc1ccccc1] |
| 435 | 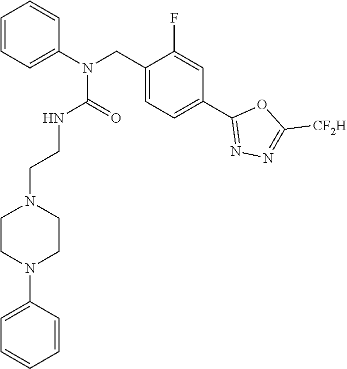 [SMILES: O=C(NCCN1CCN(c2ccccc2)CC1)N(Cc1ccc(-c2nnc(C(F)F)o2)cc1F)c1ccccc1] |
| 436 | 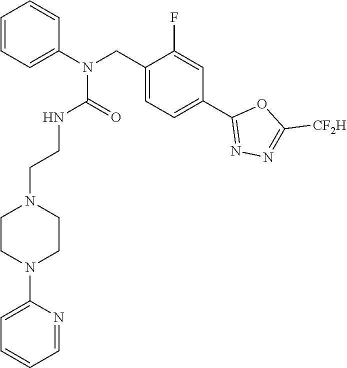 [SMILES: O=C(NCCN1CCN(c2ccccn2)CC1)N(Cc1ccc(-c2nnc(C(F)F)o2)cc1F)c1ccccc1] |
| 437 | 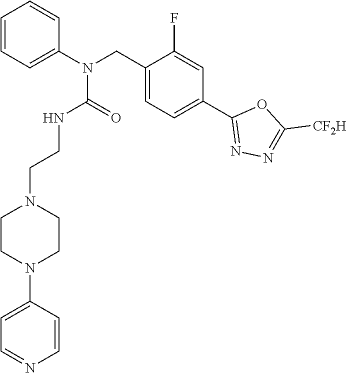 [SMILES: O=C(NCCN1CCN(c2ccncc2)CC1)N(Cc1ccc(-c2nnc(C(F)F)o2)cc1F)c1ccccc1] |
| 438 | 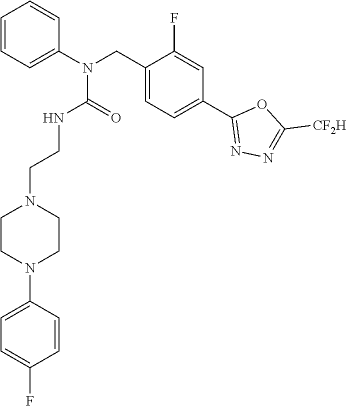 [SMILES: O=C(NCCN1CCN(c2ccc(F)cc2)CC1)N(Cc1ccc(-c2nnc(C(F)F)o2)cc1F)c1ccccc1] |
| 439 | 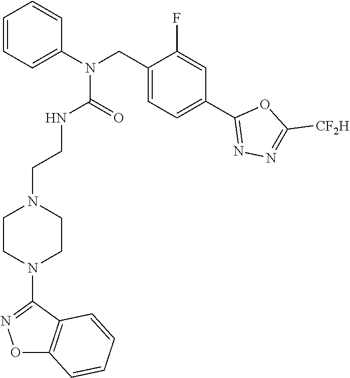 [SMILES: O=C(NCCN1CCN(c2noc3ccccc23)CC1)N(Cc1ccc(-c2nnc(C(F)F)o2)cc1F)c1ccccc1] |
| 440 | 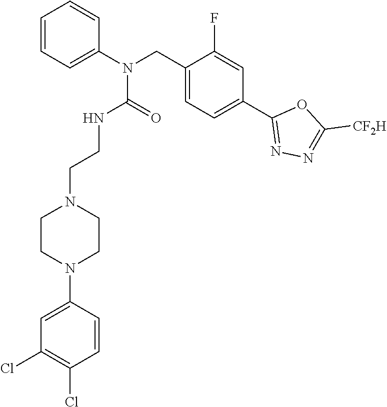 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 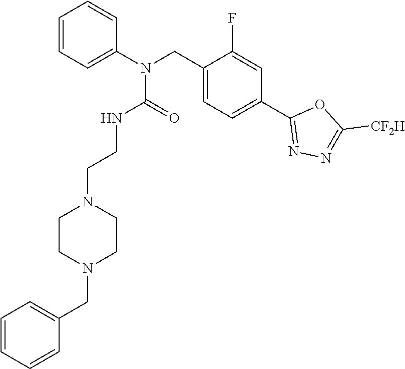 [SMILES: O=C(NCCN1CCN(Cc2ccccc2)CC1)N(Cc1ccc(-c2nnc(C(F)F)o2)cc1F)c1ccccc1] |
| 442 | 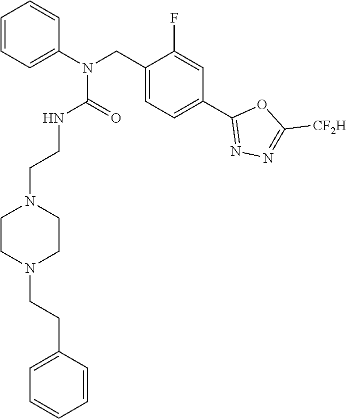 [SMILES: O=C(NCCN1CCN(CCc2ccccc2)CC1)N(Cc1ccc(-c2nnc(C(F)F)o2)cc1F)c1ccccc1] |
| 443 | 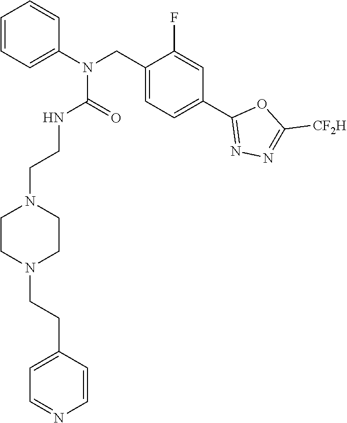 [SMILES: O=C(NCCN1CCN(CCc2ccncc2)CC1)N(Cc1ccc(-c2nnc(C(F)F)o2)cc1F)c1ccccc1] |
| 444 | 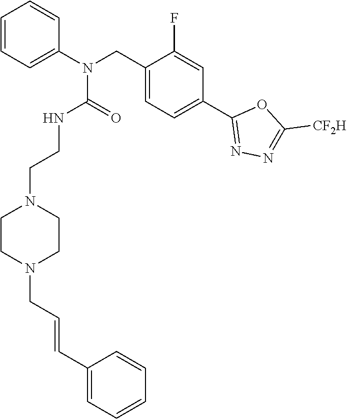 [SMILES: O=C(NCCN1CCN(C/C=C/c2ccccc2)CC1)N(Cc1ccc(-c2nnc(C(F)F)o2)cc1F)c1ccccc1] |
| 445 | 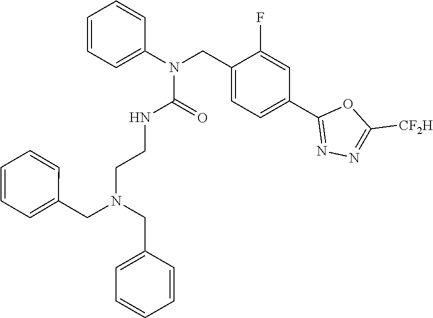 [SMILES: O=C(NCCN(Cc1ccccc1)Cc1ccccc1)N(Cc1ccc(-c2nnc(C(F)F)o2)cc1F)c1ccccc1] |
| 446 | 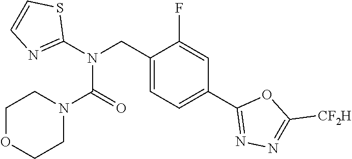 [SMILES: O=C(N1CCOCC1)N(Cc1ccc(-c2nnc(C(F)F)o2)cc1F)c1nccs1] |
| 447 | 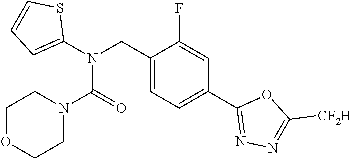 [SMILES: O=C(N1CCOCC1)N(Cc1ccc(-c2nnc(C(F)F)o2)cc1F)c1cccs1] |
| 448 | 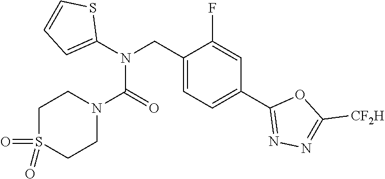 [SMILES: O=C(N1CCS(=O)(=O)CC1)N(Cc1ccc(-c2nnc(C(F)F)o2)cc1F)c1cccs1] |
| 449 | 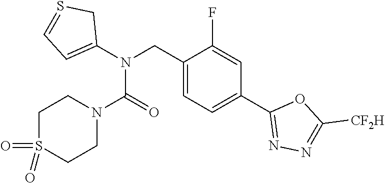 |
| 450 | 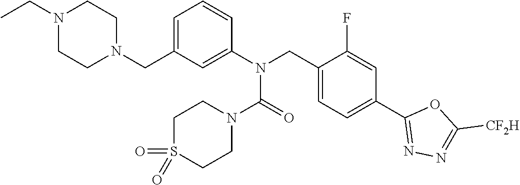 [SMILES: CCN1CCN(Cc2cccc(N(Cc3ccc(-c4nnc(C(F)F)o4)cc3F)C(=O)N3CCS(=O)(=O)CC3)c2)CC1] |

In example embodiments of the present invention, the pharmaceutical composition including a compound of Table A, optical isomers thereof or pharmaceutically acceptable salts thereof as an active ingredient may prevent or treat pulmonary arterial hypertension.

In the pharmaceutical composition according to the disclosure, the compounds represented by formula I may be shown in Table B below:

TABLE B

| Com- | |
|---|---|
| pound | Structure |
| 40 | 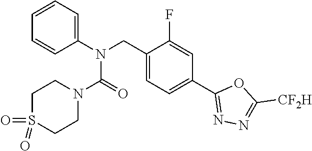 [SMILES: O=C(N1CCS(=O)(=O)CC1)N(Cc1ccc(-c2nnc(C(F)F)o2)cc1F)c1ccccc1] |
| 43 | 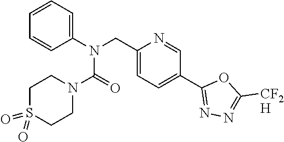 [SMILES: O=C(N1CCS(=O)(=O)CC1)N(Cc1ccc(-c2nnc(C(F)F)o2)cn1)c1ccccc1] |
| 239 | 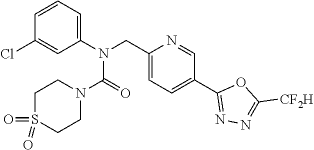 [SMILES: O=C(N1CCS(=O)(=O)CC1)N(Cc1ccc(-c2nnc(C(F)F)o2)cn1)c1cccc(Cl)c1] |
| 285 | 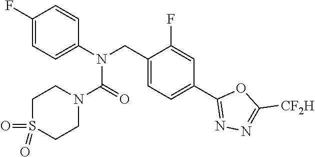 [SMILES: O=C(N1CCS(=O)(=O)CC1)N(Cc1ccc(-c2nnc(C(F)F)o2)cc1F)c1ccc(F)cc1] |
| 295 | 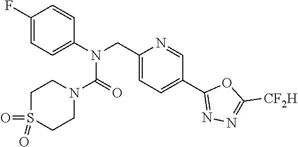 [SMILES: O=C(N1CCS(=O)(=O)CC1)N(Cc1ccc(-c2nnc(C(F)F)o2)cn1)c1ccc(F)cc1] |
| 296 | 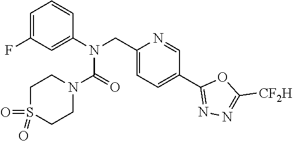 [SMILES: O=C(N1CCS(=O)(=O)CC1)N(Cc1ccc(-c2nnc(C(F)F)o2)cn1)c1cccc(F)c1] |

In example embodiment of the present invention, the pharmaceutical composition including a compound of Table B, optical isomers thereof or pharmaceutically acceptable salts thereof as an active ingredient may prevent or treat pulmonary arterial hypertension.

In the present disclosure, the compound represented by above formula I may be prepared by a method disclosed in Korean Unexamined Patent Application Publication No. 10-2017-0017792, but is not limited thereto.

In the pharmaceutical composition according to the present disclosure, the compound represented by the above formula I may contain at least one asymmetric carbon, and thus may be present as a racemic mixture, a single enantiomer (optical isomer), a mixture of diastereomers, and a single diastereomer. Such isomer may be separated by being split according to the prior art, for example, column chromatography, HPLC or the like. Alternatively, the isomer may be stereospecifically synthesized with a known array of optically pure starting materials and/or reagents. Particularly, said isomer may be an optical isomer (enantiomer).

In the present disclosure, the term “pharmaceutically acceptable” may refer to the one which is physiologically acceptable and does not conventionally cause gastrointestinal disturbance, an allergic response such as dizziness or other responses similar thereto, when being administered to an individual.

The pharmaceutically acceptable salts according to the embodiments of the present invention may be prepared by a conventional method known to those skilled in the art.

The pharmaceutically acceptable salts according to the embodiment of the present invention may include, for example, inorganic ion salts prepared from calcium, potassium, sodium, magnesium, etc.; inorganic acid salts prepared from hydrochloric acid, nitric acid, phosphoric acid, bromic acid, iodic acid, perchloric acid, sulfuric acid, hydroiodic acid, etc.; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, etc.; sulfonic acid salts prepared from methanesulfonic acid, ethanesulfonic acid, benzenesulfonic acid, p-toluenesulfonic acid, naphthalene sulfonic acid, etc.; amino acid salts prepared from glycine, arginine, lysine, etc.; amine salts prepared from trimethylamine, triethylamine, ammonia, pyridine, picoline, etc.; and the like, but are not limited thereto. In the embodiments of the present invention, salts may include hydrochloric acid, trifluoroacetic acid, citric acid, bromic acid, maleic acid, phosphoric acid, sulfuric acid, tartaric acid or a mixture thereof.

As used herein, the term “pulmonary arterial hypertension (PAH)” may refer to a disease in which a blood pressure of blood vessels for supplying blood to the lungs increases.

In embodiments of the present invention, pulmonary arterial hypertension may refer to a state in which a mean pulmonary arterial pressure increases to 25 mmHg or more, and particularly may refer to a state in which the mean pulmonary arterial pressure at rest, as assessed through right heart catheterization (RHC), increases to 25 mmHg or more, but is not limited thereto, and the occurrence of pulmonary arterial hypertension may be determined according to a patient's gender, age, weight, health status, type of disease, severity of disease, drug activity, drug sensitivity, and the like.

In embodiments of the present invention, the pulmonary arterial hypertension may cause endothelial cell dysfunction inside the pulmonary artery, reduced flexibility in the pulmonary vessel, or constriction of the lumen of the vessel, or may lead to symptoms such as the right ventricular failure.

In embodiments of the present invention, symptoms associated with the pulmonary arterial hypertension may include dyspnea during exercise, fatigue, angina pectoris, fainting, dry cough, vomiting accompanying during exercise, cyanosis, peripheral edema, and the like.

In embodiments of the present invention, pulmonary arterial hypertension may correspond to any one of functional classes I to IV of the WHO.

In embodiments of the present invention, pulmonary arterial hypertension may include: idiopathic pulmonary arterial hypertension (IPAH), hereditary pulmonary arterial hypertension, drug- and toxin-induced pulmonary arterial hypertension, disease-associated pulmonary arterial hypertension (APAH), pulmonary arterial hypertension showing a long-term response to calcium channel blockers, pulmonary arterial hypertension with definite signs of venous/capillary invasions, persistent pulmonary arterial hypertension in neonates, or a combination thereof.

Particularly, the idiopathic pulmonary arterial hypertension may include a case in which a pulmonary arterial pressure, etc. rises without a specific cause, and hereditary pulmonary arterial hypertension may include a case in which a pulmonary arterial pressure, etc. rises while genetic abnormalities such as bone morphogenetic protein receptor-2 (BMPR2) mutation, etc., are observed.

The pharmaceutical composition including the compound of formula I, optical isomers thereof or pharmaceutically acceptable salts thereof according to the present disclosure may have remarkably excellent preventive and therapeutic effects on pulmonary arterial hypertension.

The pharmaceutical composition including the compound of formula I, optical isomers thereof or pharmaceutically acceptable salts thereof according to the present disclosure may have excellent preventive and therapeutic effects on idiopathic pulmonary arterial hypertension (IPAH), hereditary pulmonary arterial hypertension, drug- and toxin-induced pulmonary arterial hypertension, disease-associated pulmonary arterial hypertension (APAH), pulmonary arterial hypertension showing a long-term response to calcium channel blockers, pulmonary arterial hypertension with definite signs of venous/capillary invasions, persistent pulmonary arterial hypertension in neonates, or a combination thereof.

The pharmaceutical composition including the compound of formula I, optical isomers thereof or pharmaceutically acceptable salts thereof according to the present disclosure may have excellent preventive or therapeutic effects on all WHO functional classes 1 to 4 of pulmonary arterial hypertension, may show an excellent therapeutic effect not only in the low-risk group of pulmonary arterial hypertension, but also in the intermediate- or high-risk group thereof, may slow down the progression of the disease, such as preventing the disease from progressing to a severe level at an early stage of the disease, and remarkably increase a survival period of a subject suffering from pulmonary arterial hypertension after diagnosis of the disease.

As used herein, the term “prevention” may refer to all the acts, which inhibit or delay the occurrence of the pulmonary arterial hypertension by administering the compound of formula I, optical isomers thereof or pharmaceutically acceptable salts thereof according to the present disclosure, and may also mean all the cases, in which pulmonary arterial hypertension is slightly expressed in terms of a degree of symptoms compared to a case without the administration of the compound of formula I, optical isomers thereof or pharmaceutically acceptable salts thereof.

In embodiments of the present invention, prevention may include a case in which a pulmonary arterial pressure does not increase or a time point at which a pulmonary arterial pressure begins to increase is delayed, or a case in which clinical symptoms (for example, dyspnea during exercise, fatigue, angina pectoris, fainting, dry cough, vomiting accompanying during exercise, cyanosis, peripheral edema, etc.) are not expressed or a time point at which the symptoms begin to express is delayed. For example, the prevention may include a case in which a pulmonary arterial pressure is maintained at a normal degree or a time point at which a pulmonary arterial pressure begins to rise is delayed.

In embodiments of the present invention, the prevention may include a case in which a thickness of the pulmonary artery vessel is maintained normally or a time point at which the thickness of the pulmonary artery vessel begins to increase is delayed as in histological examination.

In the present disclosure, the term “treatment” may refer to all the acts, by which a suspicious symptom of an individual likely to develop a disease and a symptom of an individual suffering from a disease gets better or takes a favorable turn by administering the compound of formula I, optical isomers thereof or pharmaceutically acceptable salts thereof according to the present disclosure. In embodiments of the present invention, the treatment of the present disclosure may include all cases in which a pulmonary arterial pressure is lowered, clinical symptoms are weakened, a WHO functional class is lowered, or a risk level is lowered in risk assessment.

In embodiments of the present invention, treatment may include a case in which the pulmonary artery pressure is 20 mmHg or less, or is lowered to a normal pulmonary artery pressure. Alternatively, in embodiments of the present invention, the treatment may include a case in which clinical symptoms (for example, dyspnea during exercise, fatigue, angina pectoris, fainting, dry cough, vomiting accompanying during exercise, cyanosis, peripheral edema, and the like) are removed or weakened.

In embodiments of the present invention, it has been confirmed that the compound represented by formula I, optical isomers thereof or pharmaceutically acceptable salts thereof increase a survival rate and inhibits a weight of the lung in rats with induced pulmonary arterial hypertension, thereby showing an excellent therapeutic effect on pulmonary arterial hypertension (FIGS. 4 and 5).

In embodiments of the present invention, it has been confirmed through weight measurement that the compound represented by formula I, optical isomers thereof or pharmaceutically acceptable salts thereof ameliorate hypertrophy of the right ventricle in rats with induced pulmonary arterial hypertension (FIG. 6).

In embodiments of the present invention, it has been confirmed through electrocardiogram that the compound represented by formula I, optical isomers thereof or pharmaceutically acceptable salts thereof according to the present disclosure ameliorate heart beat, ventricular bradycardia and hypertrophy of the right ventricle in rats with induced pulmonary arterial hypertension (FIGS. 7 to 16).

The pharmaceutical composition of the present disclosure may further include at least one pharmaceutically acceptable carrier, in addition to the compound represented by above formula I, optical isomers thereof or pharmaceutically acceptable salts thereof. The pharmaceutically acceptable carrier may be the one which is conventionally used in the art, specifically including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidine, cellulose, water, syrup, methylcellulose, methyl hydroxybenzoate, propyl hydroxybenzoate, talc, magnesium stearate, mineral, or oil. The pharmaceutical composition of the present invention may further include lubricants, humectants, sweetening agents, flavoring agents, emulsifiers, suspending agents, preservatives, dispersing agents, stabilizing agents, etc., in addition to the above ingredients. In addition, the pharmaceutical composition of the present invention may be formulated into an oral dosage form such as a tablet, powder, granule, pill, capsule, suspension, emulsion, liquid for internal use, oiling agent, syrup, etc., as well as a form of external application, suppository or sterile solution for injection, by using pharmaceutically acceptable carriers and excipients and thus may be prepared in a unit dose form or prepared by being inserted into a multi-dose container. Such preparations may be prepared according to a conventional method used for formulation in the art or a method disclosed in Remington's Pharmaceutical Science (19th ed., 1995), and may be formulated into various preparations depending on each disease or ingredient.

A non-limiting example of preparations for oral administration using the pharmaceutical composition of the present invention may include tablets, troches, lozenges, water-soluble suspensions, oil suspensions, prepared powders, granules, emulsions, hard capsules, soft capsules, syrups, elixirs or the like. To formulate the pharmaceutical composition according to the embodiments of the present invention into preparation for oral administration, the followings may be used: binders such as lactose, saccharose, sorbitol, mannitol, starch, amylopectin, cellulose, gelatin or the like; excipients such as dicalcium phosphate, etc.; disintegrants such as maize starch, sweet potato starch or the like; lubricants such as magnesium stearate, calcium stearate, sodium stearyl fumarate, polyethylene glycol wax, or the like; etc., in which sweetening agents, flavoring agents, syrups, etc. may also be used. Furthermore, in the case of the capsules, liquid carriers such as fatty oil, etc. may be further used in addition to the above-mentioned materials.

A non-limiting example of parenteral preparations using the pharmaceutical composition according to the embodiments of the present invention may include injectable solutions, suppositories, powders for respiratory inhalation, aerosols for spray, ointments, powders for application, oils, creams, etc. To formulate the pharmaceutical composition according to the embodiments of the present invention into preparation for parenteral administration, the following may be used: sterilized aqueous solutions, non-aqueous solvents, suspensions, emulsions, freeze-dried preparations, external preparations, etc. As said non-aqueous solvents and suspensions, the following may be used, but without limitation thereto: propylene glycol, polyethylene glycol, vegetable oils such as olive oil, injectable esters such as ethyl oleate, etc.

The pharmaceutical composition according to the embodiments of the present invention may be subjected to oral administration or parenteral administration according to a targeted method, for example, intravenous, subcutaneous, intraperitoneal or local administration, particularly oral administration, but is not limited thereto.

A daily dosage of the compound represented by formula I, optical isomers thereof or pharmaceutically acceptable salts thereof according to the present disclosure may be particularly about 0.1 to about 10,000 mg/kg, about 1 to about 8,000 mg/kg, about 5 to about 6,000 mg/kg, or about 10 to about 4,000 mg/kg, and more particularly about 50 to about 2,000 mg/kg, but is not limited thereto and may be also administered once a day or several times a day by dividing the daily dosage of the compound.

A pharmaceutically effective dose and an effective dosage of the pharmaceutical composition according to the embodiments of the present invention may vary depending on a method for formulating the pharmaceutical composition, an administration mode, an administration time, an administration route, and/or the like, and may be diversified according to various factors including a type and degree of reaction to be achieved by administration of the pharmaceutical composition, a type of an individual for administration, the individual's age, weight, general health condition, disease symptom or severity, gender, diet and excretion, ingredients of other drug compositions to be used for the corresponding individual at the same time or different times, etc., as well as other similar factors well known in a pharmaceutical field, and those skilled in the art may easily determine and prescribe an effective dosage for the intended treatment.

The pharmaceutical composition according to the embodiments of the present invention may be administered once a day or several times a day by dividing the daily dosage of the composition.

The pharmaceutical composition of the present invention may be administered as an individual therapeutic agent or in combination with other therapeutic agents, and may be administered sequentially or simultaneously with a conventional therapeutic agent. Considering all the above factors, the pharmaceutical composition of the present invention may be administered in such an amount that a maximum effect may be achieved by a minimum amount without a side effect, and such amount may be easily determined by those skilled in the art to which the present invention pertains.

The pharmaceutical composition including the compound of formula I, optical isomers thereof or pharmaceutically acceptable salts thereof according to the embodiments of the present invention may be administered in combination with one or more other therapeutic agents.

The pharmaceutical composition including the compound of formula I, optical isomers thereof or pharmaceutically acceptable salts thereof according to the embodiments of the present invention may show an excellent effect even when solely used, but may be further used in combination with various methods such as hormone therapy, drug treatment, etc. to increase therapeutic efficiency.

The present disclosure may provide a method for preventing or treating pulmonary arterial hypertension, including administering a compound represented by above formula I, optical isomers thereof or pharmaceutically acceptable salts thereof into an individual.

The present disclosure may provide a method for preventing or treating pulmonary arterial hypertension, including administering a compound of the above Table A, optical isomers thereof or pharmaceutically acceptable salts thereof into an individual.

The present disclosure may provide a method for preventing or treating pulmonary arterial hypertension, including administering a compound of the above Table B, optical isomers thereof or pharmaceutically acceptable salts thereof into an individual.

Said terms “pulmonary arterial hypertension” “prevention” and “treatment” may be the same as described above.

In the present disclosure, the term “administration” may refer to introducing a predetermined substance into an individual by an appropriate method.

In the present disclosure, the term “individual” may refer to all the animals such as rats, mice, livestock, etc., including humans, who have developed or are likely to develop pulmonary arterial hypertension, and may be particularly mammals including humans, but is not limited thereto.

The method for preventing or treating pulmonary arterial hypertension according to the embodiments of the present invention may include administering a therapeutically effective amount of the compound represented by above formula I, optical isomers thereof or pharmaceutically acceptable salts thereof.

In the present disclosure, the term “therapeutically effective amount” may refer to an amount enough to treat a disease at a reasonable risk/benefit ratio applicable to medical treatment and not to cause a side effect, and may be determined by those skilled in the art according to factors including a patient's gender, age, weight and health condition, a type of disease, severity, the activity of a drug, sensitivity to a drug, an administration method, an administration time, an administration route, an excretion rate, a treatment period, a drug combined or concurrently used, as well as other factors well known in a pharmaceutical field. It is preferable to differently apply a particular therapeutically effective amount for a certain patient depending on various factors including a type and degree of reaction to be achieved therefrom, a particular composition including a presence of other preparations used in some cases, a patient's age, weight, general health condition, gender and diet, an administration time, an administration route, an excretion rate of the composition, a treatment period and a drug used together with the particular composition or simultaneously therewith, as well as other similar factors well known in a pharmaceutical field.

The method for preventing or treating the pulmonary arterial hypertension of the present disclosure may include not only dealing with the disease per se before expression of its symptoms, but also inhibiting or avoiding such symptoms by administering the compound represented by above formula I, isomers thereof or pharmaceutically acceptable salts thereof. In managing the disease, a preventive or therapeutic dose of a certain active ingredient may vary depending on the characteristics and severity of the disease or conditions, and a route in which the active ingredient is administered. A dose and a frequency thereof may vary depending on an individual patient's age, weight and reactions. A suitable dose and usage may be easily selected by those skilled in the art, naturally considering such factors.

In addition, the method for preventing or treating pulmonary arterial hypertension of the present disclosure may further include administering a therapeutically effective amount of an additional active agent, which helps prevent or treat the disease, along with the compound represented by above formula I, optical isomers thereof or pharmaceutically acceptable salts thereof, and the additional active agent may show a synergy effect or an additive effect together with the compound represented by above formula I, optical isomers thereof or pharmaceutically acceptable salts thereof.

The present disclosure may provide a use of the compound represented by the above formula I, optical isomers thereof or pharmaceutically acceptable salts thereof for preventing or treating pulmonary arterial hypertension.

The present disclosure may provide a use of the compound of the above Table A, optical isomers thereof or pharmaceutically acceptable salts thereof for preventing or treating pulmonary arterial hypertension.

The present disclosure may provide a use of the compound of the above Table B, optical isomers thereof or pharmaceutically acceptable salts thereof for preventing or treating pulmonary arterial hypertension.

The present disclosure may provide a use of the compound represented by above formula I, optical isomers thereof or pharmaceutically acceptable salts thereof in preparing a medicament for preventing or treating pulmonary arterial hypertension.

The present disclosure may provide a use of the compound of the above Table A, optical isomers thereof or pharmaceutically acceptable salts thereof in preparing a medicament for preventing or treating pulmonary arterial hypertension.

The present disclosure may provide a use of the compound of the above Table B, optical isomers thereof or pharmaceutically acceptable salts thereof in preparing a medicament for preventing or treating pulmonary arterial hypertension.

Said terms “pulmonary arterial hypertension,” “prevention” and “treatment” may be the same as described above.

For the preparation of the medicament, the compound represented by above formula I, optical isomers thereof or pharmaceutically acceptable salts thereof may be mixed with pharmaceutically acceptable adjuvants, diluents, carriers, etc., and may be prepared into a complex preparation together with other active agents, thus providing a synergy action.

Matters mentioned in the pharmaceutical composition, treatment method and use of the present disclosure are applied the same, if not contradictory to each other.

### Advantageous Effects

The compound represented by formula I, optical isomers thereof or pharmaceutically acceptable salts thereof and the pharmaceutical composition including the same as an active ingredient according to the present disclosure may be advantageously used in preventing or treating pulmonary arterial hypertension.

### BRIEF DESCRIPTION OF THE DRAWINGS

FIGS. 1 to 3 are views showing an effect of ameliorating pulmonary arterial hypertension according to treatment with the compound of the present disclosure in a cellular model of pulmonary arterial hypertension.

FIG. 4 is a view showing a survival rate of individuals in an animal model of pulmonary arterial hypertension for four weeks according to the administration of the compound of the present disclosure.

FIG. 5 is a view showing a weight of lung of individuals in an animal model of pulmonary arterial hypertension for four weeks according to the administration of the compound of the present disclosure.

FIG. 6 is a view showing a ratio of right ventricle weight to left ventricle weight in an animal model of pulmonary arterial hypertension according to the administration of the compound of the present disclosure.

FIGS. 7 to 16 are views showing results of ECG measurement in an animal model of pulmonary arterial hypertension according to the administration of the compound of the present disclosure.

### MODE FOR INVENTION

The present disclosure will be described in detail with reference to Examples hereinafter. However, the Examples are only for the purpose of illustrating the present invention and it is obvious to those skilled in the art that the scope of the present invention is not limited to the Examples disclosed hereinafter.

### Synthesis Example 1. Synthesis of compound 43, N-((5-(5-(difluoromethyl)-1,3,4-oxadiazol-2-yl)pyridin-2-yl)methyl)-N-phenylthiomorpholine-4-carboxamide 1,1-dioxide

### [Step 1] N-phenylthiomorpholine-4-carboxamide 1,1-dioxide

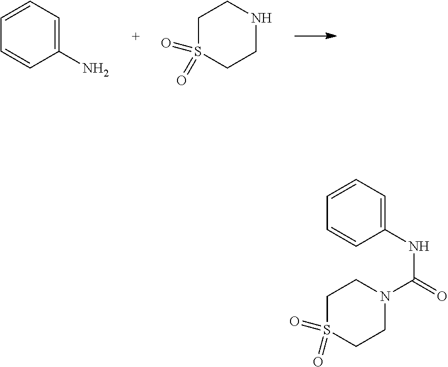 [SMILES: Nc1ccccc1 ; O=S1(=O)CCNCC1 ; O=C(Nc1ccccc1)N1CCS(=O)(=O)CC1]

To a solution of aniline (3.000 g, 32,213 mmol) and N,N-diisopropylethylamine (33.439 mL, 193.278 mmol) in dichloromethane (100 mL) was added at 0° C. triphosgene (4.780 g, 16.107 mmol) and was stirred at the same temperature. Thiomorpholine 1,1-dioxide (4.790 g, 35.434 mmol) was added to the reaction mixture and stirred for an additional 16 hr at room temperature. Then, water was added to the reaction mixture, followed by extraction with ethyl acetate. The organic layer was washed with brine, dried (anhydrous MgSO₄), filtered, and concentrated under the reduced pressure. The concentrate was purified and concentrated by column chromatography (SiO₂, 40 g cartridge; methanol/dichloromethane=2%) to give the title compound as yellow solid (1.325 g, 16.2%).

### [Step 2] Synthesis of Methyl 6-((1,1-dioxido-N-phenylthiomorpholine-4-carboxamido)methyl)nicotinate

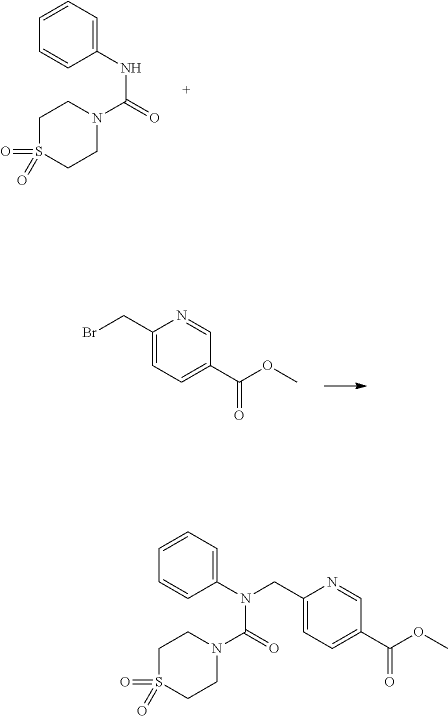 [SMILES: O=C(Nc1ccccc1)N1CCS(=O)(=O)CC1 ; COC(=O)c1ccc(CBr)nc1 ; COC(=O)c1ccc(CN(C(=O)N2CCS(=O)(=O)CC2)c2ccccc2)nc1]

A solution of N-phenylthiomorpholine-4-carboxamide 1,1-dioxide (1.000 g, 3.932 mmol) prepared in Step 1 and sodium hydride (60.00%, 0.157 g, 3.932 mmol) in N,N-dimethylformamide (10 mL) was stirred at 0° C. for 1 hr, and mixed with methyl 4-(bromomethyl)-3-fluorobenzoate (0.905 g, 3.932 mmol). The reaction mixture was stirred at room temperature for an additional 2 hr. The reaction mixture was concentrated under the reduced pressure to remove the solvent, and water was added to the concentrate, followed by extraction with ethyl acetate. The organic layer was washed with brine, dried (anhydrous MgSO₄), filtered, and concentrated under the reduced pressure. The crude product was crystallized at room temperature using methanol (20 mL). The resulting precipitates obtained by filtration were washed by methanol, and dried to give the title compound as brown solid (0.816 g. 51.4%).

### [Step 3] Synthesis of N-((5-(hydrazinecarbonyl)pyridin-2-yl)methyl)-N-phenylthiomorpholine-4-carboxamide 1,1-dioxide

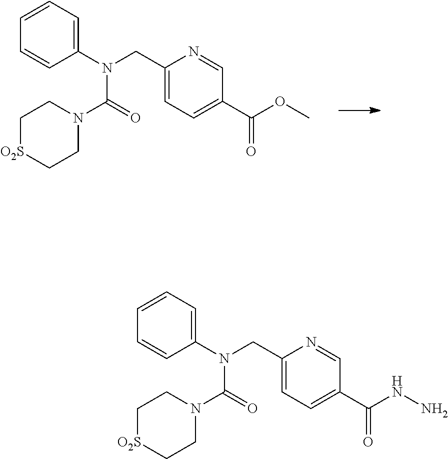 [SMILES: COC(=O)c1ccc(CN(C(=O)N2CCS(=O)(=O)CC2)c2ccccc2)nc1 ; NNC(=O)c1ccc(CN(C(=O)N2CCS(=O)(=O)CC2)c2ccccc2)nc1]

Methyl 6-((1,1-dioxido-N-phenylthiomorpholine-4-carboxamido)methyl)nicotinate (0.816 g, 2.023 mmol) prepared in Step 2 and hydrazine monohydrate (1.910 mL, 40.451 mmol) was mixed in ethanol (10 mL) at the room temperature and then heated at 100° C. under the microwaves for 1 hr, and cooled down to the room temperature to terminate the reaction. The reaction mixture was concentrated under the reduced pressure to remove the solvent. The crude product was crystallized at room temperature using dichloromethane (20 mL). The resulting precipitates obtained by filtration were washed by dichloromethane, and dried to give the title compound as light brown solid (0.560 g, 68.6%).

### [Step 4] Synthesis of N-((5-(2-(2,2-difluoroacetyl)hydrazine-1-carbonyl)pyridin-2-yl)methyl)-N-phenylthiomorpholine-4-carboxamide 1,1-dioxide

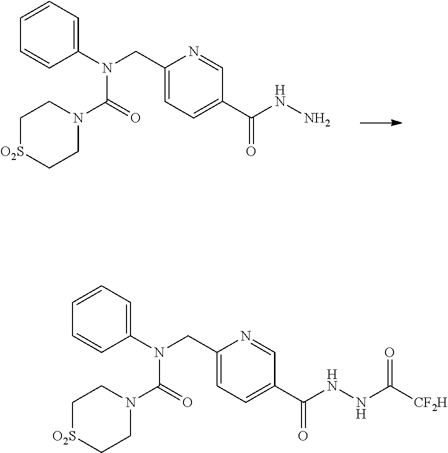 [SMILES: NNC(=O)c1ccc(CN(C(=O)N2CCS(=O)(=O)CC2)c2ccccc2)nc1 ; O=C(NNC(=O)C(F)F)c1ccc(CN(C(=O)N2CCS(=O)(=O)CC2)c2ccccc2)nc1]

A solution of N-((5-(hydrazinecarbonyl)pyridin-2-yl)methyl)-N-phenylthiomorpholine-4-carboxamide 1,1-dioxide (0.260 g, 0.644 mmol) prepared in Step 3 and triethylamine (0.178 mL, 1.289 mmol) in dichloromethane (2 mL) was mixed with Difluoroacetic Anhydride (0.087 mL, 0.580 mmol) at the room temperature. The reaction mixture was stirred at the same temperature for 16 hr. Then, water was added to the reaction mixture, followed by extraction with dichloromethane. The mixture was passed through a plastic frit to remove solid residues and an aqueous layer, and the organic layer collected was concentrated under the reduced pressure. The concentrate was purified and concentrated by column chromatography (SiO₂, 4 g cartridge; methanol/dichloromethane=0% to 5%) to give the title compound as white foam (0.156 g, 50.3%).

### [Step 5] Synthesis of Compound 43

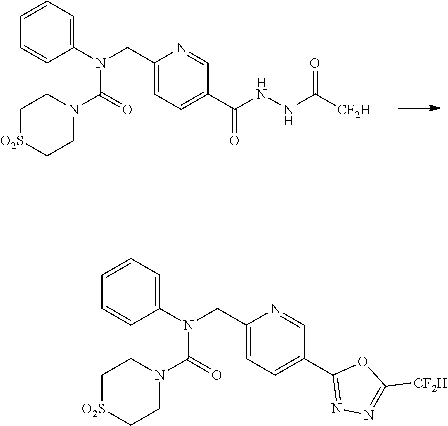 [SMILES: O=C(NNC(=O)C(F)F)c1ccc(CN(C(=O)N2CCS(=O)(=O)CC2)c2ccccc2)nc1 ; O=C(N1CCS(=O)(=O)CC1)N(Cc1ccc(-c2nnc(C(F)F)o2)cn1)c1ccccc1]

A mixture of N-((5-(2-(2,2-difluoroacetyl)hydrazine-1-carbonyl)pyridin-2-yl)methyl)-N-phenylthiomorpholine-4-carboxamide 1,1-dioxide (0.156 g, 0.324 mmol) prepared in Step 4 and 1-methoxy-N-triethylammoniosulfonyl-methanimidate (Burgess reagent, 0.116 g, 0.486 mmol) in tetrahydrofuran (2 mL) was heated at 150° C. for 30 min under the microwaves, and cooled down to the room temperature to terminate the reaction. Then, water was added to the reaction mixture, followed by extraction with dichloromethane. The biphasic mixture was passed through a plastic frit to remove solid residues and an aqueous layer, and the organic layer collected was concentrated under the reduced pressure. The concentrate was purified and concentrated by column chromatography (SiO₂, 4 g cartridge; methanol/dichloromethane=3%) to give the title compound as colorless oil (0.078 g, 51.9%).

¹H NMR (400 MHZ, CDCl₃) δ 9.23 (d, 1H, J=2.2 Hz), 8.38 (dd, 1H, J=8.2, 2.2 Hz), 7.54 (d, 1H, J=8.2 Hz), 7.41-7.31 (m, 2H), 7.19 (ddd, 3H, J=6.4, 3.0, 1.6 Hz), 6.94 (m, 1H), 5.10 (s, 2H), 3.72 (dd, 4H, J=6.9, 3.7 Hz), 2.97-2.90 (m, 4H); LRMS(ES) m/z 464.2 (M⁺+1).

### Synthesis Example 2. Synthesis of compound 40, N-(4-(5-(difluoromethyl)-1,3,4-oxadiazol-2-yl)-2-fluorobenzyl)-N-phenylthiomorpholine-4-carboxamide 1,1-dioxide

### [Step 1] Methyl 4-((1,1-dioxido-N-phenylthiomorpholine-4-carboxamido)methyl)-3-fluorobenzoate

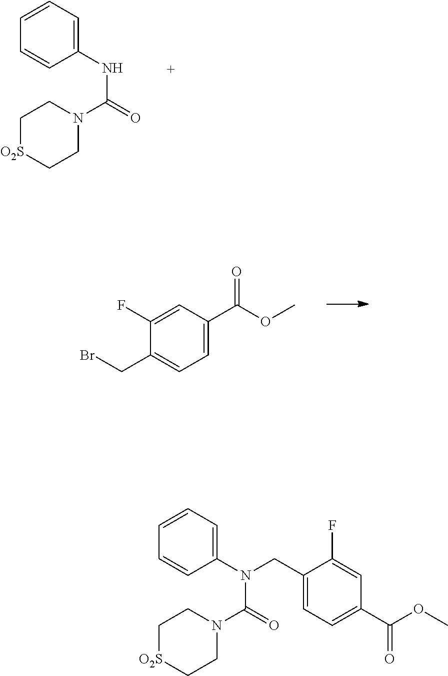 [SMILES: O=C(Nc1ccccc1)N1CCS(=O)(=O)CC1 ; COC(=O)c1ccc(CBr)c(F)c1 ; COC(=O)c1ccc(CN(C(=O)N2CCS(=O)(=O)CC2)c2ccccc2)c(F)c1]

A solution of N-phenylthiomorpholine-4-carboxamide 1,1-dioxide (1.000 g, 3.932 mmol) and sodium hydride (60.00%, 0.189 g, 4.719 mmol) in N,N-dimethylformamide (30 mL) was mixed at 0° C. with methyl 4-(bromomethyl)-3-fluorobenzoate (1.020 g, 4.129 mmol), and stirred at the room temperature for 18 hr. Then, saturated aqueous sodium bicarbonate solution was added to the reaction mixture, followed by extraction with ethyl acetate. The organic layer was washed with brine, dried (anhydrous MgSO₄), filtered, and concentrated in vacuo. The concentrate was purified and concentrated by column chromatography (SiO₂, 40 g cartridge; ethyl acetate/hexane=0% to 50%) to give the title compound methyl 4-((1,1-dioxido-N-phenylthiomorpholine-4-carboxamido)methyl)-3-fluorobenzoate as white solid (1.240 g, 75.0%).

### [Step 2] N-(2-fluoro-4-(hydrazinecarbonyl)benzyl)-N-phenylthiomorpholine-4-carboxamide 1,1-dioxide

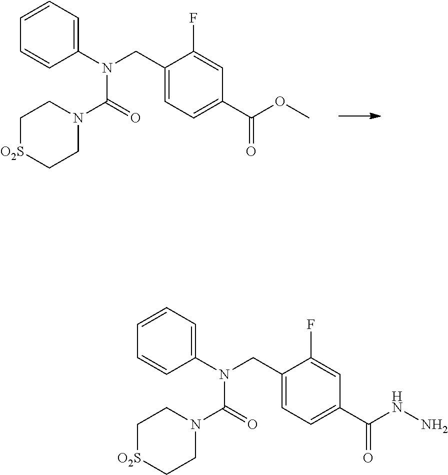 [SMILES: COC(=O)c1ccc(CN(C(=O)N2CCS(=O)(=O)CC2)c2ccccc2)c(F)c1 ; NNC(=O)c1ccc(CN(C(=O)N2CCS(=O)(=O)CC2)c2ccccc2)c(F)c1]

A solution of methyl 4-((1,1-dioxido-N-phenylthiomorpholine-4-carboxamido)methyl)-3-fluorobenzoate (1.240 g, 2.949 mmol) prepared in Step 1 and hydrazine monohydrate (2.786 mL, 58.983 mmol) in ethanol (15 mL) was stirred at 120° C. for 1 hr, and cooled down to the room temperature to terminate the reaction. The reaction mixture was concentrated under the reduced pressure to remove the solvent, and saturated aqueous sodium bicarbonate solution was added to the concentrate, followed by extraction with dichloromethane. The bi-phasic mixture was passed through a plastic frit to remove solid residues and aqueous layer, and the organic layer collected was concentrated in vacuo. The crude title compound N-(2-fluoro-4-(hydrazinecarbonyl)benzyl)-N-phenylthiomorpholine-4-carboxamide 1,1-dioxide was used without further purification (1.240 g, 100.0%, white solid).

### [Step 3] N-(4-(2-(2,2-difluoroacetyl)hydrazine-1-carbonyl)-2-fluorobenzyl)-N-phenylthiomorpholine-4-carboxamide 1,1-dioxide

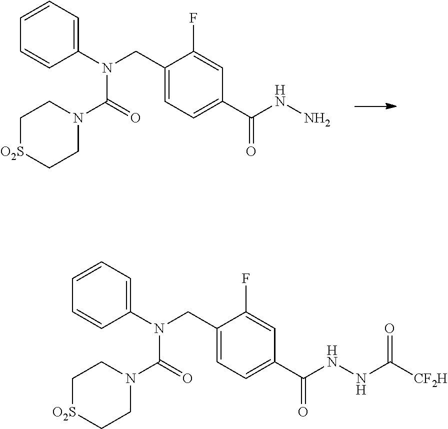 [SMILES: NNC(=O)c1ccc(CN(C(=O)N2CCS(=O)(=O)CC2)c2ccccc2)c(F)c1 ; O=C(NNC(=O)C(F)F)c1ccc(CN(C(=O)N2CCS(=O)(=O)CC2)c2ccccc2)c(F)c1]

A solution of N-(2-fluoro-4-(hydrazinecarbonyl)benzyl)-N-phenylthiomorpholine-4-carboxamide 1,1-dioxide (0.615 g, 1.463 mmol) prepared in Step 2, triethylamine (0.304 mL, 2.194 mmol) and difluoroacetic anhydride (0.164 mL, 1.316 mmol) in dichloromethane (10 mL) was stirred at the room temperature for 18 hr. Then, saturated aqueous sodium bicarbonate solution was added to the reaction mixture, followed by extraction with ethyl acetate. The organic layer was washed with brine, dried (anhydrous MgSO₄), filtered, and concentrated in vacuo. The concentrate was purified and concentrated by column chromatography (SiO₂, 24 g cartridge; methanol/dichloromethane=0% to 3%) to give the title compound N-(4-(2-(2,2-difluoroacetyl)hydrazine-1-carbonyl)-2-fluorobenzyl)-N-phenylthiomorpholine-4-carboxamide 1,1-dioxide as white solid (0.462 g, 63.4%).

### [Step 4] Synthesis of Compound 40

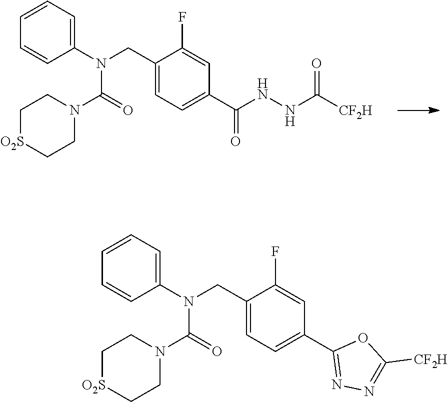 [SMILES: O=C(NNC(=O)C(F)F)c1ccc(CN(C(=O)N2CCS(=O)(=O)CC2)c2ccccc2)c(F)c1 ; O=C(N1CCS(=O)(=O)CC1)N(Cc1ccc(-c2nnc(C(F)F)o2)cc1F)c1ccccc1]

A mixture of N-(4-(2-(2,2-difluoroacetyl)hydrazine-1-carbonyl)-2-fluorobenzyl)-N-phenylthiomorpholine-4-carboxamide 1,1-dioxide (0.462 g, 0.927 mmol) prepared in Step 3 and 1-methoxy-N-triethylammoniosulfonyl-methanimidate (burgess reagent, 0.331 g, 1.390 mmol) in tetrahydrofuran (10 mL) was heated at 150° C. for 30 min under the microwaves, and cooled down to the room temperature to terminate the reaction. Then, saturated aqueous sodium bicarbonate solution was added to the reaction mixture, followed by extraction with dichloromethane. The bi-phasic mixture was passed through a plastic frit to remove solid residues and aqueous layer, and the organic layer collected was concentrated in vacuo. The concentrate was purified and concentrated by column chromatography (SiO₂, 12 g cartridge; ethyl acetate/hexane=0% to 50%) to give the title compound N-(4-(5-(difluoromethyl)-1,3,4-oxadiazol-2-yl)-2-fluorobenzyl)-N-phenylthiomorpholine-4-carboxamide 1,1-dioxide as white solid (0.337 g, 75.7%).

¹H NMR (400 MHZ, CDCl₃) δ 7.87-7.85 (m, 1H), 7.75-7.72 (m, 1H), 7.67-7.64 (m, 1H), 7.38-7.34 (m, 2H), 7.25-7.20 (m, 1H), 7.13-7.10 (m, 2H), 7.03-6.77 (m, 1H), 4.92 (s, 2H), 3.71-3.67 (m, 4H), 2.77-2.74 (m, 4H); LRMS(ES) m/z 481.1 (M⁺+1).

### Synthesis Example 3. Synthesis of the compound 239, N-(3-chlorophenyl)-N-((5-(5-(difluoromethyl)-1,3,4-oxadiazol-2-yl)pyridin-2-yl)methyl)thiomorpholine-4-carboxamide 1,1-dioxide

### [Step 1] Synthesis of N-(3-chlorophenyl)thiomorpholine-4-carboxamide 1,1-dioxide

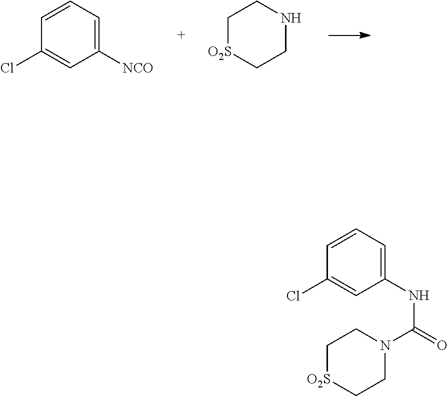

A solution of 1-chloro-3-isocyanatobenzene (1.000 g, 6.512 mmol) and thiomorpholine 1,1-dioxide (0.871 g, 6.447 mmol) in diethyl ether (20 mL) was stirred at the room temperature for 18 hr. The precipitates were filtered, washed by diethyl ether, and dried to give the title compound as white solid (1.811 g, 96.3%).

### [Step 2] Synthesis of Methyl 6-((N-(3-chlorophenyl)-1,1-dioxidothiomorpholine-4-carboxamido)methyl)nicotinate

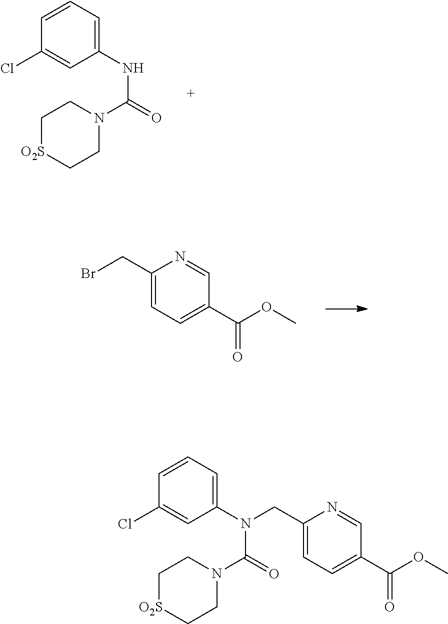 [SMILES: O=C(Nc1cccc(Cl)c1)N1CCS(=O)(=O)CC1 ; COC(=O)c1ccc(CBr)nc1 ; COC(=O)c1ccc(CN(C(=O)N2CCS(=O)(=O)CC2)c2cccc(Cl)c2)nc1]

To a solution of N-(3-chlorophenyl)thiomorpholine-4-carboxamide 1,1-dioxide (0.200 g, 0.693 mmol) prepared in Step 1 in N,N-dimethylformamide (5 mL) was added at 0° C. sodium hydride (60.00%, 0.028 g, 0.693 mmol). The reaction mixture was stirred at the same temperature for 1 hr, added at the same temperature with methyl 6-(bromomethyl) nicotinate (0.159 g, 0.693 mmol), and stirred for additional 2 hr. Then, water was added to the reaction mixture, followed by extraction with ethyl acetate. The organic layer was washed with aqueous saturated sodium chloride solution, dried (anhydrous MgSO₄), filtered, and concentrated under the reduced pressure. The residue was purified and concentrated by chromatography (SiO₂, 12 g cartridge; methanol/dichloromethane=0% to 5%) to give the title compound as brown oil (0.261 g, 86.0%).

### [Step 3] Synthesis of N-(3-chlorophenyl)-N-((5-(hydrazinecarbonyl)pyridin-2-yl)methyl)thiomorpholine-4-carboxamide 1,1-dioxide

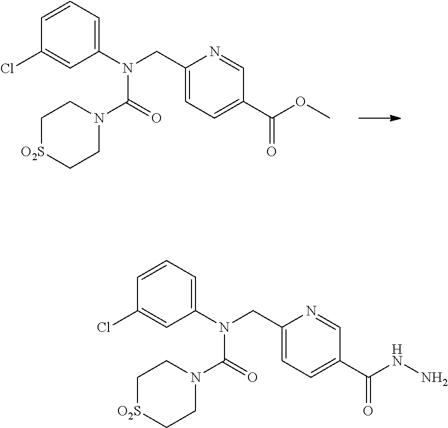 [SMILES: COC(=O)c1ccc(CN(C(=O)N2CCS(=O)(=O)CC2)c2cccc(Cl)c2)nc1 ; NNC(=O)c1ccc(CN(C(=O)N2CCS(=O)(=O)CC2)c2cccc(Cl)c2)nc1]

Methyl 6-((N-(3-chlorophenyl)-1,1-dioxidothiomorpholine-4-carboxamido)methyl)nicotinate (0.261 g, 0.596 mmol) prepared in Step 2 and hydrazine monohydrate (0.290 mL, 5.958 mmol) were mixed at the room temperature in ethanol (2 mL) and then stirred at 110° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=5% to 15%) to give the title compound as brown oil (0.261 g, 100.0%).

### [Step 4] Synthesis of Compound 239

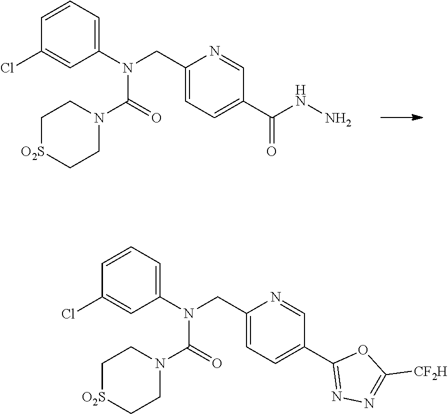 [SMILES: NNC(=O)c1ccc(CN(C(=O)N2CCS(=O)(=O)CC2)c2cccc(Cl)c2)nc1 ; O=C(N1CCS(=O)(=O)CC1)N(Cc1ccc(-c2nnc(C(F)F)o2)cn1)c1cccc(Cl)c1]

N-(3-chlorophenyl)-N-((5-(hydrazinecarbonyl)pyridin-2-yl)methyl)thiomorpholine-4-carboxamide 1,1-dioxide (0.261 g, 0.596 mmol) prepared in Step 3, triethylamine (0.415 mL, 2.980 mmol) and 2,2-difluoroacetic anhydride (0.195 mL, 1.788 mmol) were mixed at the room temperature in tetrahydrofuran (2 mL) and then the obtained solution was stirred at 80° C. for 18 hr and cooled down to the room temperature to terminate the reaction. The reaction mixture was concentrated under the reduced pressure to remove the solvent. Then, water was added to the obtained concentrate, followed by extraction with dichloromethane. The biphasic mixture was passed through a plastic frit to remove the solid residues and aqueous layer, and the organic layer collected was concentrated under the reduced pressure. The residue was purified and concentrated by chromatography (SiO₂, 4 g cartridge; methanol/dichloromethane=0% to 3%) to give the title compound as yellow foam (0.087 g, 29.3%).

¹H NMR (400 MHZ, CDCl₃) δ 9.27 (dd, 1H, J=2.2, 0.8 Hz), 8.43 (dd, 1H, J=8.2, 2.2 Hz), 7.55 (dd, 1H, J=8.2, 0.9 Hz), 7.31 (t, 1H, J=8.0 Hz), 7.23 (t, 1H, J=2.1 Hz), 7.21-7.10 (m, 2H), 7.10 (t, 1H), 5.12 (s, 2H), 3.75 (t, 4H, J=5.3 Hz), 3.06-2.99 (m, 4H); LRMS(ES) m/z 498.3 (M⁺+1).

### Synthesis Example 4. Synthesis of the compound 285: N-(4-(5-(difluoromethyl)-1,3,4-oxadiazol-2-yl)-2-fluorobenzyl)-N-(4-fluorophenyl)thiomorpholine-4-carboxamide 1,1-dioxide

### [Step 1] N-(4-fluorophenyl)thiomorpholine-4-carboxamide 1,1-dioxide

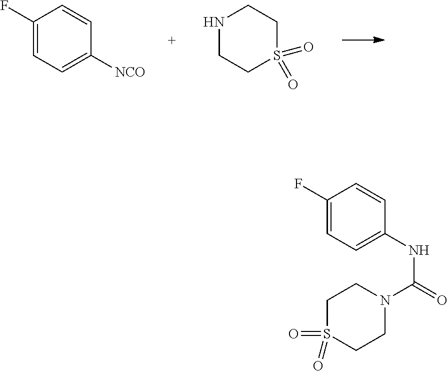

A solution of 1-fluoro-4-isocyanatobenzene (0.500 g, 3.647 mmol) in diethylether (10 mL) was mixed at 0° C. with thiomorpholine 1,1-dioxide (0.493 g, 3.647 mmol), and stirred at the same temperature for 1 hr. The reaction mixture was stirred at the room temperature for additional 4 hr. The precipitates were collected by filtration, washed by diethylether, and dried to give N-(4-fluorophenyl)thiomorpholine-4-carboxamide 1,1-dioxide as white solid (0.920 g, 92.7%).

### [Step 2] Methyl 3-fluoro-4-((N-(4-fluorophenyl)-1,1-dioxidothiomorpholine-4-carboxamido)methyl)benzoate

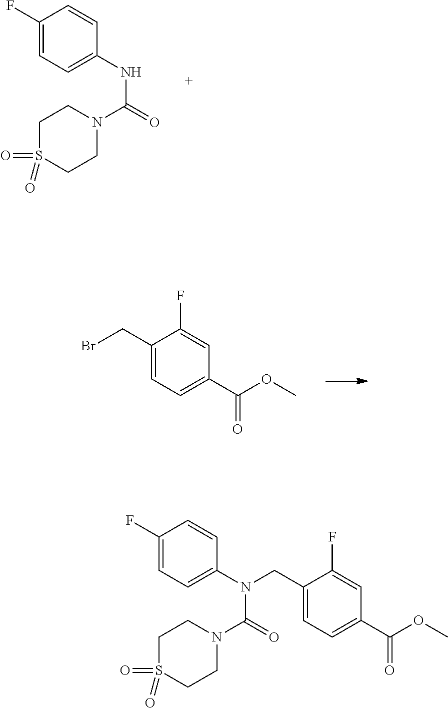 [SMILES: O=C(Nc1ccc(F)cc1)N1CCS(=O)(=O)CC1 ; COC(=O)c1ccc(CBr)c(F)c1 ; COC(=O)c1ccc(CN(C(=O)N2CCS(=O)(=O)CC2)c2ccc(F)cc2)c(F)c1]

A solution of N-(4-fluorophenyl)thiomorpholine-4-carboxamide 1,1-dioxide (0.300 g, 1.102 mmol) prepared in Step 1 and sodium hydride (60.00%, 0.048 g, 1.212 mmol) in N,N-dimethylformamide (5 mL) was stirred at 0° C. for 2 hr, and mixed with methyl 4-(bromomethyl)-3-fluorobenzoate (0.299 g, 1.212 mmol). The reaction mixture was stirred at the room temperature for additional 17 hr, quenched at the room temperature by the addition of water (2 mL, 10 min stirring). Then, water was added to the reaction mixture, followed by extraction with dichloromethane. The bi-phasic mixture was passed through a plastic frit to remove the solid residues and aqueous layer, and the organic layer collected was concentrated in vacuo. The crude product was crystallized at the room temperature using dichloromethane (3 mL). The resulting precipitates were filtered, washed by dichloromethane, and dried to give methyl 3-fluoro-4-((N-(4-fluorophenyl)-1,1-dioxidothiomorpholine-4-carboxamido)methyl)benzoate as white solid (0.212 g, 43.9%).

### [Step 3] N-(2-fluoro-4-(hydrazinecarbonyl)benzyl)-N-(4-fluorophenyl)thiomorpholine-4-carboxamide 1,1-dioxide

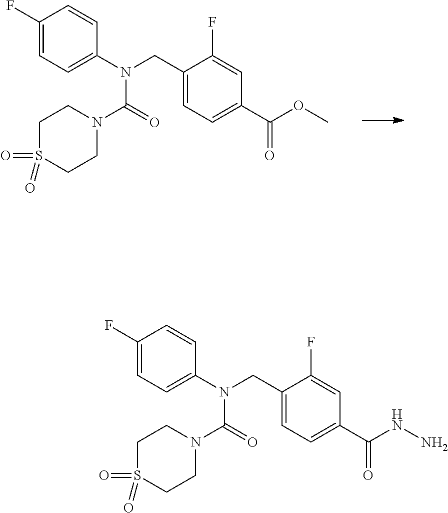 [SMILES: COC(=O)c1ccc(CN(C(=O)N2CCS(=O)(=O)CC2)c2ccc(F)cc2)c(F)c1 ; NNC(=O)c1ccc(CN(C(=O)N2CCS(=O)(=O)CC2)c2ccc(F)cc2)c(F)c1]

Methyl 3-fluoro-4-((N-(4-fluorophenyl)-1,1-dioxidothiomorpholine-4-carboxamido)methyl)benzoate (0.212 g, 0.484 mmol) prepared in Step 2 and hydrazine monohydrate (0.470 mL, 9.670 mmol) in ethanol (4 mL) was mixed at the room temperature and then heated at 120° C. under the microwaves for 1 hr and cooled down to the room temperature to terminate the reaction. The reaction mixture was concentrated under the reduced pressure to remove the solvent. Then, water was added to the reaction mixture, followed by extraction with dichloromethane. The bi-phasic mixture was passed through a plastic frit to remove the solid residues and aqueous layer, and the organic layer collected was concentrated in vacuo. The residue was diluted with diethylether (5 mL) and ethyl acetate (1 mL) and stirred at the ambient temperature. The resulting precipitates were collected by filtration, washed by hexane, and dried to give N-(2-fluoro-4-(hydrazinecarbonyl)benzyl)-N-(4-fluorophenyl)thiomorpholine-4-carboxamide 1,1-dioxide as white solid (0.179 g, 84.4%).

### [Step 4] Synthesis of Compound 285

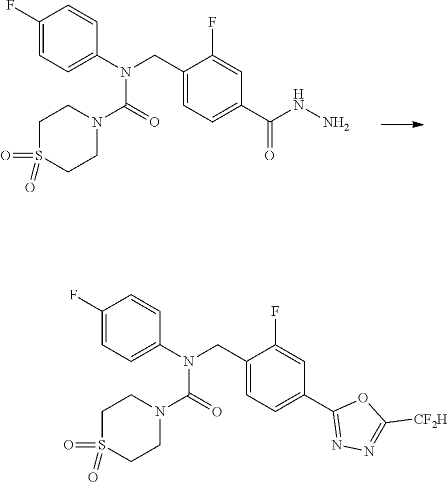 [SMILES: NNC(=O)c1ccc(CN(C(=O)N2CCS(=O)(=O)CC2)c2ccc(F)cc2)c(F)c1 ; O=C(N1CCS(=O)(=O)CC1)N(Cc1ccc(-c2nnc(C(F)F)o2)cc1F)c1ccc(F)cc1]

A solution of N-(2-fluoro-4-(hydrazinecarbonyl)benzyl)-N-(4-fluorophenyl)thiomorpholine-4-carboxamide 1,1-dioxide (0.100 g, 0.228 mmol) prepared in Step 3 and triethylamine (0.095 mL, 0.684 mmol) in dichloromethane (4 mL) was mixed at the room temperature with 2,2-difluoroacetic anhydride (0.028 mL, 0.228 mmol), and stirred at the same temperature for 17 hr. Then, saturated aqueous sodium bicarbonate solution was added to the reaction mixture, followed by extraction with dichloromethane. The bi-phasic mixture was passed through a plastic frit to remove the solid residues and aqueous layer, and the organic layer collected was concentrated in vacuo. The residue was chromatographed (SiO₂, 4 g cartridge; ethyl acetate/hexane=20% to 50%) to give N-(4-(5-(difluoromethyl)-1,3,4-oxadiazol-2-yl)-2-fluorobenzyl)-N-(4-fluorophenyl)thiomorpholine-4-carboxamide 1,1-dioxide as white solid (0.053 g, 46.6%).

¹H NMR (400 MHZ, CDCl₃) δ 7.90 (dd, 1H, J=8.0, 1.6 Hz), 7.77 (dd, 1H, J=10.1, 1.6 Hz), 7.69 (t, 1H, J=7.6 Hz), 7.14-6.81 (m, 5H), 4.90 (s, 2H), 3.74-3.71 (m, 4H), 2.85-2.82 (m, 4H); LRMS(ES) m/z 499.3 (M⁺+1).

### Synthesis Example 5. Synthesis of the compound 295, N-((5-(5-(difluoromethyl)-1,3,4-oxadiazol-2-yl)pyridin-2-yl)methyl)-N-(4-fluorophenyl)thiomorpholine-4-carboxamide 1,1-dioxide

### [Step 1] Methyl 6-((N-(4-fluorophenyl)-1,1-dioxidothiomorpholine-4-carboxamido)methyl)nicotinate

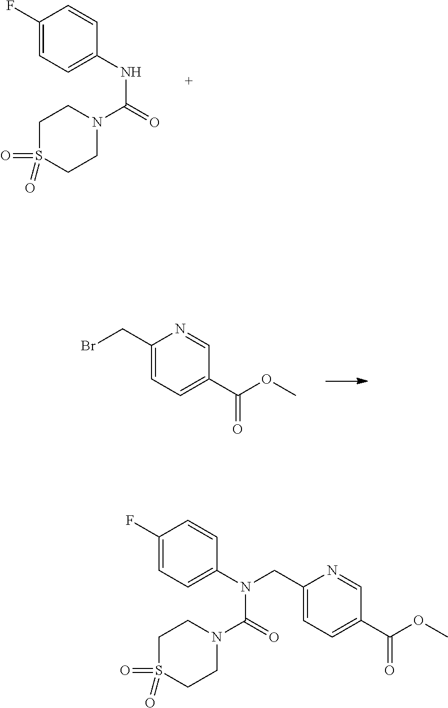 [SMILES: O=C(Nc1ccc(F)cc1)N1CCS(=O)(=O)CC1 ; COC(=O)c1ccc(CBr)nc1 ; COC(=O)c1ccc(CN(C(=O)N2CCS(=O)(=O)CC2)c2ccc(F)cc2)nc1]

A solution of N-(4-fluorophenyl)thiomorpholine-4-carboxamide 1,1-dioxide (0.500 g, 1.836 mmol) prepared in Step 1 of Synthesis Example 4 (Compound 285) and sodium hydride (60.00%, 0.081 g, 2.020 mmol) in N,N-dimethylformamide (10 mL) was stirred at 0° C. for 30 min, and mixed with methyl 6-(bromomethyl) nicotinate (0.465 g, 2.020 mmol). The reaction mixture was stirred at the room temperature for additional 5 hr, quenched at the room temperature by the addition of water (5 mL, 10 min stirring). Then, water was added to the reaction mixture, followed by extraction with ethyl acetate. The organic layer was washed with aqueous saturated sodium chloride solution, dried (anhydrous MgSO₄), filtered, and concentrated in vacuo. methyl 6-((N-(4-fluorophenyl)-1,1-dioxidothiomorpholine-4-carboxamido)methyl)nicotinate was used without further purification (0.450 g, 58.1%, brown solid).

### [Step 2] N-(4-fluorophenyl)-N-((5-(hydrazinecarbonyl)pyridin-2-yl)methyl)thiomorpholine-4-carboxamide 1,1-dioxide

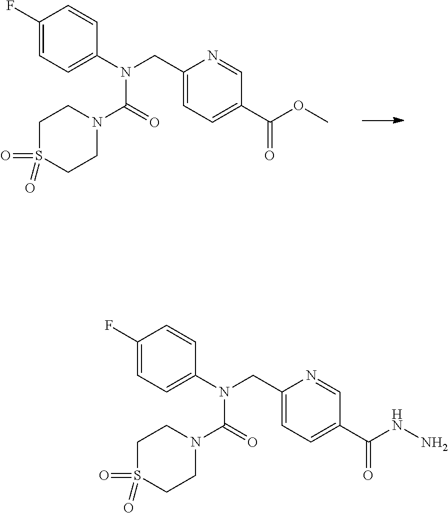 [SMILES: COC(=O)c1ccc(CN(C(=O)N2CCS(=O)(=O)CC2)c2ccc(F)cc2)nc1 ; NNC(=O)c1ccc(CN(C(=O)N2CCS(=O)(=O)CC2)c2ccc(F)cc2)nc1]

Methyl 6-((N-(4-fluorophenyl)-1,1-dioxidothiomorpholine-4-carboxamido)methyl)nicotinate (0.150 g, 0.356 mmol) prepared in Step 1 and hydrazine monohydrate (0.346 mL, 7.118 mmol) were mixed at the room temperature in ethanol (5 mL) and then stirred at 100° C. for 17 hr, cooled down to the room temperature. The precipitates were collected by filtration, washed by ethanol, and dried to give N-(4-fluorophenyl)-N-((5-(hydrazinecarbonyl)pyridin-2-yl)methyl)thiomorpholine-4-carboxamide 1,1-dioxide as pale yellow solid (0.111 g, 74.0%).

### [Step 3] N-((5-(2-(2,2-difluoroacetyl)hydrazine-1-carbonyl)pyridin-2-yl)methyl)-N-(4-fluorophenyl)thiomorpholine-4-carboxamide 1,1-dioxide

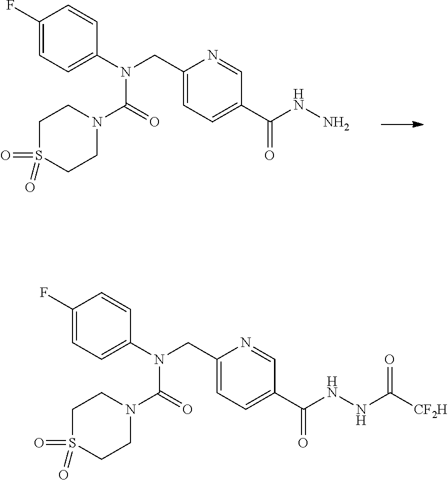 [SMILES: NNC(=O)c1ccc(CN(C(=O)N2CCS(=O)(=O)CC2)c2ccc(F)cc2)nc1 ; O=C(NNC(=O)C(F)F)c1ccc(CN(C(=O)N2CCS(=O)(=O)CC2)c2ccc(F)cc2)nc1]

A solution of N-(4-fluorophenyl)-N-((5-(hydrazinecarbonyl)pyridin-2-yl)methyl)thiomorpholine-4-carboxamide 1,1-dioxide (0.111 g, 0.263 mmol) prepared in Step 2 and triethylamine (0.110 mL, 0.790 mmol) in dichloromethane (5 mL) was mixed at the room temperature with 2,2-difluoroacetic anhydride (0.065 mL, 0.527 mmol), and stirred at the same temperature for 1 hr. Then, water was added to the reaction mixture, followed by extraction with dichloromethane. The bi-phasic mixture was passed through a plastic frit to remove the solid residues and aqueous layer, and the organic layer collected was concentrated in vacuo. The crude product was used without further purification (0.082 g, 62.3%, yellow solid).

### [Step 4] Synthesis of Compound 295

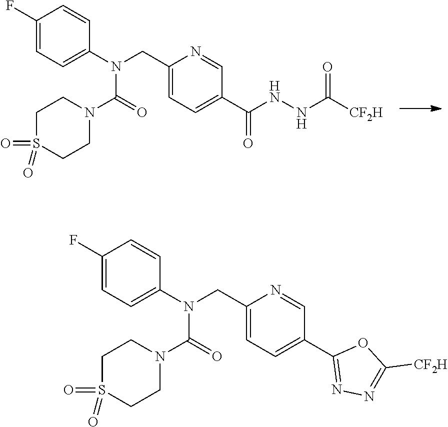 [SMILES: O=C(NNC(=O)C(F)F)c1ccc(CN(C(=O)N2CCS(=O)(=O)CC2)c2ccc(F)cc2)nc1 ; O=C(N1CCS(=O)(=O)CC1)N(Cc1ccc(-c2nnc(C(F)F)o2)cn1)c1ccc(F)cc1]

N-((5-(2-(2,2-difluoroacetyl)hydrazine-1-carbonyl)pyridin-2-yl)methyl)-N-(4-fluorophenyl)thiomorpholine-4-carboxamide 1,1-dioxide (0.082 g, 0.164 mmol) prepared in Step 3 and 1-methoxy-N-triethylammoniosulfonyl-methanimidate (Burgess reagent, 0.117 g, 0.493 mmol) were mixed at the room temperature in tetrahydrofuran (5 mL) and then stirred at 70° C. for 5 hr, cooled down to the room temperature, filtered to remove solids, and concentrated under the reduced pressure. The residue was chromatographed (SiO₂, 4 g cartridge; methanol/dichloromethane=0% to 10%) to give N-((5-(5-(difluoromethyl)-1,3,4-oxadiazol-2-yl)pyridin-2-yl)methyl)-N-(4-fluorophenyl)thiomorpholine-4-carboxamide 1,1-dioxide as white solid (0.015 g, 19.0%).

¹H NMR (400 MHZ, CDCl₃) δ 9.27 (d, 1H, J=1.6 Hz), 8.43 (dd, 1H, J=8.2, 2.2 Hz), 7.58 (d, 2H, J=8.2 Hz), 7.25-7.21 (m, 2H), 7.10-6.84 (m, 3H), 5.08 (s, 2H), 3.73 (t, 4H, J=5.1 Hz), 2.98 (t, 4H, J=5.2 Hz); LRMS(ES) m/z 482.1 (M⁺+1).

### Synthesis Example 6. Synthesis of the compound 296, N-((5-(5-(difluoromethyl)-1,3,4-oxadiazol-2-yl)pyridin-2-yl)methyl)-N-(3-fluorophenyl)thiomorpholine-4-carboxamide 1,1-dioxide

### [Step 1] Methyl 6-((N-(3-fluorophenyl)-1,1-dioxidothiomorpholine-4-carboxamido)methyl)nicotinate

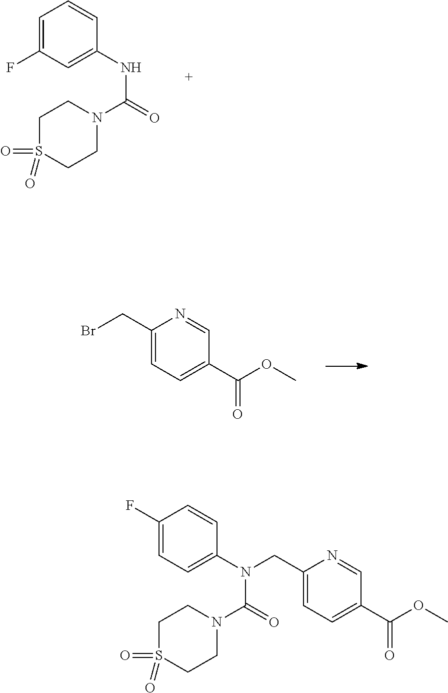 [SMILES: O=C(Nc1cccc(F)c1)N1CCS(=O)(=O)CC1 ; COC(=O)c1ccc(CBr)nc1 ; COC(=O)c1ccc(CN(C(=O)N2CCS(=O)(=O)CC2)c2ccc(F)cc2)nc1]

A solution of 1-fluoro-3-isocyanatobenzene (0.500 g, 3.647 mmol) in diethylether (10 mL) was mixed at 0° C. with thiomorpholine 1,1-dioxide (0.493 g, 3.647 mmol), and stirred at the same temperature for 1 hr. The reaction mixture was stirred at the room temperature for additional 4 hr. The precipitates were collected by filtration, washed by diethylether, and dried to give N-(3-fluorophenyl)thiomorpholine-4-carboxamide 1,1-dioxide as white solid (0.870 g, 87.6%).

A solution of N-(3-fluorophenyl)thiomorpholine-4-carboxamide 1,1-dioxide (0.500 g, 1.836 mmol) prepared above and sodium hydride (60.00%, 0.081 g, 2.020 mmol) in N,N-dimethylformamide (10 mL) was stirred at 0° C. for 30 min, and mixed with methyl 6-(bromomethyl) nicotinate (0.465 g, 2.020 mmol). The reaction mixture was stirred at the room temperature for additional 5 hr, quenched at the room temperature by the addition of water (5 mL, 10 min stirring). Then, water was added to the reaction mixture, followed by extraction with ethyl acetate. The organic layer was washed with aqueous saturated sodium chloride solution, dried (anhydrous MgSO₄), filtered, and concentrated in vacuo. Methyl 6-((N-(3-fluorophenyl)-1,1-dioxidothiomorpholine-4-carboxamido)methyl)nicotinate was used without further purification (0.450 g, 58.1%, brown solid).

### [Step 2] N-(3-fluorophenyl)-N-((5-(hydrazinecarbonyl)pyridin-2-yl)methyl)thiomorpholine-4-carboxamide 1,1-dioxide

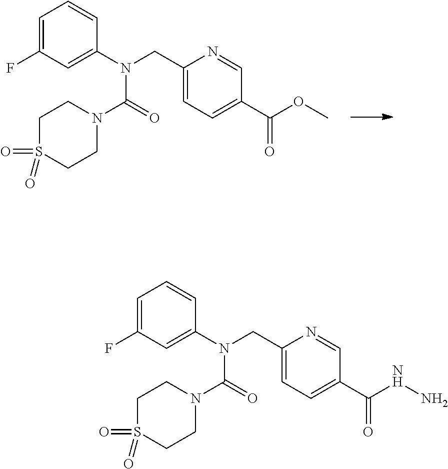 [SMILES: COC(=O)c1ccc(CN(C(=O)N2CCS(=O)(=O)CC2)c2cccc(F)c2)nc1 ; NNC(=O)c1ccc(CN(C(=O)N2CCS(=O)(=O)CC2)c2cccc(F)c2)nc1]

Methyl 6-((N-(3-fluorophenyl)-1,1-dioxidothiomorpholine-4-carboxamido)methyl)nicotinate (0.150 g, 0.356 mmol) prepared in Step 1 and hydrazine monohydrate (0.346 mL, 7.118 mmol) were mixed at the room temperature in ethanol (5 mL) and then stirred at 100° C. for 17 hr, cooled down to the room temperature. The precipitates were collected by filtration, washed by ethanol, and dried to give N-(3-fluorophenyl)-N-((5-(hydrazinecarbonyl)pyridin-2-yl)methyl)thiomorpholine-4-carboxamide 1,1-dioxide as pale yellow solid (0.113 g, 75.3%).

### [Step 3] N-((5-(2-(2,2-difluoroacetyl)hydrazine-1-carbonyl)pyridin-2-yl)methyl)-N-(3-fluorophenyl)thiomorpholine-4-carboxamide 1,1-dioxide

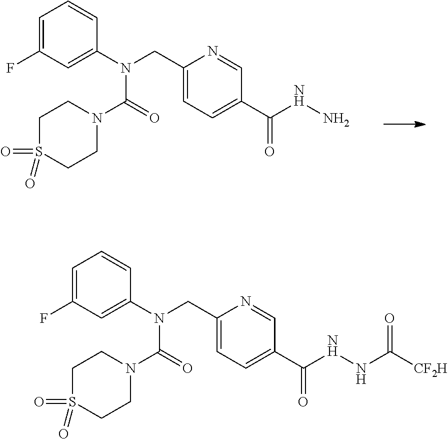 [SMILES: NNC(=O)c1ccc(CN(C(=O)N2CCS(=O)(=O)CC2)c2cccc(F)c2)nc1 ; O=C(NNC(=O)C(F)F)c1ccc(CN(C(=O)N2CCS(=O)(=O)CC2)c2cccc(F)c2)nc1]

A solution of N-(3-fluorophenyl)-N-((5-(hydrazinecarbonyl)pyridin-2-yl)methyl)thiomorpholine-4-carboxamide 1,1-dioxide (0.113 g, 0.268 mmol) prepared in Step 2 and triethylamine (0.112 mL, 0.804 mmol) in dichloromethane (5 mL) was mixed at the room temperature with 2,2-difluoroacetic anhydride (0.067 mL, 0.536 mmol), and stirred at the same temperature for 1 hr. Then, water was added to the reaction mixture, followed by extraction with dichloromethane. The bi-phasic mixture was passed through a plastic frit to remove the solid residues and aqueous layer, and the organic layer collected was concentrated in vacuo. N-((5-(2-(2,2-difluoroacetyl)hydrazine-1-carbonyl)pyridin-2-yl)methyl)-N-(3-fluorophenyl)thiomorpholine-4-carboxamide 1,1-dioxide was used without further purification (0.090 g, 67.2%, yellow solid).

### [Step 4] Synthesis of Compound 296

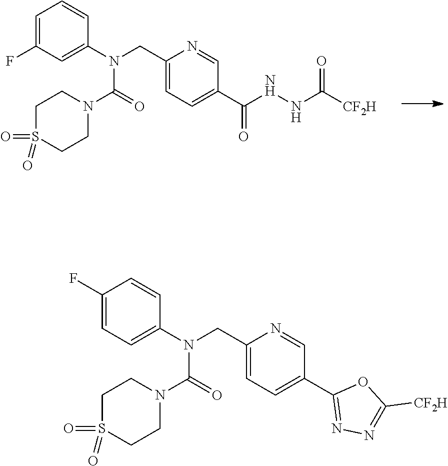 [SMILES: O=C(NNC(=O)C(F)F)c1ccc(CN(C(=O)N2CCS(=O)(=O)CC2)c2cccc(F)c2)nc1 ; O=C(N1CCS(=O)(=O)CC1)N(Cc1ccc(-c2nnc(C(F)F)o2)cn1)c1ccc(F)cc1]

N-((5-(2-(2,2-difluoroacetyl)hydrazine-1-carbonyl)pyridin-2-yl)methyl)-N-(3-fluorophenyl)thiomorpholine-4-carboxamide 1,1-dioxide (0.090 g, 0.180 mmol) prepared in Step 3 and 1-methoxy-N-triethylammoniosulfonyl-methanimidate (Burgess reagent, 0.129 g, 0.541 mmol) were mixed at the room temperature in tetrahydrofuran (5 mL) and then stirred at 70° C. for 5 hr, cooled down to the room temperature, filtered to remove solids, and concentrated under the reduced pressure. The residue was chromatographed (SiO₂, 4 g cartridge; methanol/dichloromethane=0% to 10%) to give N-((5-(5-(difluoromethyl)-1,3,4-oxadiazol-2-yl)pyridin-2-yl)methyl)-N-(3-fluorophenyl)thiomorpholine-4-carboxamide 1,1-dioxide as white solid (0.044 g, 50.7%).

¹H NMR (400 MHZ, CDCl₃) δ 9.28 (d, 1H, J=1.6 Hz), 8.46 (dd, 1H, J=8.2, 2.2 Hz), 7.58 (d, 1H, J=8.2 Hz), 7.37-7.32 (m, 1H), 7.10-6.92 (m, 4H), 5.14 (s, 2H), 3.76 (t, 4H, J=5.1 Hz), 3.03 (t, 4H, J=5.2 Hz); LRMS(ES) m/z 482.3 (M⁺+1).

### <Example> Confirmation of Therapeutic Effect of Compound of the Present Disclosure on Pulmonary Arterial Hypertension (PAH)

### Example 1

The therapeutic effect of the compound of the present disclosure on pulmonary arterial hypertension (PAH) was confirmed through cellular experiments.

### Example 1-1

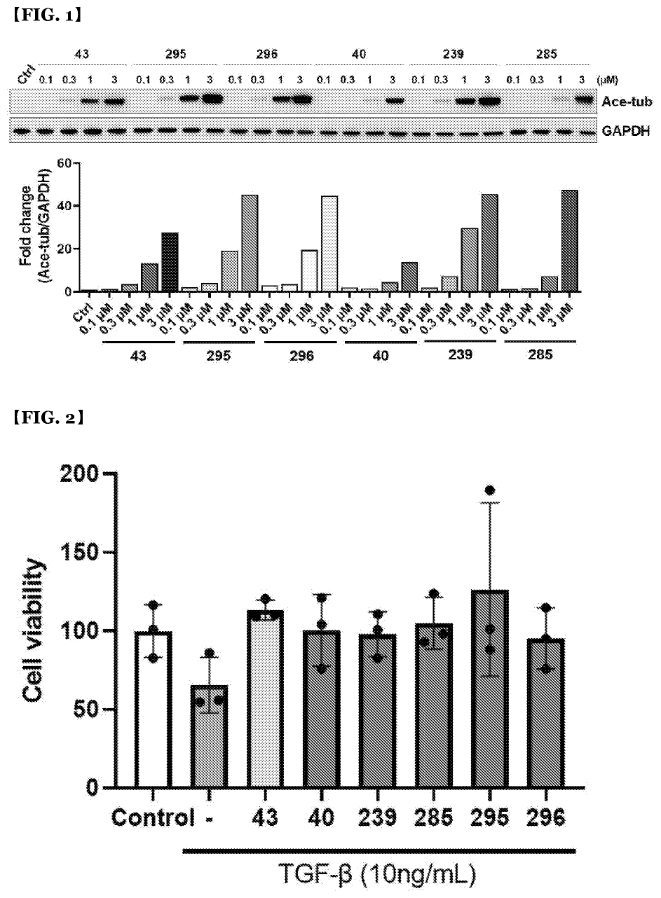

HPAEC (3.0×10⁵ cells/well, Human pulmonary artery endothelial cells) pulmonary artery endothelial cells were seeded in a six-well plate, and then treated with drugs (compounds 43, 295, 296, 40, 239, and 285) at each concentration. In four hours later at 37° C., proteins were extracted with lysis buffer and quantified by Bradford method. 5 μg of proteins were dissolved in sample buffer, subjected to electrophoresis on 4-12% gradient gel, transferred to a nitrocellulose membrane for seven minutes, and blocked in a 3% BSA solution for one hour. Anti-acetyl tubulin (1:1,000) and GAPDH (1:2,000) were added to the 3% BSA solution, after which the membrane was immersed therein and reacted at 4° C. for 10 hours, and then washed three times with 1×TBST for 10 minutes, respectively. IgG-HRP antibody (1:5,000) was added to 5% BSA, after which the membrane was immersed therein and reacted at room temperature for one hour, and then washed three times with 1×TBST for 10 minutes, respectively. An expression level of proteins was confirmed using an ECL solution, and the results thereof are shown in FIG. 1. In above FIG. 1, a control group indicates cells not treated with the compounds of Synthesis Examples.

As shown in FIG. 1, it could be confirmed that tubulin acetylation was expressed at a low concentration of 0.1μ of the compounds 43, 295, 296, 40, 239, and 285, respectively, thus all of the compounds 43, 295, 296, 40, 239 and 285 showed excellent activity even at a low concentration.

### Example 1-2

TGF-β stimulation to HPAEC cells, which are a model of pulmonary arterial hypertension disease, and the survival rate according to the compounds of Synthesis Examples 1 to 6 were confirmed through cell viability measurement.

HPAEC (2.0×10³ cells/well) cells were seeded in 96-well plate, and cultured for 24 hours in a serum free medium as starvation. 1 μM concentration of drugs (compounds 43, 295, 296, 40, 239, 285) were simultaneously treated with TGF-β (10 ng/ml) and cultured for 48 hours, after which cell viability was measured through CCK-8 assay, and the results thereof are shown in FIG. 2. In above FIG. 2, a control group indicates cells not treated with TGF-β and the compounds of Synthesis Examples, and “-” indicates cells treated only with TGF-β.

As shown in FIG. 2, when treated with compounds 43, 295, 296, 40, 239 and 285 after TGF-β stimulation, it was observed that a survival rate is all improved compared to a case where the compound is not treated after TGF-β stimulation, and thus it could be confirmed that the compound of the present disclosure indicates a HPAEC cell protection effect is excellent.

### Example 1-3

TGF-β stimulation to HPASMC (human pulmonary artery smooth muscle) cells, which are a model of pulmonary arterial hypertension disease, and a cell proliferation inhibition rate according to treatment with six compounds of Synthesis Examples 1 to 6 were confirmed through cell viability measurement.

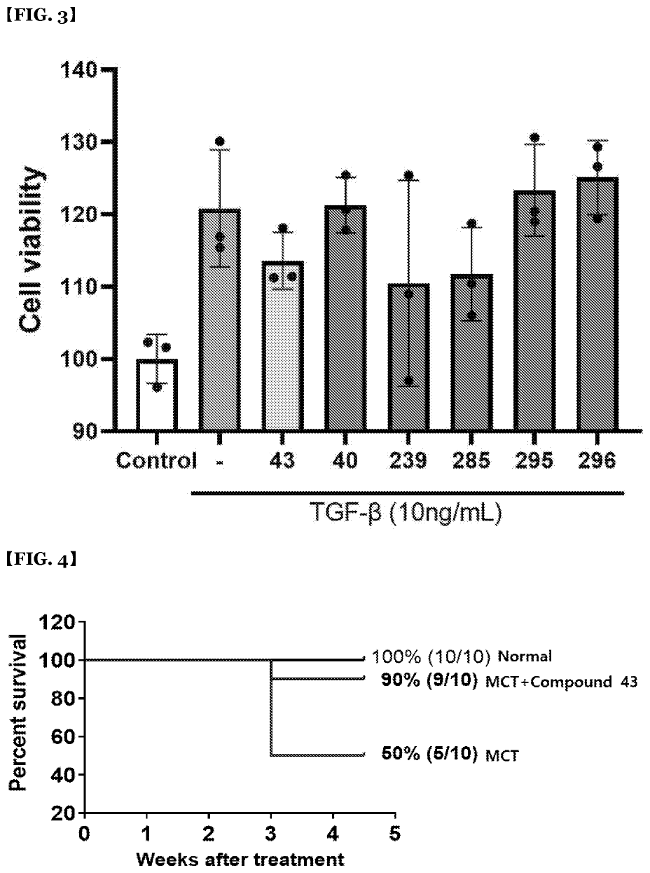

HPASMC (5.0×10³ cells/well) cells were seeded in 96-well plate, and cultured for 24 hours in a serum free medium as starvation. 1 μM concentration of drugs (compounds 43, 295, 296, 40, 239, 285) were simultaneously treated with TGF-β (10 ng/ml) and cultured for 24 hours, after which cell viability was measured through CCK-8 assay, and the results thereof are shown in FIG. 3. In above FIG. 3, a control group indicates cells not treated with TGF-β and the compounds of Synthesis Examples, and “-” indicates cells treated only with TGF-β.

As shown in FIG. 3, when treated with compounds 43, 295, 296, 40, 239, and 285 after TGF-β stimulation, it was observed that a proliferation rate is all inhibited compared to a case where the compound is not treated after TGF-β stimulation, and thus it could be confirmed that the compound of the present disclosure indicates an excessive HPASMC cell proliferation inhibition effect is excellent.

### Example 2

The effect of the compound of the present disclosure on treating pulmonary arterial hypertension (PAH) was confirmed by administering the compound of the present disclosure into an animal model for the PAH.

### 1) Test Animal Preparation and Drug Administration

SD rats having the following conditions were prepared.

| | | | | Acclimation |
|---|---|---|---|---|
| Type | Source | Gender | Age | period |
| SPF SD rat | Orient Bio | Male | Six weeks | One week |

Rats were supplied from Orient Bio and fed on a standard diet (Central Lab Animal, Inc.) and kept under the conditions of constant temperature (22±2° C.), humidity (44-56%) and lighting (12 h light-dark cycle) with a free access to drinking. All experimental procedures were approved and performed according to the Institutional Animal Care and Use Committee (IACUC) of the Korea CKD Laboratory Animal Center (with the IACUC animal study protocol approval number: CE22368-1).

Rats, experimental animals, were divided into groups as follows in consideration of a body weight on the day before drug administration began.

| | | | Number |
|---|---|---|---|
| Group | Dosage | Usage | of Animal |
| Normal | Saline solution | | 10 |
| Control (vehicle) | MCT aqueous | One subcutaneous | 10 |
| | solution at 50 mg/kg | administration | |
| Drug administration | MCT aqueous | One subcutaneous | 10 |
| group (Compound | solution at 50 mg/kg | administration | |
| 43) | Compound 43 | Oral administration | |
| | at 20 mg/kg | twice a day | |

During a test period, the normal group was dosed with saline, and the control group (MCT+Vehicle) was subcutaneously dosed with a monocrotaline (MCT) aqueous solution once at 3 ml/Kg, and then dosed with a vehicle consisting of a mixed solution of cremophor EL, ethanol, and saline at a volume ratio of 1:1:8. A MCT aqueous solution was prepared to reach a concentration of 20 mg/mL in a solvent adjusted to pH 7.4 using a 1 N NAOH solution in 1 N HCL. After that, subcutaneous administration was performed by adjusting to 50 mg/kg.

The drug administration group was first subcutaneously dosed with the MCT aqueous solution at 3 ml/Kg once, and then orally dosed with compound 43 (the compound of Synthesis Example 1) at 20 mg/Kg twice a day during the test period (four weeks). The compound 43 was dissolved in a mixed solution of cremophor EL, ethanol, and saline at a volume ratio of 1:1:8, and then orally administered.

### 2) Test Results

All data were expressed as mean±standard error, and each group and the control group were compared using an unpaired T test (p<0.05) to determine the effect of each experimental group.

### 2-1) Individual Survival Rate

After group separation and then administration of the MCT aqueous solution, the survival rate was evaluated by confirming a dead individual during the test period.

The animal model with induced pulmonary arterial hypertension is a model in which an inner diameter of overall smooth muscle is narrowed due to the proliferation and contraction of endothelial cells and smooth muscle cells constituting the pulmonary artery delivered from the heart to the lungs, and shows a problem in the circulation of blood delivered from the heart to the lungs. Such a problem with blood circulation decreases the supply of oxygen and nutrients to the whole body, and thus causes overall body abnormalities. Therefore, the survival rate are indicators for best reflecting an overall condition of the animal.

An individual survival rate during the test period of four weeks is shown in FIG. 4.

The individual survival rate was calculated by reflecting the number of animals surviving in the corresponding week from the first 10 animals participating in the experiment for each group. Each number on the left side of the line graph means an individual survival rate % (number of surviving individuals/total number of animals participating in experiment).

As confirmed in FIG. 4, no individual in the normal group died for four weeks, but in the control group dosed with MCT and vehicle, death occurred from a third week of the experiment, and a total of five individuals died just with an individual survival rate of 50%. On contrary, in the group dosed with compound 43, only one out of 10 animals died, thus showing a significant individual survival rate of 90%.

### 2-2) Measurement of Lung and Ventricle Weights

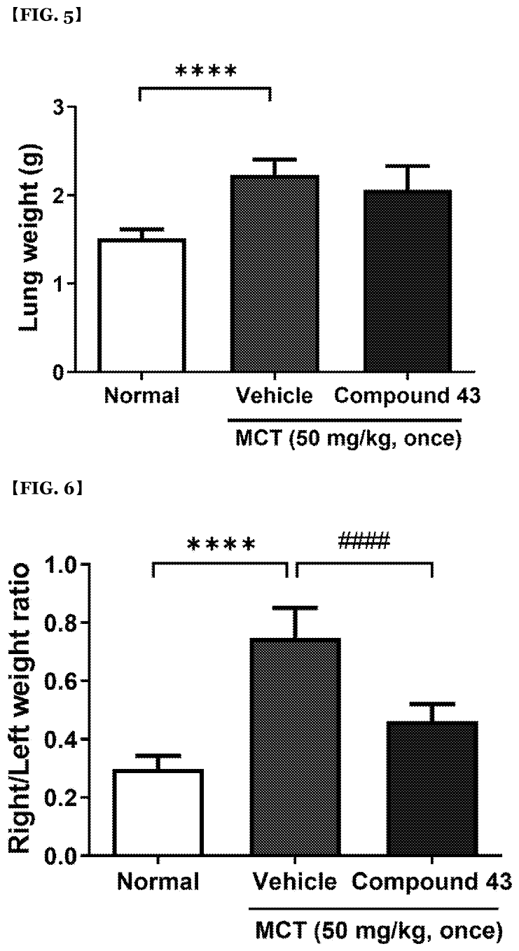

After four weeks of the test period, the lung and the heart were removed to measure the weight of the lung and the weight of left ventricle and right ventricle in each group, and the results are shown in FIGS. 5 and 6.

The heart of the animal model of pulmonary arterial hypertension was subjected to overload under pressure due to a high blood pressure in blood vessels delivered from the right ventricle to the lungs, and accordingly the volume of the right ventricle side may increase and the total heart weight may increase. In other words, in the case of an animal model of pulmonary arterial hypertension by MCT, after an increase in right ventricle volume is primarily induced, the symptoms become severe and the survival rate may be increased.

In FIGS. 5 and 6, all result values were expressed as mean±standard error, and statistical significance was analyzed using an unpaired T test for comparison between the control group dosed with vehicle and other groups. **** indicates comparison between normal group and control group as P<0.0001, whereas #### indicates comparison between control group and drug administration group as P<0.0001.

FIG. 5 shows a weight of the lung. As confirmed in FIG. 5, the weight of the lung was significantly increased in the control group dosed with vehicle compared to that in the normal group.

FIG. 6 shows a ratio of right ventricle weight to left ventricle weight, which is a value obtained by dividing the weight of the right ventricle by the weight of the left ventricle. As confirmed in above FIG. 6, a ratio of right ventricle weight to left ventricle weight in the control group dosed with vehicle was greatly increased compared to that of the normal group. However, the ratio of right ventricle weight to left ventricle weight in the drug administration group dosed with compound 43 was significantly reduced compared to the control group.

From this, it can be seen that the compound of the present disclosure ameliorates the hypertrophy of the right ventricle side caused by the pressure load of the pulmonary artery.

### 2-3) ECG Test

After four weeks of the test period, the animal was subjected to inhalation anesthesia with isoflurane, and electrodes were attached to the limbs to measure the animal's electrocardiogram for more than 30 seconds. The result was measured using a Bio Amp ECG meter and analysis was performed using LabChart 8 software.

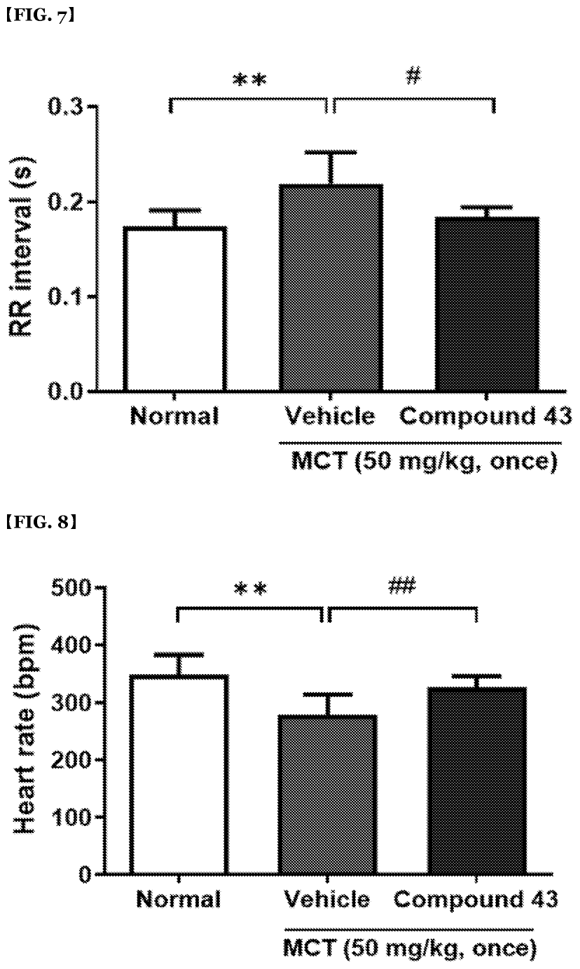

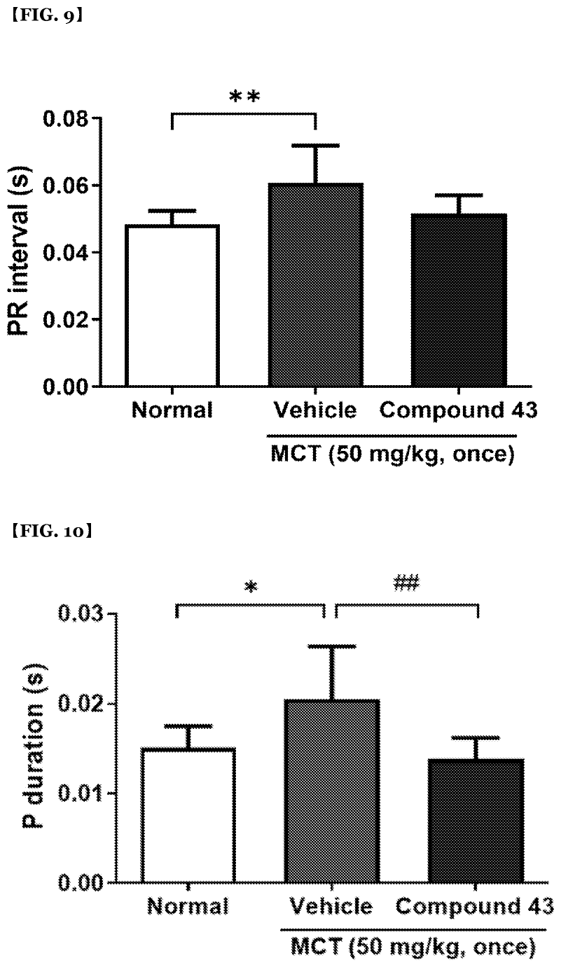

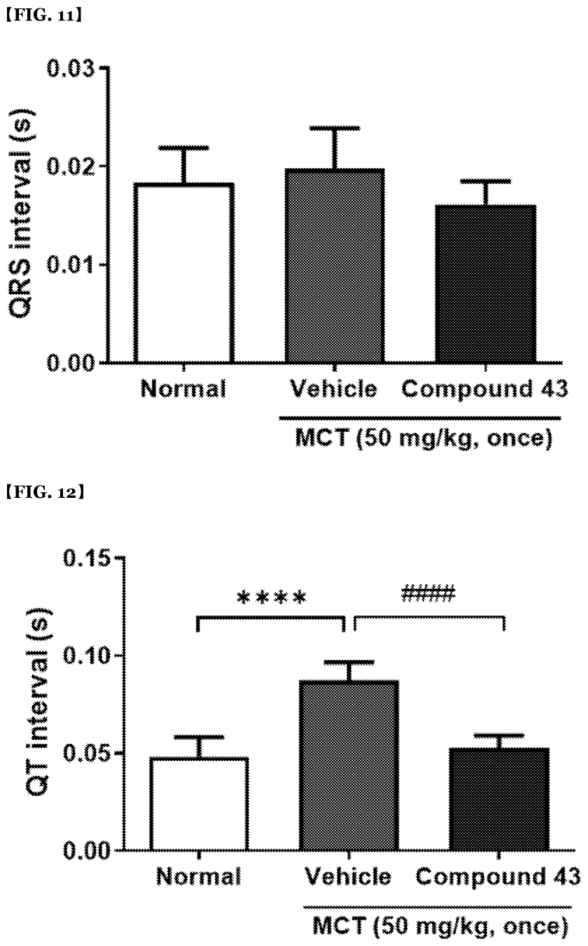

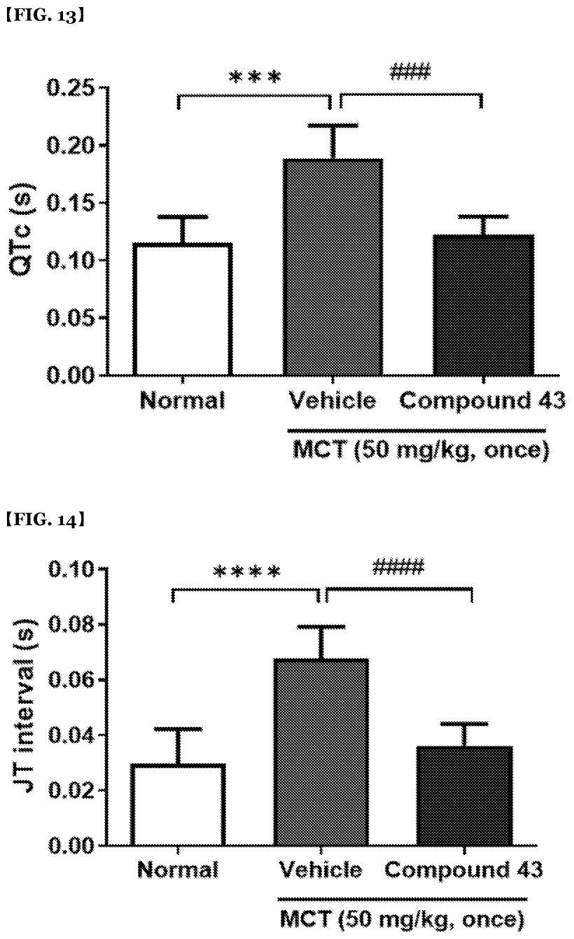

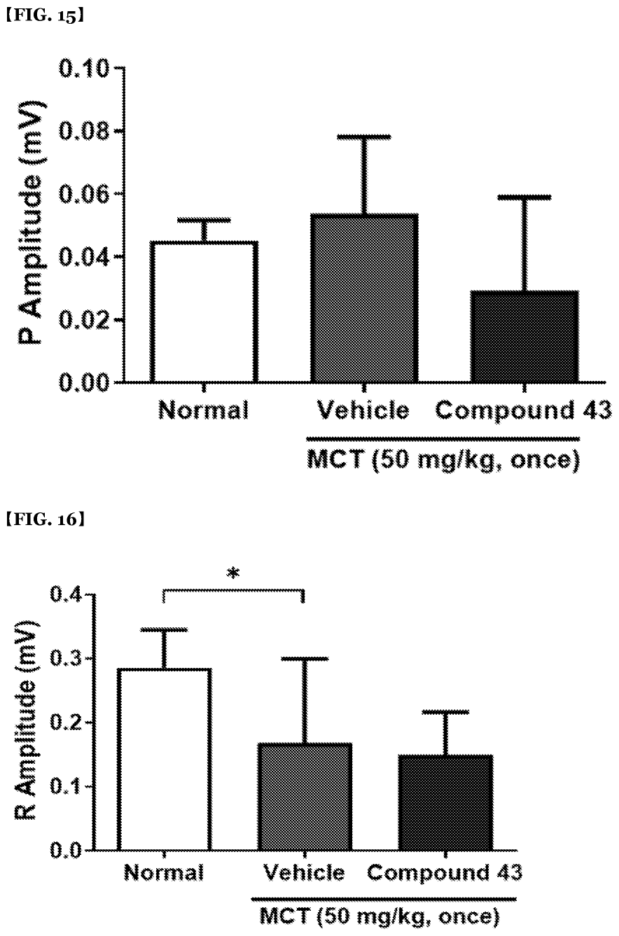

The results of ECG measurement are shown in FIGS. 7 to 16. In above FIGS. 7 to 16, all result values were expressed as mean±standard error, and statistical significance was analyzed using an unpaired T test for the control group and each other groups. * may mean P<0.05, ** may mean P<0.01, *** may mean P<0.001 and **** may mean P<0.0001, and *, ** *** and **** may indicate comparison between normal group and control group. # may mean P<0.05, ## may mean P<0.01, ### may mean P<0.001 and #### may mean P<0.0001, and #, ##, ### and #### may indicate comparison between control group and drug administration group. In above FIGS. 7 to 16, Normal may mean the normal group, Vehicle may mean the control group, and Compound 43 may mean the drug administration group.

As confirmed in above FIGS. 7 to 16, it was confirmed that the control group dosed with the vehicle showed a significant change in overall heart rate and RR interval compared to the normal group. This phenomenon seemed to be highly related to prolongation of a QT interval during an electrocardiogram process. It was well known that an increase in QT interval is highly related to hypertrophy of the right ventricle, and this was confirmed through a study of clinical patients that the QT interval is significantly increased with hypertrophy of the right ventricle in patients with actual pulmonary arterial hypertension (Int J Cardiol. QTc prolongation is associated with impaired right ventricular function and predicts mortality in pulmonary hypertension. 2013; 167(3):669-676)

Meanwhile, it was confirmed that the QT interval, QTc, and JT interval was significantly reduced in the drug administration group dosed with compound 43 compared to the control group.

Therefore, it can be seen that the compound of the present disclosure ameliorates hypertrophy of the right ventricle side, heart rate and ventricular bradycardia caused by pressure load of the pulmonary artery.

The present disclosure provides a pharmaceutical composition, a method, and a use as follow:

Item 1. A pharmaceutical composition for preventing or treating pulmonary arterial hypertension, comprising a compound represented by the above-mentioned formula I the above, optical isomers thereof or pharmaceutically acceptable salts thereof as an active ingredient.

Item 2. The pharmaceutical composition of item 1, wherein the compound represented by formula I is at least one selected from the group consisting of the above-mentioned compound 1 to 450 which is described in the above-mentioned Table A.

Item 3. The pharmaceutical composition of item 1 or 2, wherein the compound represented by formula I is at least one selected from the group consisting of the compound 40, the compound 43, the compound 239, the compound 285, the compound 295 and the compound 296 which is described in the above-mentioned Table B.

Item 4. A method for preventing or treating pulmonary arterial hypertension, including administering a compound represented by above formula I, optical isomers thereof or pharmaceutically acceptable salts thereof described in item 1 to 3 into an individual.

Item 5. A use of the compound represented by the above formula I, optical isomers thereof or pharmaceutically acceptable salts thereof described in items 1 to 3 for preventing or treating pulmonary arterial hypertension.

Item 6. A use of the compound represented by above formula I, optical isomers thereof or pharmaceutically acceptable salts thereof described in items 1 to 3 in preparing a medicament for preventing or treating pulmonary arterial hypertension.

Item 7. The pharmaceutical composition according to any one of items 1 to 3, the method according to item 4, or the use according to item 5 or 6, wherein the pulmonary arterial hypertension is at least one selected from the group consisting of pulmonary arterial hypertension is at least one selected from the group consisting of idiopathic pulmonary arterial hypertension (IPAH), hereditary pulmonary arterial hypertension, drug- and toxin-induced pulmonary arterial hypertension, disease-associated pulmonary arterial hypertension (APAH), pulmonary arterial hypertension showing a long-term response to calcium channel blockers, pulmonary arterial hypertension with definite signs of venous/capillary invasions, and persistent pulmonary arterial hypertension in neonates.

Item 8. The pharmaceutical composition according to any one of items 1 to 3, the method according to item 4, or the use according to item 5 or 6, wherein the pulmonary arterial hypertension has pulmonary arterial pressure of 25 mmHg or more.

Item 9. The pharmaceutical composition according to any one of items 1 to 3, 7, and 8, wherein the pharmaceutical composition is orally administered.

Item 10. The method according to any one of items 4, 7, and 8, or the use according to any one of items 5 to 8, wherein the compound represented by above formula I, optical isomers thereof or pharmaceutically acceptable salts thereof described in item 1 to 3 is orally administered.

While specific portions of the present invention have been described in detail above, it is apparent to those skilled in the art that such detailed descriptions are set forth to illustrate exemplary embodiments only, but are not construed to limit the scope of the present invention. Thus, it should be understood that the substantial scope of the present invention is defined by 5 the accompanying claims and equivalents thereto.

1. A pharmaceutical composition for preventing or treating pulmonary arterial hypertension, comprising a compound represented by formula I below, optical isomers thereof or pharmaceutically acceptable salts thereof as an active ingredient: 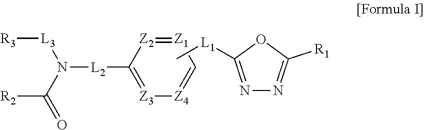
in formula I,
wherein L₁, L₂ or L₃ are each independently a bond or —(C₁-C₂ alkylene)-;
R₁ is —CX₂H or —CX₃;
R₂ is —NR^{A}R^{B}, —OR^{C}, 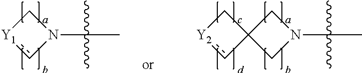
wherein, at least one H of 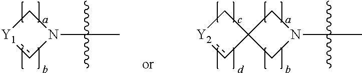
may be substituted with —X, —OH, —O(C₁-C₄ alkyl), —NR^{D}R^{E}, —(C₁-C₄ alkyl), —CF₃, —CF₂H, —CN, -aryl, -heteroaryl, —(C₁-C₄ alkyl)-aryl or —(C₁-C₄ alkyl)-heteroaryl, [wherein at least one H of the -aryl, -heteroaryl, —(C₁-C₄ alkyl)-aryl or —(C₁-C₄ alkyl)-heteroaryl may be substituted with —X, —OH, —CF₃ or —CF₂H];
R₃ is —H, —(C₁-C₄ alkyl), —(C₁-C₄ alkyl)-O(C₁-C₄ alkyl), —(C₁-C₄ alkyl)-C(═O)—O(C₁-C₄ alkyl), —(C₃-C₇ cycloalkyl), —(C₂-C₆ cycloheteroalkyl), -aryl, -heteroaryl, -adamantyl, 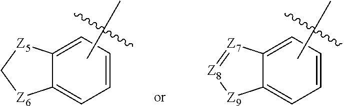
wherein, at least one H of —(C₁-C₄ alkyl) may be substituted with —X or —OH,
at least one H of -aryl or -heteroaryl each independently may be substituted with —X, —OH, —O(C₁-C₄ alkyl), —OCF₃, —O-aryl, —NR^{D}R^{E}, —(C₁-C₄ alkyl), —CF₃, —CF₂H, —C(═O)—(C₁-C₄ alkyl), —C(═O)—O(C₁-C₄ alkyl), —C(═O)—NR^{D}R^{E}, —S(═O)₂—(C₁-C₄ alkyl), aryl, heteroaryl, 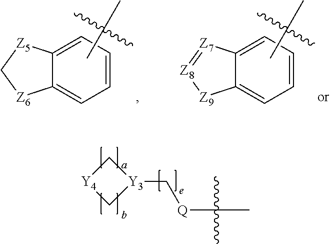
wherein, at least one H of 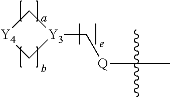
may be substituted with —X, —(C₁-C₄ alkyl), —NR^{D}R^{E}, —CF₃ or —CF₂H,
at least one H of —(C₃-C₇ cycloalkyl), —(C₂-C₆ cycloheteroalkyl), adamantyl, 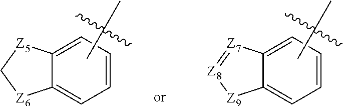
may be each independently substituted with —X, —OH or —(C₁-C₄ alkyl);
Y₁, Y₂ and Y₄ are each independently —CH₂—, —NR^{F}—, —O—, —C(═O)— or —S(═O)₂—;
Y₃ is —CH— or —N—;
Z₁ to Z₄ are each independently N or CR^{Z}, wherein at least three of Z₁ to Z₄ may not be simultaneously N, and R^{Z} is —H, —X or —O(C₁-C₄ alkyl);
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, -heteroaryl, —(C₁-C₄ aryl)-heteroaryl, —(C₃-C₇ cycloalkyl), —(C₂-C₆ heterocycloalkyl) or 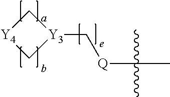
wherein, at least one H of the —(C₁-C₄ alkyl), —(C₁-C₄ alkyl)-OH or —(C₁-C₄ alkyl)-NR^{D}R^{E} may be substituted with —X,
at least one H of the -aryl, —(C₁-C₄ alkyl)-aryl, -heteroaryl, —(C₁-C₄ alkyl)-heteroaryl, —(C₃-C₇ cycloalkyl) or —(C₂-C₆ heterocycloalkyl) may be substituted with —X, —OH, —O(C₁-C₄ alkyl), —(C₁-C₄ alkyl), —CF₃, —CF₂H or —CN,
at least one H of 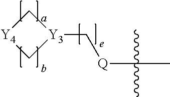
may be substituted with n-X, —OH, —O(C₁-C₄ alkyl), —(C₁-C₄ alkyl), —CF₃, —CF₂H, —CN, —(C₂-C₆ heterocycloalkyl), -aryl, —(C₁-C₄ alkyl)-aryl, -heteroaryl or -heteroaryl-(C₁-C₄ alkyl);
R^{C} is —(C₁-C₄ alkyl), -aryl, —(C₁-C₄ alkyl)-aryl, -heteroaryl or —(C₁-C₄ alkyl)-heteroaryl, wherein, at least one H of —(C₁-C₄ alkyl) may be substituted with —X or —OH, at least one H of -aryl, —(C₁-C₄ alkyl)-aryl, -heteroaryl or —(C₁-C₄ alkyl)-heteroaryl may be substituted with —X, —OH, —CF₃ or —CF₂H;
R^{D} and R^{E} are each independently —H, —(C₁-C₄ alkyl), -aryl or —(C₁-C₄ alkyl)-aryl, wherein, at least one H of —(C₁-C₄ alkyl) may be substituted with —X or —OH, at least one H of -aryl or —(C₁-C₄ alkyl)-aryl may be substituted with —X, —OH, —CF₃ or —CF₂H;
R^{F} is —H, —(C₁-C₆ alkyl), —(C₁-C₄ alkyl)-OH, —(C₁-C₄ alkyl)-O—(C₁-C₄ alkyl), —C(═O)—(C₁-C₄ alkyl), —C(═O)-0 (C₁-C₄ alkyl), —(C₁-C₄ alkyl)-C(═O)—O(C₁-C₄ alkyl), —(C₁-C₄ alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇cycloalkyl), —(C₂-C₆ heterocycloalkyl) or —(C₁-C₄ alkyl)-C(═O)—(C₂-C₆ heterocycloalkyl)
wherein at least one H of —(C₁-C₄ alkyl), —(C₁-C₄ alkyl)-OH, —(C₁-C₄ alkyl)-O—(C₁-C₄ alkyl), —C(═O)—(C₁-C₄ alkyl), —C(═O)—O(C₁-C₄ alkyl), —(C₁-C₄ alkyl)-C(═O)—O(C₁-C₄ alkyl), —(C₁-C₄ alkyl)-NR^{D}R^{E} or —S(═O)₂—(C₁-C₄ alkyl) may be substituted with —X,
at least one H of -aryl, —(C₁-C₄alkyl)-aryl, —(C₂-C₄ alkenyl)-aryl, -heteroaryl, —(C₁-C₄ alkyl)-heteroaryl, —C(═O)—(C₃-C₇ cycloalkyl), —C₂-C₆ heterocycloalkyl or —(C₁-C₄alkyl)-C(═O)—(C₂-C₆heterocycloalkyl) may be substituted with —X, —OH, —CF₃ or —CF₂H;
R^{G} is —H or —(C₁-C₄ alkyl);
Q is —O— or a bond; [symbol]
is a single bond or double bond, provided that, [symbol] is a double bond, Y₁ is ═CH—;
a to e are each independently an integer of 0, 1, 2, 3 or 4 provided that, a and b may not be simultaneously 0, and c and d may not be simultaneously 0;
X is each independently F, Cl, Br or I.

2. The pharmaceutical composition of claim 1, wherein in the compound represented by formula I,
L₁, L₂ or L₃ are each independently a bond or —(C₁-C₂alkylene)-;
R₁ is —CX₂H or —CX₃;
R₂ is —NR^{A}R^{B}, —OR^{C}, 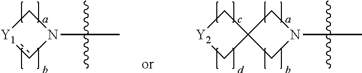
wherein at least one of H of 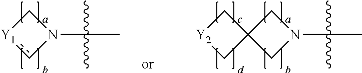
may be substituted with —X, —OH, —NR^{D}R^{E}, —(C₁-C₄ alkyl);
R₃ is —(C₁-C₄ alkyl), —(C₃-C₇ cycloalkyl), -aryl, -heteroaryl, -adamantyl, 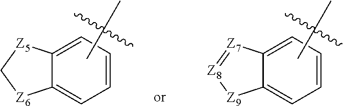
wherein at least one H of -aryl or -heteroaryl may be each independently substituted with —X, —O(C₁-C₄alkyl), —OCF₃, —O-aryl, —NR^{D}R^{E}, —(C₁-C₄ alkyl), —CF₃, —S(═O)₂—(C₁-C₄alkyl), -aryl, -heteroaryl, 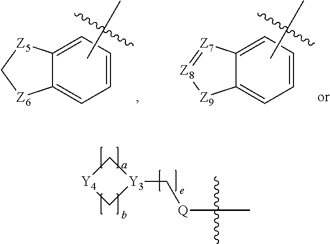
wherein, at least one H of 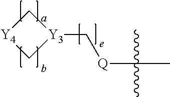
may be substituted with —NR^{D}R^{E} or (C₁-C₄ alkyl),
at least one H of 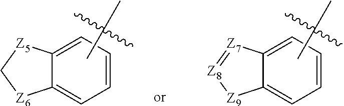
maybe each independently substituted with —(C₁-C₄ alkyl);
Y₁, Y₂ and Y₄ are each independently —CH₂—, —NR^{F}—, —O—, —C(═O)— or —S(═O)₂—;
Y₃ is —CH— or —N—;
Z₁ to Z₄ is each independently N or CR^{Z} wherein at least three of Z₁ to Z₄ may not be simultaneously N, and R^{Z} is —H, —X or —O(C₁-C₄alkyl);
Z₅ and Z₆ are each independently —CH₂— or —O—;
Z₇ and Z₈ are each independently ═CH— or ═N—;
Z₉ is —NR^{G}— or —S—;
R^{A} and R^{B} are each independently —H, —(C₁-C₄ alkyl), —(C₁-C₄ alkyl)-OH, —(C₁-C₄ alkyl)-NR^{D}R^{E}, -aryl, —(C₁-C₄ alkyl)-aryl, —(C₃-C₇ cycloalkyl) or 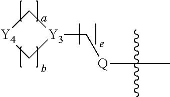
wherein, at least one H of 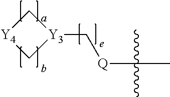
may be substituted with —X, —(C₁-C₄alkyl), —CF₃, —(C₂-C₆ heterocycloalkyl), —(C₁-C₄ alkyl)-aryl, -heteroaryl or heteroaryl-(C₁-C₄ alkyl);
R^{C} is-(C₁-C₄ alkyl) or -aryl;
R^{D} and R^{E} are each independently —H, —(C₁-C₄alkyl) or —(C₁-C₄ alkyl)-aryl;
R^{F} is —H, —(C₁-C₆alkyl), —(C₁-C₄alkyl)-OH, —(C₁-C₄ alkyl)-O—(C₁-C₄ alkyl), —C(═O)—(C₁-C₄alkyl), —C(═O)—O(C₁-C₄ alkyl), —(C₁-C₄ alkyl)-C(═O)—O(C₁-C₄alkyl), —(C₁-C₄alkyl)-NR^{D}R^{E}, —S(═O)₂—(C₁-C₄ alkyl), -aryl, —(C₁-C₄ alkyl)-aryl, —(C₂-C₄ alkenyl)-aryl, -heteroaryl, —(C₁-C₄ alkyl)-heteroaryl, —C(═O)—(C₃-C₇ cycloalkyl), —(C₂-C₆ heterocycloalkyl) or —(C₁-C₄ alkyl)-C(═O)—(C₂-C₆ heterocycloalkyl)
wherein at least one H of —(C₁-C₄alkyl) or —C(═O)—O(C₁-C₄alkyl) may be substituted with —X,
at least one H of -aryl may be substituted with —X;
R^{G} is —(C₁-C₄ alkyl);
Q is —O— or a bond; [symbol]
is a single bond or a double bond provided that [symbol] is a double bond, Y₁ is —CH—;
a to e are each independently an integer of 0, 1, 2, 3 or 4 provided that a and b may not be simultaneously 0, and c and d may not be simultaneously 0;
X is each independently F, Cl, Br or I.

3. The pharmaceutical composition of claim 1, wherein the compound represented by formula I is the compound represented by formula Ia: 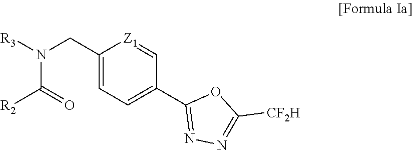
in formula Ia, wherein,
R₂ is 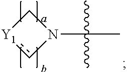
R₃ is -aryl wherein, at least one H of -aryl may be each independently substituted with —X;
Y₁ is —O— or —S(═O)₂—;
Z₁ is N or CR^{Z} wherein, R^{Z} is —X;
a and b are each independently an integer of 0, 1, 2, 3 or 4 wherein, a and b may not be simultaneously 0;
X is each independently F, Cl, Br or I.

4. The pharmaceutical composition of claim 3, wherein in the compound represented by formula Ia,
R₂ is 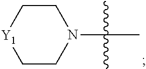
R₃ is -phenyl wherein, at least one H of -phenyl each independently is substituted with —F or —Cl;
Y₁ is —O— or —S(═O)₂—;
Z₁ is N or CF.

5. A pharmaceutical composition for preventing or treating pulmonary arterial hypertension, comprising a compound, optical isomers thereof or pharmaceutically acceptable salts thereof as an active ingredient, wherein the compound has the following structure:

| Compound | Structure |
|---|---|
| 1 | 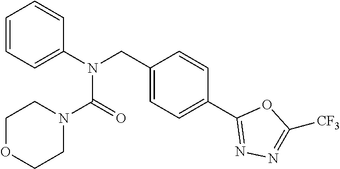 [SMILES: O=C(N1CCOCC1)N(Cc1ccc(-c2nnc(C(F)(F)F)o2)cc1)c1ccccc1] |
| 2 | 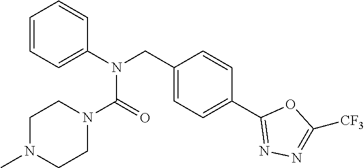 [SMILES: CN1CCN(C(=O)N(Cc2ccc(-c3nnc(C(F)(F)F)o3)cc2)c2ccccc2)CC1] |
| 3 | 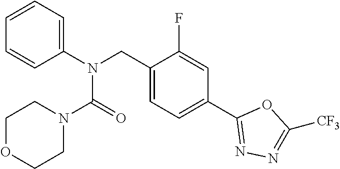 [SMILES: O=C(N1CCOCC1)N(Cc1ccc(-c2nnc(C(F)(F)F)o2)cc1F)c1ccccc1] |
| 4 | 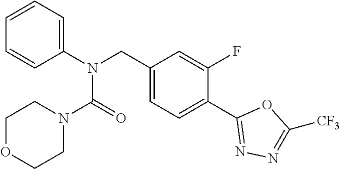 [SMILES: O=C(N1CCOCC1)N(Cc1ccc(-c2nnc(C(F)(F)F)o2)c(F)c1)c1ccccc1] |
| 5 | 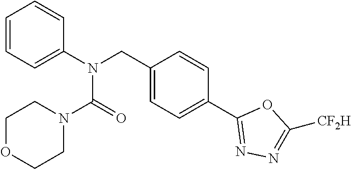 [SMILES: O=C(N1CCOCC1)N(Cc1ccc(-c2nnc(C(F)F)o2)cc1)c1ccccc1] |
| 6 | 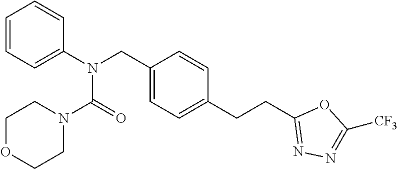 [SMILES: O=C(N1CCOCC1)N(Cc1ccc(CCc2nnc(C(F)(F)F)o2)cc1)c1ccccc1] |
| 7 | 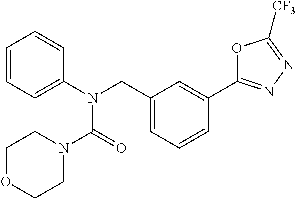 [SMILES: O=C(N1CCOCC1)N(Cc1cccc(-c2nnc(C(F)(F)F)o2)c1)c1ccccc1] |
| 8 | 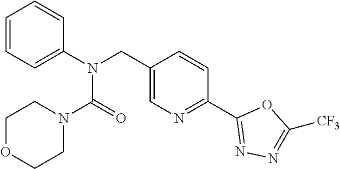 [SMILES: O=C(N1CCOCC1)N(Cc1ccc(-c2nnc(C(F)(F)F)o2)nc1)c1ccccc1] |
| 9 | 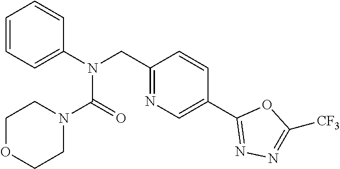 [SMILES: O=C(N1CCOCC1)N(Cc1ccc(-c2nnc(C(F)(F)F)o2)cn1)c1ccccc1] |
| 10 | 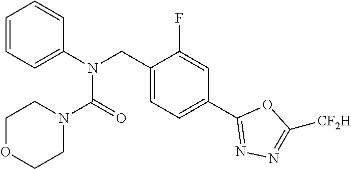 [SMILES: O=C(N1CCOCC1)N(Cc1ccc(-c2nnc(C(F)F)o2)cc1F)c1ccccc1] |
| 11 | 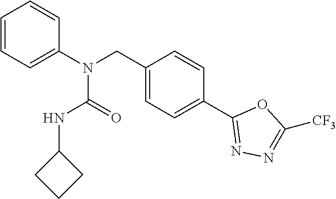 [SMILES: O=C(NC1CCC1)N(Cc1ccc(-c2nnc(C(F)(F)F)o2)cc1)c1ccccc1] |
| 12 | 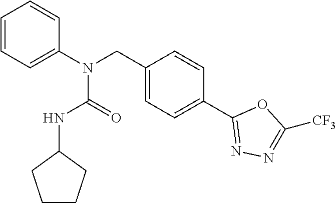 [SMILES: O=C(NC1CCCC1)N(Cc1ccc(-c2nnc(C(F)(F)F)o2)cc1)c1ccccc1] |
| 13 | 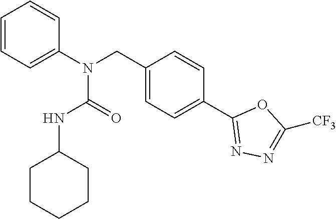 [SMILES: O=C(NC1CCCCC1)N(Cc1ccc(-c2nnc(C(F)(F)F)o2)cc1)c1ccccc1] |
| 14 | 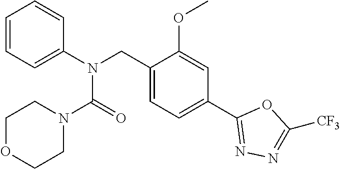 [SMILES: COc1cc(-c2nnc(C(F)(F)F)o2)ccc1CN(C(=O)N1CCOCC1)c1ccccc1] |
| 15 | 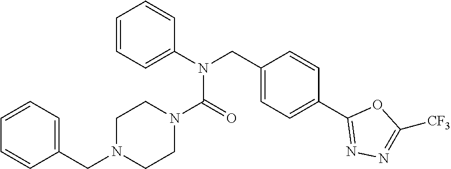 [SMILES: O=C(N1CCN(Cc2ccccc2)CC1)N(Cc1ccc(-c2nnc(C(F)(F)F)o2)cc1)c1ccccc1] |
| 16 | 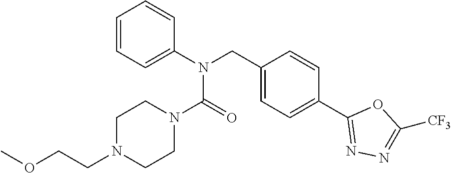 [SMILES: COCCN1CCN(C(=O)N(Cc2ccc(-c3nnc(C(F)(F)F)o3)cc2)c2ccccc2)CC1] |
| 17 | 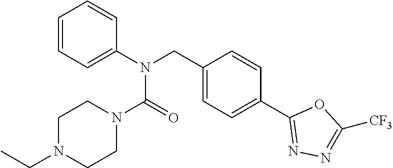 [SMILES: CCN1CCN(C(=O)N(Cc2ccc(-c3nnc(C(F)(F)F)o3)cc2)c2ccccc2)CC1] |
| 18 | 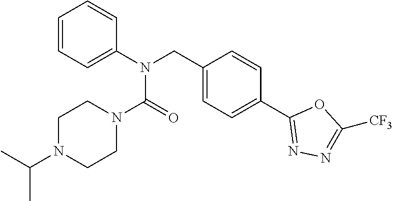 [SMILES: CC(C)N1CCN(C(=O)N(Cc2ccc(-c3nnc(C(F)(F)F)o3)cc2)c2ccccc2)CC1] |
| 19 | 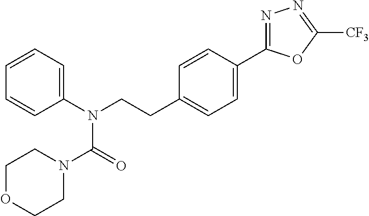 [SMILES: O=C(N1CCOCC1)N(CCc1ccc(-c2nnc(C(F)(F)F)o2)cc1)c1ccccc1] |
| 20 | 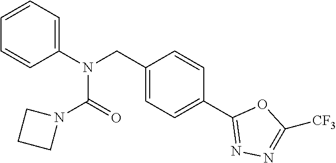 [SMILES: O=C(N1CCC1)N(Cc1ccc(-c2nnc(C(F)(F)F)o2)cc1)c1ccccc1] |
| 21 | 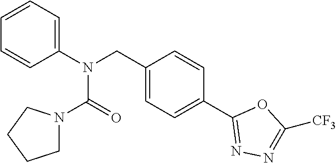 [SMILES: O=C(N1CCCC1)N(Cc1ccc(-c2nnc(C(F)(F)F)o2)cc1)c1ccccc1] |
| 22 | 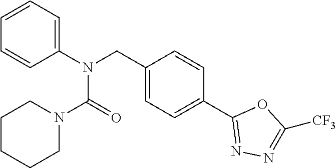 [SMILES: O=C(N1CCCCC1)N(Cc1ccc(-c2nnc(C(F)(F)F)o2)cc1)c1ccccc1] |
| 23 | 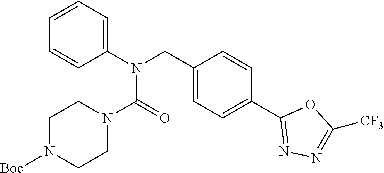 [SMILES: CC(C)(C)OC(=O)N1CCN(C(=O)N(Cc2ccc(-c3nnc(C(F)(F)F)o3)cc2)c2ccccc2)CC1] |
| 24 | 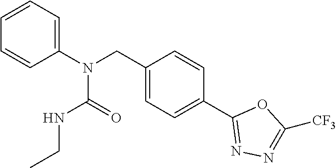 [SMILES: CCNC(=O)N(Cc1ccc(-c2nnc(C(F)(F)F)o2)cc1)c1ccccc1] |
| 25 | 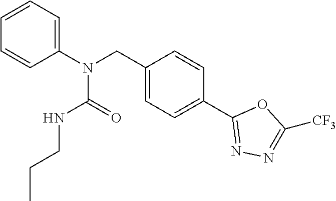 [SMILES: CCCNC(=O)N(Cc1ccc(-c2nnc(C(F)(F)F)o2)cc1)c1ccccc1] |
| 26 | 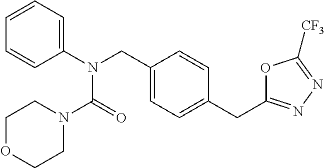 [SMILES: O=C(N1CCOCC1)N(Cc1ccc(Cc2nnc(C(F)(F)F)o2)cc1)c1ccccc1] |
| 27 | 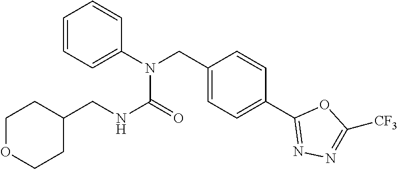 [SMILES: O=C(NCC1CCOCC1)N(Cc1ccc(-c2nnc(C(F)(F)F)o2)cc1)c1ccccc1] |
| 28 | 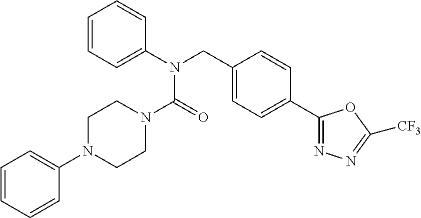 [SMILES: O=C(N1CCN(c2ccccc2)CC1)N(Cc1ccc(-c2nnc(C(F)(F)F)o2)cc1)c1ccccc1] |
| 29 | 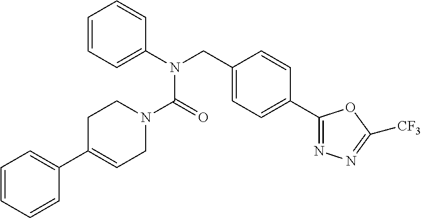 [SMILES: O=C(N1CC=C(c2ccccc2)CC1)N(Cc1ccc(-c2nnc(C(F)(F)F)o2)cc1)c1ccccc1] |
| 30 | 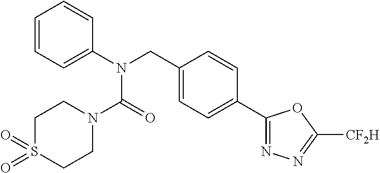 [SMILES: O=C(N1CCS(=O)(=O)CC1)N(Cc1ccc(-c2nnc(C(F)F)o2)cc1)c1ccccc1] |
| 31 | 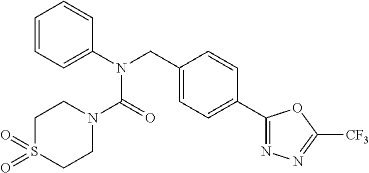 [SMILES: O=C(N1CCS(=O)(=O)CC1)N(Cc1ccc(-c2nnc(C(F)(F)F)o2)cc1)c1ccccc1] |
| 32 | 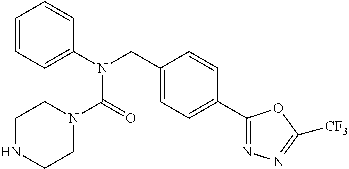 [SMILES: O=C(N1CCNCC1)N(Cc1ccc(-c2nnc(C(F)(F)F)o2)cc1)c1ccccc1] |
| 33 | 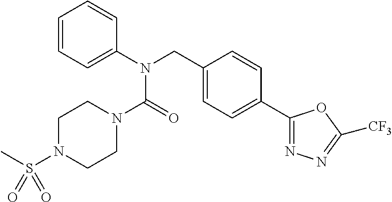 [SMILES: CS(=O)(=O)N1CCN(C(=O)N(Cc2ccc(-c3nnc(C(F)(F)F)o3)cc2)c2ccccc2)CC1] |
| 34 | 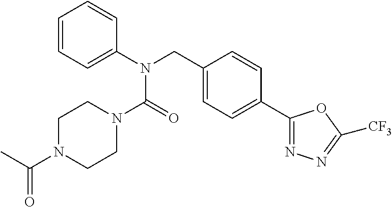 [SMILES: CC(=O)N1CCN(C(=O)N(Cc2ccc(-c3nnc(C(F)(F)F)o3)cc2)c2ccccc2)CC1] |
| 35 | 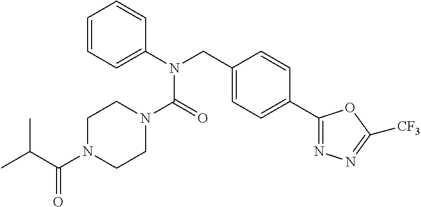 [SMILES: CC(C)C(=O)N1CCN(C(=O)N(Cc2ccc(-c3nnc(C(F)(F)F)o3)cc2)c2ccccc2)CC1] |
| 36 | 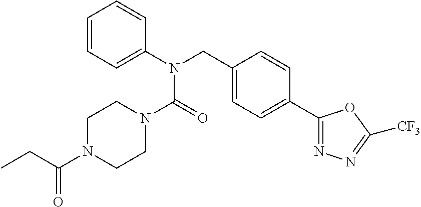 [SMILES: CCC(=O)N1CCN(C(=O)N(Cc2ccc(-c3nnc(C(F)(F)F)o3)cc2)c2ccccc2)CC1] |
| 37 | 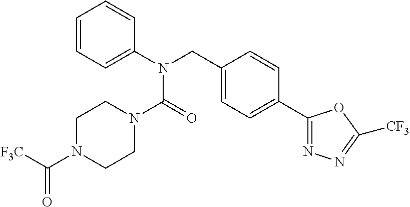 [SMILES: O=C(N1CCN(C(=O)C(F)(F)F)CC1)N(Cc1ccc(-c2nnc(C(F)(F)F)o2)cc1)c1ccccc1] |
| 38 | 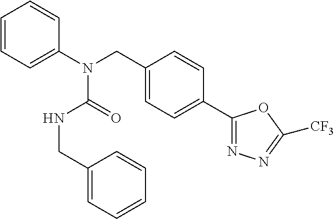 [SMILES: O=C(NCc1ccccc1)N(Cc1ccc(-c2nnc(C(F)(F)F)o2)cc1)c1ccccc1] |
| 39 | 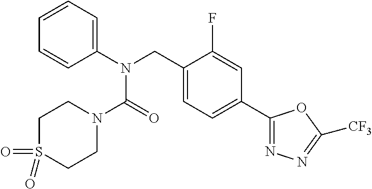 [SMILES: O=C(N1CCS(=O)(=O)CC1)N(Cc1ccc(-c2nnc(C(F)(F)F)o2)cc1F)c1ccccc1] |
| 40 | 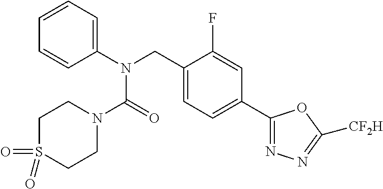 [SMILES: O=C(N1CCS(=O)(=O)CC1)N(Cc1ccc(-c2nnc(C(F)F)o2)cc1F)c1ccccc1] |
| 41 | 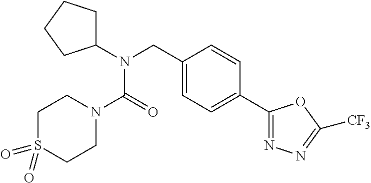 [SMILES: O=C(N1CCS(=O)(=O)CC1)N(Cc1ccc(-c2nnc(C(F)(F)F)o2)cc1)C1CCCC1] |
| 42 | 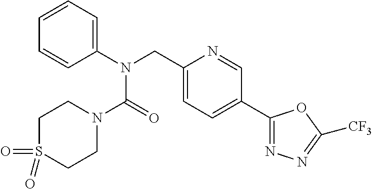 [SMILES: O=C(N1CCS(=O)(=O)CC1)N(Cc1ccc(-c2nnc(C(F)(F)F)o2)cn1)c1ccccc1] |
| 43 | 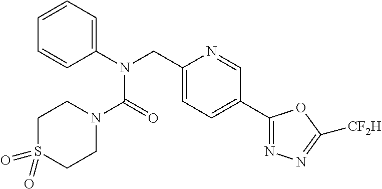 [SMILES: O=C(N1CCS(=O)(=O)CC1)N(Cc1ccc(-c2nnc(C(F)F)o2)cn1)c1ccccc1] |
| 44 | 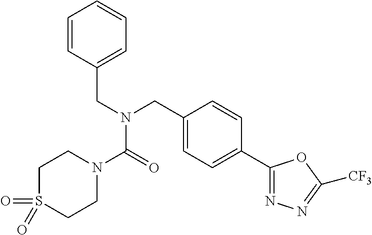 [SMILES: O=C(N1CCS(=O)(=O)CC1)N(Cc1ccccc1)Cc1ccc(-c2nnc(C(F)(F)F)o2)cc1] |
| 45 | 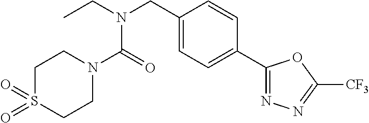 [SMILES: CCN(Cc1ccc(-c2nnc(C(F)(F)F)o2)cc1)C(=O)N1CCS(=O)(=O)CC1] |
| 46 | 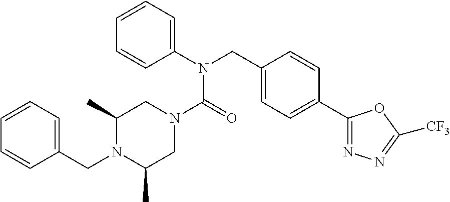 [SMILES: C[C@@H]1CN(C(=O)N(Cc2ccc(-c3nnc(C(F)(F)F)o3)cc2)c2ccccc2)C[C@H](C)N1Cc1ccccc1] |
| 47 | 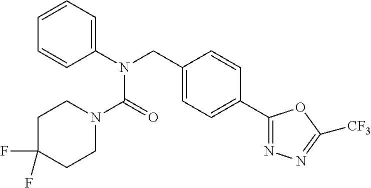 [SMILES: O=C(N1CCC(F)(F)CC1)N(Cc1ccc(-c2nnc(C(F)(F)F)o2)cc1)c1ccccc1] |
| 48 | 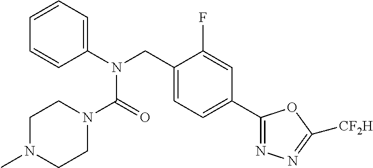 [SMILES: CN1CCN(C(=O)N(Cc2ccc(-c3nnc(C(F)F)o3)cc2F)c2ccccc2)CC1] |
| 49 | 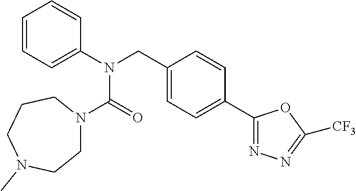 [SMILES: CN1CCCN(C(=O)N(Cc2ccc(-c3nnc(C(F)(F)F)o3)cc2)c2ccccc2)CC1] |
| 50 | 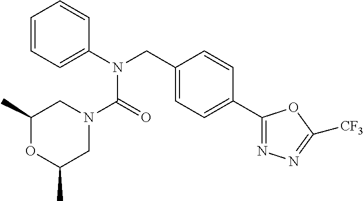 [SMILES: C[C@@H]1CN(C(=O)N(Cc2ccc(-c3nnc(C(F)(F)F)o3)cc2)c2ccccc2)C[C@H](C)O1] |
| 51 | 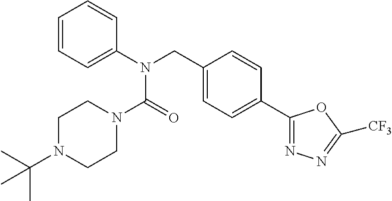 [SMILES: CC(C)(C)N1CCN(C(=O)N(Cc2ccc(-c3nnc(C(F)(F)F)o3)cc2)c2ccccc2)CC1] |
| 52 | 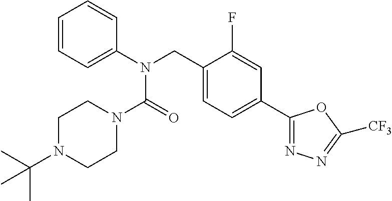 [SMILES: CC(C)(C)N1CCN(C(=O)N(Cc2ccc(-c3nnc(C(F)(F)F)o3)cc2F)c2ccccc2)CC1] |
| 53 | 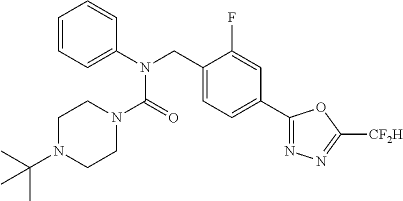 [SMILES: CC(C)(C)N1CCN(C(=O)N(Cc2ccc(-c3nnc(C(F)F)o3)cc2F)c2ccccc2)CC1] |
| 54 | 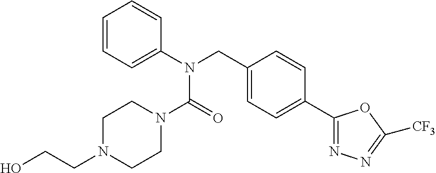 [SMILES: O=C(N1CCN(CCO)CC1)N(Cc1ccc(-c2nnc(C(F)(F)F)o2)cc1)c1ccccc1] |
| 55 | 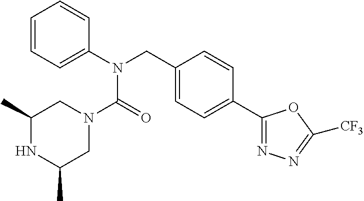 [SMILES: C[C@@H]1CN(C(=O)N(Cc2ccc(-c3nnc(C(F)(F)F)o3)cc2)c2ccccc2)C[C@H](C)N1] |
| 56 | 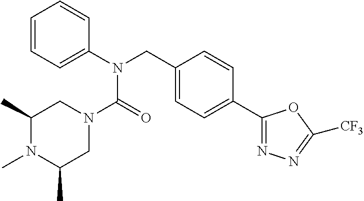 [SMILES: C[C@@H]1CN(C(=O)N(Cc2ccc(-c3nnc(C(F)(F)F)o3)cc2)c2ccccc2)C[C@H](C)N1C] |
| 57 | 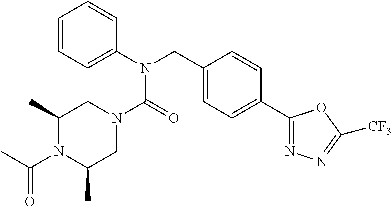 [SMILES: CC(=O)N1[C@H](C)CN(C(=O)N(Cc2ccc(-c3nnc(C(F)(F)F)o3)cc2)c2ccccc2)C[C@@H]1C] |
| 58 | 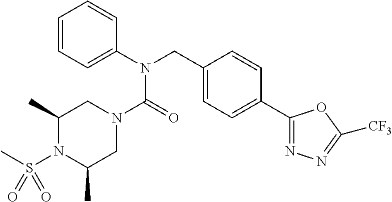 [SMILES: C[C@@H]1CN(C(=O)N(Cc2ccc(-c3nnc(C(F)(F)F)o3)cc2)c2ccccc2)C[C@H](C)N1S(C)(=O)=O] |
| 59 | 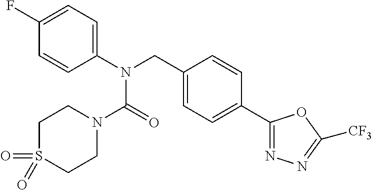 [SMILES: O=C(N1CCS(=O)(=O)CC1)N(Cc1ccc(-c2nnc(C(F)(F)F)o2)cc1)c1ccc(F)cc1] |
| 60 | 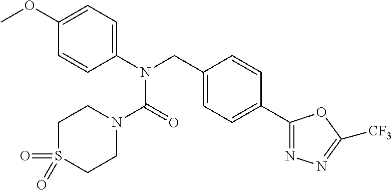 [SMILES: COc1ccc(N(Cc2ccc(-c3nnc(C(F)(F)F)o3)cc2)C(=O)N2CCS(=O)(=O)CC2)cc1] |
| 61 | 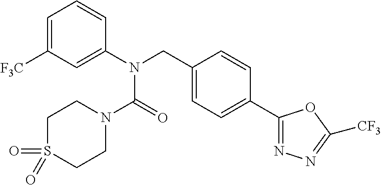 [SMILES: O=C(N1CCS(=O)(=O)CC1)N(Cc1ccc(-c2nnc(C(F)(F)F)o2)cc1)c1cccc(C(F)(F)F)c1] |
| 62 | 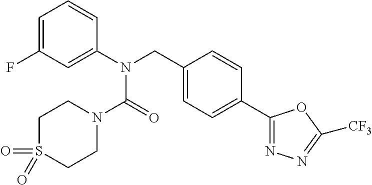 [SMILES: O=C(N1CCS(=O)(=O)CC1)N(Cc1ccc(-c2nnc(C(F)(F)F)o2)cc1)c1cccc(F)c1] |
| 63 | 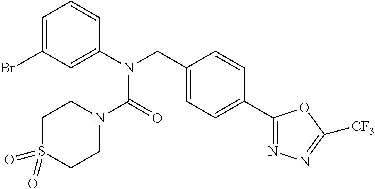 [SMILES: O=C(N1CCS(=O)(=O)CC1)N(Cc1ccc(-c2nnc(C(F)(F)F)o2)cc1)c1cccc(Br)c1] |
| 64 | 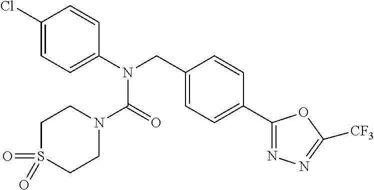 [SMILES: O=C(N1CCS(=O)(=O)CC1)N(Cc1ccc(-c2nnc(C(F)(F)F)o2)cc1)c1ccc(Cl)cc1] |
| 65 | 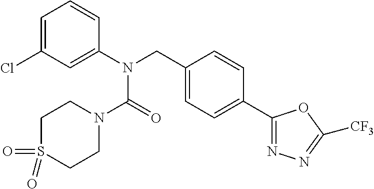 [SMILES: O=C(N1CCS(=O)(=O)CC1)N(Cc1ccc(-c2nnc(C(F)(F)F)o2)cc1)c1cccc(Cl)c1] |
| 66 | 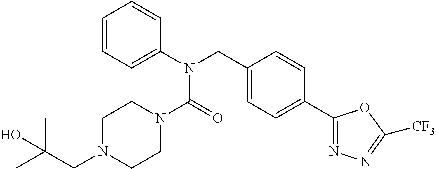 [SMILES: CC(C)(O)CN1CCN(C(=O)N(Cc2ccc(-c3nnc(C(F)(F)F)o3)cc2)c2ccccc2)CC1] |
| 67 | 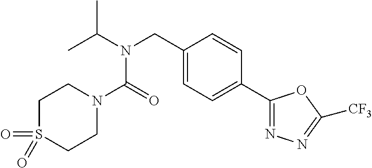 [SMILES: CC(C)N(Cc1ccc(-c2nnc(C(F)(F)F)o2)cc1)C(=O)N1CCS(=O)(=O)CC1] |
| 68 | 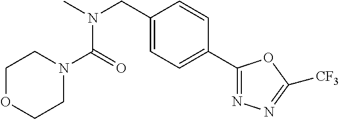 [SMILES: CN(Cc1ccc(-c2nnc(C(F)(F)F)o2)cc1)C(=O)N1CCOCC1] |
| 69 | 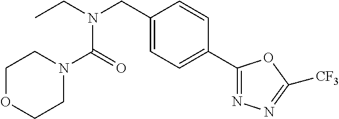 [SMILES: CCN(Cc1ccc(-c2nnc(C(F)(F)F)o2)cc1)C(=O)N1CCOCC1] |
| 70 | 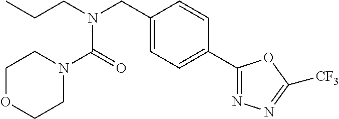 [SMILES: CCCN(Cc1ccc(-c2nnc(C(F)(F)F)o2)cc1)C(=O)N1CCOCC1] |
| 71 | 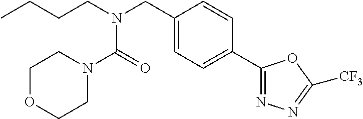 [SMILES: CCCCN(Cc1ccc(-c2nnc(C(F)(F)F)o2)cc1)C(=O)N1CCOCC1] |
| 72 | 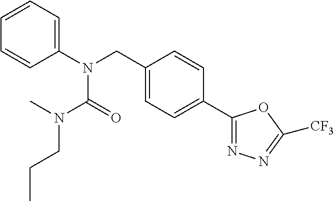 [SMILES: CCCN(C)C(=O)N(Cc1ccc(-c2nnc(C(F)(F)F)o2)cc1)c1ccccc1] |
| 73 | 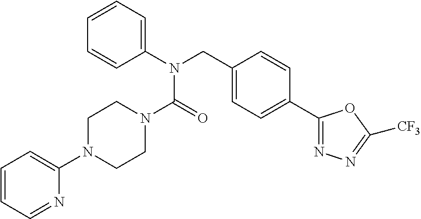 [SMILES: O=C(N1CCN(c2ccccn2)CC1)N(Cc1ccc(-c2nnc(C(F)(F)F)o2)cc1)c1ccccc1] |
| 74 | 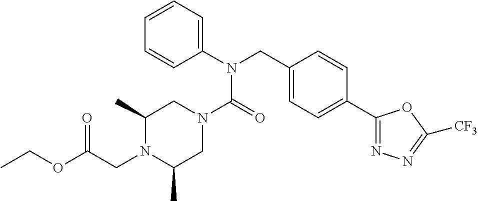 [SMILES: CCOC(=O)CN1[C@H](C)CN(C(=O)N(Cc2ccc(-c3nnc(C(F)(F)F)o3)cc2)c2ccccc2)C[C@@H]1C] |
| 75 | 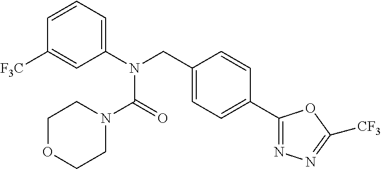 [SMILES: O=C(N1CCOCC1)N(Cc1ccc(-c2nnc(C(F)(F)F)o2)cc1)c1cccc(C(F)(F)F)c1] |
| 76 | 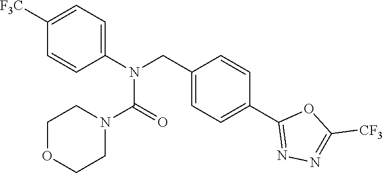 [SMILES: O=C(N1CCOCC1)N(Cc1ccc(-c2nnc(C(F)(F)F)o2)cc1)c1ccc(C(F)(F)F)cc1] |
| 77 | 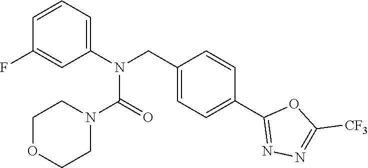 [SMILES: O=C(N1CCOCC1)N(Cc1ccc(-c2nnc(C(F)(F)F)o2)cc1)c1cccc(F)c1] |
| 78 | 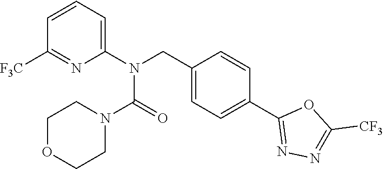 [SMILES: O=C(N1CCOCC1)N(Cc1ccc(-c2nnc(C(F)(F)F)o2)cc1)c1cccc(C(F)(F)F)n1] |
| 79 | 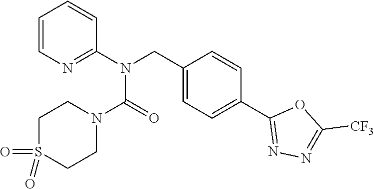 [SMILES: O=C(N1CCS(=O)(=O)CC1)N(Cc1ccc(-c2nnc(C(F)(F)F)o2)cc1)c1ccccn1] |
| 80 | 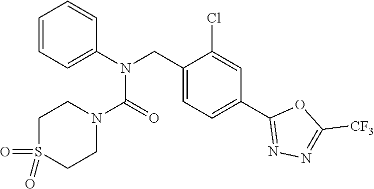 [SMILES: O=C(N1CCS(=O)(=O)CC1)N(Cc1ccc(-c2nnc(C(F)(F)F)o2)cc1Cl)c1ccccc1] |
| 81 | 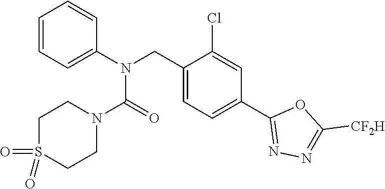 [SMILES: O=C(N1CCS(=O)(=O)CC1)N(Cc1ccc(-c2nnc(C(F)F)o2)cc1Cl)c1ccccc1] |
| 82 | 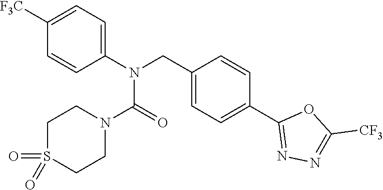 [SMILES: O=C(N1CCS(=O)(=O)CC1)N(Cc1ccc(-c2nnc(C(F)(F)F)o2)cc1)c1ccc(C(F)(F)F)cc1] |
| 83 | 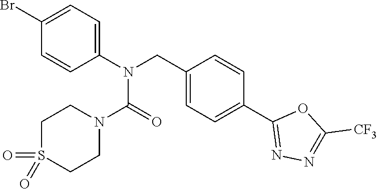 [SMILES: O=C(N1CCS(=O)(=O)CC1)N(Cc1ccc(-c2nnc(C(F)(F)F)o2)cc1)c1ccc(Br)cc1] |
| 84 | 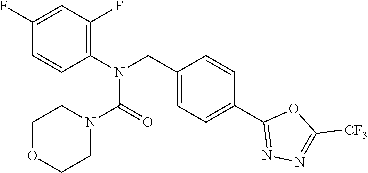 [SMILES: O=C(N1CCOCC1)N(Cc1ccc(-c2nnc(C(F)(F)F)o2)cc1)c1ccc(F)cc1F] |
| 85 | 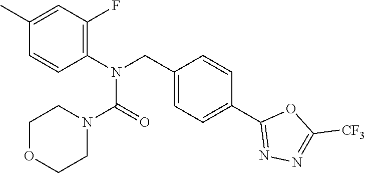 [SMILES: Cc1ccc(N(Cc2ccc(-c3nnc(C(F)(F)F)o3)cc2)C(=O)N2CCOCC2)c(F)c1] |
| 86 | 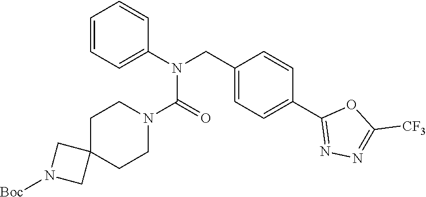 [SMILES: CC(C)(C)OC(=O)N1CC2(CCN(C(=O)N(Cc3ccc(-c4nnc(C(F)(F)F)o4)cc3)c3ccccc3)CC2)C1] |
| 87 | 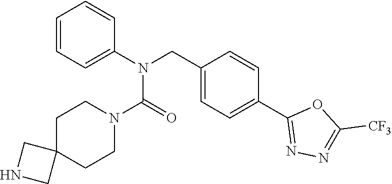 [SMILES: O=C(N1CCC2(CC1)CNC2)N(Cc1ccc(-c2nnc(C(F)(F)F)o2)cc1)c1ccccc1] |
| 88 | 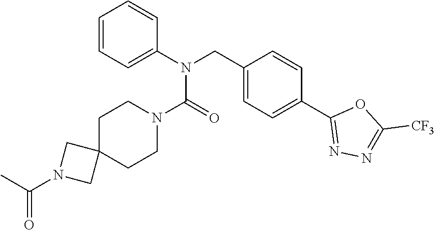 [SMILES: CC(=O)N1CC2(CCN(C(=O)N(Cc3ccc(-c4nnc(C(F)(F)F)o4)cc3)c3ccccc3)CC2)C1] |
| 89 | 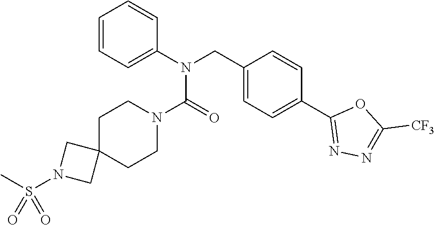 [SMILES: CS(=O)(=O)N1CC2(CCN(C(=O)N(Cc3ccc(-c4nnc(C(F)(F)F)o4)cc3)c3ccccc3)CC2)C1] |
| 90 | 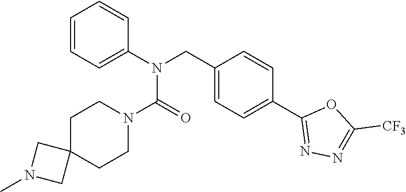 [SMILES: CN1CC2(CCN(C(=O)N(Cc3ccc(-c4nnc(C(F)(F)F)o4)cc3)c3ccccc3)CC2)C1] |
| 91 | 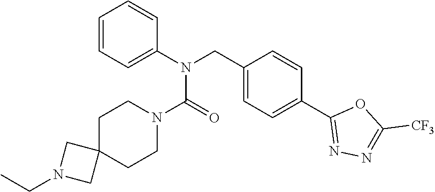 [SMILES: CCN1CC2(CCN(C(=O)N(Cc3ccc(-c4nnc(C(F)(F)F)o4)cc3)c3ccccc3)CC2)C1] |
| 92 | 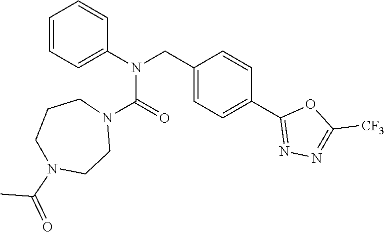 [SMILES: CC(=O)N1CCCN(C(=O)N(Cc2ccc(-c3nnc(C(F)(F)F)o3)cc2)c2ccccc2)CC1] |
| 93 | 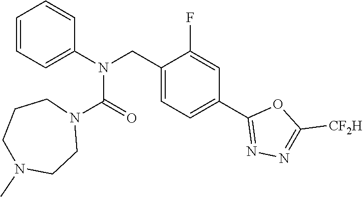 [SMILES: CN1CCCN(C(=O)N(Cc2ccc(-c3nnc(C(F)F)o3)cc2F)c2ccccc2)CC1] |
| 94 | 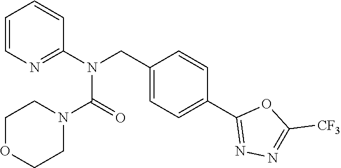 [SMILES: O=C(N1CCOCC1)N(Cc1ccc(-c2nnc(C(F)(F)F)o2)cc1)c1ccccn1] |
| 95 | 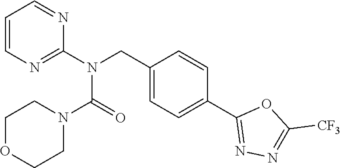 [SMILES: O=C(N1CCOCC1)N(Cc1ccc(-c2nnc(C(F)(F)F)o2)cc1)c1ncccn1] |
| 96 | 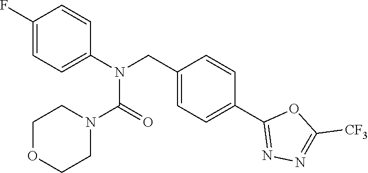 [SMILES: O=C(N1CCOCC1)N(Cc1ccc(-c2nnc(C(F)(F)F)o2)cc1)c1ccc(F)cc1] |
| 97 | 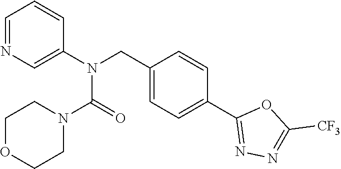 [SMILES: O=C(N1CCOCC1)N(Cc1ccc(-c2nnc(C(F)(F)F)o2)cc1)c1cccnc1] |
| 98 | 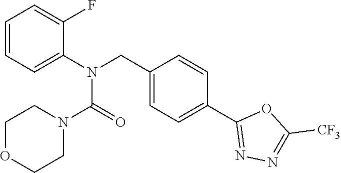 [SMILES: O=C(N1CCOCC1)N(Cc1ccc(-c2nnc(C(F)(F)F)o2)cc1)c1ccccc1F] |
| 99 | 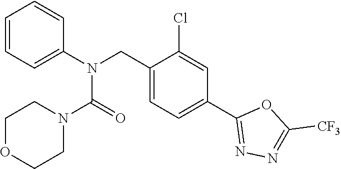 [SMILES: O=C(N1CCOCC1)N(Cc1ccc(-c2nnc(C(F)(F)F)o2)cc1Cl)c1ccccc1] |
| 100 | 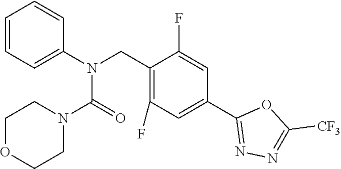 [SMILES: O=C(N1CCOCC1)N(Cc1c(F)cc(-c2nnc(C(F)(F)F)o2)cc1F)c1ccccc1] |
| 101 | 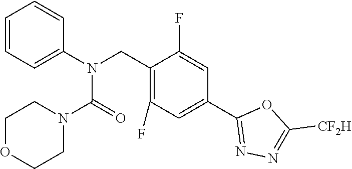 [SMILES: O=C(N1CCOCC1)N(Cc1c(F)cc(-c2nnc(C(F)F)o2)cc1F)c1ccccc1] |
| 102 | 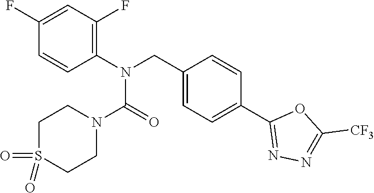 [SMILES: O=C(N1CCS(=O)(=O)CC1)N(Cc1ccc(-c2nnc(C(F)(F)F)o2)cc1)c1ccc(F)cc1F] |
| 103 | 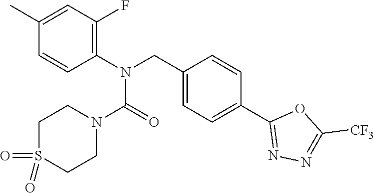 [SMILES: Cc1ccc(N(Cc2ccc(-c3nnc(C(F)(F)F)o3)cc2)C(=O)N2CCS(=O)(=O)CC2)c(F)c1] |
| 104 | 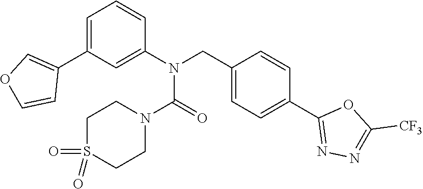 [SMILES: O=C(N1CCS(=O)(=O)CC1)N(Cc1ccc(-c2nnc(C(F)(F)F)o2)cc1)c1cccc(-c2ccoc2)c1] |
| 105 | 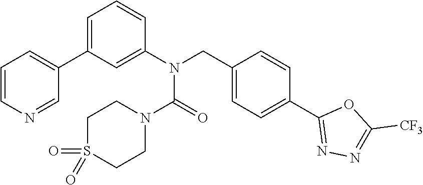 [SMILES: O=C(N1CCS(=O)(=O)CC1)N(Cc1ccc(-c2nnc(C(F)(F)F)o2)cc1)c1cccc(-c2cccnc2)c1] |
| 106 | 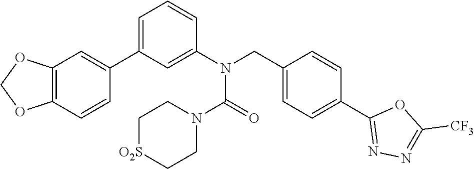 [SMILES: O=C(N1CCS(=O)(=O)CC1)N(Cc1ccc(-c2nnc(C(F)(F)F)o2)cc1)c1cccc(-c2ccc3c(c2)OCO3)c1] |
| 107 | 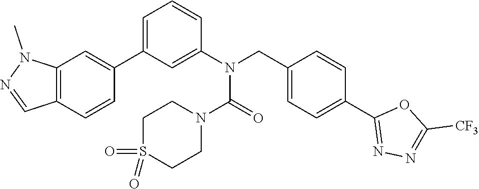 [SMILES: Cn1ncc2ccc(-c3cccc(N(Cc4ccc(-c5nnc(C(F)(F)F)o5)cc4)C(=O)N4CCS(=O)(=O)CC4)c3)cc21] |
| 108 | 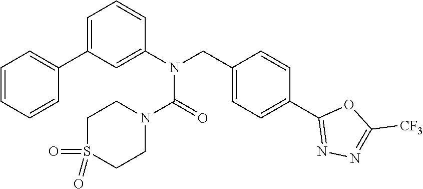 [SMILES: O=C(N1CCS(=O)(=O)CC1)N(Cc1ccc(-c2nnc(C(F)(F)F)o2)cc1)c1cccc(-c2ccccc2)c1] |
| 109 | 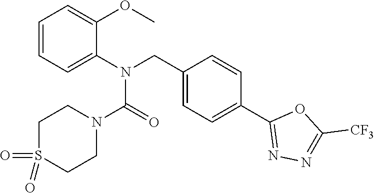 [SMILES: COc1ccccc1N(Cc1ccc(-c2nnc(C(F)(F)F)o2)cc1)C(=O)N1CCS(=O)(=O)CC1] |
| 110 | 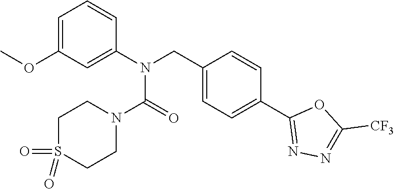 [SMILES: COc1cccc(N(Cc2ccc(-c3nnc(C(F)(F)F)o3)cc2)C(=O)N2CCS(=O)(=O)CC2)c1] |
| 111 | 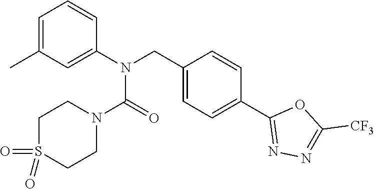 [SMILES: Cc1cccc(N(Cc2ccc(-c3nnc(C(F)(F)F)o3)cc2)C(=O)N2CCS(=O)(=O)CC2)c1] |
| 112 | 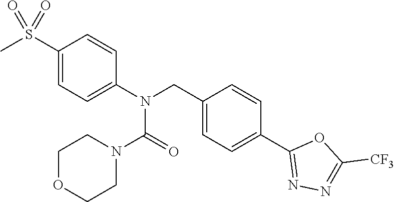 [SMILES: CS(=O)(=O)c1ccc(N(Cc2ccc(-c3nnc(C(F)(F)F)o3)cc2)C(=O)N2CCOCC2)cc1] |
| 113 | 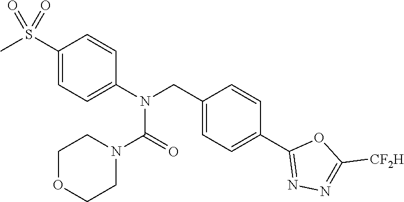 [SMILES: CS(=O)(=O)c1ccc(N(Cc2ccc(-c3nnc(C(F)F)o3)cc2)C(=O)N2CCOCC2)cc1] |
| 114 | 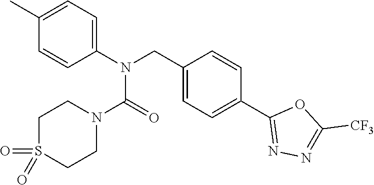 [SMILES: Cc1ccc(N(Cc2ccc(-c3nnc(C(F)(F)F)o3)cc2)C(=O)N2CCS(=O)(=O)CC2)cc1] |
| 115 | 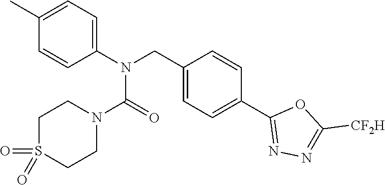 [SMILES: Cc1ccc(N(Cc2ccc(-c3nnc(C(F)F)o3)cc2)C(=O)N2CCS(=O)(=O)CC2)cc1] |
| 116 | 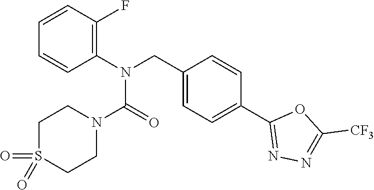 [SMILES: O=C(N1CCS(=O)(=O)CC1)N(Cc1ccc(-c2nnc(C(F)(F)F)o2)cc1)c1ccccc1F] |
| 117 | 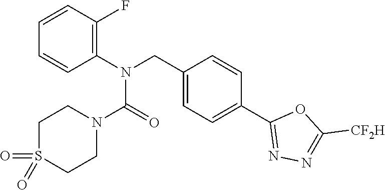 [SMILES: O=C(N1CCS(=O)(=O)CC1)N(Cc1ccc(-c2nnc(C(F)F)o2)cc1)c1ccccc1F] |
| 118 | 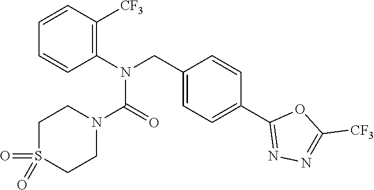 [SMILES: O=C(N1CCS(=O)(=O)CC1)N(Cc1ccc(-c2nnc(C(F)(F)F)o2)cc1)c1ccccc1C(F)(F)F] |
| 119 | 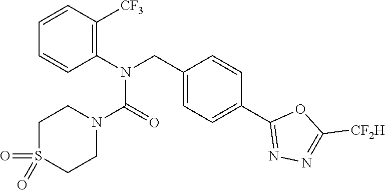 [SMILES: O=C(N1CCS(=O)(=O)CC1)N(Cc1ccc(-c2nnc(C(F)F)o2)cc1)c1ccccc1C(F)(F)F] |
| 120 | 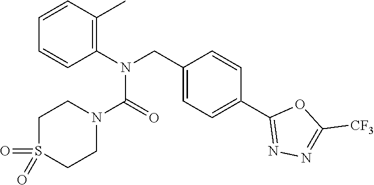 [SMILES: Cc1ccccc1N(Cc1ccc(-c2nnc(C(F)(F)F)o2)cc1)C(=O)N1CCS(=O)(=O)CC1] |
| 121 | 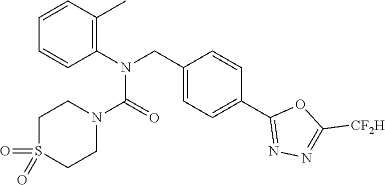 [SMILES: Cc1ccccc1N(Cc1ccc(-c2nnc(C(F)F)o2)cc1)C(=O)N1CCS(=O)(=O)CC1] |
| 122 | 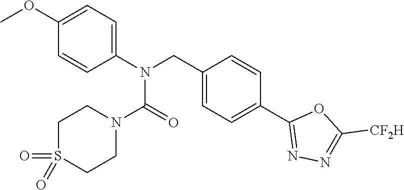 [SMILES: COc1ccc(N(Cc2ccc(-c3nnc(C(F)F)o3)cc2)C(=O)N2CCS(=O)(=O)CC2)cc1] |
| 123 | 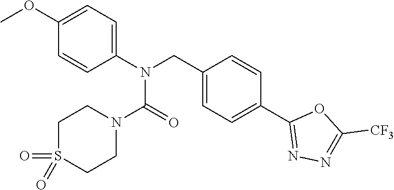 [SMILES: COc1ccc(N(Cc2ccc(-c3nnc(C(F)(F)F)o3)cc2)C(=O)N2CCS(=O)(=O)CC2)cc1] |
| 124 | 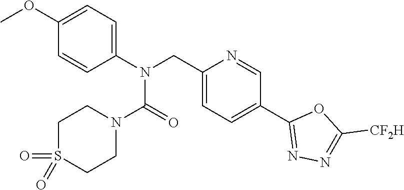 [SMILES: COc1ccc(N(Cc2ccc(-c3nnc(C(F)F)o3)cn2)C(=O)N2CCS(=O)(=O)CC2)cc1] |
| 125 | 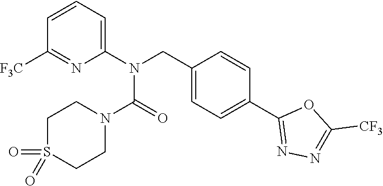 [SMILES: O=C(N1CCS(=O)(=O)CC1)N(Cc1ccc(-c2nnc(C(F)(F)F)o2)cc1)c1cccc(C(F)(F)F)n1] |
| 126 | 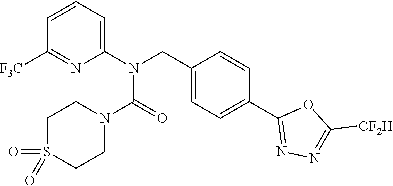 [SMILES: O=C(N1CCS(=O)(=O)CC1)N(Cc1ccc(-c2nnc(C(F)F)o2)cc1)c1cccc(C(F)(F)F)n1] |
| 127 | 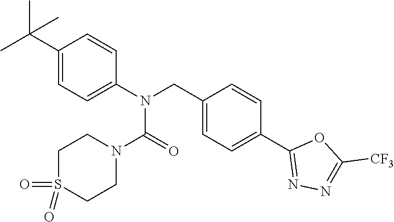 [SMILES: CC(C)(C)c1ccc(N(Cc2ccc(-c3nnc(C(F)(F)F)o3)cc2)C(=O)N2CCS(=O)(=O)CC2)cc1] |
| 128 | 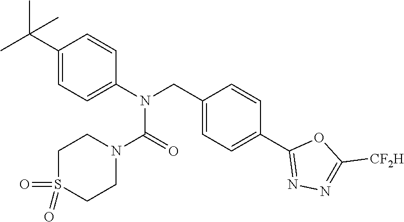 [SMILES: CC(C)(C)c1ccc(N(Cc2ccc(-c3nnc(C(F)F)o3)cc2)C(=O)N2CCS(=O)(=O)CC2)cc1] |
| 129 | 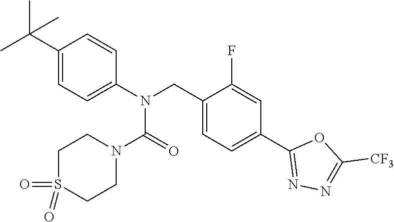 [SMILES: CC(C)(C)c1ccc(N(Cc2ccc(-c3nnc(C(F)(F)F)o3)cc2F)C(=O)N2CCS(=O)(=O)CC2)cc1] |
| 130 | 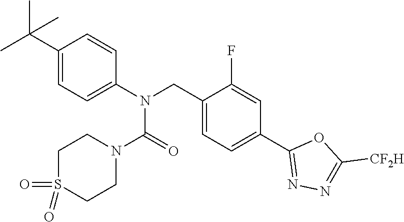 [SMILES: CC(C)(C)c1ccc(N(Cc2ccc(-c3nnc(C(F)F)o3)cc2F)C(=O)N2CCS(=O)(=O)CC2)cc1] |
| 131 | 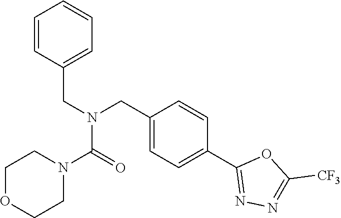 [SMILES: O=C(N1CCOCC1)N(Cc1ccccc1)Cc1ccc(-c2nnc(C(F)(F)F)o2)cc1] |
| 132 | 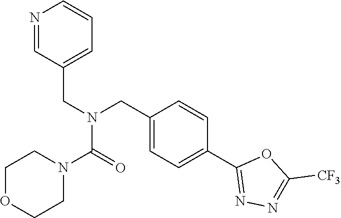 [SMILES: O=C(N1CCOCC1)N(Cc1ccc(-c2nnc(C(F)(F)F)o2)cc1)Cc1cccnc1] |
| 133 | 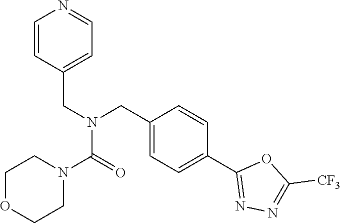 [SMILES: O=C(N1CCOCC1)N(Cc1ccncc1)Cc1ccc(-c2nnc(C(F)(F)F)o2)cc1] |
| 134 | 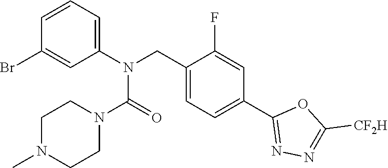 [SMILES: CN1CCN(C(=O)N(Cc2ccc(-c3nnc(C(F)F)o3)cc2F)c2cccc(Br)c2)CC1] |
| 135 | 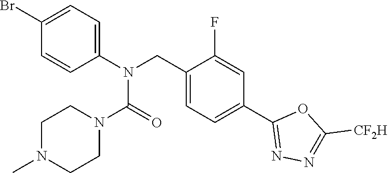 [SMILES: CN1CCN(C(=O)N(Cc2ccc(-c3nnc(C(F)F)o3)cc2F)c2ccc(Br)cc2)CC1] |
| 136 | 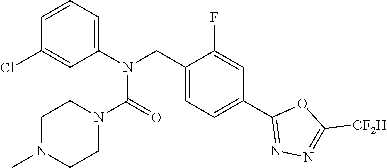 [SMILES: CN1CCN(C(=O)N(Cc2ccc(-c3nnc(C(F)F)o3)cc2F)c2cccc(Cl)c2)CC1] |
| 137 | 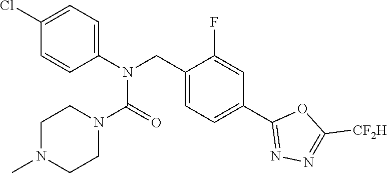 [SMILES: CN1CCN(C(=O)N(Cc2ccc(-c3nnc(C(F)F)o3)cc2F)c2ccc(Cl)cc2)CC1] |
| 138 | 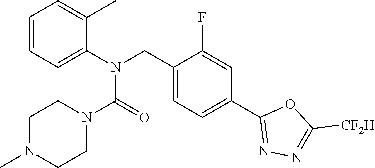 [SMILES: Cc1ccccc1N(Cc1ccc(-c2nnc(C(F)F)o2)cc1F)C(=O)N1CCN(C)CC1] |
| 139 | 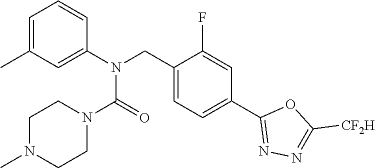 [SMILES: Cc1cccc(N(Cc2ccc(-c3nnc(C(F)F)o3)cc2F)C(=O)N2CCN(C)CC2)c1] |
| 140 | 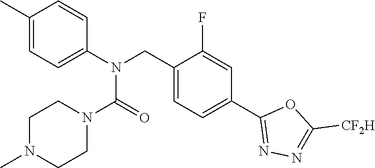 [SMILES: Cc1ccc(N(Cc2ccc(-c3nnc(C(F)F)o3)cc2F)C(=O)N2CCN(C)CC2)cc1] |
| 141 | 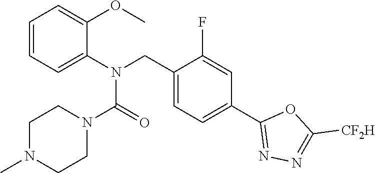 [SMILES: COc1ccccc1N(Cc1ccc(-c2nnc(C(F)F)o2)cc1F)C(=O)N1CCN(C)CC1] |
| 142 | 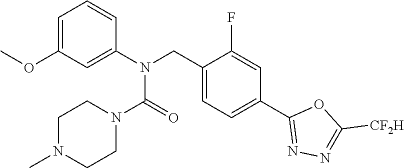 [SMILES: COc1cccc(N(Cc2ccc(-c3nnc(C(F)F)o3)cc2F)C(=O)N2CCN(C)CC2)c1] |
| 143 | 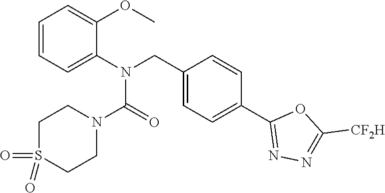 [SMILES: COc1ccccc1N(Cc1ccc(-c2nnc(C(F)F)o2)cc1)C(=O)N1CCS(=O)(=O)CC1] |
| 144 | 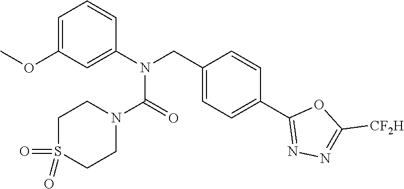 [SMILES: COc1cccc(N(Cc2ccc(-c3nnc(C(F)F)o3)cc2)C(=O)N2CCS(=O)(=O)CC2)c1] |
| 145 | 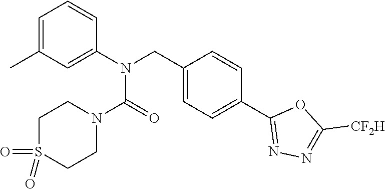 [SMILES: Cc1cccc(N(Cc2ccc(-c3nnc(C(F)F)o3)cc2)C(=O)N2CCS(=O)(=O)CC2)c1] |
| 146 | 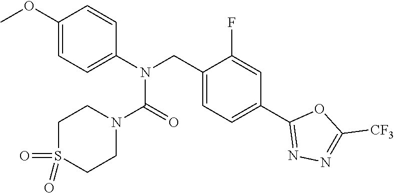 [SMILES: COc1ccc(N(Cc2ccc(-c3nnc(C(F)(F)F)o3)cc2F)C(=O)N2CCS(=O)(=O)CC2)cc1] |
| 147 | 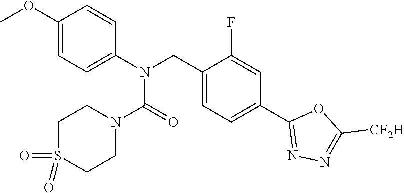 [SMILES: COc1ccc(N(Cc2ccc(-c3nnc(C(F)F)o3)cc2F)C(=O)N2CCS(=O)(=O)CC2)cc1] |
| 148 | 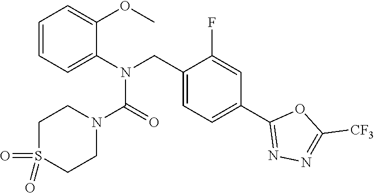 [SMILES: COc1ccccc1N(Cc1ccc(-c2nnc(C(F)(F)F)o2)cc1F)C(=O)N1CCS(=O)(=O)CC1] |
| 149 | 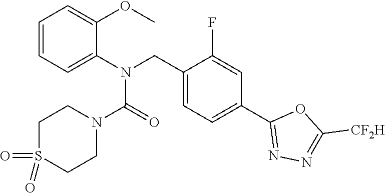 [SMILES: COc1ccccc1N(Cc1ccc(-c2nnc(C(F)F)o2)cc1F)C(=O)N1CCS(=O)(=O)CC1] |
| 150 | 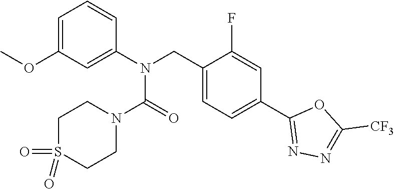 [SMILES: COc1cccc(N(Cc2ccc(-c3nnc(C(F)(F)F)o3)cc2F)C(=O)N2CCS(=O)(=O)CC2)c1] |
| 151 | 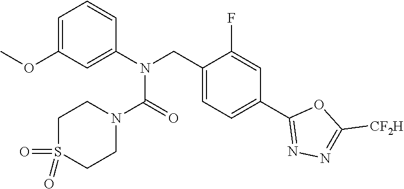 [SMILES: COc1cccc(N(Cc2ccc(-c3nnc(C(F)F)o3)cc2F)C(=O)N2CCS(=O)(=O)CC2)c1] |
| 152 | 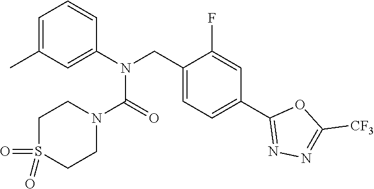 [SMILES: Cc1cccc(N(Cc2ccc(-c3nnc(C(F)(F)F)o3)cc2F)C(=O)N2CCS(=O)(=O)CC2)c1] |
| 153 | 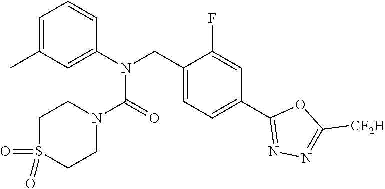 [SMILES: Cc1cccc(N(Cc2ccc(-c3nnc(C(F)F)o3)cc2F)C(=O)N2CCS(=O)(=O)CC2)c1] |
| 154 | 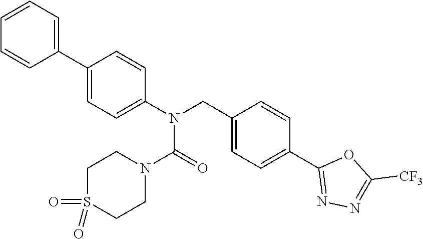 [SMILES: O=C(N1CCS(=O)(=O)CC1)N(Cc1ccc(-c2nnc(C(F)(F)F)o2)cc1)c1ccc(-c2ccccc2)cc1] |
| 155 | 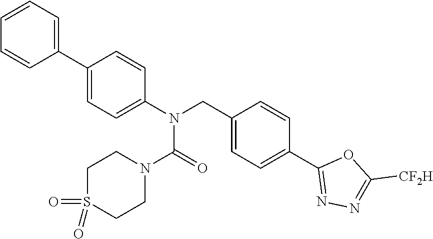 [SMILES: O=C(N1CCS(=O)(=O)CC1)N(Cc1ccc(-c2nnc(C(F)F)o2)cc1)c1ccc(-c2ccccc2)cc1] |
| 156 | 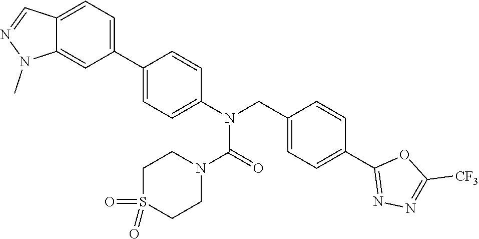 [SMILES: Cn1ncc2ccc(-c3ccc(N(Cc4ccc(-c5nnc(C(F)(F)F)o5)cc4)C(=O)N4CCS(=O)(=O)CC4)cc3)cc21] |
| 157 | 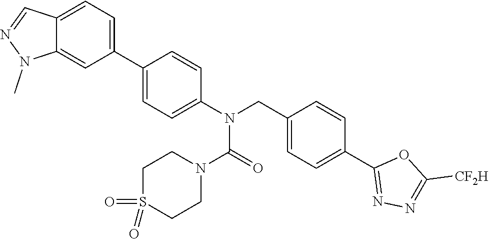 [SMILES: Cn1ncc2ccc(-c3ccc(N(Cc4ccc(-c5nnc(C(F)F)o5)cc4)C(=O)N4CCS(=O)(=O)CC4)cc3)cc21] |
| 158 | 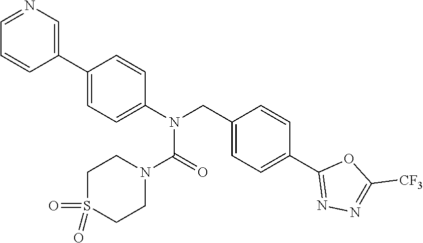 [SMILES: O=C(N1CCS(=O)(=O)CC1)N(Cc1ccc(-c2nnc(C(F)(F)F)o2)cc1)c1ccc(-c2cccnc2)cc1] |
| 159 | 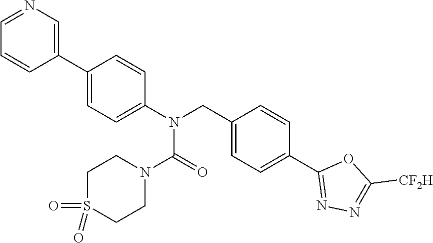 [SMILES: O=C(N1CCS(=O)(=O)CC1)N(Cc1ccc(-c2nnc(C(F)F)o2)cc1)c1ccc(-c2cccnc2)cc1] |
| 160 | 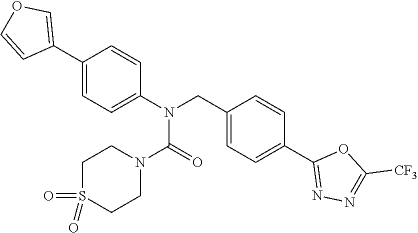 [SMILES: O=C(N1CCS(=O)(=O)CC1)N(Cc1ccc(-c2nnc(C(F)(F)F)o2)cc1)c1ccc(-c2ccoc2)cc1] |
| 161 | 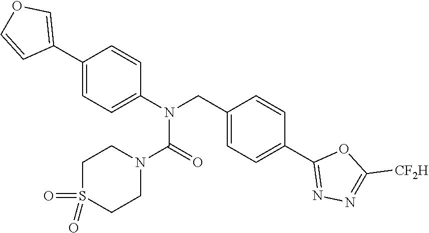 [SMILES: O=C(N1CCS(=O)(=O)CC1)N(Cc1ccc(-c2nnc(C(F)F)o2)cc1)c1ccc(-c2ccoc2)cc1] |
| 162 | 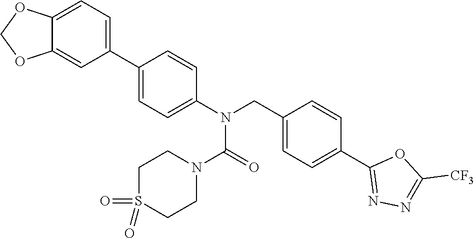 [SMILES: O=C(N1CCS(=O)(=O)CC1)N(Cc1ccc(-c2nnc(C(F)(F)F)o2)cc1)c1ccc(-c2ccc3c(c2)OCO3)cc1] |
| 163 | 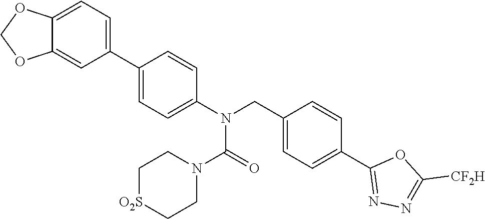 [SMILES: O=C(N1CCS(=O)(=O)CC1)N(Cc1ccc(-c2nnc(C(F)F)o2)cc1)c1ccc(-c2ccc3c(c2)OCO3)cc1] |
| 164 | 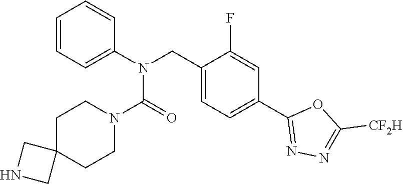 [SMILES: O=C(N1CCC2(CC1)CNC2)N(Cc1ccc(-c2nnc(C(F)F)o2)cc1F)c1ccccc1] |
| 165 | 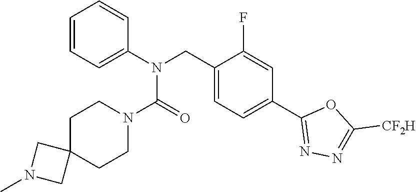 [SMILES: CN1CC2(CCN(C(=O)N(Cc3ccc(-c4nnc(C(F)F)o4)cc3F)c3ccccc3)CC2)C1] |
| 166 | 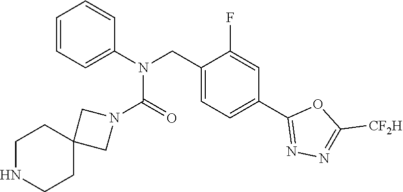 [SMILES: O=C(N1CC2(CCNCC2)C1)N(Cc1ccc(-c2nnc(C(F)F)o2)cc1F)c1ccccc1] |
| 167 | 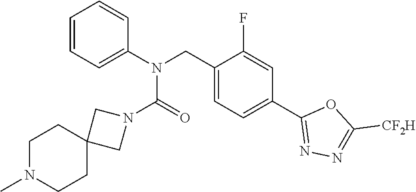 [SMILES: CN1CCC2(CC1)CN(C(=O)N(Cc1ccc(-c3nnc(C(F)F)o3)cc1F)c1ccccc1)C2] |
| 168 | 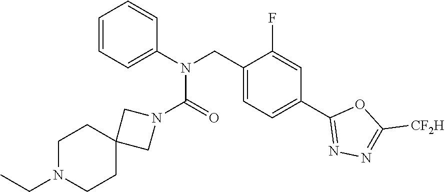 [SMILES: CCN1CCC2(CC1)CN(C(=O)N(Cc1ccc(-c3nnc(C(F)F)o3)cc1F)c1ccccc1)C2] |
| 169 | 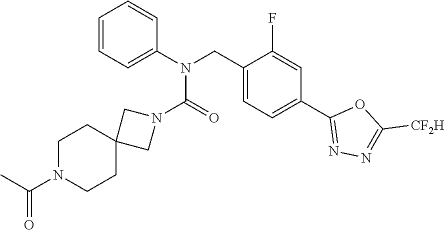 [SMILES: CC(=O)N1CCC2(CC1)CN(C(=O)N(Cc1ccc(-c3nnc(C(F)F)o3)cc1F)c1ccccc1)C2] |
| 170 | 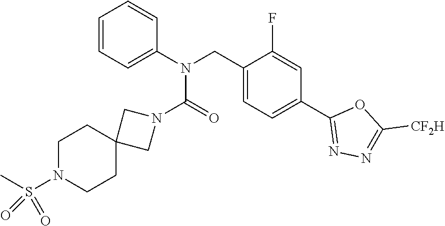 [SMILES: CS(=O)(=O)N1CCC2(CC1)CN(C(=O)N(Cc1ccc(-c3nnc(C(F)F)o3)cc1F)c1ccccc1)C2] |
| 171 | 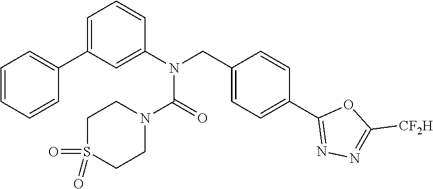 [SMILES: O=C(N1CCS(=O)(=O)CC1)N(Cc1ccc(-c2nnc(C(F)F)o2)cc1)c1cccc(-c2ccccc2)c1] |
| 172 | 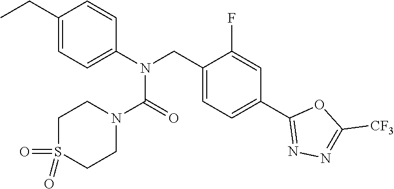 [SMILES: CCc1ccc(N(Cc2ccc(-c3nnc(C(F)(F)F)o3)cc2F)C(=O)N2CCS(=O)(=O)CC2)cc1] |
| 173 | 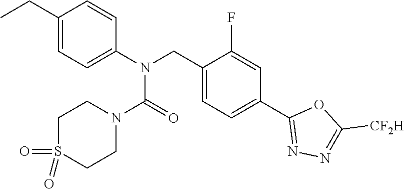 [SMILES: CCc1ccc(N(Cc2ccc(-c3nnc(C(F)F)o3)cc2F)C(=O)N2CCS(=O)(=O)CC2)cc1] |
| 174 | 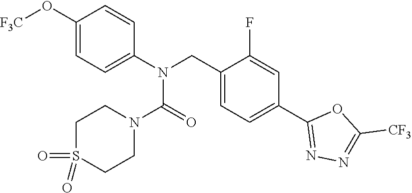 [SMILES: O=C(N1CCS(=O)(=O)CC1)N(Cc1ccc(-c2nnc(C(F)(F)F)o2)cc1F)c1ccc(OC(F)(F)F)cc1] |
| 175 | 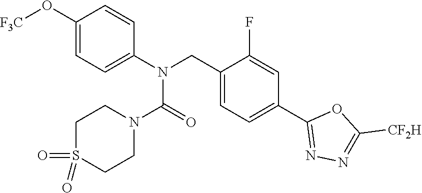 [SMILES: O=C(N1CCS(=O)(=O)CC1)N(Cc1ccc(-c2nnc(C(F)F)o2)cc1F)c1ccc(OC(F)(F)F)cc1] |
| 176 | 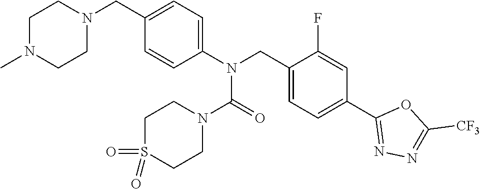 [SMILES: CN1CCN(Cc2ccc(N(Cc3ccc(-c4nnc(C(F)(F)F)o4)cc3F)C(=O)N3CCS(=O)(=O)CC3)cc2)CC1] |
| 177 | 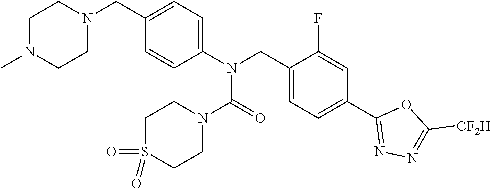 [SMILES: CN1CCN(Cc2ccc(N(Cc3ccc(-c4nnc(C(F)F)o4)cc3F)C(=O)N3CCS(=O)(=O)CC3)cc2)CC1] |
| 178 | 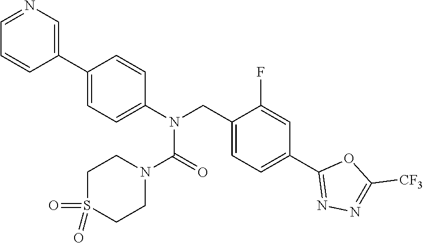 [SMILES: O=C(N1CCS(=O)(=O)CC1)N(Cc1ccc(-c2nnc(C(F)(F)F)o2)cc1F)c1ccc(-c2cccnc2)cc1] |
| 179 | 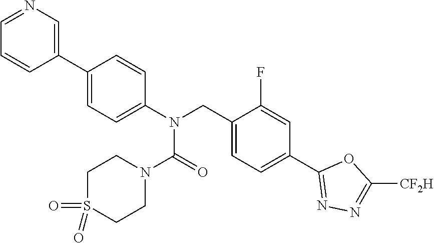 [SMILES: O=C(N1CCS(=O)(=O)CC1)N(Cc1ccc(-c2nnc(C(F)F)o2)cc1F)c1ccc(-c2cccnc2)cc1] |
| 180 | 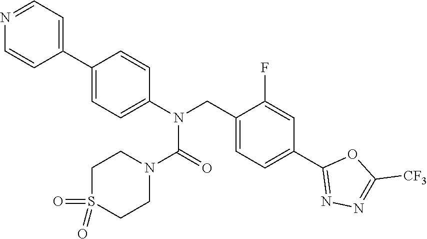 [SMILES: O=C(N1CCS(=O)(=O)CC1)N(Cc1ccc(-c2nnc(C(F)(F)F)o2)cc1F)c1ccc(-c2ccncc2)cc1] |
| 181 | 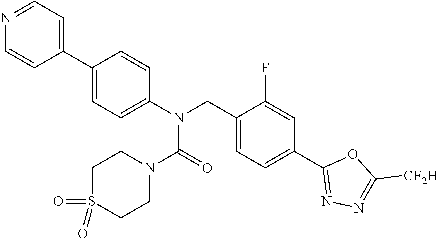 [SMILES: O=C(N1CCS(=O)(=O)CC1)N(Cc1ccc(-c2nnc(C(F)F)o2)cc1F)c1ccc(-c2ccncc2)cc1] |
| 182 | 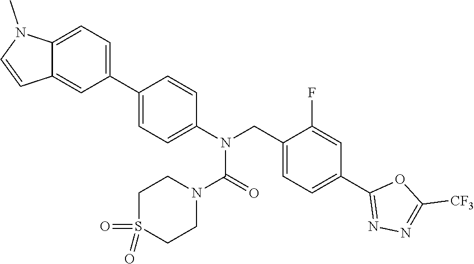 [SMILES: Cn1ccc2cc(-c3ccc(N(Cc4ccc(-c5nnc(C(F)(F)F)o5)cc4F)C(=O)N4CCS(=O)(=O)CC4)cc3)ccc21] |
| 183 | 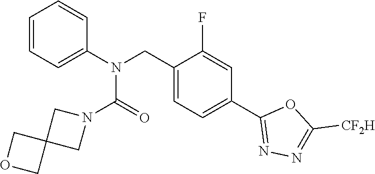 [SMILES: O=C(N1CC2(COC2)C1)N(Cc1ccc(-c2nnc(C(F)F)o2)cc1F)c1ccccc1] |
| 184 | 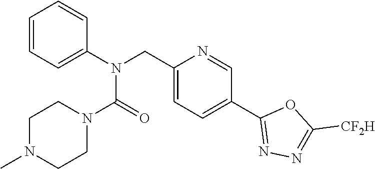 [SMILES: CN1CCN(C(=O)N(Cc2ccc(-c3nnc(C(F)F)o3)cn2)c2ccccc2)CC1] |
| 185 | 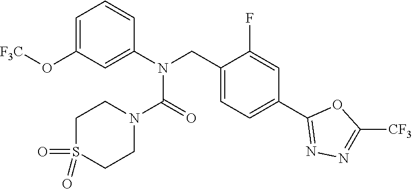 [SMILES: O=C(N1CCS(=O)(=O)CC1)N(Cc1ccc(-c2nnc(C(F)(F)F)o2)cc1F)c1cccc(OC(F)(F)F)c1] |
| 186 | 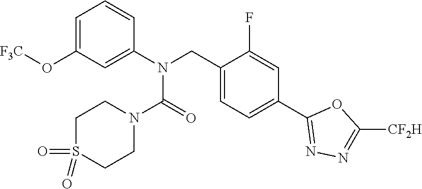 [SMILES: O=C(N1CCS(=O)(=O)CC1)N(Cc1ccc(-c2nnc(C(F)F)o2)cc1F)c1cccc(OC(F)(F)F)c1] |
| 187 | 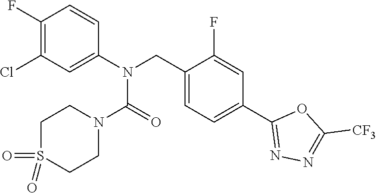 [SMILES: O=C(N1CCS(=O)(=O)CC1)N(Cc1ccc(-c2nnc(C(F)(F)F)o2)cc1F)c1ccc(F)c(Cl)c1] |
| 188 | 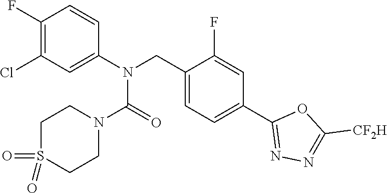 [SMILES: O=C(N1CCS(=O)(=O)CC1)N(Cc1ccc(-c2nnc(C(F)F)o2)cc1F)c1ccc(F)c(Cl)c1] |
| 189 | 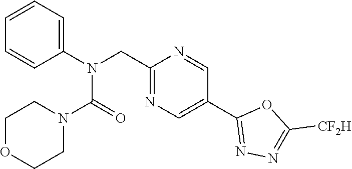 [SMILES: O=C(N1CCOCC1)N(Cc1ncc(-c2nnc(C(F)F)o2)cn1)c1ccccc1] |
| 190 | 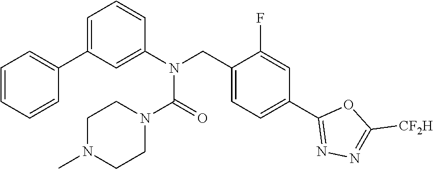 [SMILES: CN1CCN(C(=O)N(Cc2ccc(-c3nnc(C(F)F)o3)cc2F)c2cccc(-c3ccccc3)c2)CC1] |
| 191 | 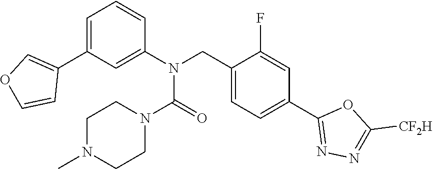 [SMILES: CN1CCN(C(=O)N(Cc2ccc(-c3nnc(C(F)F)o3)cc2F)c2cccc(-c3ccoc3)c2)CC1] |
| 192 | 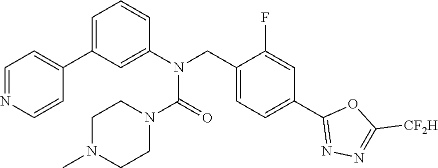 [SMILES: CN1CCN(C(=O)N(Cc2ccc(-c3nnc(C(F)F)o3)cc2F)c2cccc(-c3ccncc3)c2)CC1] |
| 193 | 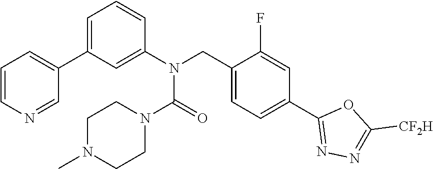 [SMILES: CN1CCN(C(=O)N(Cc2ccc(-c3nnc(C(F)F)o3)cc2F)c2cccc(-c3cccnc3)c2)CC1] |
| 194 | 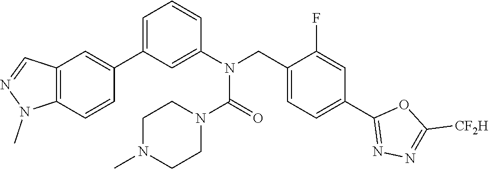 [SMILES: CN1CCN(C(=O)N(Cc2ccc(-c3nnc(C(F)F)o3)cc2F)c2cccc(-c3ccc4c(cnn4C)c3)c2)CC1] |
| 195 | 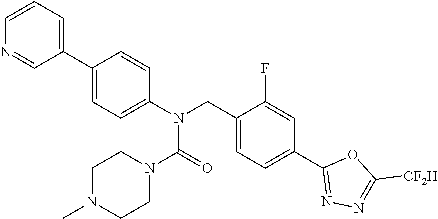 [SMILES: CN1CCN(C(=O)N(Cc2ccc(-c3nnc(C(F)F)o3)cc2F)c2ccc(-c3cccnc3)cc2)CC1] |
| 196 | 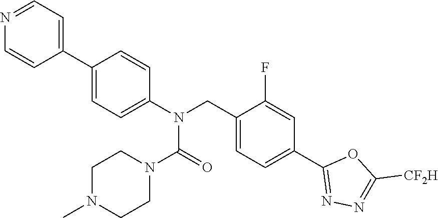 [SMILES: CN1CCN(C(=O)N(Cc2ccc(-c3nnc(C(F)F)o3)cc2F)c2ccc(-c3ccncc3)cc2)CC1] |
| 197 | 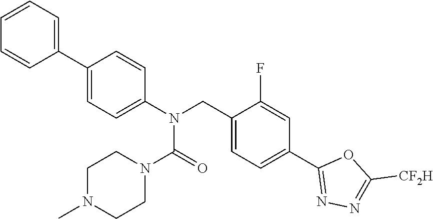 [SMILES: CN1CCN(C(=O)N(Cc2ccc(-c3nnc(C(F)F)o3)cc2F)c2ccc(-c3ccccc3)cc2)CC1] |
| 198 | 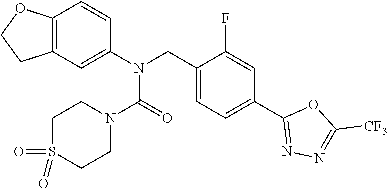 [SMILES: O=C(N1CCS(=O)(=O)CC1)N(Cc1ccc(-c2nnc(C(F)(F)F)o2)cc1F)c1ccc2c(c1)CCO2] |
| 199 | 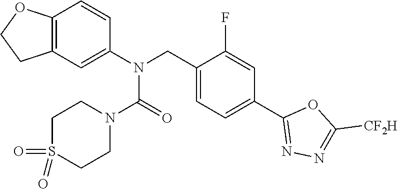 [SMILES: O=C(N1CCS(=O)(=O)CC1)N(Cc1ccc(-c2nnc(C(F)F)o2)cc1F)c1ccc2c(c1)CCO2] |
| 200 | 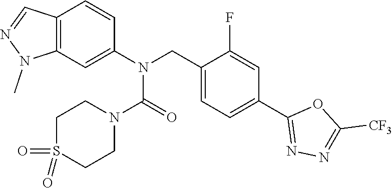 [SMILES: Cn1ncc2ccc(N(Cc3ccc(-c4nnc(C(F)(F)F)o4)cc3F)C(=O)N3CCS(=O)(=O)CC3)cc21] |
| 201 | 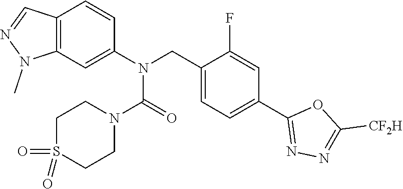 [SMILES: Cn1ncc2ccc(N(Cc3ccc(-c4nnc(C(F)F)o4)cc3F)C(=O)N3CCS(=O)(=O)CC3)cc21] |
| 202 | 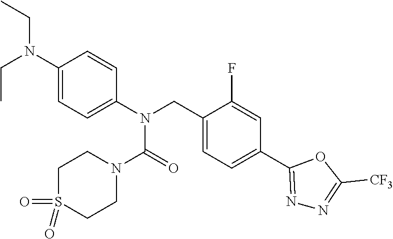 [SMILES: CCN(CC)c1ccc(N(Cc2ccc(-c3nnc(C(F)(F)F)o3)cc2F)C(=O)N2CCS(=O)(=O)CC2)cc1] |
| 203 | 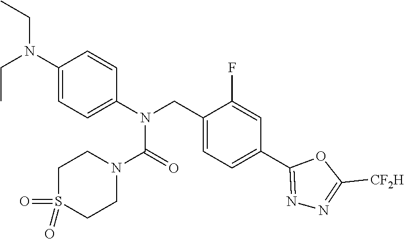 [SMILES: CCN(CC)c1ccc(N(Cc2ccc(-c3nnc(C(F)F)o3)cc2F)C(=O)N2CCS(=O)(=O)CC2)cc1] |
| 204 | 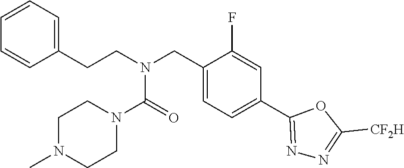 [SMILES: CN1CCN(C(=O)N(CCc2ccccc2)Cc2ccc(-c3nnc(C(F)F)o3)cc2F)CC1] |
| 205 | 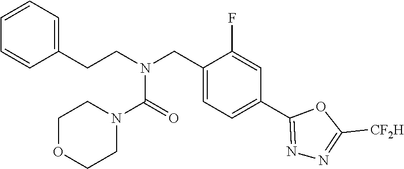 [SMILES: O=C(N1CCOCC1)N(CCc1ccccc1)Cc1ccc(-c2nnc(C(F)F)o2)cc1F] |
| 206 | 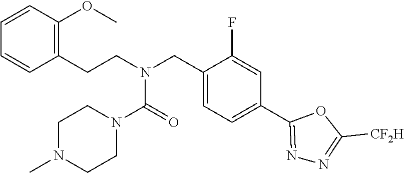 [SMILES: COc1ccccc1CCN(Cc1ccc(-c2nnc(C(F)F)o2)cc1F)C(=O)N1CCN(C)CC1] |
| 207 | 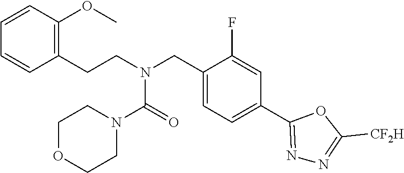 [SMILES: COc1ccccc1CCN(Cc1ccc(-c2nnc(C(F)F)o2)cc1F)C(=O)N1CCOCC1] |
| 208 | 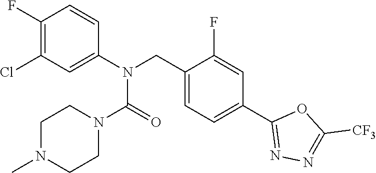 [SMILES: CN1CCN(C(=O)N(Cc2ccc(-c3nnc(C(F)(F)F)o3)cc2F)c2ccc(F)c(Cl)c2)CC1] |
| 209 | 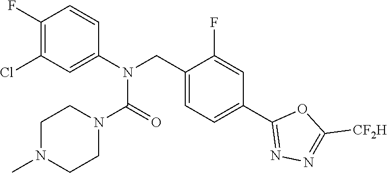 [SMILES: CN1CCN(C(=O)N(Cc2ccc(-c3nnc(C(F)F)o3)cc2F)c2ccc(F)c(Cl)c2)CC1] |
| 210 | 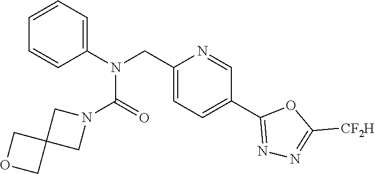 [SMILES: O=C(N1CC2(COC2)C1)N(Cc1ccc(-c2nnc(C(F)F)o2)cn1)c1ccccc1] |
| 211 | 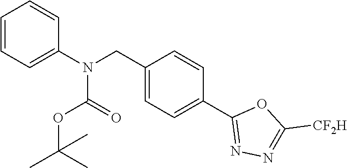 [SMILES: CC(C)(C)OC(=O)N(Cc1ccc(-c2nnc(C(F)F)o2)cc1)c1ccccc1] |
| 212 | 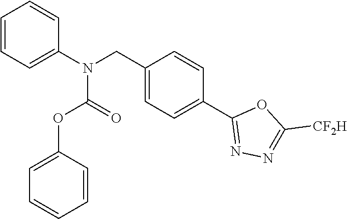 [SMILES: O=C(Oc1ccccc1)N(Cc1ccc(-c2nnc(C(F)F)o2)cc1)c1ccccc1] |
| 213 | 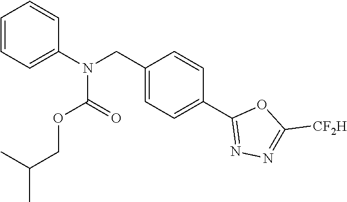 [SMILES: CC(C)COC(=O)N(Cc1ccc(-c2nnc(C(F)F)o2)cc1)c1ccccc1] |
| 214 | 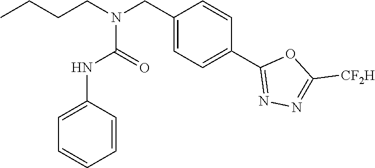 [SMILES: CCCCN(Cc1ccc(-c2nnc(C(F)F)o2)cc1)C(=O)Nc1ccccc1] |
| 215 | 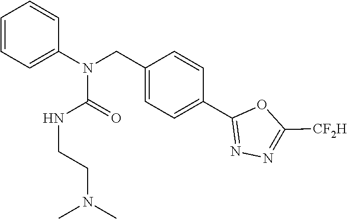 [SMILES: CN(C)CCNC(=O)N(Cc1ccc(-c2nnc(C(F)F)o2)cc1)c1ccccc1] |
| 216 | 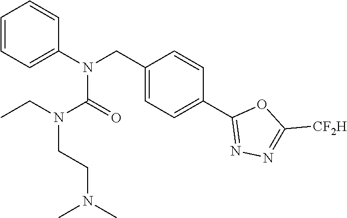 [SMILES: CCN(CCN(C)C)C(=O)N(Cc1ccc(-c2nnc(C(F)F)o2)cc1)c1ccccc1] |
| 217 | 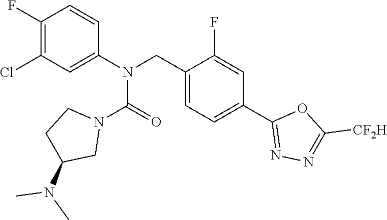 [SMILES: CN(C)[C@H]1CCN(C(=O)N(Cc2ccc(-c3nnc(C(F)F)o3)cc2F)c2ccc(F)c(Cl)c2)C1] |
| 218 | 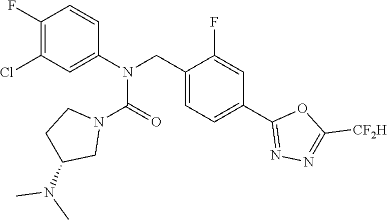 [SMILES: CN(C)[C@@H]1CCN(C(=O)N(Cc2ccc(-c3nnc(C(F)F)o3)cc2F)c2ccc(F)c(Cl)c2)C1] |
| 219 | 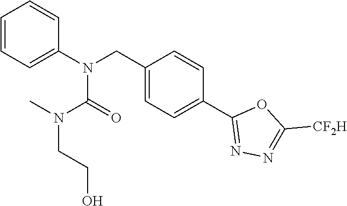 [SMILES: CN(CCO)C(=O)N(Cc1ccc(-c2nnc(C(F)F)o2)cc1)c1ccccc1] |
| 220 | 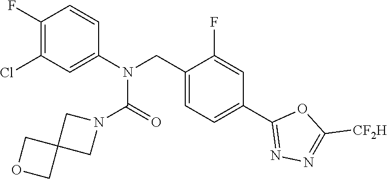 [SMILES: O=C(N1CC2(COC2)C1)N(Cc1ccc(-c2nnc(C(F)F)o2)cc1F)c1ccc(F)c(Cl)c1] |
| 221 | 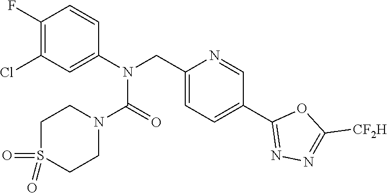 [SMILES: O=C(N1CCS(=O)(=O)CC1)N(Cc1ccc(-c2nnc(C(F)F)o2)cn1)c1ccc(F)c(Cl)c1] |
| 222 | 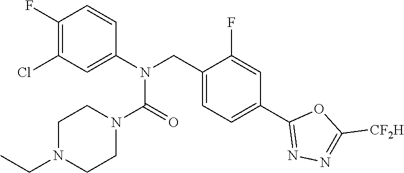 [SMILES: CCN1CCN(C(=O)N(Cc2ccc(-c3nnc(C(F)F)o3)cc2F)c2ccc(F)c(Cl)c2)CC1] |
| 223 | 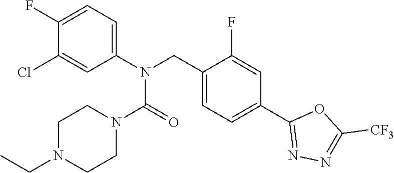 [SMILES: CCN1CCN(C(=O)N(Cc2ccc(-c3nnc(C(F)(F)F)o3)cc2F)c2ccc(F)c(Cl)c2)CC1] |
| 224 | 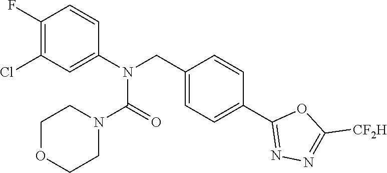 [SMILES: O=C(N1CCOCC1)N(Cc1ccc(-c2nnc(C(F)F)o2)cc1)c1ccc(F)c(Cl)c1] |
| 225 | 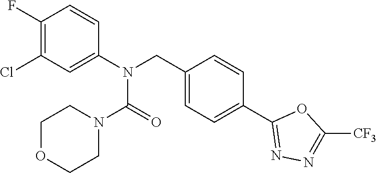 [SMILES: O=C(N1CCOCC1)N(Cc1ccc(-c2nnc(C(F)(F)F)o2)cc1)c1ccc(F)c(Cl)c1] |
| 226 | 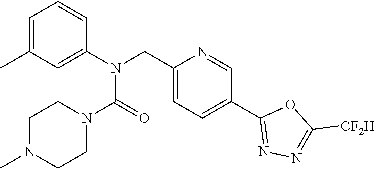 [SMILES: Cc1cccc(N(Cc2ccc(-c3nnc(C(F)F)o3)cn2)C(=O)N2CCN(C)CC2)c1] |
| 227 | 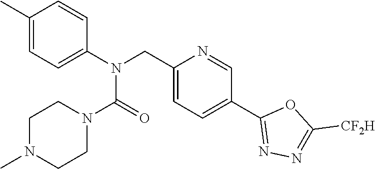 [SMILES: Cc1ccc(N(Cc2ccc(-c3nnc(C(F)F)o3)cn2)C(=O)N2CCN(C)CC2)cc1] |
| 228 | 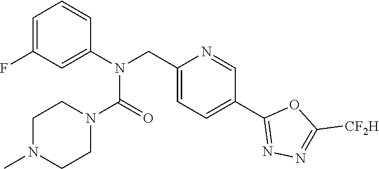 [SMILES: CN1CCN(C(=O)N(Cc2ccc(-c3nnc(C(F)F)o3)cn2)c2cccc(F)c2)CC1] |
| 229 | 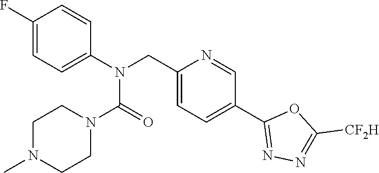 [SMILES: CN1CCN(C(=O)N(Cc2ccc(-c3nnc(C(F)F)o3)cn2)c2ccc(F)cc2)CC1] |
| 230 | 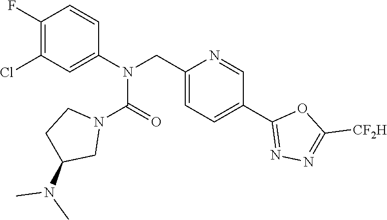 [SMILES: CN(C)[C@H]1CCN(C(=O)N(Cc2ccc(-c3nnc(C(F)F)o3)cn2)c2ccc(F)c(Cl)c2)C1] |
| 231 | 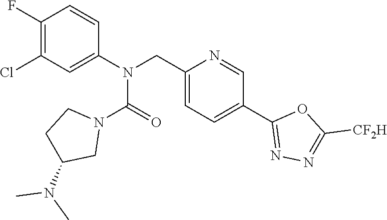 [SMILES: CN(C)[C@@H]1CCN(C(=O)N(Cc2ccc(-c3nnc(C(F)F)o3)cn2)c2ccc(F)c(Cl)c2)C1] |
| 232 | 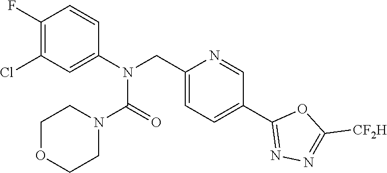 [SMILES: O=C(N1CCOCC1)N(Cc1ccc(-c2nnc(C(F)F)o2)cn1)c1ccc(F)c(Cl)c1] |
| 233 | 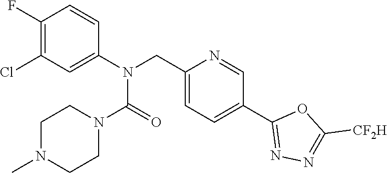 [SMILES: CN1CCN(C(=O)N(Cc2ccc(-c3nnc(C(F)F)o3)cn2)c2ccc(F)c(Cl)c2)CC1] |
| 234 | 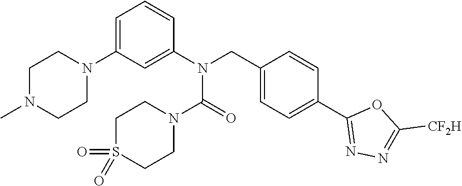 [SMILES: CN1CCN(c2cccc(N(Cc3ccc(-c4nnc(C(F)F)o4)cc3)C(=O)N3CCS(=O)(=O)CC3)c2)CC1] |
| 235 | 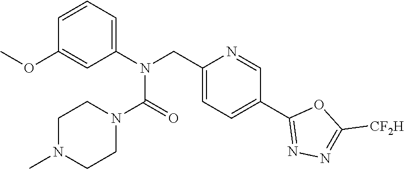 [SMILES: COc1cccc(N(Cc2ccc(-c3nnc(C(F)F)o3)cn2)C(=O)N2CCN(C)CC2)c1] |
| 236 | 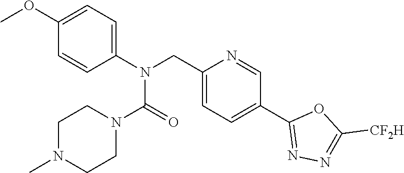 [SMILES: COc1ccc(N(Cc2ccc(-c3nnc(C(F)F)o3)cn2)C(=O)N2CCN(C)CC2)cc1] |
| 237 | 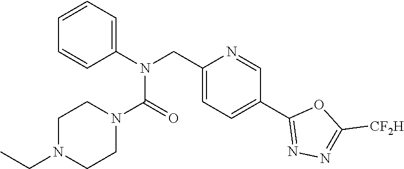 [SMILES: CCN1CCN(C(=O)N(Cc2ccc(-c3nnc(C(F)F)o3)cn2)c2ccccc2)CC1] |
| 238 | 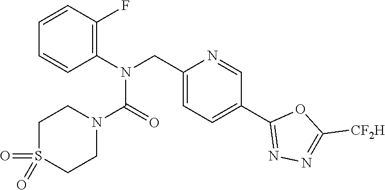 [SMILES: O=C(N1CCS(=O)(=O)CC1)N(Cc1ccc(-c2nnc(C(F)F)o2)cn1)c1ccccc1F] |
| 239 | 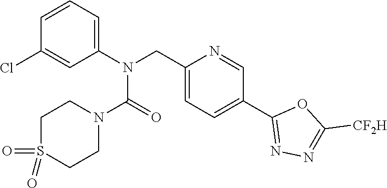 [SMILES: O=C(N1CCS(=O)(=O)CC1)N(Cc1ccc(-c2nnc(C(F)F)o2)cn1)c1cccc(Cl)c1] |
| 240 | 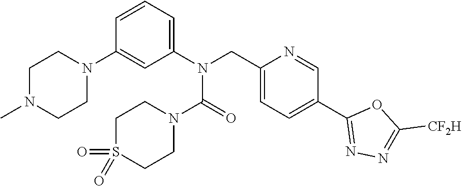 [SMILES: CN1CCN(c2cccc(N(Cc3ccc(-c4nnc(C(F)F)o4)cn3)C(=O)N3CCS(=O)(=O)CC3)c2)CC1] |
| 241 | 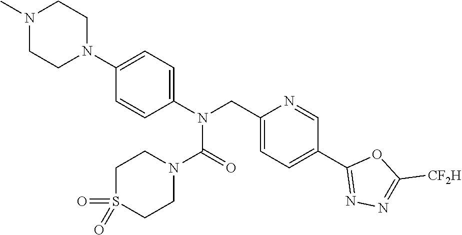 [SMILES: CN1CCN(c2ccc(N(Cc3ccc(-c4nnc(C(F)F)o4)cn3)C(=O)N3CCS(=O)(=O)CC3)cc2)CC1] |
| 242 | 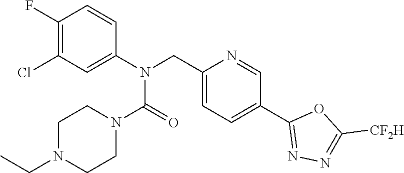 [SMILES: CCN1CCN(C(=O)N(Cc2ccc(-c3nnc(C(F)F)o3)cn2)c2ccc(F)c(Cl)c2)CC1] |
| 243 | 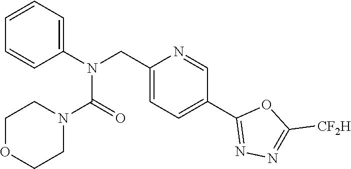 [SMILES: O=C(N1CCOCC1)N(Cc1ccc(-c2nnc(C(F)F)o2)cn1)c1ccccc1] |
| 244 | 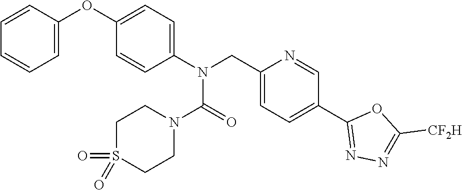 [SMILES: O=C(N1CCS(=O)(=O)CC1)N(Cc1ccc(-c2nnc(C(F)F)o2)cn1)c1ccc(Oc2ccccc2)cc1] |
| 245 | 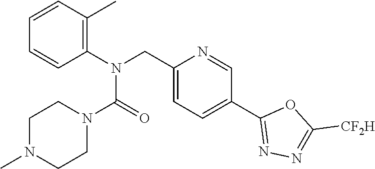 [SMILES: Cc1ccccc1N(Cc1ccc(-c2nnc(C(F)F)o2)cn1)C(=O)N1CCN(C)CC1] |
| 246 | 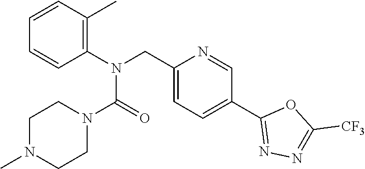 [SMILES: Cc1ccccc1N(Cc1ccc(-c2nnc(C(F)(F)F)o2)cn1)C(=O)N1CCN(C)CC1] |
| 247 | 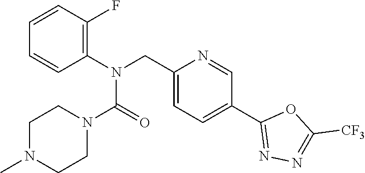 [SMILES: CN1CCN(C(=O)N(Cc2ccc(-c3nnc(C(F)(F)F)o3)cn2)c2ccccc2F)CC1] |
| 248 | 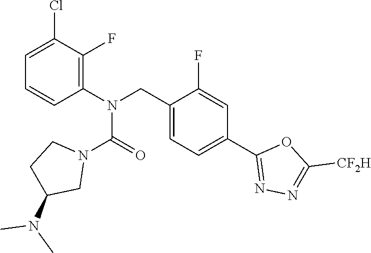 [SMILES: CN(C)[C@H]1CCN(C(=O)N(Cc2ccc(-c3nnc(C(F)F)o3)cc2F)c2cccc(Cl)c2F)C1] |
| 249 | 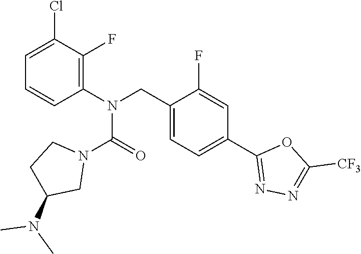 [SMILES: CN(C)[C@H]1CCN(C(=O)N(Cc2ccc(-c3nnc(C(F)(F)F)o3)cc2F)c2cccc(Cl)c2F)C1] |
| 250 | 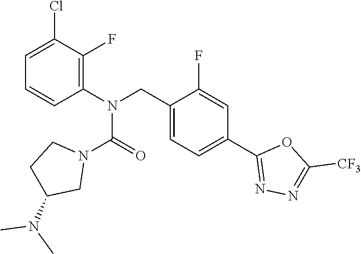 [SMILES: CN(C)[C@@H]1CCN(C(=O)N(Cc2ccc(-c3nnc(C(F)(F)F)o3)cc2F)c2cccc(Cl)c2F)C1] |
| 251 | 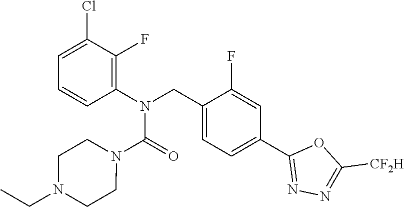 [SMILES: CCN1CCN(C(=O)N(Cc2ccc(-c3nnc(C(F)F)o3)cc2F)c2cccc(Cl)c2F)CC1] |
| 252 | 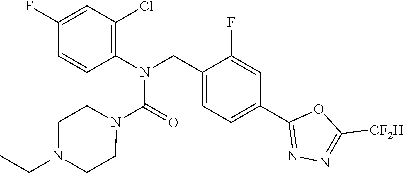 [SMILES: CCN1CCN(C(=O)N(Cc2ccc(-c3nnc(C(F)F)o3)cc2F)c2ccc(F)cc2Cl)CC1] |
| 253 | 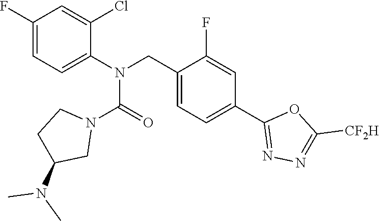 [SMILES: CN(C)[C@H]1CCN(C(=O)N(Cc2ccc(-c3nnc(C(F)F)o3)cc2F)c2ccc(F)cc2Cl)C1] |
| 254 | 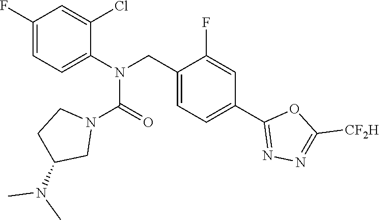 [SMILES: CN(C)[C@@H]1CCN(C(=O)N(Cc2ccc(-c3nnc(C(F)F)o3)cc2F)c2ccc(F)cc2Cl)C1] |
| 255 | 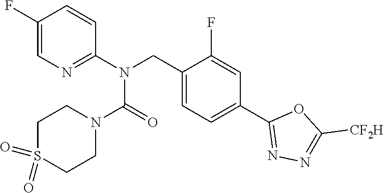 [SMILES: O=C(N1CCS(=O)(=O)CC1)N(Cc1ccc(-c2nnc(C(F)F)o2)cc1F)c1ccc(F)cn1] |
| 256 | 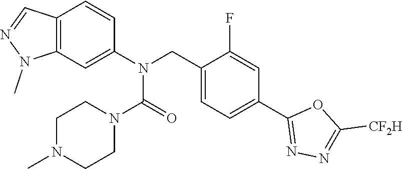 [SMILES: CN1CCN(C(=O)N(Cc2ccc(-c3nnc(C(F)F)o3)cc2F)c2ccc3cnn(C)c3c2)CC1] |
| 257 | 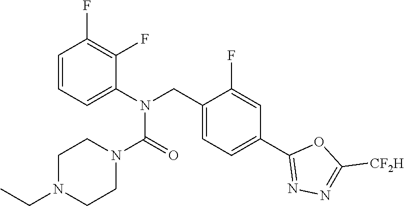 [SMILES: CCN1CCN(C(=O)N(Cc2ccc(-c3nnc(C(F)F)o3)cc2F)c2cccc(F)c2F)CC1] |
| 258 | 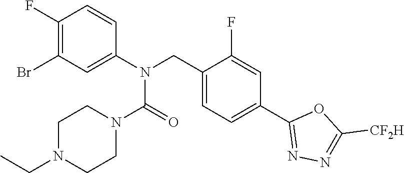 [SMILES: CCN1CCN(C(=O)N(Cc2ccc(-c3nnc(C(F)F)o3)cc2F)c2ccc(F)c(Br)c2)CC1] |
| 259 | 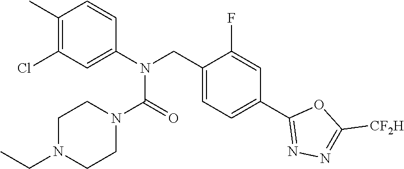 [SMILES: CCN1CCN(C(=O)N(Cc2ccc(-c3nnc(C(F)F)o3)cc2F)c2ccc(C)c(Cl)c2)CC1] |
| 260 | 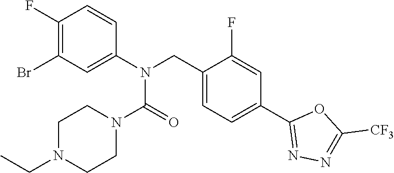 [SMILES: CCN1CCN(C(=O)N(Cc2ccc(-c3nnc(C(F)(F)F)o3)cc2F)c2ccc(F)c(Br)c2)CC1] |
| 261 | 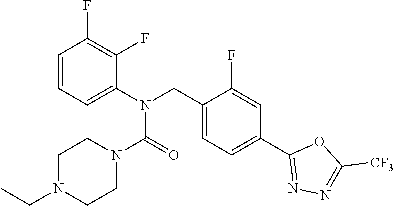 [SMILES: CCN1CCN(C(=O)N(Cc2ccc(-c3nnc(C(F)(F)F)o3)cc2F)c2cccc(F)c2F)CC1] |
| 262 | 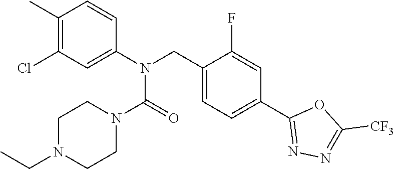 [SMILES: CCN1CCN(C(=O)N(Cc2ccc(-c3nnc(C(F)(F)F)o3)cc2F)c2ccc(C)c(Cl)c2)CC1] |
| 263 | 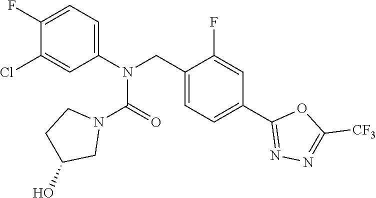 [SMILES: O=C(N1CC[C@@H](O)C1)N(Cc1ccc(-c2nnc(C(F)(F)F)o2)cc1F)c1ccc(F)c(Cl)c1] |
| 264 | 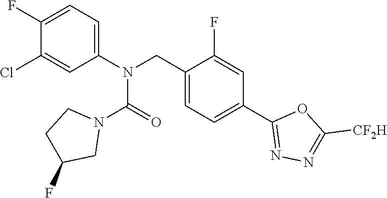 [SMILES: O=C(N1CC[C@H](F)C1)N(Cc1ccc(-c2nnc(C(F)F)o2)cc1F)c1ccc(F)c(Cl)c1] |
| 265 | 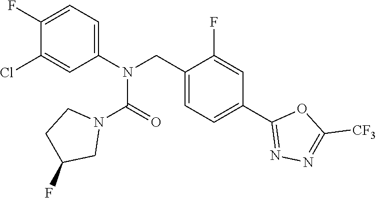 [SMILES: O=C(N1CC[C@H](F)C1)N(Cc1ccc(-c2nnc(C(F)(F)F)o2)cc1F)c1ccc(F)c(Cl)c1] |
| 266 | 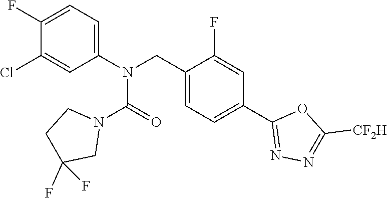 [SMILES: O=C(N1CCC(F)(F)C1)N(Cc1ccc(-c2nnc(C(F)F)o2)cc1F)c1ccc(F)c(Cl)c1] |
| 267 | 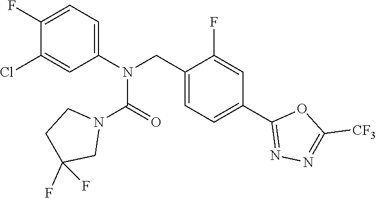 [SMILES: O=C(N1CCC(F)(F)C1)N(Cc1ccc(-c2nnc(C(F)(F)F)o2)cc1F)c1ccc(F)c(Cl)c1] |
| 268 | 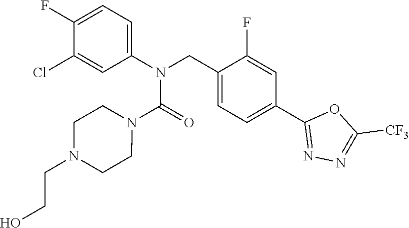 [SMILES: O=C(N1CCN(CCO)CC1)N(Cc1ccc(-c2nnc(C(F)(F)F)o2)cc1F)c1ccc(F)c(Cl)c1] |
| 269 | 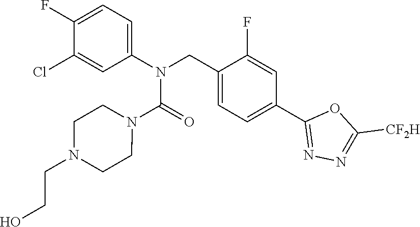 [SMILES: O=C(N1CCN(CCO)CC1)N(Cc1ccc(-c2nnc(C(F)F)o2)cc1F)c1ccc(F)c(Cl)c1] |
| 270 | 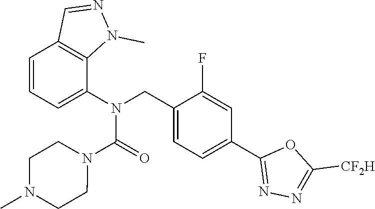 [SMILES: CN1CCN(C(=O)N(Cc2ccc(-c3nnc(C(F)F)o3)cc2F)c2cccc3cnn(C)c23)CC1] |
| 271 | 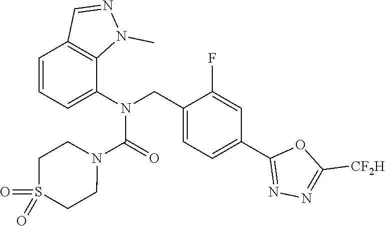 [SMILES: Cn1ncc2cccc(N(Cc3ccc(-c4nnc(C(F)F)o4)cc3F)C(=O)N3CCS(=O)(=O)CC3)c21] |
| 272 | 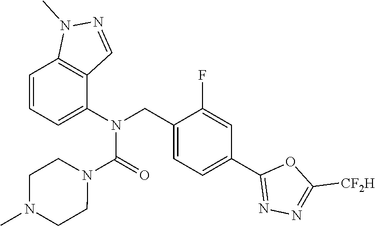 [SMILES: CN1CCN(C(=O)N(Cc2ccc(-c3nnc(C(F)F)o3)cc2F)c2cccc3c2cnn3C)CC1] |
| 273 | 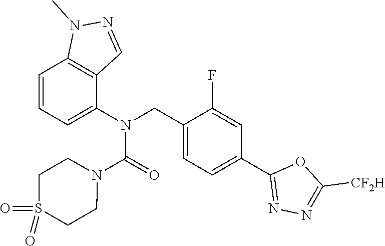 [SMILES: Cn1ncc2c(N(Cc3ccc(-c4nnc(C(F)F)o4)cc3F)C(=O)N3CCS(=O)(=O)CC3)cccc21] |
| 274 | 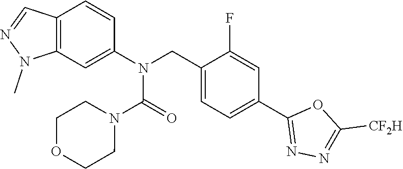 [SMILES: Cn1ncc2ccc(N(Cc3ccc(-c4nnc(C(F)F)o4)cc3F)C(=O)N3CCOCC3)cc21] |
| 275 | 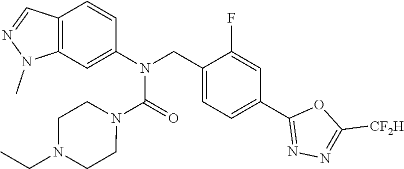 [SMILES: CCN1CCN(C(=O)N(Cc2ccc(-c3nnc(C(F)F)o3)cc2F)c2ccc3cnn(C)c3c2)CC1] |
| 276 | 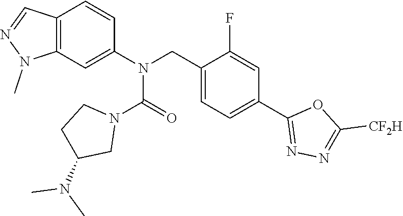 [SMILES: CN(C)[C@@H]1CCN(C(=O)N(Cc2ccc(-c3nnc(C(F)F)o3)cc2F)c2ccc3cnn(C)c3c2)C1] |
| 277 | 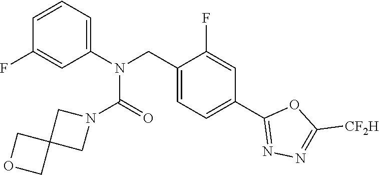 [SMILES: O=C(N1CC2(COC2)C1)N(Cc1ccc(-c2nnc(C(F)F)o2)cc1F)c1cccc(F)c1] |
| 278 | 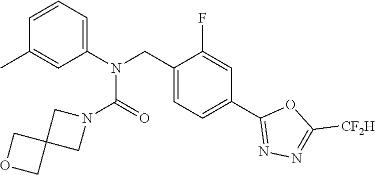 [SMILES: Cc1cccc(N(Cc2ccc(-c3nnc(C(F)F)o3)cc2F)C(=O)N2CC3(COC3)C2)c1] |
| 279 | 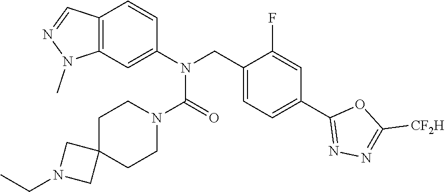 [SMILES: CCN1CC2(CCN(C(=O)N(Cc3ccc(-c4nnc(C(F)F)o4)cc3F)c3ccc4cnn(C)c4c3)CC2)C1] |
| 280 | 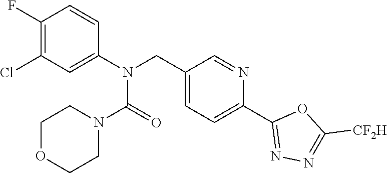 [SMILES: O=C(N1CCOCC1)N(Cc1ccc(-c2nnc(C(F)F)o2)nc1)c1ccc(F)c(Cl)c1] |
| 281 | 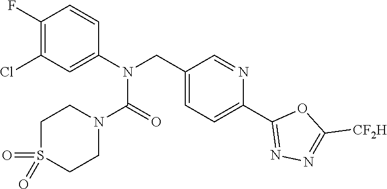 [SMILES: O=C(N1CCS(=O)(=O)CC1)N(Cc1ccc(-c2nnc(C(F)F)o2)nc1)c1ccc(F)c(Cl)c1] |
| 282 | 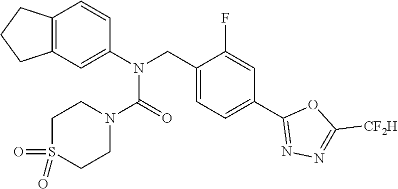 [SMILES: O=C(N1CCS(=O)(=O)CC1)N(Cc1ccc(-c2nnc(C(F)F)o2)cc1F)c1ccc2c(c1)CCC2] |
| 283 | 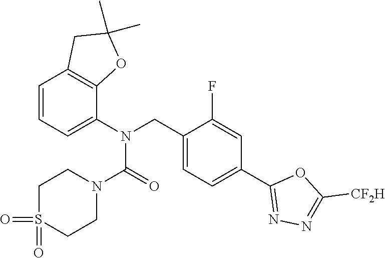 [SMILES: CC1(C)Cc2cccc(N(Cc3ccc(-c4nnc(C(F)F)o4)cc3F)C(=O)N3CCS(=O)(=O)CC3)c2O1] |
| 284 | 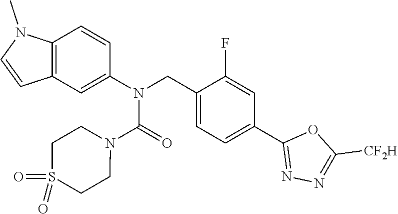 [SMILES: Cn1ccc2cc(N(Cc3ccc(-c4nnc(C(F)F)o4)cc3F)C(=O)N3CCS(=O)(=O)CC3)ccc21] |
| 285 | 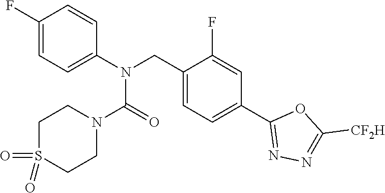 [SMILES: O=C(N1CCS(=O)(=O)CC1)N(Cc1ccc(-c2nnc(C(F)F)o2)cc1F)c1ccc(F)cc1] |
| 286 | 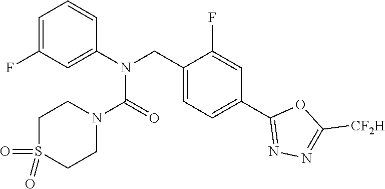 [SMILES: O=C(N1CCS(=O)(=O)CC1)N(Cc1ccc(-c2nnc(C(F)F)o2)cc1F)c1cccc(F)c1] |
| 287 | 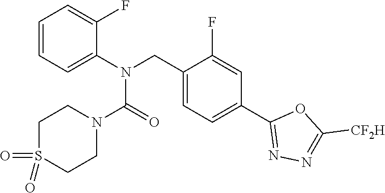 [SMILES: O=C(N1CCS(=O)(=O)CC1)N(Cc1ccc(-c2nnc(C(F)F)o2)cc1F)c1ccccc1F] |
| 288 | 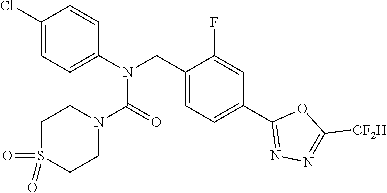 [SMILES: O=C(N1CCS(=O)(=O)CC1)N(Cc1ccc(-c2nnc(C(F)F)o2)cc1F)c1ccc(Cl)cc1] |
| 289 | 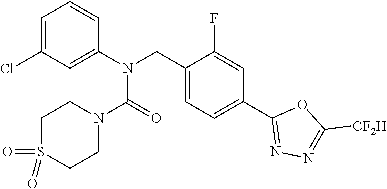 [SMILES: O=C(N1CCS(=O)(=O)CC1)N(Cc1ccc(-c2nnc(C(F)F)o2)cc1F)c1cccc(Cl)c1] |
| 290 | 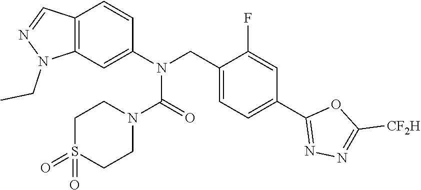 [SMILES: CCn1ncc2ccc(N(Cc3ccc(-c4nnc(C(F)F)o4)cc3F)C(=O)N3CCS(=O)(=O)CC3)cc21] |
| 291 | 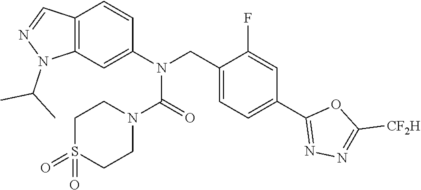 [SMILES: CC(C)n1ncc2ccc(N(Cc3ccc(-c4nnc(C(F)F)o4)cc3F)C(=O)N3CCS(=O)(=O)CC3)cc21] |
| 292 | 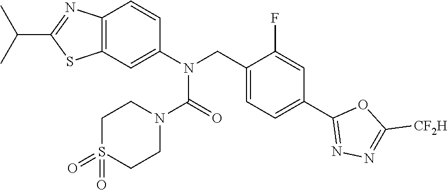 [SMILES: CC(C)c1nc2ccc(N(Cc3ccc(-c4nnc(C(F)F)o4)cc3F)C(=O)N3CCS(=O)(=O)CC3)cc2s1] |
| 293 | 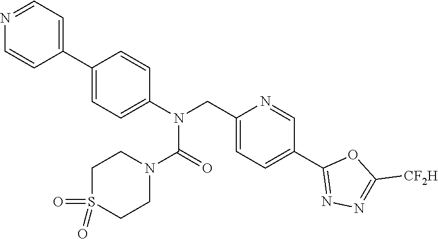 [SMILES: O=C(N1CCS(=O)(=O)CC1)N(Cc1ccc(-c2nnc(C(F)F)o2)cn1)c1ccc(-c2ccncc2)cc1] |
| 294 | 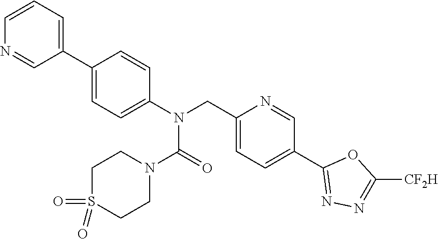 [SMILES: O=C(N1CCS(=O)(=O)CC1)N(Cc1ccc(-c2nnc(C(F)F)o2)cn1)c1ccc(-c2cccnc2)cc1] |
| 295 | 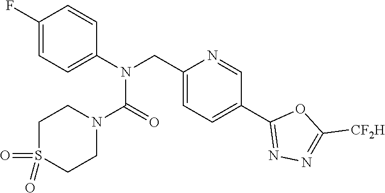 [SMILES: O=C(N1CCS(=O)(=O)CC1)N(Cc1ccc(-c2nnc(C(F)F)o2)cn1)c1ccc(F)cc1] |
| 296 | 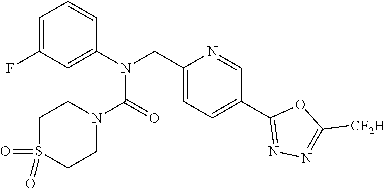 [SMILES: O=C(N1CCS(=O)(=O)CC1)N(Cc1ccc(-c2nnc(C(F)F)o2)cn1)c1cccc(F)c1] |
| 297 | 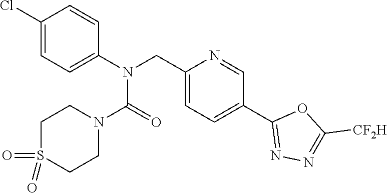 [SMILES: O=C(N1CCS(=O)(=O)CC1)N(Cc1ccc(-c2nnc(C(F)F)o2)cn1)c1ccc(Cl)cc1] |
| 298 | 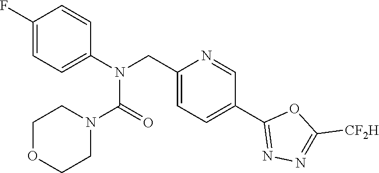 [SMILES: O=C(N1CCOCC1)N(Cc1ccc(-c2nnc(C(F)F)o2)cn1)c1ccc(F)cc1] |
| 299 | 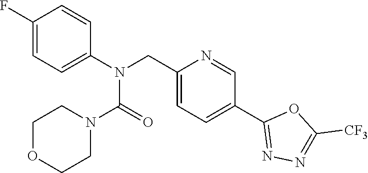 [SMILES: O=C(N1CCOCC1)N(Cc1ccc(-c2nnc(C(F)(F)F)o2)cn1)c1ccc(F)cc1] |
| 300 | 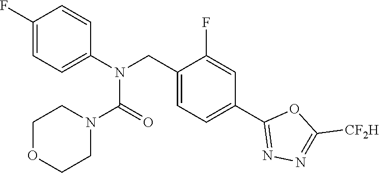 [SMILES: O=C(N1CCOCC1)N(Cc1ccc(-c2nnc(C(F)F)o2)cc1F)c1ccc(F)cc1] |
| 301 | 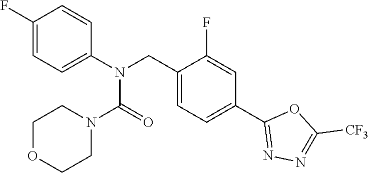 [SMILES: O=C(N1CCOCC1)N(Cc1ccc(-c2nnc(C(F)(F)F)o2)cc1F)c1ccc(F)cc1] |
| 302 | 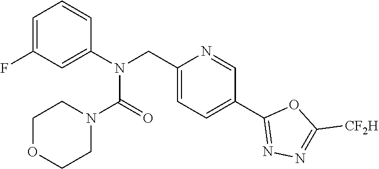 [SMILES: O=C(N1CCOCC1)N(Cc1ccc(-c2nnc(C(F)F)o2)cn1)c1cccc(F)c1] |
| 303 | 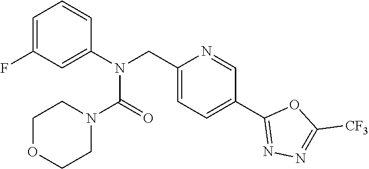 [SMILES: O=C(N1CCOCC1)N(Cc1ccc(-c2nnc(C(F)(F)F)o2)cn1)c1cccc(F)c1] |
| 304 | 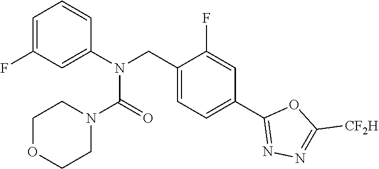 [SMILES: O=C(N1CCOCC1)N(Cc1ccc(-c2nnc(C(F)F)o2)cc1F)c1cccc(F)c1] |
| 305 | 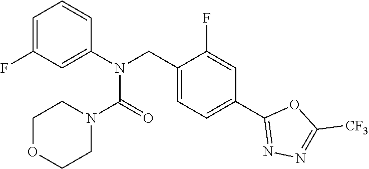 [SMILES: O=C(N1CCOCC1)N(Cc1ccc(-c2nnc(C(F)(F)F)o2)cc1F)c1cccc(F)c1] |
| 306 | 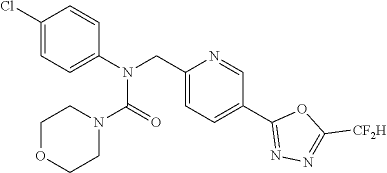 [SMILES: O=C(N1CCOCC1)N(Cc1ccc(-c2nnc(C(F)F)o2)cn1)c1ccc(Cl)cc1] |
| 307 | 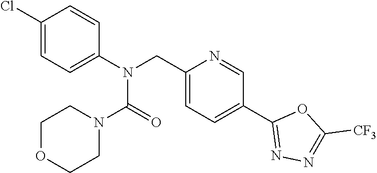 [SMILES: O=C(N1CCOCC1)N(Cc1ccc(-c2nnc(C(F)(F)F)o2)cn1)c1ccc(Cl)cc1] |
| 308 | 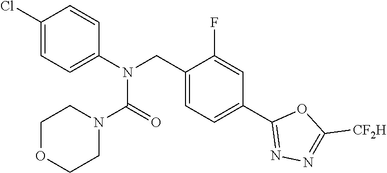 [SMILES: O=C(N1CCOCC1)N(Cc1ccc(-c2nnc(C(F)F)o2)cc1F)c1ccc(Cl)cc1] |
| 309 | 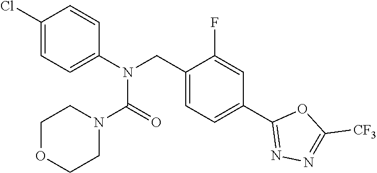 [SMILES: O=C(N1CCOCC1)N(Cc1ccc(-c2nnc(C(F)(F)F)o2)cc1F)c1ccc(Cl)cc1] |
| 310 | 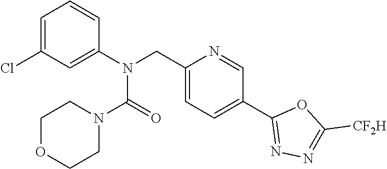 [SMILES: O=C(N1CCOCC1)N(Cc1ccc(-c2nnc(C(F)F)o2)cn1)c1cccc(Cl)c1] |
| 311 | 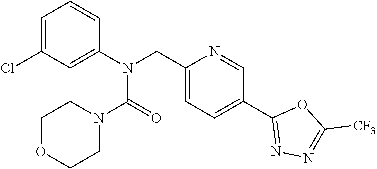 [SMILES: O=C(N1CCOCC1)N(Cc1ccc(-c2nnc(C(F)(F)F)o2)cn1)c1cccc(Cl)c1] |
| 312 | 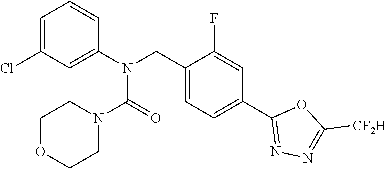 [SMILES: O=C(N1CCOCC1)N(Cc1ccc(-c2nnc(C(F)F)o2)cc1F)c1cccc(Cl)c1] |
| 313 | 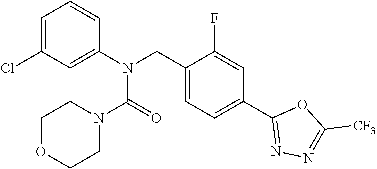 [SMILES: O=C(N1CCOCC1)N(Cc1ccc(-c2nnc(C(F)(F)F)o2)cc1F)c1cccc(Cl)c1] |
| 314 | 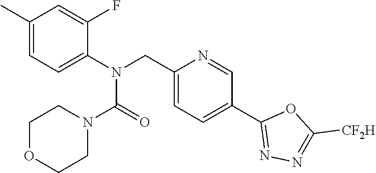 [SMILES: Cc1ccc(N(Cc2ccc(-c3nnc(C(F)F)o3)cn2)C(=O)N2CCOCC2)c(F)c1] |
| 315 | 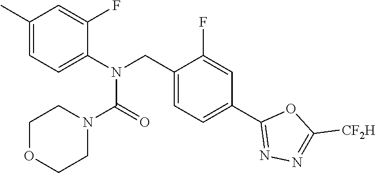 [SMILES: Cc1ccc(N(Cc2ccc(-c3nnc(C(F)F)o3)cc2F)C(=O)N2CCOCC2)c(F)c1] |
| 316 | 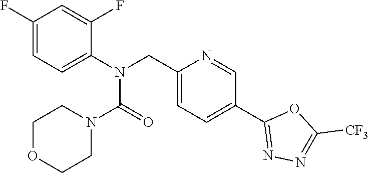 [SMILES: O=C(N1CCOCC1)N(Cc1ccc(-c2nnc(C(F)(F)F)o2)cn1)c1ccc(F)cc1F] |
| 317 | 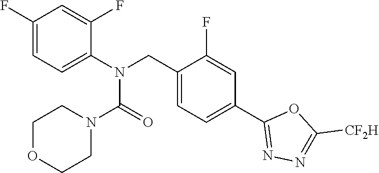 [SMILES: O=C(N1CCOCC1)N(Cc1ccc(-c2nnc(C(F)F)o2)cc1F)c1ccc(F)cc1F] |
| 318 | 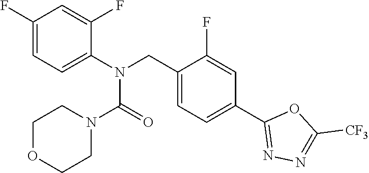 [SMILES: O=C(N1CCOCC1)N(Cc1ccc(-c2nnc(C(F)(F)F)o2)cc1F)c1ccc(F)cc1F] |
| 319 | 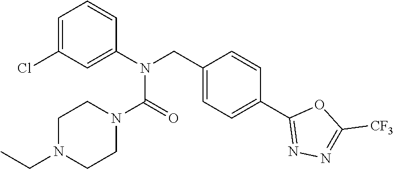 [SMILES: CCN1CCN(C(=O)N(Cc2ccc(-c3nnc(C(F)(F)F)o3)cc2)c2cccc(Cl)c2)CC1] |
| 320 | 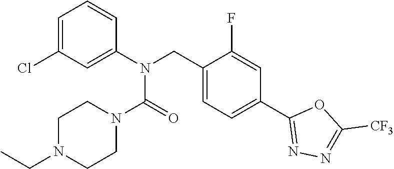 [SMILES: CCN1CCN(C(=O)N(Cc2ccc(-c3nnc(C(F)(F)F)o3)cc2F)c2cccc(Cl)c2)CC1] |
| 321 | 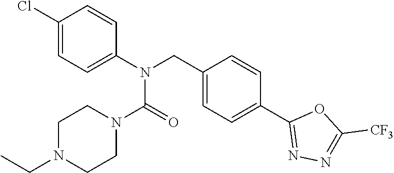 [SMILES: CCN1CCN(C(=O)N(Cc2ccc(-c3nnc(C(F)(F)F)o3)cc2)c2ccc(Cl)cc2)CC1] |
| 322 | 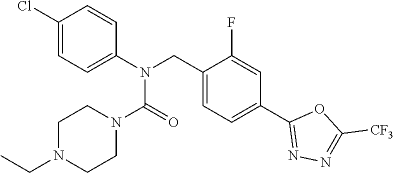 [SMILES: CCN1CCN(C(=O)N(Cc2ccc(-c3nnc(C(F)(F)F)o3)cc2F)c2ccc(Cl)cc2)CC1] |
| 323 | 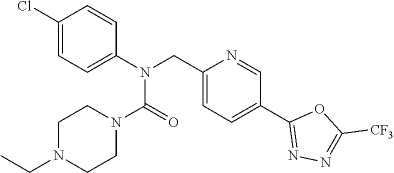 [SMILES: CCN1CCN(C(=O)N(Cc2ccc(-c3nnc(C(F)(F)F)o3)cn2)c2ccc(Cl)cc2)CC1] |
| 324 | 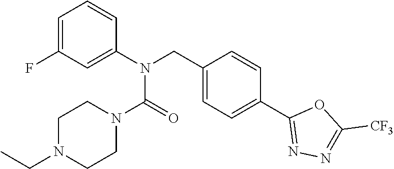 [SMILES: CCN1CCN(C(=O)N(Cc2ccc(-c3nnc(C(F)(F)F)o3)cc2)c2cccc(F)c2)CC1] |
| 325 | 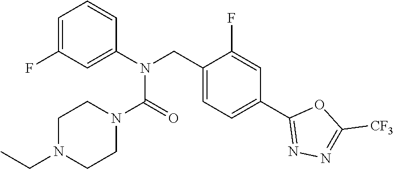 [SMILES: CCN1CCN(C(=O)N(Cc2ccc(-c3nnc(C(F)(F)F)o3)cc2F)c2cccc(F)c2)CC1] |
| 326 | 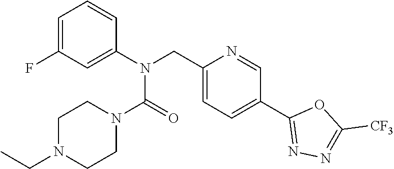 [SMILES: CCN1CCN(C(=O)N(Cc2ccc(-c3nnc(C(F)(F)F)o3)cn2)c2cccc(F)c2)CC1] |
| 327 | 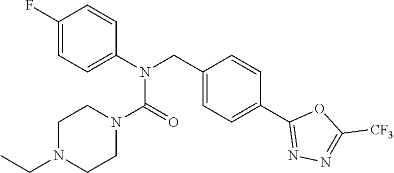 [SMILES: CCN1CCN(C(=O)N(Cc2ccc(-c3nnc(C(F)(F)F)o3)cc2)c2ccc(F)cc2)CC1] |
| 328 | 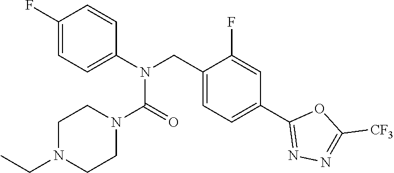 [SMILES: CCN1CCN(C(=O)N(Cc2ccc(-c3nnc(C(F)(F)F)o3)cc2F)c2ccc(F)cc2)CC1] |
| 329 | 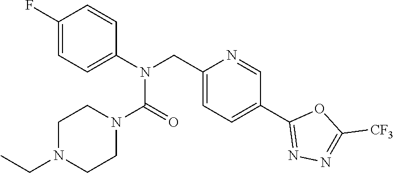 [SMILES: CCN1CCN(C(=O)N(Cc2ccc(-c3nnc(C(F)(F)F)o3)cn2)c2ccc(F)cc2)CC1] |
| 330 | 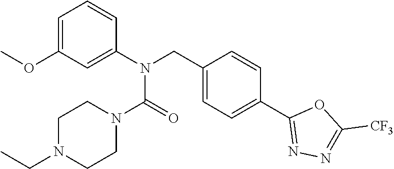 [SMILES: CCN1CCN(C(=O)N(Cc2ccc(-c3nnc(C(F)(F)F)o3)cc2)c2cccc(OC)c2)CC1] |
| 331 | 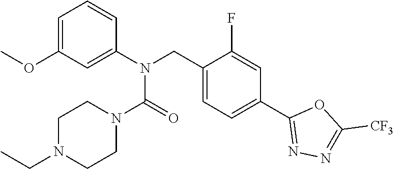 [SMILES: CCN1CCN(C(=O)N(Cc2ccc(-c3nnc(C(F)(F)F)o3)cc2F)c2cccc(OC)c2)CC1] |
| 332 | 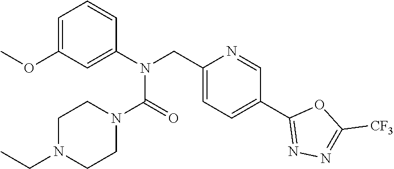 [SMILES: CCN1CCN(C(=O)N(Cc2ccc(-c3nnc(C(F)(F)F)o3)cn2)c2cccc(OC)c2)CC1] |
| 333 | 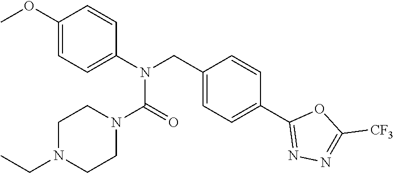 [SMILES: CCN1CCN(C(=O)N(Cc2ccc(-c3nnc(C(F)(F)F)o3)cc2)c2ccc(OC)cc2)CC1] |
| 334 | 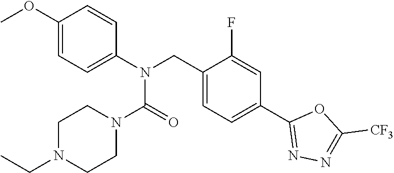 [SMILES: CCN1CCN(C(=O)N(Cc2ccc(-c3nnc(C(F)(F)F)o3)cc2F)c2ccc(OC)cc2)CC1] |
| 335 | 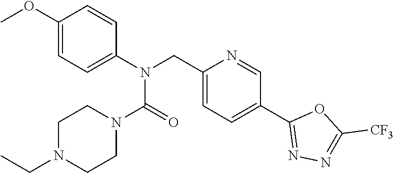 [SMILES: CCN1CCN(C(=O)N(Cc2ccc(-c3nnc(C(F)(F)F)o3)cn2)c2ccc(OC)cc2)CC1] |
| 336 | 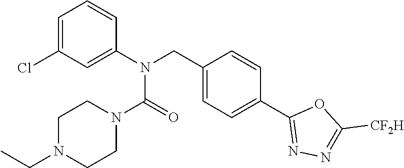 [SMILES: CCN1CCN(C(=O)N(Cc2ccc(-c3nnc(C(F)F)o3)cc2)c2cccc(Cl)c2)CC1] |
| 337 | 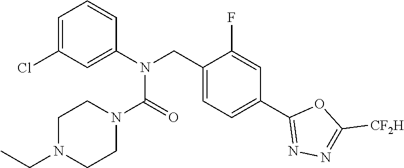 [SMILES: CCN1CCN(C(=O)N(Cc2ccc(-c3nnc(C(F)F)o3)cc2F)c2cccc(Cl)c2)CC1] |
| 338 | 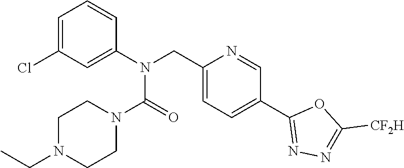 [SMILES: CCN1CCN(C(=O)N(Cc2ccc(-c3nnc(C(F)F)o3)cn2)c2cccc(Cl)c2)CC1] |
| 339 | 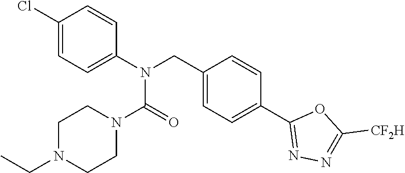 [SMILES: CCN1CCN(C(=O)N(Cc2ccc(-c3nnc(C(F)F)o3)cc2)c2ccc(Cl)cc2)CC1] |
| 340 | 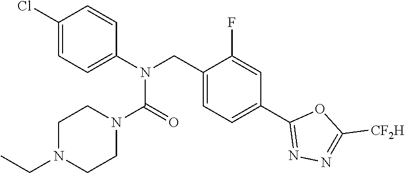 [SMILES: CCN1CCN(C(=O)N(Cc2ccc(-c3nnc(C(F)F)o3)cc2F)c2ccc(Cl)cc2)CC1] |
| 341 | 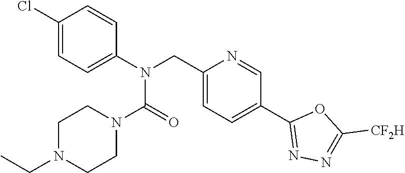 [SMILES: CCN1CCN(C(=O)N(Cc2ccc(-c3nnc(C(F)F)o3)cn2)c2ccc(Cl)cc2)CC1] |
| 342 | 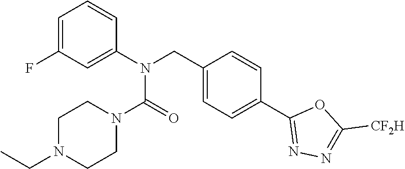 [SMILES: CCN1CCN(C(=O)N(Cc2ccc(-c3nnc(C(F)F)o3)cc2)c2cccc(F)c2)CC1] |
| 343 | 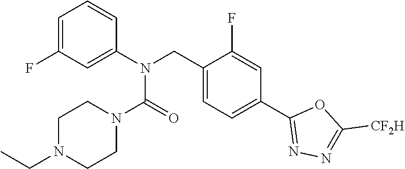 [SMILES: CCN1CCN(C(=O)N(Cc2ccc(-c3nnc(C(F)F)o3)cc2F)c2cccc(F)c2)CC1] |
| 344 | 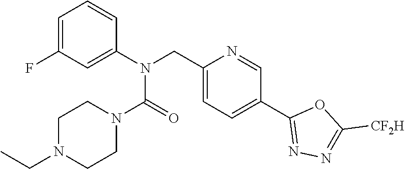 [SMILES: CCN1CCN(C(=O)N(Cc2ccc(-c3nnc(C(F)F)o3)cn2)c2cccc(F)c2)CC1] |
| 345 | 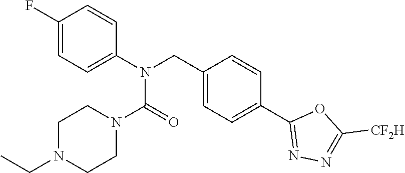 [SMILES: CCN1CCN(C(=O)N(Cc2ccc(-c3nnc(C(F)F)o3)cc2)c2ccc(F)cc2)CC1] |
| 346 | 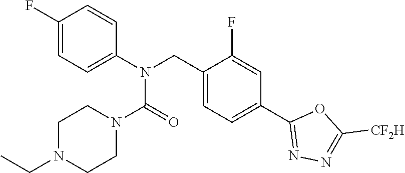 [SMILES: CCN1CCN(C(=O)N(Cc2ccc(-c3nnc(C(F)F)o3)cc2F)c2ccc(F)cc2)CC1] |
| 347 | 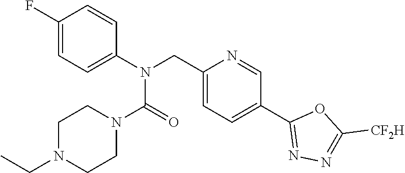 [SMILES: CCN1CCN(C(=O)N(Cc2ccc(-c3nnc(C(F)F)o3)cn2)c2ccc(F)cc2)CC1] |
| 348 | 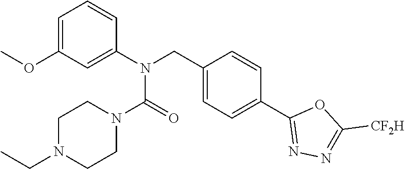 [SMILES: CCN1CCN(C(=O)N(Cc2ccc(-c3nnc(C(F)F)o3)cc2)c2cccc(OC)c2)CC1] |
| 349 | 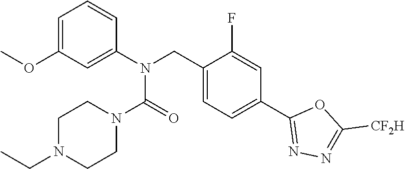 [SMILES: CCN1CCN(C(=O)N(Cc2ccc(-c3nnc(C(F)F)o3)cc2F)c2cccc(OC)c2)CC1] |
| 350 | 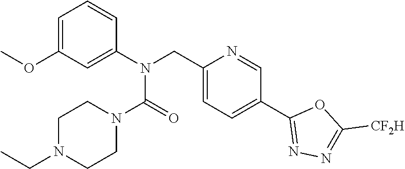 [SMILES: CCN1CCN(C(=O)N(Cc2ccc(-c3nnc(C(F)F)o3)cn2)c2cccc(OC)c2)CC1] |
| 351 | 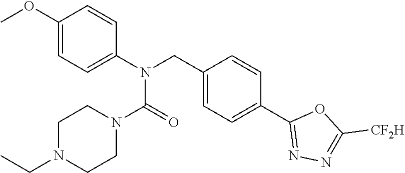 [SMILES: CCN1CCN(C(=O)N(Cc2ccc(-c3nnc(C(F)F)o3)cc2)c2ccc(OC)cc2)CC1] |
| 352 | 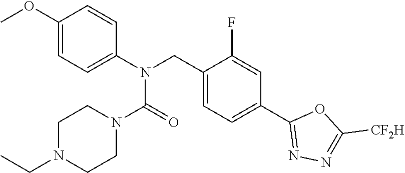 [SMILES: CCN1CCN(C(=O)N(Cc2ccc(-c3nnc(C(F)F)o3)cc2F)c2ccc(OC)cc2)CC1] |
| 353 | 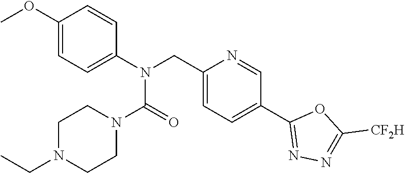 [SMILES: CCN1CCN(C(=O)N(Cc2ccc(-c3nnc(C(F)F)o3)cn2)c2ccc(OC)cc2)CC1] |
| 354 | 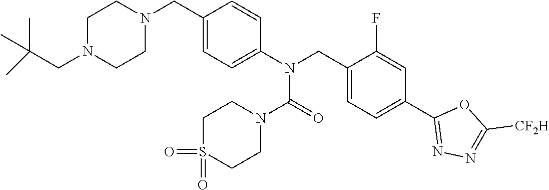 [SMILES: CC(C)(C)CN1CCN(Cc2ccc(N(Cc3ccc(-c4nnc(C(F)F)o4)cc3F)C(=O)N3CCS(=O)(=O)CC3)cc2)CC1] |
| 355 | 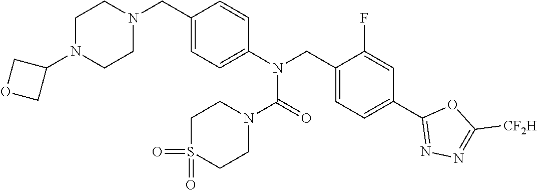 [SMILES: O=C(N1CCS(=O)(=O)CC1)N(Cc1ccc(-c2nnc(C(F)F)o2)cc1F)c1ccc(CN2CCN(C3COC3)CC2)cc1] |
| 356 | 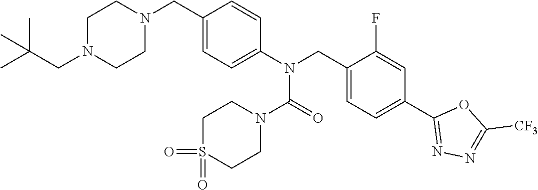 [SMILES: CC(C)(C)CN1CCN(Cc2ccc(N(Cc3ccc(-c4nnc(C(F)(F)F)o4)cc3F)C(=O)N3CCS(=O)(=O)CC3)cc2)CC1] |
| 357 | 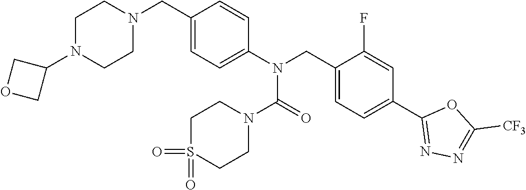 [SMILES: O=C(N1CCS(=O)(=O)CC1)N(Cc1ccc(-c2nnc(C(F)(F)F)o2)cc1F)c1ccc(CN2CCN(C3COC3)CC2)cc1] |
| 358 | 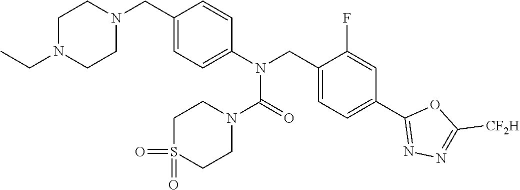 [SMILES: CCN1CCN(Cc2ccc(N(Cc3ccc(-c4nnc(C(F)F)o4)cc3F)C(=O)N3CCS(=O)(=O)CC3)cc2)CC1] |
| 359 | 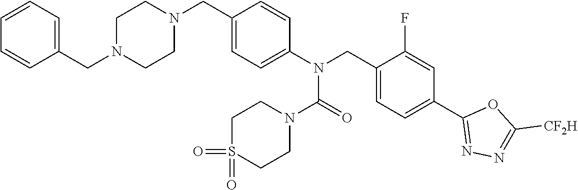 [SMILES: O=C(N1CCS(=O)(=O)CC1)N(Cc1ccc(-c2nnc(C(F)F)o2)cc1F)c1ccc(CN2CCN(Cc3ccccc3)CC2)cc1] |
| 360 | 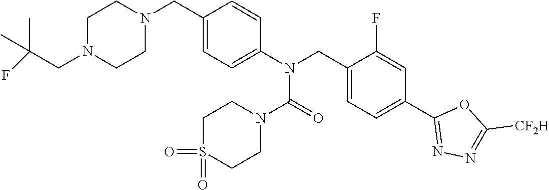 [SMILES: CC(C)(F)CN1CCN(Cc2ccc(N(Cc3ccc(-c4nnc(C(F)F)o4)cc3F)C(=O)N3CCS(=O)(=O)CC3)cc2)CC1] |
| 361 | 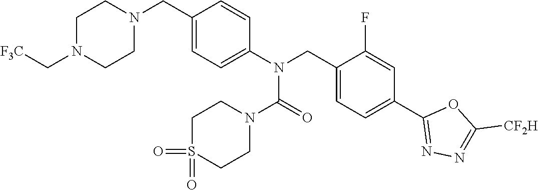 [SMILES: O=C(N1CCS(=O)(=O)CC1)N(Cc1ccc(-c2nnc(C(F)F)o2)cc1F)c1ccc(CN2CCN(CC(F)(F)F)CC2)cc1] |
| 362 | 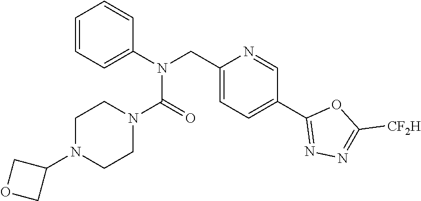 [SMILES: O=C(N1CCN(C2COC2)CC1)N(Cc1ccc(-c2nnc(C(F)F)o2)cn1)c1ccccc1] |
| 363 | 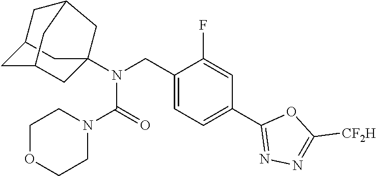 [SMILES: O=C(N1CCOCC1)N(Cc1ccc(-c2nnc(C(F)F)o2)cc1F)C12CC3CC(CC(C3)C1)C2] |
| 364 | 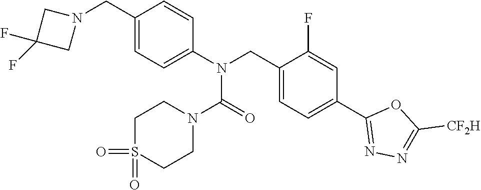 [SMILES: O=C(N1CCS(=O)(=O)CC1)N(Cc1ccc(-c2nnc(C(F)F)o2)cc1F)c1ccc(CN2CC(F)(F)C2)cc1] |
| 365 | 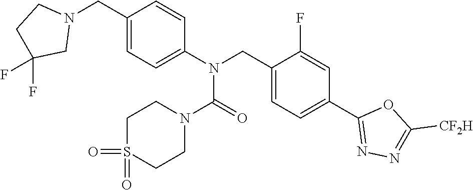 [SMILES: O=C(N1CCS(=O)(=O)CC1)N(Cc1ccc(-c2nnc(C(F)F)o2)cc1F)c1ccc(CN2CCC(F)(F)C2)cc1] |
| 366 | 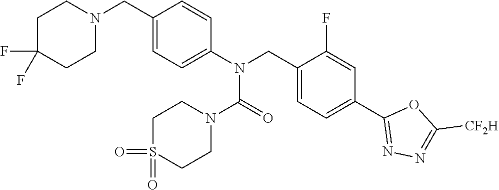 [SMILES: O=C(N1CCS(=O)(=O)CC1)N(Cc1ccc(-c2nnc(C(F)F)o2)cc1F)c1ccc(CN2CCC(F)(F)CC2)cc1] |
| 367 | 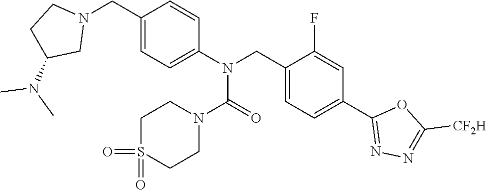 [SMILES: CN(C)[C@@H]1CCN(Cc2ccc(N(Cc3ccc(-c4nnc(C(F)F)o4)cc3F)C(=O)N3CCS(=O)(=O)CC3)cc2)C1] |
| 368 | 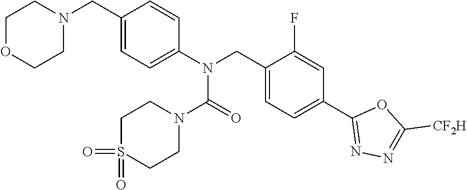 [SMILES: O=C(N1CCS(=O)(=O)CC1)N(Cc1ccc(-c2nnc(C(F)F)o2)cc1F)c1ccc(CN2CCOCC2)cc1] |
| 369 | 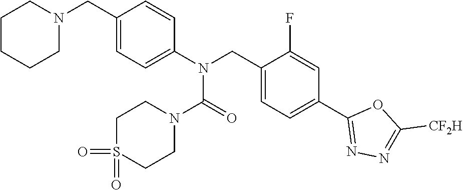 [SMILES: O=C(N1CCS(=O)(=O)CC1)N(Cc1ccc(-c2nnc(C(F)F)o2)cc1F)c1ccc(CN2CCCCC2)cc1] |
| 370 | 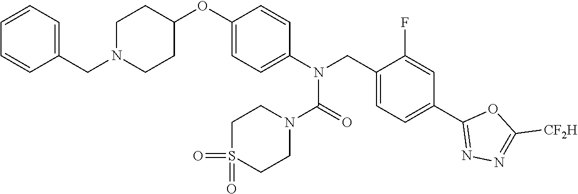 [SMILES: O=C(N1CCS(=O)(=O)CC1)N(Cc1ccc(-c2nnc(C(F)F)o2)cc1F)c1ccc(OC2CCN(Cc3ccccc3)CC2)cc1] |
| 371 | 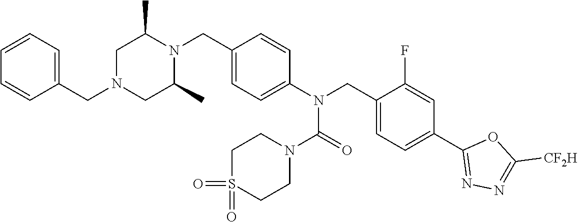 [SMILES: C[C@@H]1CN(Cc2ccccc2)C[C@H](C)N1Cc1ccc(N(Cc2ccc(-c3nnc(C(F)F)o3)cc2F)C(=O)N2CCS(=O)(=O)CC2)cc1] |
| 372 | 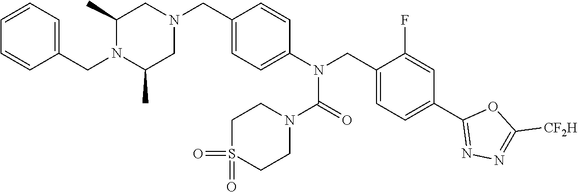 [SMILES: C[C@@H]1CN(Cc2ccc(N(Cc3ccc(-c4nnc(C(F)F)o4)cc3F)C(=O)N3CCS(=O)(=O)CC3)cc2)C[C@H](C)N1Cc1ccccc1] |
| 373 | 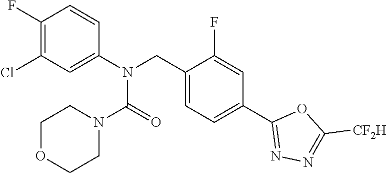 [SMILES: O=C(N1CCOCC1)N(Cc1ccc(-c2nnc(C(F)F)o2)cc1F)c1ccc(F)c(Cl)c1] |
| 374 | 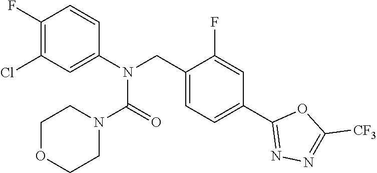 [SMILES: O=C(N1CCOCC1)N(Cc1ccc(-c2nnc(C(F)(F)F)o2)cc1F)c1ccc(F)c(Cl)c1] |
| 375 | 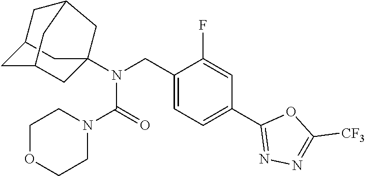 [SMILES: O=C(N1CCOCC1)N(Cc1ccc(-c2nnc(C(F)(F)F)o2)cc1F)C12CC3CC(CC(C3)C1)C2] |
| 376 | 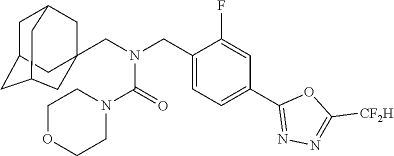 [SMILES: O=C(N1CCOCC1)N(Cc1ccc(-c2nnc(C(F)F)o2)cc1F)CC12CC3CC(CC(C3)C1)C2] |
| 377 | 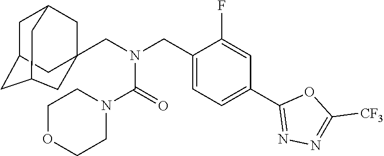 [SMILES: O=C(N1CCOCC1)N(Cc1ccc(-c2nnc(C(F)(F)F)o2)cc1F)CC12CC3CC(CC(C3)C1)C2] |
| 378 | 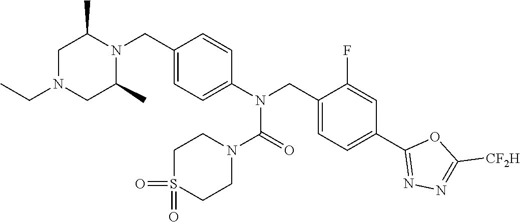 [SMILES: CCN1C[C@@H](C)N(Cc2ccc(N(Cc3ccc(-c4nnc(C(F)F)o4)cc3F)C(=O)N3CCS(=O)(=O)CC3)cc2)[C@@H](C)C1] |
| 379 | 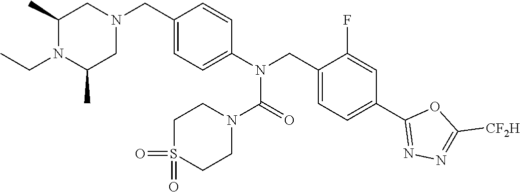 [SMILES: CCN1[C@H](C)CN(Cc2ccc(N(Cc3ccc(-c4nnc(C(F)F)o4)cc3F)C(=O)N3CCS(=O)(=O)CC3)cc2)C[C@@H]1C] |
| 380 | 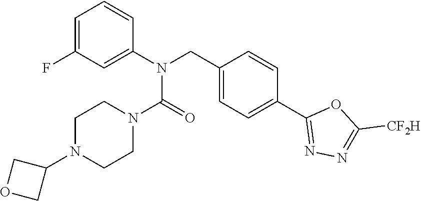 [SMILES: O=C(N1CCN(C2COC2)CC1)N(Cc1ccc(-c2nnc(C(F)F)o2)cc1)c1cccc(F)c1] |
| 381 | 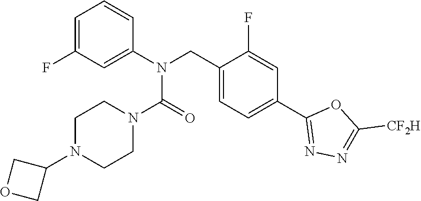 [SMILES: O=C(N1CCN(C2COC2)CC1)N(Cc1ccc(-c2nnc(C(F)F)o2)cc1F)c1cccc(F)c1] |
| 382 | 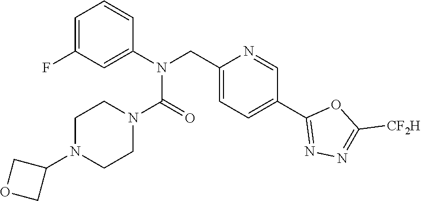 [SMILES: O=C(N1CCN(C2COC2)CC1)N(Cc1ccc(-c2nnc(C(F)F)o2)cn1)c1cccc(F)c1] |
| 383 | 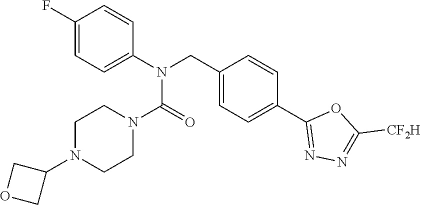 [SMILES: O=C(N1CCN(C2COC2)CC1)N(Cc1ccc(-c2nnc(C(F)F)o2)cc1)c1ccc(F)cc1] |
| 384 | 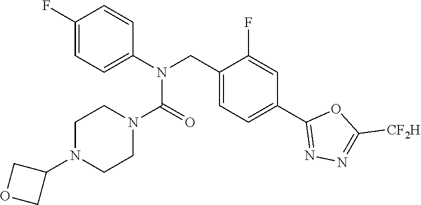 [SMILES: O=C(N1CCN(C2COC2)CC1)N(Cc1ccc(-c2nnc(C(F)F)o2)cc1F)c1ccc(F)cc1] |
| 385 | 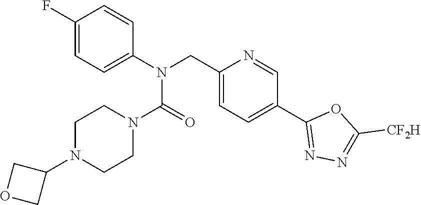 [SMILES: O=C(N1CCN(C2COC2)CC1)N(Cc1ccc(-c2nnc(C(F)F)o2)cn1)c1ccc(F)cc1] |
| 386 | 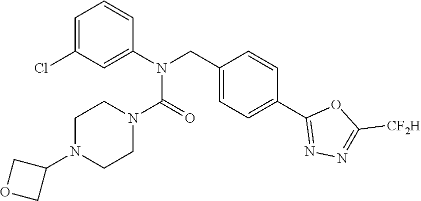 [SMILES: O=C(N1CCN(C2COC2)CC1)N(Cc1ccc(-c2nnc(C(F)F)o2)cc1)c1cccc(Cl)c1] |
| 387 | 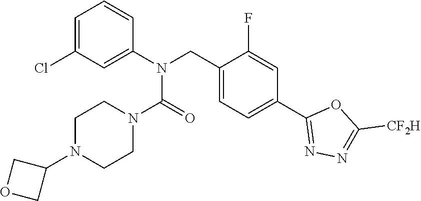 [SMILES: O=C(N1CCN(C2COC2)CC1)N(Cc1ccc(-c2nnc(C(F)F)o2)cc1F)c1cccc(Cl)c1] |
| 388 | 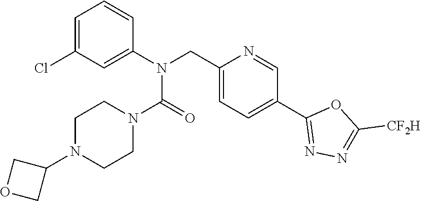 [SMILES: O=C(N1CCN(C2COC2)CC1)N(Cc1ccc(-c2nnc(C(F)F)o2)cn1)c1cccc(Cl)c1] |
| 389 | 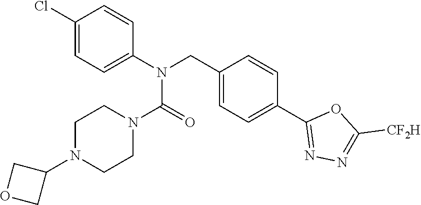 [SMILES: O=C(N1CCN(C2COC2)CC1)N(Cc1ccc(-c2nnc(C(F)F)o2)cc1)c1ccc(Cl)cc1] |
| 390 | 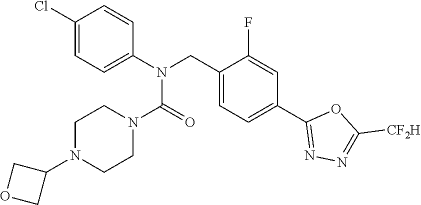 [SMILES: O=C(N1CCN(C2COC2)CC1)N(Cc1ccc(-c2nnc(C(F)F)o2)cc1F)c1ccc(Cl)cc1] |
| 391 | 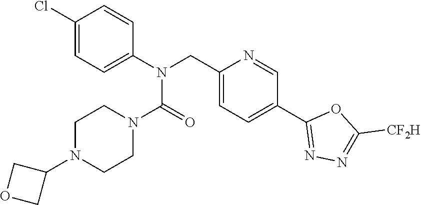 [SMILES: O=C(N1CCN(C2COC2)CC1)N(Cc1ccc(-c2nnc(C(F)F)o2)cn1)c1ccc(Cl)cc1] |
| 392 | 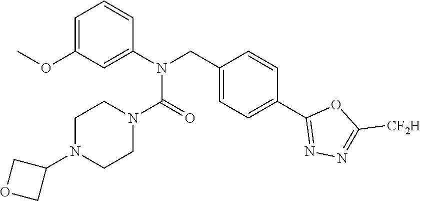 [SMILES: COc1cccc(N(Cc2ccc(-c3nnc(C(F)F)o3)cc2)C(=O)N2CCN(C3COC3)CC2)c1] |
| 393 | 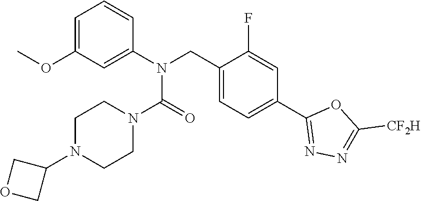 [SMILES: COc1cccc(N(Cc2ccc(-c3nnc(C(F)F)o3)cc2F)C(=O)N2CCN(C3COC3)CC2)c1] |
| 394 | 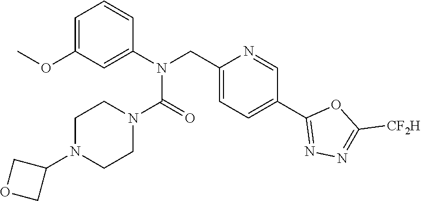 [SMILES: COc1cccc(N(Cc2ccc(-c3nnc(C(F)F)o3)cn2)C(=O)N2CCN(C3COC3)CC2)c1] |
| 395 | 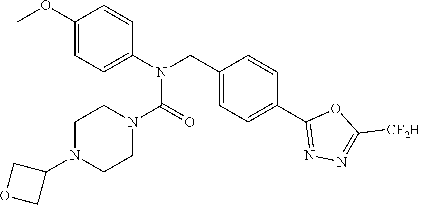 [SMILES: COc1ccc(N(Cc2ccc(-c3nnc(C(F)F)o3)cc2)C(=O)N2CCN(C3COC3)CC2)cc1] |
| 396 | 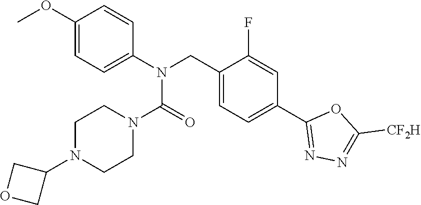 [SMILES: COc1ccc(N(Cc2ccc(-c3nnc(C(F)F)o3)cc2F)C(=O)N2CCN(C3COC3)CC2)cc1] |
| 397 | 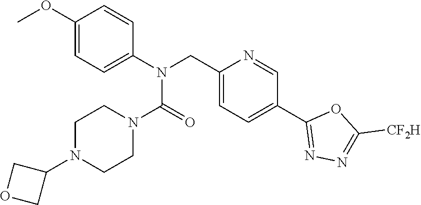 [SMILES: COc1ccc(N(Cc2ccc(-c3nnc(C(F)F)o3)cn2)C(=O)N2CCN(C3COC3)CC2)cc1] |
| 398 | 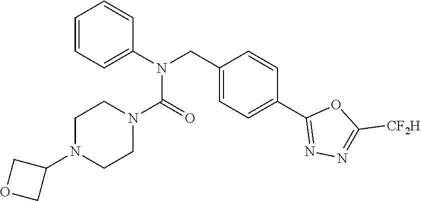 [SMILES: O=C(N1CCN(C2COC2)CC1)N(Cc1ccc(-c2nnc(C(F)F)o2)cc1)c1ccccc1] |
| 399 | 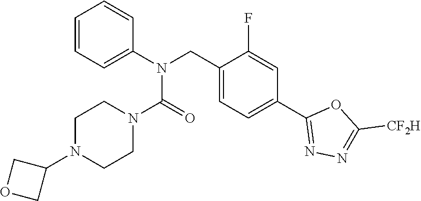 [SMILES: O=C(N1CCN(C2COC2)CC1)N(Cc1ccc(-c2nnc(C(F)F)o2)cc1F)c1ccccc1] |
| 400 | 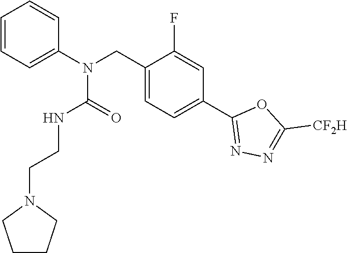 [SMILES: O=C(NCCN1CCCC1)N(Cc1ccc(-c2nnc(C(F)F)o2)cc1F)c1ccccc1] |
| 401 | 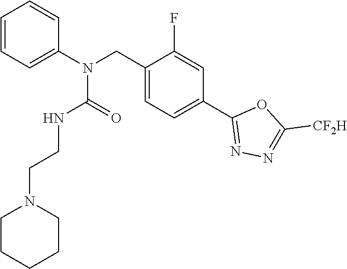 [SMILES: O=C(NCCN1CCCCC1)N(Cc1ccc(-c2nnc(C(F)F)o2)cc1F)c1ccccc1] |
| 402 | 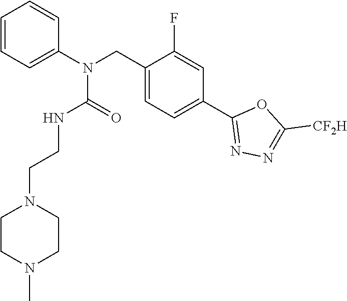 [SMILES: CN1CCN(CCNC(=O)N(Cc2ccc(-c3nnc(C(F)F)o3)cc2F)c2ccccc2)CC1] |
| 403 | 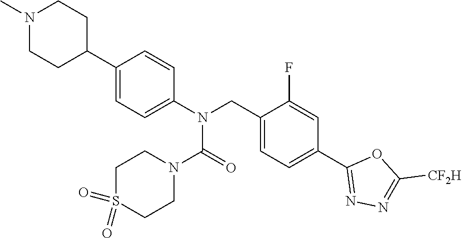 [SMILES: CN1CCC(c2ccc(N(Cc3ccc(-c4nnc(C(F)F)o4)cc3F)C(=O)N3CCS(=O)(=O)CC3)cc2)CC1] |
| 404 | 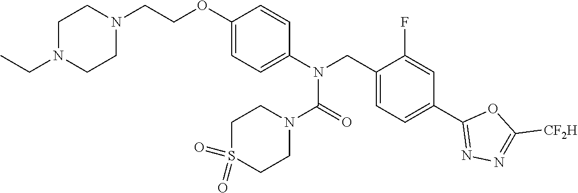 [SMILES: CCN1CCN(CCOc2ccc(N(Cc3ccc(-c4nnc(C(F)F)o4)cc3F)C(=O)N3CCS(=O)(=O)CC3)cc2)CC1] |
| 405 | 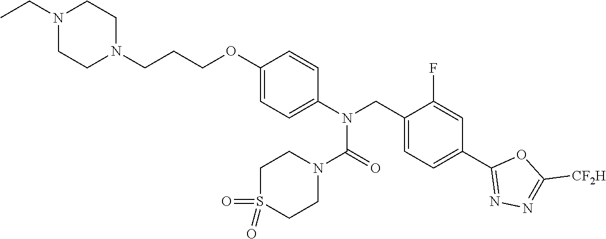 [SMILES: CCN1CCN(CCCOc2ccc(N(Cc3ccc(-c4nnc(C(F)F)o4)cc3F)C(=O)N3CCS(=O)(=O)CC3)cc2)CC1] |
| 406 | 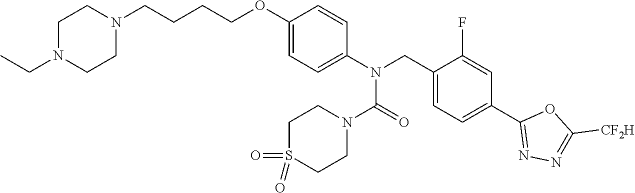 [SMILES: CCN1CCN(CCCCOc2ccc(N(Cc3ccc(-c4nnc(C(F)F)o4)cc3F)C(=O)N3CCS(=O)(=O)CC3)cc2)CC1] |
| 407 | 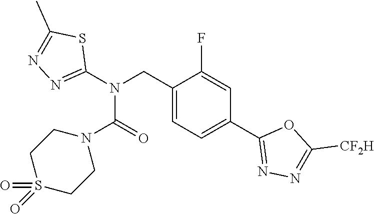 [SMILES: Cc1nnc(N(Cc2ccc(-c3nnc(C(F)F)o3)cc2F)C(=O)N2CCS(=O)(=O)CC2)s1] |
| 408 | 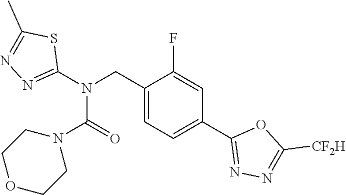 [SMILES: Cc1nnc(N(Cc2ccc(-c3nnc(C(F)F)o3)cc2F)C(=O)N2CCOCC2)s1] |
| 409 | 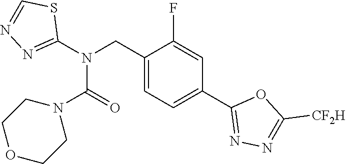 [SMILES: O=C(N1CCOCC1)N(Cc1ccc(-c2nnc(C(F)F)o2)cc1F)c1nncs1] |
| 410 | 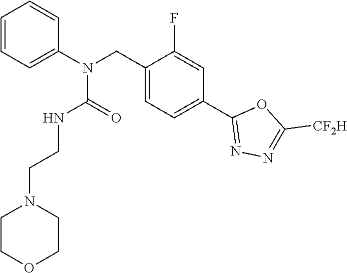 [SMILES: O=C(NCCN1CCOCC1)N(Cc1ccc(-c2nnc(C(F)F)o2)cc1F)c1ccccc1] |
| 411 | 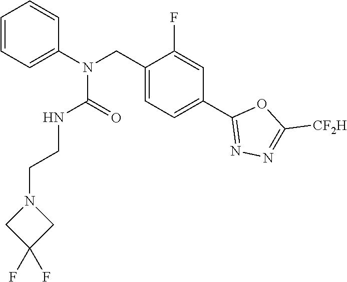 [SMILES: O=C(NCCN1CC(F)(F)C1)N(Cc1ccc(-c2nnc(C(F)F)o2)cc1F)c1ccccc1] |
| 412 | 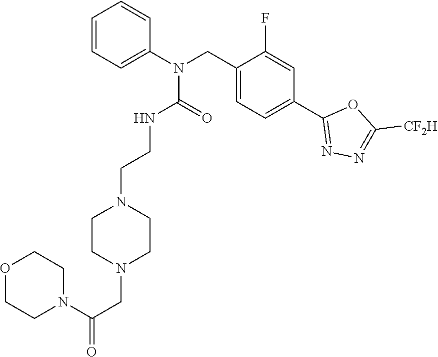 [SMILES: O=C(CN1CCN(CCNC(=O)N(Cc2ccc(-c3nnc(C(F)F)o3)cc2F)c2ccccc2)CC1)N1CCOCC1] |
| 413 | 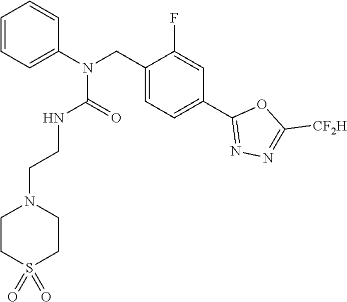 [SMILES: O=C(NCCN1CCS(=O)(=O)CC1)N(Cc1ccc(-c2nnc(C(F)F)o2)cc1F)c1ccccc1] |
| 414 | 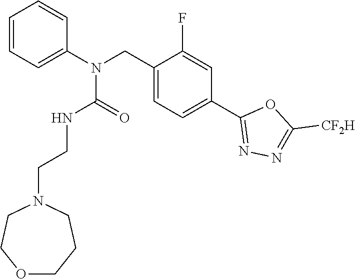 [SMILES: O=C(NCCN1CCCOCC1)N(Cc1ccc(-c2nnc(C(F)F)o2)cc1F)c1ccccc1] |
| 415 | 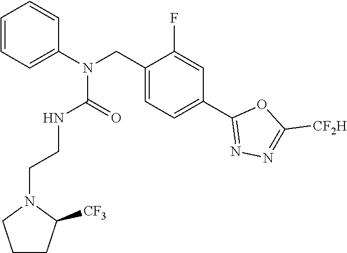 [SMILES: O=C(NCCN1CCC[C@@H]1C(F)(F)F)N(Cc1ccc(-c2nnc(C(F)F)o2)cc1F)c1ccccc1] |
| 416 | 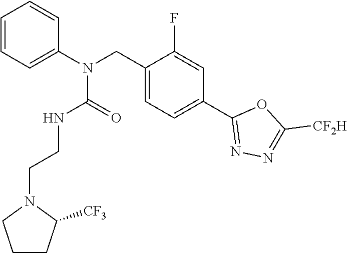 [SMILES: O=C(NCCN1CCC[C@H]1C(F)(F)F)N(Cc1ccc(-c2nnc(C(F)F)o2)cc1F)c1ccccc1] |
| 417 | 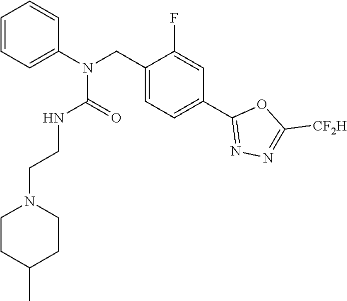 [SMILES: CC1CCN(CCNC(=O)N(Cc2ccc(-c3nnc(C(F)F)o3)cc2F)c2ccccc2)CC1] |
| 418 | 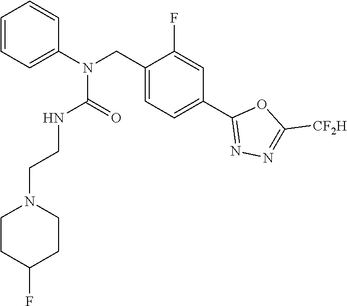 [SMILES: O=C(NCCN1CCC(F)CC1)N(Cc1ccc(-c2nnc(C(F)F)o2)cc1F)c1ccccc1] |
| 419 | 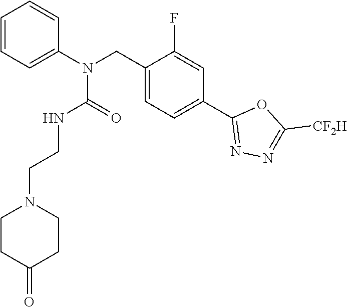 [SMILES: O=C1CCN(CCNC(=O)N(Cc2ccc(-c3nnc(C(F)F)o3)cc2F)c2ccccc2)CC1] |
| 420 | 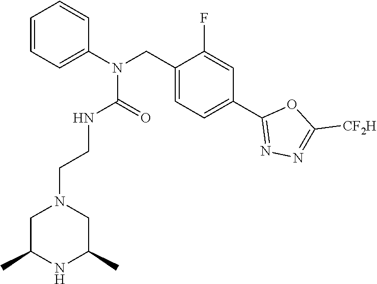 [SMILES: C[C@@H]1CN(CCNC(=O)N(Cc2ccc(-c3nnc(C(F)F)o3)cc2F)c2ccccc2)C[C@H](C)N1] |
| 421 | 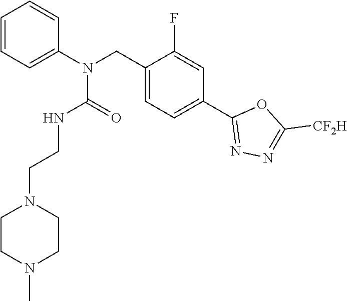 [SMILES: CN1CCN(CCNC(=O)N(Cc2ccc(-c3nnc(C(F)F)o3)cc2F)c2ccccc2)CC1] |
| 422 | 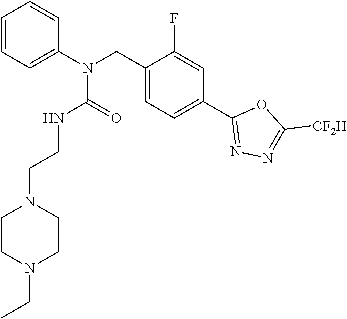 [SMILES: CCN1CCN(CCNC(=O)N(Cc2ccc(-c3nnc(C(F)F)o3)cc2F)c2ccccc2)CC1] |
| 423 | 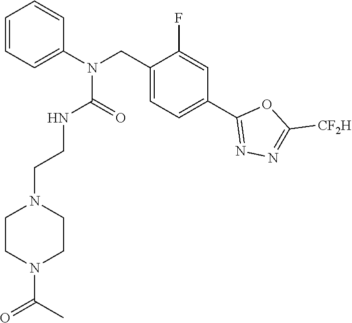 [SMILES: CC(=O)N1CCN(CCNC(=O)N(Cc2ccc(-c3nnc(C(F)F)o3)cc2F)c2ccccc2)CC1] |
| 424 | 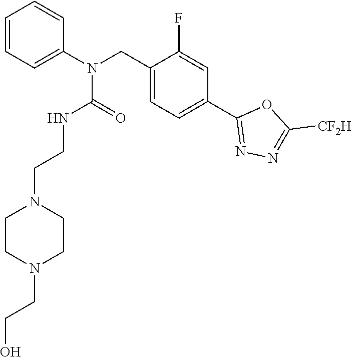 [SMILES: O=C(NCCN1CCN(CCO)CC1)N(Cc1ccc(-c2nnc(C(F)F)o2)cc1F)c1ccccc1] |
| 425 | 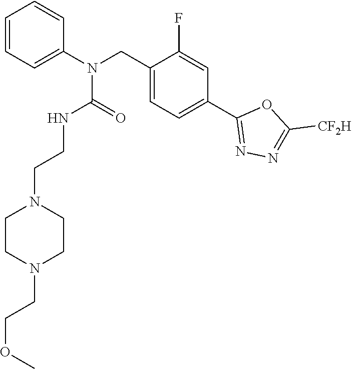 [SMILES: COCCN1CCN(CCNC(=O)N(Cc2ccc(-c3nnc(C(F)F)o3)cc2F)c2ccccc2)CC1] |
| 426 | 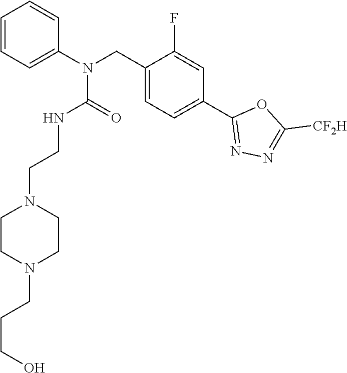 [SMILES: O=C(NCCN1CCN(CCCO)CC1)N(Cc1ccc(-c2nnc(C(F)F)o2)cc1F)c1ccccc1] |
| 427 | 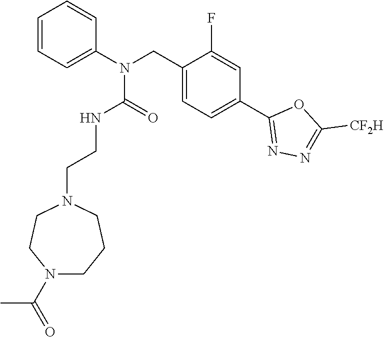 [SMILES: CC(=O)N1CCCN(CCNC(=O)N(Cc2ccc(-c3nnc(C(F)F)o3)cc2F)c2ccccc2)CC1] |
| 428 | 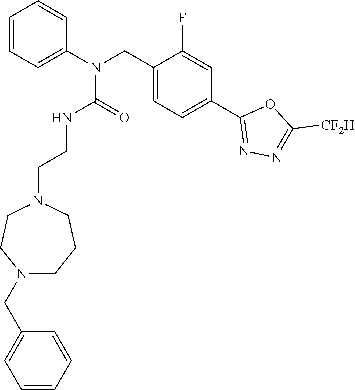 [SMILES: O=C(NCCN1CCCN(Cc2ccccc2)CC1)N(Cc1ccc(-c2nnc(C(F)F)o2)cc1F)c1ccccc1] |
| 429 | 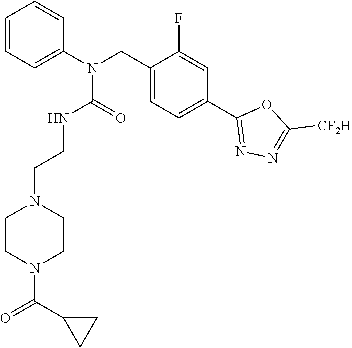 [SMILES: O=C(C1CC1)N1CCN(CCNC(=O)N(Cc2ccc(-c3nnc(C(F)F)o3)cc2F)c2ccccc2)CC1] |
| 430 | 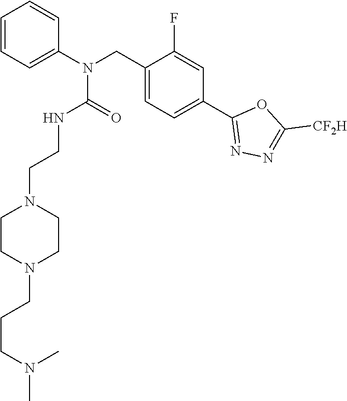 [SMILES: CN(C)CCCN1CCN(CCNC(=O)N(Cc2ccc(-c3nnc(C(F)F)o3)cc2F)c2ccccc2)CC1] |
| 431 | 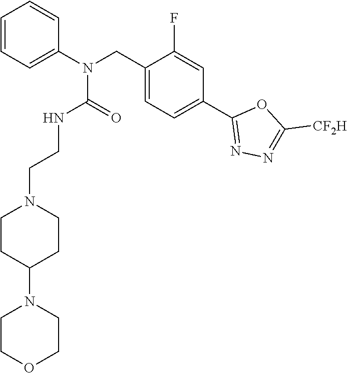 [SMILES: O=C(NCCN1CCC(N2CCOCC2)CC1)N(Cc1ccc(-c2nnc(C(F)F)o2)cc1F)c1ccccc1] |
| 432 | 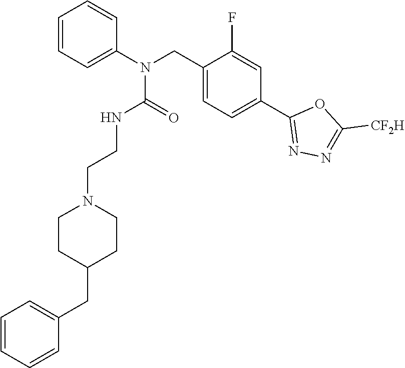 [SMILES: O=C(NCCN1CCC(Cc2ccccc2)CC1)N(Cc1ccc(-c2nnc(C(F)F)o2)cc1F)c1ccccc1] |
| 433 | 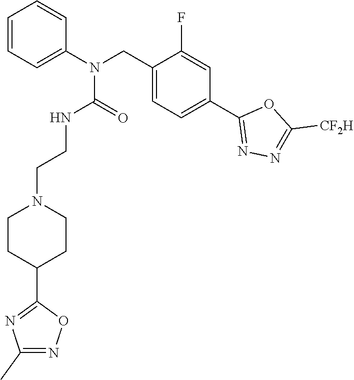 [SMILES: Cc1noc(C2CCN(CCNC(=O)N(Cc3ccc(-c4nnc(C(F)F)o4)cc3F)c3ccccc3)CC2)n1] |
| 434 | 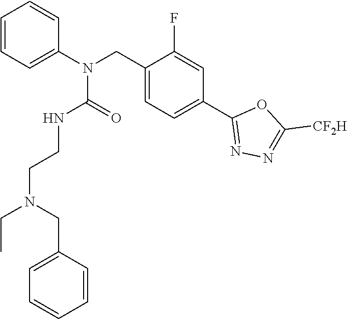 [SMILES: CCN(CCNC(=O)N(Cc1ccc(-c2nnc(C(F)F)o2)cc1F)c1ccccc1)Cc1ccccc1] |
| 435 | 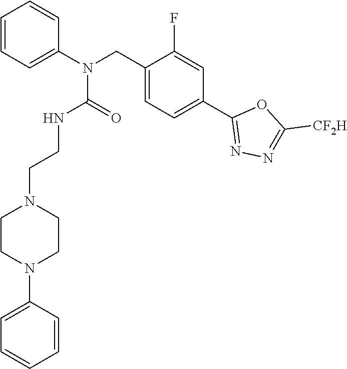 [SMILES: O=C(NCCN1CCN(c2ccccc2)CC1)N(Cc1ccc(-c2nnc(C(F)F)o2)cc1F)c1ccccc1] |
| 436 | 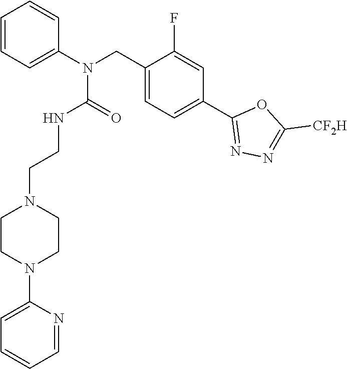 [SMILES: O=C(NCCN1CCN(c2ccccn2)CC1)N(Cc1ccc(-c2nnc(C(F)F)o2)cc1F)c1ccccc1] |
| 437 | 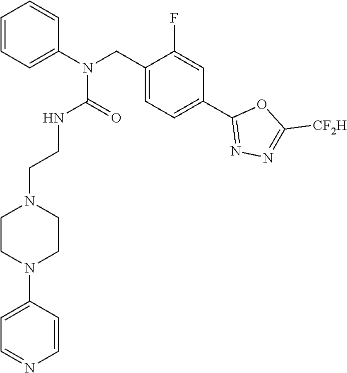 [SMILES: O=C(NCCN1CCN(c2ccncc2)CC1)N(Cc1ccc(-c2nnc(C(F)F)o2)cc1F)c1ccccc1] |
| 438 | 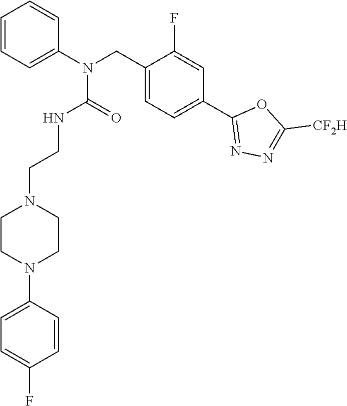 [SMILES: O=C(NCCN1CCN(c2ccc(F)cc2)CC1)N(Cc1ccc(-c2nnc(C(F)F)o2)cc1F)c1ccccc1] |
| 439 | 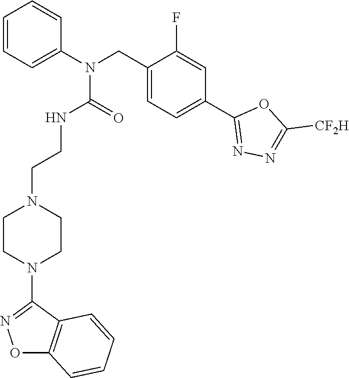 [SMILES: O=C(NCCN1CCN(c2noc3ccccc23)CC1)N(Cc1ccc(-c2nnc(C(F)F)o2)cc1F)c1ccccc1] |
| 440 | 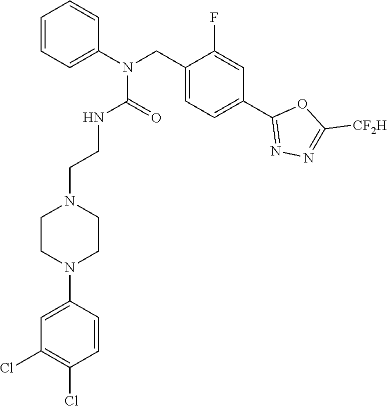 [SMILES: O=C(NCCN1CCN(c2ccc(Cl)c(Cl)c2)CC1)N(Cc1ccc(-c2nnc(C(F)F)o2)cc1F)c1ccccc1] |
| 441 | 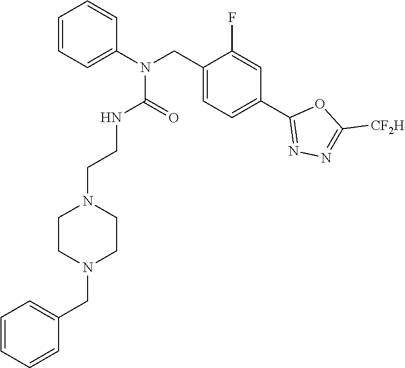 [SMILES: O=C(NCCN1CCN(Cc2ccccc2)CC1)N(Cc1ccc(-c2nnc(C(F)F)o2)cc1F)c1ccccc1] |
| 442 | 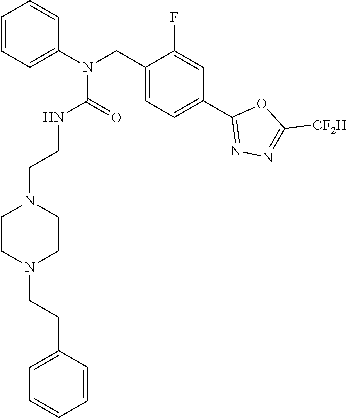 [SMILES: O=C(NCCN1CCN(CCc2ccccc2)CC1)N(Cc1ccc(-c2nnc(C(F)F)o2)cc1F)c1ccccc1] |
| 443 | 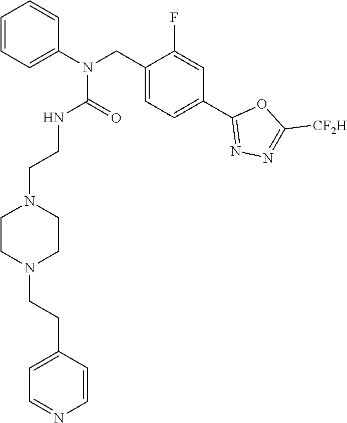 [SMILES: O=C(NCCN1CCN(CCc2ccncc2)CC1)N(Cc1ccc(-c2nnc(C(F)F)o2)cc1F)c1ccccc1] |
| 444 | 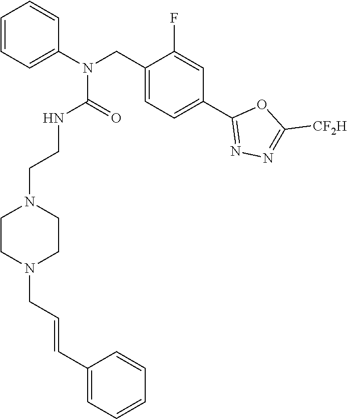 [SMILES: O=C(NCCN1CCN(C/C=C/c2ccccc2)CC1)N(Cc1ccc(-c2nnc(C(F)F)o2)cc1F)c1ccccc1] |
| 445 | 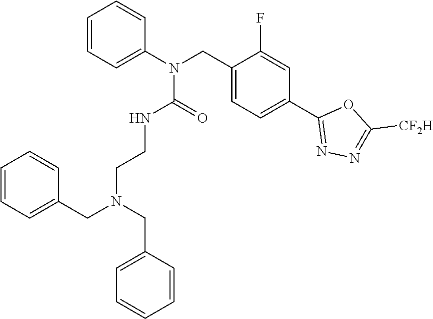 [SMILES: O=C(NCCN(Cc1ccccc1)Cc1ccccc1)N(Cc1ccc(-c2nnc(C(F)F)o2)cc1F)c1ccccc1] |
| 446 | 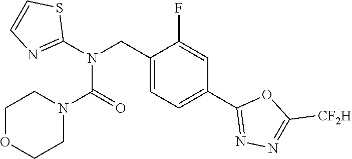 [SMILES: O=C(N1CCOCC1)N(Cc1ccc(-c2nnc(C(F)F)o2)cc1F)c1nccs1] |
| 447 | 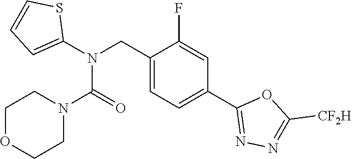 [SMILES: O=C(N1CCOCC1)N(Cc1ccc(-c2nnc(C(F)F)o2)cc1F)c1cccs1] |
| 448 | 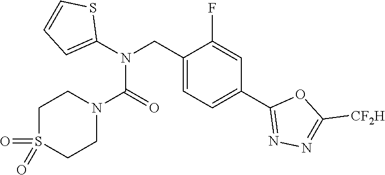 [SMILES: O=C(N1CCS(=O)(=O)CC1)N(Cc1ccc(-c2nnc(C(F)F)o2)cc1F)c1cccs1] |
| 449 | 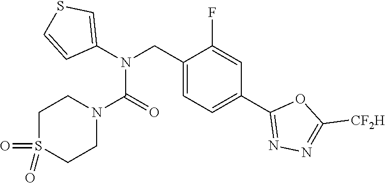 [SMILES: O=C(N1CCS(=O)(=O)CC1)N(Cc1ccc(-c2nnc(C(F)F)o2)cc1F)c1ccsc1] |
| 450 | 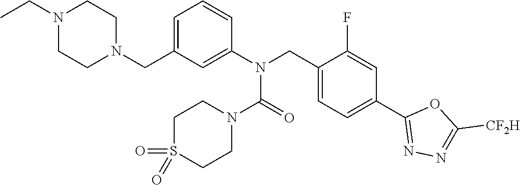 [SMILES: CCN1CCN(Cc2cccc(N(Cc3ccc(-c4nnc(C(F)F)o4)cc3F)C(=O)N3CCS(=O)(=O)CC3)c2)CC1] |

6. A pharmaceutical composition for preventing or treating pulmonary arterial hypertension, comprising a compound, optical isomers thereof or pharmaceutically acceptable salts thereof as an active ingredient, wherein the compound has the following structure:

| Com- | |
|---|---|
| pound | Structure |
| 40 | 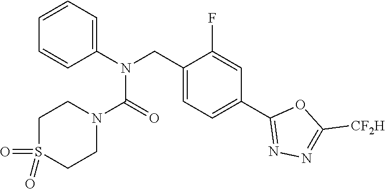 [SMILES: O=C(N1CCS(=O)(=O)CC1)N(Cc1ccc(-c2nnc(C(F)F)o2)cc1F)c1ccccc1] |
| 43 | 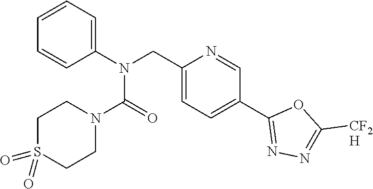 [SMILES: O=C(N1CCS(=O)(=O)CC1)N(Cc1ccc(-c2nnc(C(F)F)o2)cn1)c1ccccc1] |
| 239 | 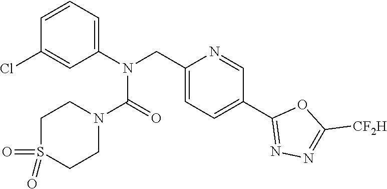 [SMILES: O=C(N1CCS(=O)(=O)CC1)N(Cc1ccc(-c2nnc(C(F)F)o2)cn1)c1cccc(Cl)c1] |
| 285 | 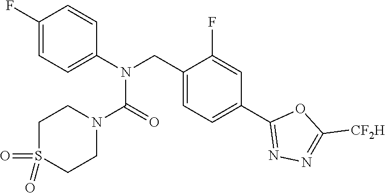 [SMILES: O=C(N1CCS(=O)(=O)CC1)N(Cc1ccc(-c2nnc(C(F)F)o2)cc1F)c1ccc(F)cc1] |
| 295 | 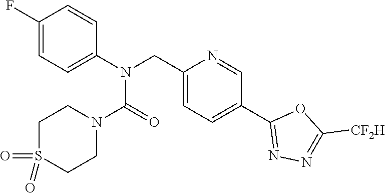 [SMILES: O=C(N1CCS(=O)(=O)CC1)N(Cc1ccc(-c2nnc(C(F)F)o2)cn1)c1ccc(F)cc1] |
| 296 | 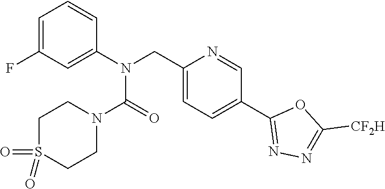 [SMILES: O=C(N1CCS(=O)(=O)CC1)N(Cc1ccc(-c2nnc(C(F)F)o2)cn1)c1cccc(F)c1] |

7. The pharmaceutical composition of claim 1, wherein the pulmonary arterial hypertension is at least one selected from the group consisting of idiopathic pulmonary arterial hypertension (IPAH), hereditary pulmonary arterial hypertension, drug- and toxin-induced pulmonary arterial hypertension, disease-associated pulmonary arterial hypertension (APAH), pulmonary arterial hypertension showing a long-term response to calcium channel blockers, pulmonary arterial hypertension with definite signs of venous/capillary invasions, and persistent pulmonary arterial hypertension in neonates.

8. The pharmaceutical composition of claim 1, wherein the pulmonary arterial hypertension has pulmonary arterial pressure of 25 mmHg or more.

9. The pharmaceutical composition of claim 1, wherein the pharmaceutical composition is orally administered.

10. A method for preventing or treating pulmonary arterial hypertension, including administering a compound represented by formula I, optical isomers thereof or pharmaceutically acceptable salts thereof into an individual, wherein the formula I is the same as in claim 1.

11. A method for preventing or treating pulmonary arterial hypertension, including administering a compound, optical isomers thereof or pharmaceutically acceptable salts thereof into an individual, wherein the compound has the following structure:

| Com- | |
|---|---|
| pound | Structure |
| 40 | 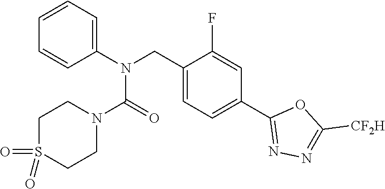 [SMILES: O=C(N1CCS(=O)(=O)CC1)N(Cc1ccc(-c2nnc(C(F)F)o2)cc1F)c1ccccc1] |
| 43 | 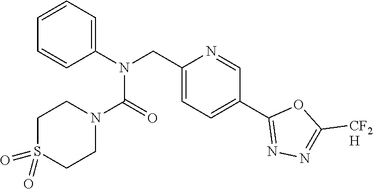 [SMILES: O=C(N1CCS(=O)(=O)CC1)N(Cc1ccc(-c2nnc(C(F)F)o2)cn1)c1ccccc1] |
| 239 | 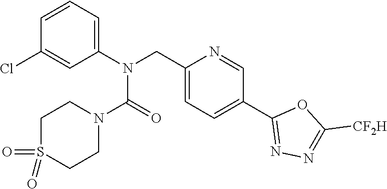 [SMILES: O=C(N1CCS(=O)(=O)CC1)N(Cc1ccc(-c2nnc(C(F)F)o2)cn1)c1cccc(Cl)c1] |
| 285 | 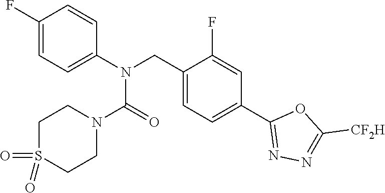 [SMILES: O=C(N1CCS(=O)(=O)CC1)N(Cc1ccc(-c2nnc(C(F)F)o2)cc1F)c1ccc(F)cc1] |
| 295 | 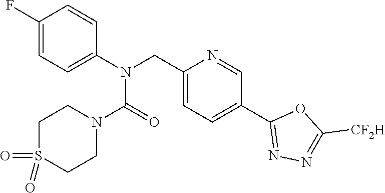 [SMILES: O=C(N1CCS(=O)(=O)CC1)N(Cc1ccc(-c2nnc(C(F)F)o2)cn1)c1ccc(F)cc1] |
| 296 | 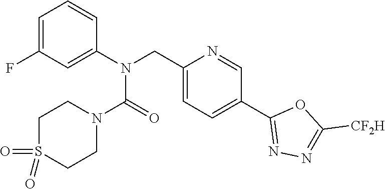 [SMILES: O=C(N1CCS(=O)(=O)CC1)N(Cc1ccc(-c2nnc(C(F)F)o2)cn1)c1cccc(F)c1] |

12. A use of a compound represented by the above formula I, optical isomers thereof or pharmaceutically acceptable salts thereof for preventing or treating pulmonary arterial hypertension, wherein the formula I is the same as in claim 1.

13. A use of a compound, optical isomers thereof or pharmaceutically acceptable salts thereof for preventing or treating pulmonary arterial hypertension, wherein the compound has the following structure:

| Com- | |
|---|---|
| pound | Structure |
| 40 | 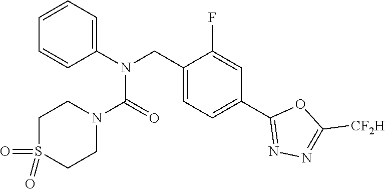 [SMILES: O=C(N1CCS(=O)(=O)CC1)N(Cc1ccc(-c2nnc(C(F)F)o2)cc1F)c1ccccc1] |
| 43 | 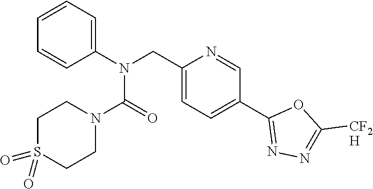 [SMILES: O=C(N1CCS(=O)(=O)CC1)N(Cc1ccc(-c2nnc(C(F)F)o2)cn1)c1ccccc1] |
| 239 | 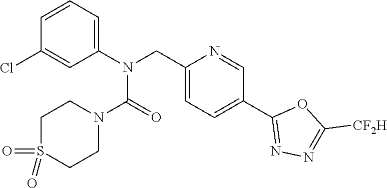 [SMILES: O=C(N1CCS(=O)(=O)CC1)N(Cc1ccc(-c2nnc(C(F)F)o2)cn1)c1cccc(Cl)c1] |
| 285 | 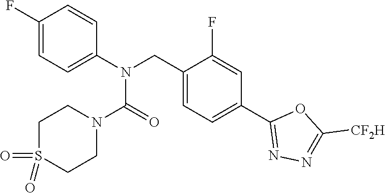 [SMILES: O=C(N1CCS(=O)(=O)CC1)N(Cc1ccc(-c2nnc(C(F)F)o2)cc1F)c1ccc(F)cc1] |
| 295 | 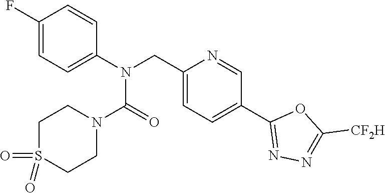 [SMILES: O=C(N1CCS(=O)(=O)CC1)N(Cc1ccc(-c2nnc(C(F)F)o2)cn1)c1ccc(F)cc1] |
| 296 | 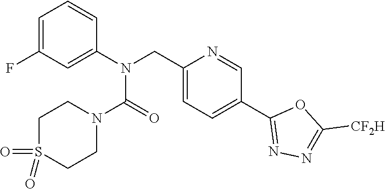 [SMILES: O=C(N1CCS(=O)(=O)CC1)N(Cc1ccc(-c2nnc(C(F)F)o2)cn1)c1cccc(F)c1] |

14. A use of the compound represented by above formula I, optical isomers thereof or pharmaceutically acceptable salts thereof in preparing a medicament for preventing or treating pulmonary arterial hypertension, wherein the formula I is the same as in claim 1.

15. A use of a compound, optical isomers thereof or pharmaceutically acceptable salts thereof in preparing a medicament for preventing or treating pulmonary arterial hypertension, wherein the compound has the following structure:

| Com- | |
|---|---|
| pound | Structure |
| 40 | 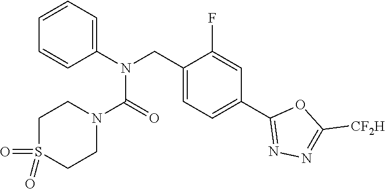 [SMILES: O=C(N1CCS(=O)(=O)CC1)N(Cc1ccc(-c2nnc(C(F)F)o2)cc1F)c1ccccc1] |
| 43 | 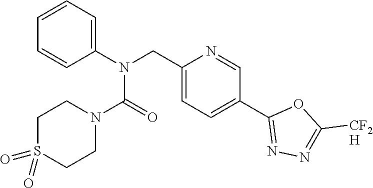 [SMILES: O=C(N1CCS(=O)(=O)CC1)N(Cc1ccc(-c2nnc(C(F)F)o2)cn1)c1ccccc1] |
| 239 | 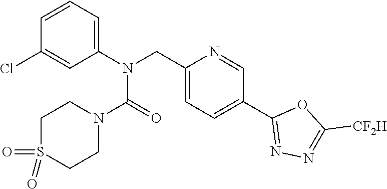 [SMILES: O=C(N1CCS(=O)(=O)CC1)N(Cc1ccc(-c2nnc(C(F)F)o2)cn1)c1cccc(Cl)c1] |
| 285 | 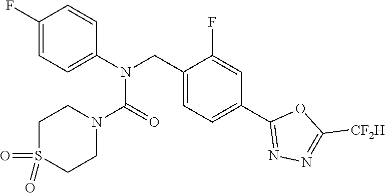 [SMILES: O=C(N1CCS(=O)(=O)CC1)N(Cc1ccc(-c2nnc(C(F)F)o2)cc1F)c1ccc(F)cc1] |
| 295 | 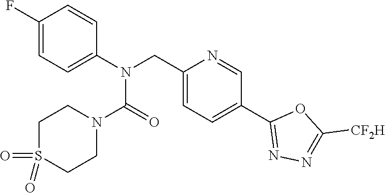 [SMILES: O=C(N1CCS(=O)(=O)CC1)N(Cc1ccc(-c2nnc(C(F)F)o2)cn1)c1ccc(F)cc1] |
| 296 | 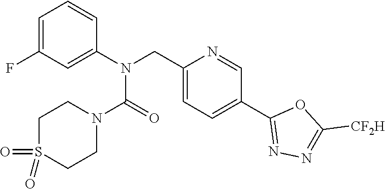 [SMILES: O=C(N1CCS(=O)(=O)CC1)N(Cc1ccc(-c2nnc(C(F)F)o2)cn1)c1cccc(F)c1] |
